(19) United States
(12) Patent Application Publication
(10) Pub. No.: US 2025/0027066 A1
(43) Pub. Date: Jan. 23, 2025

(54) RNA ENCODING PEPTIDOGLYCAN HYDROLASE AND USE THEREOF FOR TREATING BACTERIAL INFECTION

(71) Applicant: BIONTECH SE, Mainz (DE)

(72) Inventors: Ugur Sahin, Mainz (DE); Andreas Henning, Mainz (DE); Stephanie Erbar, Mainz (DE); Stefan Albrecht Schille, Mainz (DE); Anna Spier, Mainz (DE); Lorenzo Corsini, Mainz (DE); Zehra Visram, Mainz (DE)

(21) Appl. No.: 18/708,333

(22) PCT Filed: Nov. 9, 2022
(86) PCT No.: PCT/EP2022/081367

§ 371 Date: May 8, 2024

(30) Foreign Application Priority Data
Nov. 9, 2021 (WO) PCT/EP2021/081082

(51) Int. Cl. C12N 9/36; A61K 9/00; A61K 9/51; A61P 31/04

(52) U.S. Cl. CPC C12N 9/2462; A61K 9/0019; A61K 9/5123; A61P 31/04; C07K 2319/02; C12Y 302/01017

(57) ABSTRACT

The invention provides agents and methods for treating bacterial infections using RNA. The RNA encoding peptidoglycan hydrolases. e.g., endolysins, is formulated and administered in a way that peptidoglycan hydrolase proteins. e.g., endolysin proteins, can be produced and secreted by cells of a subject to combat bacterial infections.

### TECHNICAL FIELD

The invention provides agents and methods for treating bacterial infections using RNA. The RNA encoding peptidoglycan hydrolases, e.g., endolysins, is formulated and administered in a way that peptidoglycan hydrolase proteins, e.g., endolysin proteins, can be produced and secreted by cells of a subject to combat bacterial infections.

### BACKGROUND

Despite the widespread use of small-molecule antibiotics, humanity still suffers from more than 2 billion bacterial infections every year. While antibiotics are suitable for a range of these infections, there are several settings in which antibiotics are not able to provide adequate cure rates. Resistance rates are rising fast and for antibiotic-resistant pathogens novel therapies are urgently needed. In addition, antibiotics are often ineffective against bacterial biofilms-yet biofilms are the preferred habitation state of many bacterial pathogens. Finally, beneficial bacteria populate our microbiomes and, in many settings antibiotics do more harm than good (e.g. in the gut microbiome where antibiotic therapy can select for C. difficile). From a patient perspective, novel therapies are most urgently required when the patient suffers from a chronic infection in which antibiotics may lead to short-term resolution of symptoms but not to a lasting cure.

For all these reasons, novel anti-bacterials are urgently required.

Bacteriophage-encoded peptidoglycan hydrolases—also called endolysins or “enzybiotics”—are a promising alternative to antibiotics (Fischetti, 2018; Schmelcher et al., 2012). Produced towards the end of the lytic cycle in phage-infected bacteria, these enzymes cleave peptidoglycan (PG) in the bacterial cell wall, thus lysing the cells and releasing the progeny phages. They have been initially described for Gram-positive bacteria, which lack the Gram-negative outer membrane that might limit accessibility to the cell wall peptidoglycan. Endolysins have several advantages over antibiotics; not least, their very narrow host spectrum, which is usually limited to a single genus or even a single species (Fischetti, 2010), and their low propensity to generate resistance in their hosts (Schuch et al., 2014). Bacteriophages that invade Gram-positive bacteria encode a variety of highly diverse endolysins. In general, they have a modular structure consisting of one or more enzymatically active domains (EADs) connected by a flexible interdomain linker to at least one cell wall-binding domain (CBD), typically located at the C-terminus of the protein. Both domains can contribute to the specificity for a given genus or species of bacteria (Oliveira et al., 2013). In addition to phage-endolysins, also other bactericidal proteins are known, including autolysins and bacteriocins, whose activities can be combined with those of endolysins (Linden and Nelson, 2018). Autolysins are structurally very similar to endolysins and are used by bacteria to remodel their cell wall by partially lysing it, for growing in size or during cell division. Bacteriocins are typically produced by bacteria to kill other bacteria. They can have architectures homologous to endolysins, such as the bacteriocin lysostaphin, a metallopeptidase produced by Staphylococcus simulans to specifically kill its relative Staphylococcus aureus. Or they can be antimicrobial peptides not related to endolysins, which for example perforate the inner membrane of Gram-negative bacteria, such as nisin.

Thus far resistance to endolysins has not been reported in bacteria and these enzymes demonstrated activity against bacteria in suspension as well as in biofilms-bacterial communities notorious for conferring antibiotic resistance and causing a range of hospital-associated and recurring infections. Additionally, engineered lysin has been shown to have 10-fold higher bactericidal activity as compared to natural lysins (Landlinger C. et al. (2021) Pathogens 10 (1): 54).

There is a need of providing agents and treatments for treating bacterial infections.

### SUMMARY

Here we describe an approach for treating bacterial infections employing RNA, e.g., nucleoside modified RNA (modRNA) or self-amplifying RNA (saRNA), to express endolysins in cells of a patient. The RNA encoding endolysins is formulated and administered in a way that endolysin proteins can be produced and secreted by patient cells to combat bacterial infections.

In one aspect, the invention provides RNA encoding an amino acid sequence comprising a peptidoglycan hydrolase.

In some embodiments, the peptidoglycan hydrolase breaks down peptidoglycan in bacterial cell wall.

In some embodiments, the peptidoglycan hydrolase is derived from a bacterium.

In some embodiments, the peptidoglycan hydrolase is derived from a bacteriophage.

In some embodiments, the peptidoglycan hydrolase is or is derived from an endolysin.

In some embodiments, the endolysin comprises one or more enzymatically active domains (EADs) connected by a flexible interdomain linker to one or more cell wall-binding domains (CBD).

In some embodiments, the peptidoglycan hydrolase has a modified glycosylation pattern to improve pharmacokinetics in blood.

In some embodiments, the peptidoglycan hydrolase is modified so as to reduce glycosylation.

In some embodiments, the peptidoglycan hydrolase is modified by removing glycosylation sites.

In some embodiments, the peptidoglycan hydrolase is modified so as to reduce immunogenicity.

In some embodiments, the peptidoglycan hydrolase is modified by removing T cell epitopes.

In some embodiments, the peptidoglycan hydrolase is or is derived from endolysin Lys26A (from P. aeruginosa phage JD010).

In some embodiments, the amino acid sequence comprising a peptidoglycan hydrolase comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1.

In some embodiments, the RNA encoding an amino acid sequence comprising a peptidoglycan hydrolase comprises the nucleotide sequence of SEQ ID NO: 2, a nucleotide sequence having at least 99%, 98%, 97%, 96%, 95%, 90%, 85%, or 80% identity to the nucleotide sequence of SEQ ID NO: 2, or a fragment of the nucleotide sequence of SEQ ID NO: 2 or the nucleotide sequence having at least 99%, 98%, 97%, 96%, 95%, 90%, 85%, or 80% identity to the nucleotide sequence of SEQ ID NO: 2.

In some embodiments, the RNA encoding an amino acid sequence comprising a peptidoglycan hydrolase comprises the nucleotide sequence of SEQ ID NO: 3, a nucleotide sequence having at least 99%, 98%, 97%, 96%, 95%, 90%, 85%, or 80% identity to the nucleotide sequence of SEQ ID NO: 3, or a fragment of the nucleotide sequence of SEQ ID NO: 3 or the nucleotide sequence having at least 99%, 98%, 97%, 96%, 95%, 90%, 85%, or 80% identity to the nucleotide sequence of SEQ ID NO: 3.

In some embodiments, the peptidoglycan hydrolase is secreted peptidoglycan hydrolase.

In some embodiments, the amino acid sequence comprising a peptidoglycan hydrolase comprises a signal peptide.

In some embodiments, the peptidoglycan hydrolase is fused to a signal peptide.

In some embodiments, the signal peptide is cleaved off during secretion or export.

In some embodiments, the signal peptide comprises the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10.

In some embodiments, the amino acid sequence comprising a peptidoglycan hydrolase comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4.

In some embodiments, the RNA encoding an amino acid sequence comprising a peptidoglycan hydrolase comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a fragment of the nucleotide sequence of SEQ ID NO: 5 or the nucleotide sequence having at least 99%, 98%, 97%, 96%, 95%, 90%, 85%, or 80% identity to the nucleotide sequence of SEQ ID NO: 5.

In some embodiments, the amino acid sequence comprising a peptidoglycan hydrolase comprises an extended pharmacokinetic (PK) polypeptide.

In some embodiments, the peptidoglycan hydrolase is fused to a pharmacokinetic modifying group.

In some embodiments, the pharmacokinetic modifying group comprises a moiety which is heterologous to the peptidoglycan hydrolase.

In some embodiments, the pharmacokinetic modifying group comprises a moiety selected from the group consisting of albumin, an immunoglobulin fragment, transferrin, Fn3, a functional variant thereof, or a functional fragment of the albumin, immunoglobulin fragment, transferrin, Fn3 or the functional variant thereof.

In some embodiments, the pharmacokinetic modifying group comprises human albumin, a functional variant thereof, or a functional fragment of the human albumin or the functional variant thereof.

In some embodiments, the pharmacokinetic modifying group is fused to the N-terminus of the peptidoglycan hydrolase.

In some embodiments, the amino acid sequence comprising a peptidoglycan hydrolase comprises from N-terminus to C-terminus: N-pharmacokinetic modifying group-GS-linker-peptidoglycan hydrolase-C.

In some embodiments, the amino acid sequence comprising a peptidoglycan hydrolase is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, the RNA comprises a modified nucleoside in place of uridine.

In some embodiments, the RNA comprises a modified nucleoside in place of each uridine.

In some embodiments, the modified nucleoside is selected from pseudouridine (ψ) N1-methyl-pseudouridine (m1ψ), and 5-methyl-uridine (m5U).

In some embodiments, the modified nucleoside is N1-methyl-pseudouridine (m1ψ).

In some embodiments, the RNA is suitable for expression of the peptidoglycan hydrolase in a eukaryotic cell, e.g., a mammalian cell, such as a human cell. In some embodiments, the RNA comprises sequence elements allowing expression of the peptidoglycan hydrolase in a eukaryotic cell, e.g., a mammalian cell, such as a human cell. In some embodiments, such sequence elements comprise cap, 5′UTR, 3′UTR, and polyA.

In some embodiments, the RNA comprises a cap0 5′ cap.

In some embodiments, the RNA comprises the 5′ cap m₂^{7,2′O}G(5′)ppSp(5′) G (in particular its D1 diastereomer).

In some embodiments, the RNA comprises a cap1 5′ cap.

In some embodiments, the RNA comprises the 5′ cap m₂^{7,3′-O}Gppp(m₁^{2′-O})ApG.

In some embodiments, the RNA comprises a 5′ UTR.

In some embodiments, the 5′ UTR comprises the nucleotide sequence of SEQ ID NO: 11, or a nucleotide sequence having at least 99%, 98%, 97%, 96%, 95%, 90%, 85%, or 80% identity to the nucleotide sequence of SEQ ID NO: 11.

In some embodiments, the RNA comprises a 3′ UTR.

In some embodiments, the 3′ UTR comprises the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, the RNA comprises a poly-A sequence.

In some embodiments, the poly-A sequence comprises at least 100 nucleotides.

In some embodiments, the poly-A sequence comprises or consists of the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In some embodiments, the RNA is mRNA.

In some embodiments, the RNA encoding an amino acid sequence comprising a peptidoglycan hydrolase comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6 or the nucleotide sequence having at least 99%, 98%, 97%, 96%, 95%, 90%, 85%, or 80% identity to the nucleotide sequence of SEQ ID NO: 6.

In some embodiments, the RNA encoding an amino acid sequence comprising a peptidoglycan hydrolase comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a fragment of the nucleotide sequence of SEQ ID NO: 7 or the nucleotide sequence having at least 99%, 98%, 97%, 96%, 95%, 90%, 85%, or 80% identity to the nucleotide sequence of SEQ ID NO: 7.

In some embodiments, the RNA is self-amplifying RNA.

In some embodiments, the RNA encoding an amino acid sequence comprising a peptidoglycan hydrolase comprises the nucleotide sequence of SEQ ID NO: 8, a nucleotide sequence having at least 99%, 98%, 97%, 96%, 95%, 90%, 85%, or 80% identity to the nucleotide sequence of SEQ ID NO: 8, or a fragment of the nucleotide sequence of SEQ ID NO: 8 or the nucleotide sequence having at least 99%, 98%, 97%, 96%, 95%, 90%, 85%, or 80% identity to the nucleotide sequence of SEQ ID NO: 8.

In some embodiments, the RNA encoding an amino acid sequence comprising a peptidoglycan hydrolase comprises the nucleotide sequence of SEQ ID NO: 9, a nucleotide sequence having at least 99%, 98%, 97%, 96%, 95%, 90%, 85%, or 80% identity to the nucleotide sequence of SEQ ID NO: 9, or a fragment of the nucleotide sequence of SEQ ID NO: 9 or the nucleotide sequence having at least 99%, 98%, 97%, 96%, 95%, 90%, 85%, or 80% identity to the nucleotide sequence of SEQ ID NO: 9.

In some embodiments, the RNA is formulated as a liquid, formulated as a solid, or a combination thereof.

In some embodiments, the RNA is formulated for injection.

In some embodiments, the RNA is formulated for intravenous administration.

In some embodiments, the RNA is formulated as lipid particles.

In some embodiments, the RNA lipid particles are lipid nanoparticles (LNP).

In some embodiments, the RNA is present in a composition or medical preparation.

In some embodiments, the composition is a pharmaceutical composition.

In some embodiments, the pharmaceutical composition further comprises one or more pharmaceutically acceptable carriers, diluents and/or excipients.

In some embodiments, the medical preparation is a kit.

In some embodiments, the medical preparation further comprises instructions for use of the RNA for treating or preventing bacterial infection.

In some embodiments, the RNA is for administration to a human.

In some embodiments, the RNA is for pharmaceutical use.

In some embodiments, the pharmaceutical use comprises a therapeutic or prophylactic treatment of a disease or disorder.

In some embodiments, the therapeutic or prophylactic treatment of a disease or disorder comprises treating or preventing bacterial infection.

In one aspect, the invention provides a composition or medical preparation comprising RNA encoding an amino acid sequence comprising a peptidoglycan hydrolase.

In some embodiments, the peptidoglycan hydrolase breaks down peptidoglycan in bacterial cell wall.

In some embodiments, the peptidoglycan hydrolase is derived from a bacterium.

In some embodiments, the peptidoglycan hydrolase is derived from a bacteriophage.

In some embodiments, the peptidoglycan hydrolase is or is derived from an endolysin.

In some embodiments, the endolysin comprises one or more enzymatically active domains (EADs) connected by a flexible interdomain linker to one or more cell wall-binding domains (CBD).

In some embodiments, the peptidoglycan hydrolase has a modified glycosylation pattern to improve pharmacokinetics in blood.

In some embodiments, the peptidoglycan hydrolase is modified so as to reduce glycosylation.

In some embodiments, the peptidoglycan hydrolase is modified by removing glycosylation sites.

In some embodiments, the peptidoglycan hydrolase is modified so as to reduce immunogenicity.

In some embodiments, the peptidoglycan hydrolase is modified by removing T cell epitopes.

In some embodiments, the peptidoglycan hydrolase is or is derived from endolysin Lys26A (from P. aeruginosa phage JD010).

In some embodiments, the amino acid sequence comprising a peptidoglycan hydrolase comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1.

In some embodiments, the RNA encoding an amino acid sequence comprising a peptidoglycan hydrolase comprises the nucleotide sequence of SEQ ID NO: 2, a nucleotide sequence having at least 99%, 98%, 97%, 96%, 95%, 90%, 85%, or 80% identity to the nucleotide sequence of SEQ ID NO: 2, or a fragment of the nucleotide sequence of SEQ ID NO: 2 or the nucleotide sequence having at least 99%, 98%, 97%, 96%, 95%, 90%, 85%, or 80% identity to the nucleotide sequence of SEQ ID NO: 2.

In some embodiments, the RNA encoding an amino acid sequence comprising a peptidoglycan hydrolase comprises the nucleotide sequence of SEQ ID NO: 3, a nucleotide sequence having at least 99%, 98%, 97%, 96%, 95%, 90%, 85%, or 80% identity to the nucleotide sequence of SEQ ID NO: 3, or a fragment of the nucleotide sequence of SEQ ID NO: 3 or the nucleotide sequence having at least 99%, 98%, 97%, 96%, 95%, 90%, 85%, or 80% identity to the nucleotide sequence of SEQ ID NO: 3.

In some embodiments, the peptidoglycan hydrolase is secreted peptidoglycan hydrolase.

In some embodiments, the amino acid sequence comprising a peptidoglycan hydrolase comprises a signal peptide.

In some embodiments, the peptidoglycan hydrolase is fused to a signal peptide.

In some embodiments, the signal peptide is cleaved off during secretion or export.

In some embodiments, the signal peptide comprises the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10.

In some embodiments, the amino acid sequence comprising a peptidoglycan hydrolase comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4.

In some embodiments, the RNA encoding an amino acid sequence comprising a peptidoglycan hydrolase comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a fragment of the nucleotide sequence of SEQ ID NO: 5 or the nucleotide sequence having at least 99%, 98%, 97%, 96%, 95%, 90%, 85%, or 80% identity to the nucleotide sequence of SEQ ID NO: 5.

In some embodiments, the amino acid sequence comprising a peptidoglycan hydrolase comprises an extended pharmacokinetic (PK) polypeptide.

In some embodiments, the peptidoglycan hydrolase is fused to a pharmacokinetic modifying group.

In some embodiments, the pharmacokinetic modifying group comprises a moiety which is heterologous to the peptidoglycan hydrolase.

In some embodiments, the pharmacokinetic modifying group comprises a moiety selected from the group consisting of albumin, an immunoglobulin fragment, transferrin, Fn3, a functional variant thereof, or a functional fragment of the albumin, immunoglobulin fragment, transferrin, Fn3 or the functional variant thereof.

In some embodiments, the pharmacokinetic modifying group comprises human albumin, a functional variant thereof, or a functional fragment of the human albumin or the functional variant thereof.

In some embodiments, the pharmacokinetic modifying group is fused to the N-terminus of the peptidoglycan hydrolase.

In some embodiments, the amino acid sequence comprising a peptidoglycan hydrolase comprises from N-terminus to C-terminus: N-pharmacokinetic modifying group-GS-linker-peptidoglycan hydrolase-C.

In some embodiments, the amino acid sequence comprising a peptidoglycan hydrolase is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, the RNA comprises a modified nucleoside in place of uridine.

In some embodiments, the RNA comprises a modified nucleoside in place of each uridine.

In some embodiments, the modified nucleoside is selected from pseudouridine (ψ), N1-methyl-pseudouridine (m1ψ), and 5-methyl-uridine (m5U).

In some embodiments, the modified nucleoside is N1-methyl-pseudouridine (m1ψ).

In some embodiments, the RNA is suitable for expression of the peptidoglycan hydrolase in a eukaryotic cell, e.g., a mammalian cell, such as a human cell. In some embodiments, the RNA comprises sequence elements allowing expression of the peptidoglycan hydrolase in a eukaryotic cell, e.g., a mammalian cell, such as a human cell. In some embodiments, such sequence elements comprise cap, 5′UTR, 3′UTR, and polyA.

In some embodiments, the RNA comprises a cap0 5′ cap.

In some embodiments, the RNA comprises the 5′ cap m₂^{7,2′O}G(5′)ppSp(5′) G (in particular its D1 diastereomer).

In some embodiments, the RNA comprises a cap1 5′ cap.

In some embodiments, the RNA comprises the 5′ cap m₂^{7,3′-O}Gppp(m₁^{2′-O})ApG.

In some embodiments, the RNA comprises a 5′ UTR.

In some embodiments, the 5′ UTR comprises the nucleotide sequence of SEQ ID NO: 11, or a nucleotide sequence having at least 99%, 98%, 97%, 96%, 95%, 90%, 85%, or 80% identity to the nucleotide sequence of SEQ ID NO: 11.

In some embodiments, the RNA comprises a 3′ UTR.

In some embodiments, the 3′ UTR comprises the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, the RNA comprises a poly-A sequence.

In some embodiments, the poly-A sequence comprises at least 100 nucleotides.

In some embodiments, the poly-A sequence comprises or consists of the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In some embodiments, the RNA is mRNA.

In some embodiments, the RNA encoding an amino acid sequence comprising a peptidoglycan hydrolase comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6 or the nucleotide sequence having at least 99%, 98%, 97%, 96%, 95%, 90%, 85%, or 80% identity to the nucleotide sequence of SEQ ID NO: 6.

In some embodiments, the RNA encoding an amino acid sequence comprising a peptidoglycan hydrolase comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a fragment of the nucleotide sequence of SEQ ID NO: 7 or the nucleotide sequence having at least 99%, 98%, 97%, 96%, 95%, 90%, 85%, or 80% identity to the nucleotide sequence of SEQ ID NO: 7.

In some embodiments, the RNA is self-amplifying RNA.

In some embodiments, the RNA encoding an amino acid sequence comprising a peptidoglycan hydrolase comprises the nucleotide sequence of SEQ ID NO: 8, a nucleotide sequence having at least 99%, 98%, 97%, 96%, 95%, 90%, 85%, or 80% identity to the nucleotide sequence of SEQ ID NO: 8, or a fragment of the nucleotide sequence of SEQ ID NO: 8 or the nucleotide sequence having at least 99%, 98%, 97%, 96%, 95%, 90%, 85%, or 80% identity to the nucleotide sequence of SEQ ID NO: 8.

In some embodiments, the RNA encoding an amino acid sequence comprising a peptidoglycan hydrolase comprises the nucleotide sequence of SEQ ID NO: 9, a nucleotide sequence having at least 99%, 98%, 97%, 96%, 95%, 90%, 85%, or 80% identity to the nucleotide sequence of SEQ ID NO: 9, or a fragment of the nucleotide sequence of SEQ ID NO: 9 or the nucleotide sequence having at least 99%, 98%, 97%, 96%, 95%, 90%, 85%, or 80% identity to the nucleotide sequence of SEQ ID NO: 9.

In some embodiments, the RNA is formulated as a liquid, formulated as a solid, or a combination thereof.

In some embodiments, the RNA is formulated for injection.

In some embodiments, the RNA is formulated for intravenous administration.

In some embodiments, the RNA is formulated or is to be formulated as lipid particles.

In some embodiments, the RNA lipid particles are lipid nanoparticles (LNP).

In some embodiments, the composition is a pharmaceutical composition.

In some embodiments, the pharmaceutical composition further comprises one or more pharmaceutically acceptable carriers, diluents and/or excipients.

In some embodiments, the medical preparation is a kit.

In some embodiments, the kit further comprises instructions for use of the RNA for treating or preventing bacterial infection.

In some embodiments, the composition or medical preparation is for administration to a human.

In some embodiments, the composition or medical preparation is for pharmaceutical use.

In some embodiments, the pharmaceutical use comprises a therapeutic or prophylactic treatment of a disease or disorder.

In some embodiments, the therapeutic or prophylactic treatment of a disease or disorder comprises treating or preventing bacterial infection.

In one aspect, the invention provides a cell comprising the RNA described herein. In one aspect, the invention provides a cell transfected with the RNA described herein.

In some embodiments, the cell is a human cell

In one aspect, the invention provides a population of cells, wherein at least one cell is a cell described herein. In one aspect, the invention provides a population of cells, wherein the population of cells comprises a plurality of a cell described herein.

In some embodiments, a cell or a population of cells described herein is present in a subject. In one aspect, the invention provides a method of producing a peptidoglycan hydrolase in vivo comprising transfecting a cell or a population of cells with an RNA described herein.

In some embodiments, a cell or a population of cells described herein is present in a subject.

In some embodiments, the subject has been administered the RNA or the composition described herein

In one aspect, the invention provides a method of treating bacterial infection in a subject comprising administering RNA encoding an amino acid sequence comprising a peptidoglycan hydrolase to the subject.

In some embodiments, the peptidoglycan hydrolase breaks down peptidoglycan in bacterial cell wall.

In some embodiments, the peptidoglycan hydrolase is derived from a bacterium.

In some embodiments, the peptidoglycan hydrolase is derived from a bacteriophage.

In some embodiments, the peptidoglycan hydrolase is or is derived from an endolysin.

In some embodiments, the endolysin comprises one or more enzymatically active domains (EADs) connected by a flexible interdomain linker to one or more cell wall-binding domains (CBD).

In some embodiments, the peptidoglycan hydrolase has a modified glycosylation pattern to improve pharmacokinetics in blood.

In some embodiments, the peptidoglycan hydrolase is modified so as to reduce glycosylation.

In some embodiments, the peptidoglycan hydrolase is modified by removing glycosylation sites.

In some embodiments, the peptidoglycan hydrolase is modified so as to reduce immunogenicity.

In some embodiments, the peptidoglycan hydrolase is modified by removing T cell epitopes.

In some embodiments, the peptidoglycan hydrolase is or is derived from endolysin Lys26A (from P. aeruginosa phage JD010).

In some embodiments, the amino acid sequence comprising a peptidoglycan hydrolase comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1.

In some embodiments, the RNA encoding an amino acid sequence comprising a peptidoglycan hydrolase comprises the nucleotide sequence of SEQ ID NO: 2, a nucleotide sequence having at least 99%, 98%, 97%, 96%, 95%, 90%, 85%, or 80% identity to the nucleotide sequence of SEQ ID NO: 2, or a fragment of the nucleotide sequence of SEQ ID NO: 2 or the nucleotide sequence having at least 99%, 98%, 97%, 96%, 95%, 90%, 85%, or 80% identity to the nucleotide sequence of SEQ ID NO: 2.

In some embodiments, the RNA encoding an amino acid sequence comprising a peptidoglycan hydrolase comprises the nucleotide sequence of SEQ ID NO: 3, a nucleotide sequence having at least 99%, 98%, 97%, 96%, 95%, 90%, 85%, or 80% identity to the nucleotide sequence of SEQ ID NO: 3, or a fragment of the nucleotide sequence of SEQ ID NO: 3 or the nucleotide sequence having at least 99%, 98%, 97%, 96%, 95%, 90%, 85%, or 80% identity to the nucleotide sequence of SEQ ID NO: 3.

In some embodiments, the peptidoglycan hydrolase is secreted peptidoglycan hydrolase.

In some embodiments, the amino acid sequence comprising a peptidoglycan hydrolase comprises a signal peptide.

In some embodiments, the peptidoglycan hydrolase is fused to a signal peptide.

In some embodiments, the signal peptide is cleaved off during secretion or export.

In some embodiments, the signal peptide comprises the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10.

In some embodiments, the amino acid sequence comprising a peptidoglycan hydrolase comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4.

In some embodiments, the RNA encoding an amino acid sequence comprising a peptidoglycan hydrolase comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a fragment of the nucleotide sequence of SEQ ID NO: 5 or the nucleotide sequence having at least 99%, 98%, 97%, 96%, 95%, 90%, 85%, or 80% identity to the nucleotide sequence of SEQ ID NO: 5.

In some embodiments, the amino acid sequence comprising a peptidoglycan hydrolase comprises an extended pharmacokinetic (PK) polypeptide.

In some embodiments, the peptidoglycan hydrolase is fused to a pharmacokinetic modifying group.

In some embodiments, the pharmacokinetic modifying group comprises a moiety which is heterologous to the peptidoglycan hydrolase.

In some embodiments, the pharmacokinetic modifying group comprises a moiety selected from the group consisting of albumin, an immunoglobulin fragment, transferrin, Fn3, a functional variant thereof, or a functional fragment of the albumin, immunoglobulin fragment, transferrin, Fn3 or the functional variant thereof.

In some embodiments, the pharmacokinetic modifying group comprises human albumin, a functional variant thereof, or a functional fragment of the human albumin or the functional variant thereof.

In some embodiments, the pharmacokinetic modifying group is fused to the N-terminus of the peptidoglycan hydrolase.

In some embodiments, the amino acid sequence comprising a peptidoglycan hydrolase comprises from N-terminus to C-terminus: N-pharmacokinetic modifying group-GS-linker-peptidoglycan hydrolase-C.

In some embodiments, the amino acid sequence comprising a peptidoglycan hydrolase is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In some embodiments, the RNA comprises a modified nucleoside in place of uridine.

In some embodiments, the RNA comprises a modified nucleoside in place of each uridine.

In some embodiments, the modified nucleoside is selected from pseudouridine (ψ) N1-methyl-pseudouridine (m1ψ), and 5-methyl-uridine (m5U).

In some embodiments, the modified nucleoside is N1-methyl-pseudouridine (m1ψ).

In some embodiments, the RNA comprises a cap0 5′ cap.

In some embodiments, the RNA comprises the 5′ cap m₂^{7,2′O}G(5′)ppSp(5′) G (in particular its D1 diastereomer).

In some embodiments, the RNA comprises a cap1 5′ cap.

In some embodiments, the RNA comprises the 5′ cap m₂^{7,3′-O}Gppp(m₁^{2′-O})ApG.

In some embodiments, the RNA comprises a 5′ UTR.

In some embodiments, the 5′ UTR comprises the nucleotide sequence of SEQ ID NO: 11, or a nucleotide sequence having at least 99%, 98%, 97%, 96%, 95%, 90%, 85%, or 80% identity to the nucleotide sequence of SEQ ID NO: 11.

In some embodiments, the RNA comprises a 3′ UTR.

In some embodiments, the 3′ UTR comprises the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, the RNA comprises a poly-A sequence.

In some embodiments, the poly-A sequence comprises at least 100 nucleotides.

In some embodiments, the poly-A sequence comprises or consists of the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In some embodiments, the RNA is mRNA.

In some embodiments, the RNA encoding an amino acid sequence comprising a peptidoglycan hydrolase comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6 or the nucleotide sequence having at least 99%, 98%, 97%, 96%, 95%, 90%, 85%, or 80% identity to the nucleotide sequence of SEQ ID NO: 6.

In some embodiments, the RNA encoding an amino acid sequence comprising a peptidoglycan hydrolase comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a fragment of the nucleotide sequence of SEQ ID NO: 7 or the nucleotide sequence having at least 99%, 98%, 97%, 96%, 95%, 90%, 85%, or 80% identity to the nucleotide sequence of SEQ ID NO: 7.

In some embodiments, the RNA is self-amplifying RNA.

In some embodiments, the RNA encoding an amino acid sequence comprising a peptidoglycan hydrolase comprises the nucleotide sequence of SEQ ID NO: 8, a nucleotide sequence having at least 99%, 98%, 97%, 96%, 95%, 90%, 85%, or 80% identity to the nucleotide sequence of SEQ ID NO: 8, or a fragment of the nucleotide sequence of SEQ ID NO: 8 or the nucleotide sequence having at least 99%, 98%, 97%, 96%, 95%, 90%, 85%, or 80% identity to the nucleotide sequence of SEQ ID NO: 8.

In some embodiments, the RNA encoding an amino acid sequence comprising a peptidoglycan hydrolase comprises the nucleotide sequence of SEQ ID NO: 9, a nucleotide sequence having at least 99%, 98%, 97%, 96%, 95%, 90%, 85%, or 80% identity to the nucleotide sequence of SEQ ID NO: 9, or a fragment of the nucleotide sequence of SEQ ID NO: 9 or the nucleotide sequence having at least 99%, 98%, 97%, 96%, 95%, 90%, 85%, or 80% identity to the nucleotide sequence of SEQ ID NO: 9.

In some embodiments, the RNA is formulated as a liquid, formulated as a solid, or a combination thereof.

In some embodiments, the RNA is administered by injection.

In some embodiments, the RNA is administered by intravenous administration.

In some embodiments, the RNA is formulated as lipid particles.

In some embodiments, the RNA lipid particles are lipid nanoparticles (LNP).

In some embodiments, the RNA is formulated as a pharmaceutical composition.

In some embodiments, the pharmaceutical composition further comprises one or more pharmaceutically acceptable carriers, diluents and/or excipients.

In some embodiments, the subject is a human.

In some embodiments, the infection is a chronic infection.

In some embodiments, the bacteria causing infection are multi-resistant bacteria.

In some embodiments, the bacteria causing infection are Gram-negative bacteria, Gram-positive bacteria, or both.

In some embodiments, the bacteria are selected from Acinetobacter baumannii, Pseudomonas aeruginosa, Enterobacteriaceae, Porphyromonas gingivalis, Helicobacter pylori, Chlamydia trachomatis, Enterobacteriaceae, E. coli, Klebsiella pneumoniae, Salmonella, Shigella, Borellia, Campylobacter jejuni, Neisseria gonorrhoeae, Chlamydia trachomatis, Vibrio cholerae, and Fusobacterium nucleatum.

In some embodiments, the bacteria are resistant to carbapenem.

In some embodiments, the bacteria are selected from Staphylococci, Streptococci, Staphylococcus aureus, Streptococcus pneumoniae, Clostridioides difficile, Cutibacterium acnes, Mycobacterium tuberculosis, Gardnerella, Enterococcus faecalis, Enterococcus faecium, Lactobacillus iners, Bacillus subtilis and Bacillus anthracis.

In some embodiments, the bacteria are resistant to methicillin and vancomycin or non-susceptible to penicillin.

In some embodiments, the bacteria are methicillin-resistant Staphylococcus aureus (MRSA).

In some embodiments, the invention provides an agent described herein, e.g., the RNA, composition or medical preparation described herein, for use in a method described herein, e.g., for treating or preventing bacterial infection or a disease associated with or caused by bacterial infection.

In some embodiments, the RNA described herein is single-stranded RNA that may be translated into the respective protein upon entering cells, e.g., cells of a recipient. In addition to wildtype or codon-optimized sequences encoding an amino acid sequence comprising the amino acid sequence of a peptide or polypeptide having peptidoglycan hydrolase activity, e.g., an endolysin, the RNA may contain one or more structural elements optimized for maximal efficacy of the RNA with respect to stability and translational efficiency (5′ cap, 5′ UTR, 3′ UTR, poly(A)-tail). In some embodiments, the RNA contains all of these elements. In some embodiments, beta-S-ARCA (D1) (m₂^{7,2-O}GppSpG) or m₂^{7,3′-O}Gppp(m₁^{2′-O})ApG may be utilized as specific capping structure at the 5′-end of the RNA drug substances. As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These were identified by an ex vivo selection process for sequences that confer RNA stability and augment total protein expression (see WO 2017/060314, herein incorporated by reference). Alternatively, the 3′-UTR may be two re-iterated 3′-UTRs of the human beta-globin mRNA. Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency.

The encoded amino acid sequence may comprise amino acid sequences other than the amino acid sequence of a peptide or polypeptide having peptidoglycan hydrolase activity. Such other amino acid sequences may support the function or activity of the peptide or polypeptide having peptidoglycan hydrolase activity. In some embodiments, such other amino acid sequences comprise an amino acid sequence enhancing protein stability or persistence in the body.

Furthermore, in the RNA, a secretory signal peptide may be fused to the peptidoglycan hydrolase-encoding regions. Sequences coding for short linker peptides predominantly consisting of the amino acids glycine (G) and serine(S), as commonly used for fusion proteins may be used as GS/Linkers.

The RNA may be formulated in lipid particles to generate serum-stable formulations for intravenous (i.v.) administration. The RNA may be present in lipid nanoparticles (LNP). RNA-nanoparticles may target liver which results in an efficient expression of the encoded protein. In one embodiment, the RNA described herein is N1-methylpseudouridine modified, dsRNA-purified RNA which is formulated as lipid nanoparticles for intravenous administration.

### BRIEF DESCRIPTION OF THE FIGURES

FIG. 1. Bactericidal activity of 11 lysins expressed and secreted in P. pastoris. Supernatants of P. pastoris cultures expressing different lysin constructs were mixed with lysin buffer at the indicated dilutions and incubated with S. aureus cells. Bacterial growth was monitored by measuring the culture OD over the course of 24 hours. A.) Growth curves of S. aureus in the presence of supernatants from different lysin-expressing P. pastoris strains. L0502*: the supernatant of L0502 was diluted with a supernatant from wild type P. pastoris not expressing recombinant lysins (wt S/N). B.) Supernatants of different clones from lysin L0499 were incubated with S. aureus at the indicated dilutions and bacterial growth was monitored over time.

FIG. 2. Detection of RNA encoded LysPA26 in HEK293T cells by anti-FLAG BV241 conjugate antibody staining and flow cytometry. HEK293T cells analyzed by flow cytometry were incubated with: no RNA (non-transfected control), 9_LysPA26 and 10_LysPA26. Viability indicates cell survival post transfection, transfection rate is indicative of the percentage of cells with protein expression and expression rate is mean fluorescence of the percentage of positive cells Heights of bars indicate the means of technical triplicates.

FIG. 3. Western blot of denatured proteins detected in supernatants (culture media) and cell lysates of HEK cells transfected with 9_LysPA26 and 10_LysPA26 at concentration of 350 ng and 1 μg. Positive control used was HVV Aptamer-FLAG (not shown due to overexposure). Expected product size ^{˜}17 kDa.

FIG. 4. Schematic illustration of the general structure of RNA with 5′-cap, 5′- and 3′-untranslated regions, and poly(A)-tail. (A) Nucleoside-modified mRNA. (B) Self-amplifying mRNA additionally encodes for a viral-replicase and a subgenomic promotor. The individual elements are not drawn exactly true to scale compared to their respective sequence lengths. mRNA=messenger ribonucleic acid; SGP=subgenomic promotor; sec=secretory signal peptide; UTR=untranslated region; PolyA=poly adenosine.

FIG. 5. Expression of endolysins in HEK cells. HEK293T cells were transfected with 0.5 and 1 μg of modRNA and saRNA of construct 9_LysPA26 and construct 10_LysPA26. Extracellular protein or protein in the cell lysate was detected. Left panels show supernatant and cell lysates for modRNA. The constructs with secretion signals (10_LysPA26) are clearly detectable at both concentrations in the supernatant, while the constructs without secretion signals (9_LysPA26) cannot be detected. The same result is seen for saRNA.

FIG. 6. Expression of DNA and RNA encoded lysostaphin in cells.

A,B: Concentration and time dependent expression of L0483 in HEK293T/17 cells from mRNA (pST4-T7-AGA-dEarl-hAg-Kozak-SS (IGHV1-2)-His-3C-hL0483-FI-A30LA70) and DNA (pcDNA3.1 (+)-SS (IGHV1-2)-His-3C-hL0483). (A) 0.9×10⁶ HEK293T/17 cells were transfected with 3, 5 or 7.5 μg mRNA. In parallel 2.5 μg of DNA were transfected. Supernatants were collected after 24 (lane 1), 48 (lane 2) or 72 (lane 3) hours of expression. (B) Quantification of chemiluminescent signals from panel A using Image Lab 6.

C,D: Expression of L0483 mRNA-LNP in HEK293T/17 and CHO-K1 cells (1.5 μg). Expression was compared to mRNA transfection performed with Ribojuice (construct PL084_pST4-T7-AGA-dEarl-hAg-Kozak-SS (IGkappa)-His-3C-hL0483 (PM)-FLAG-FI-A30LA70). (C) Expression levels varied substantially between cell lines. Transfection with ribojuice resulted in lower expression. (D) Approximately 15 fold higher expression was seen from mRNA-LNPs compared to ribojuice transfection of the same mRNA.

FIG. 7. Expression of DNA and RNA encoded lysins in HEK293T/17 cells. Expression of lysin variants from mRNA or DNA transfections. 5 μg of mRNA or 2.5 μg of DNA were transfected into HEK293T/17 cells. Supernatants were analyzed by Western Blot, demonstrating expression from DNA as well as from mRNA.

FIG. 8. Bactericidal (A-D) and enzymatic (E) activity of lysins expressed from mRNA in HEK293T/17 cells. (A-D) Growth inhibition assay in duplicate. (A) Concentrated supernatant from HEK293T/17 transfection with 2.5 μg L0483 DNA in duplicate. (B) Growth control in duplicate. (C) Concentrated supernatant from HEK293T/17 transfection with L0483 mRNA-LNPs in duplicate. (D) Concentrated supernatant from HEK293T/17 transfection with Luciferase mRNA-LNPs in duplicate. (E) Enzymatic activity of lysins expressed from mRNA in HEK293T/17 cells.

FIG. 9. PK parameters of recombinant and mRNA delivered lysostaphin-Measurement of lysin levels in mouse plasma. (A) 3 mice were treated with 535 μg of recombinant lysostaphin and plasma lysin levels were measured for 24 hours. (B) 3 mice were treated with 107 μg of recombinant lysostaphin and plasma lysin levels were measured for 24 hours. (C) 5 mice were treated with 30 μg of L0483 mRNA-LNP and plasma lysin levels were measured for 72 hours.

FIG. 10. In vivo efficacy of lysostaphin in S. aureus mouse bacteremia. Shown is the reduction of bacterial burden in different organs after administration of two different doses of recombinant lysin or SOC (vancomycin).

### DESCRIPTION OF THE SEQUENCES

The following table provides a listing of certain sequences referenced herein.

TABLE 1
DESCRIPTION OF THE SEQUENCES

| | | |
|---|---|---|
| SEQ | | |
| ID | | |
| NO: | Description | SEQUENCE |

Endolysin LysPA26

| | | |
|---|---|---|
| 1 | LysPA26 | MRTNNIDAIKEHEGLRLVAYLDSVGVWTIGYGDTGPDVVKGLTITKEEAEKRLRKRLVEFEGYVNTYVKVPLKQHQFDALVSL |
| | (amino acid | VYNIGPTNFKTSTLLKKLNAGDYIGAADQFLVWNKGR VDGKLVVIKGLANRRAKERKQFLGE |
| | sequence) | |
| 2 | LysPA26 | AUGCGAACGAAUAAUAUCGACGCCAUUAAAGAGCACGAGGGGCUACGUCUUGUAGCCUACCUCGACUCUGUAGG |
| | (nucleotide | UGUAUGGACCAUUGGUUAUGGUGACACUGGCCCUGAUGUAGUUAAGGGCCUUACUAUUACCAAAGAAGAGGCC |
| | sequence, wt) | GAGAAGCGCCUUAGGAAGCGUCUGGUGGAGUUUGAAGGGUAUGUCAAUACCUACGUCAAGGUUCCGCUCAAAC |
| | | AGCAUCAAUUCGACGCUCUGGUGAGUUUGGUGUACAACAUUGGUCCUACUAACUUCAAGACCUCGACGUUGCU |
| | | UAAGAAGCUUAACGCUGGGGAUUAUAUUGGAGCGGCUGAUCAGUUCCUAGUGUGGAACAAAGGCCGAGUAGAU |
| | | GGGAAGCUUGUUGUGAUUAAGGGUCUUGCUAAUAGGCGGGCCAAAGAACGUAAACAAUUCCUUGGAGAAUGA |
| 3 | LysPA26 | AUGAGAACCAACAACAUCGAUGCCAUCAAAGAACAUGAAGGACUGAGACUGGUGGCUUACCUGGAUUCUGUGG |
| | (nucleotide | GAGUGUGGACAAUUGGAUAUGGAGACACAGGACCUGACGUGGUGAAGGGACUGACAAUCACAAAGGAAGAAGC |
| | sequence, | CGAAAAGAGACUGAGAAAGAGACUGGUGGAAUUUGAAGGAUAUGUGAACACCUAUGUGAAAGUGCCUCUGAAA |
| | opt10) | CAGCACCAGUUUGAUGCUCUGGUGAGCCUGGUGUACAACAUUGGACCCACAAACUUCAAGACAAGCACACUGCU |
| | | GAAGAAGCUGAAUGCUGGAGAUUACAUUGGAGCUGCUGAUCAGUUCCUGGUGUGGAACAAAGGCAGAGUGGA |
| | | UGGCAAACUGGUGGUGAUCAAAGGCCUGGCCAACAGGAGAGCCAAAGAAAGAAAACAGUUCCUGGGAGAAUGA |

Secreted Endolysin LysPA26

| | | |
|---|---|---|
| 4 | Secreted | MRVMAPRTLILLLSGALALTETWAGSRTNNIDAIKEHEGLRLVAYLDSVGVWTIGYGDTGPDVVKGLTITKEEAEKRLRKRLVEF |
| | LysPA26 | EGYVNTYVKVPLKQHQFDALVSLVYNIGPTNFKTSTLLKKLNAGDYIGAADQFLVWNKGRVDGKLVVIKGLANRRAKERKQFL |
| | (amino acid | GE |
| | sequence) | |
| 5 | Secreted | AUGAGAGUGAUGGCCCCCAGAACCCUGAUCCUGCUGCUGUCUGGCGCCCUGGCCCUGACAGAGACAUGGGCCGG |
| | LysPA26 | AAGCAGAACCAACAACAUCGAUGCCAUCAAAGAACAUGAAGGACUGAGACUGGUGGCUUACCUGGAUUCUGUGG |
| | (nucleotide | GAGUGUGGACAAUUGGAUAUGGAGACACAGGACCUGACGUGGUGAAGGGACUGACAAUCACAAAGGAAGAAGCC |
| | sequence) | GAAAAGAGACUGAGAAAGAGACUGGUGGAAUUUGAAGGAUAUGUGAACACCUAUGUGAAAGUGCCUCUGAAAC |
| | | AGCACCAGUUUGAUGCUCUGGUGAGCCUGGUGUACAACAUUGGACCCACAAACUUCAAGACAAGCACACUGCUG |
| | | AAGAAGCUGAAUGCUGGAGAUUACAUUGGAGCUGCUGAUCAGUUCCUGGUGUGGAACAAAGGCAGAGUGGAUG |
| | | GCAAACUGGUGGUGAUCAAAGGCCUGGCCAACAGGAGAGCCAAAGAAAGAAAACAGUUCCUGGGAGAAUGA |

pSTS-17-AGA-dEarl-hAg-LysPA26-FI-A30LA70

| | | |
|---|---|---|
| 6 | nucleotide | AGAAUAAACUAGUAUUCUUCUGGUCCCCACAGACUCAGAGAGAACCCGCCACCAUGAGAACCAACAACAUCGAUG |
| | sequence | CCAUCAAAGAACAUGAAGGACUGAGACUGGUGGCUUACCUGGAUUCUGUGGGAGUGUGGACAAUUGGAUAUGG |
| | | AGACACAGGACCUGACGUGGUGAAGGGACUGACAAUCACAAAGGAAGAAGCCGAAAAGAGACUGAGAAAGAGAC |
| | | UGGUGGAAUUUGAAGGAUAUGUGAACACCUAUGUGAAAGUGCCUCUGAAACAGCACCAGUUUGAUGCUCUGGU |
| | | GAGCCUGGUGUACAACAUUGGACCCACAAACUUCAAGACAAGCACACUGCUGAAGAAGCUGAAUGCUGGAGAUU |
| | | ACAUUGGAGCUGCUGAUCAGUUCCUGGUGUGGAACAAAGGCAGAGUGGAUGGCAAACUGGUGGUGAUCAAAGG |
| | | CCUGGCCAACAGGAGAGCCAAAGAAAGAAAACAGUUCCUGGGAGAAUGAUGACUCGAGCUGGUACUGCAUGCAC |
| | | GCAAUGCUAGCUGCCCCUUUCCCGUCCUGGGUACCCCGAGUCUCCCCCGACCUCGGGUCCCAGGUAUGCUCCCAC |
| | | CUCCACCUGCCCCACUCACCACCUCUGCUAGUUCCAGACACCUCCCAAGCACGCAGCAAUGCAGCUCAAAACGCUU |
| | | AGCCUAGCCACACCCCCACGGGAAACAGCAGUGAUUAACCUUUAGCAAUAAACGAAAGUUUAACUAAGCUAUACU |
| | | AACCCCAGGGUUGGUCAAUUUCGUGCCAGCCACACCCUGGAGCUAGCAAAAAAAAAAAAAAAAAAAAAAAAAAAA |
| | | AAGCAUAUGACUAAAAAAAAAAAAAAAAAAAAAAAAAAAAAAAAAAAAAAAAAAAAAAAAAAAAAAAAAAAAAAAA |
| | | AAAAAA |

pST5-T7-AGA-dEarl-hAg-husec(opt)-LysPA26-FI-A30LA70

| | | |
|---|---|---|
| 7 | nucleotide | AGAAUAAACUAGUAUUCUUCUGGUCCCCACAGACUCAGAGAGAACCCGCCACCAUGAGAGUGAUGGCCCCCAGAA |
| | sequence | CCCUGAUCCUGCUGCUGUCUGGCGCCCUGGCCCUGACAGAGACAUGGGCCGGAAGCAGAACCAACAACAUCGAUG |
| | | CCAUCAAAGAACAUGAAGGACUGAGACUGGUGGCUUACCUGGAUUCUGUGGGAGUGUGGACAAUUGGAUAUGG |
| | | AGACACAGGACCUGACGUGGUGAAGGGACUGACAAUCACAAAGGAAGAAGCCGAAAAGAGACUGAGAAAGAGAC |
| | | UGGUGGAAUUUGAAGGAUAUGUGAACACCUAUGUGAAAGUGCCUCUGAAACAGCACCAGUUUGAUGCUCUGGU |
| | | GAGCCUGGUGUACAACAUUGGACCCACAAACUUCAAGACAAGCACACUGCUGAAGAAGCUGAAUGCUGGAGAUU |
| | | ACAUUGGAGCUGCUGAUCAGUUCCUGGUGUGGAACAAAGGCAGAGUGGAUGGCAAACUGGUGGUGAUCAAAGG |
| | | CCUGGCCAACAGGAGAGCCAAAGAAAGAAAACAGUUCCUGGGAGAAUGAUGACUCGAGCUGGUACUGCAUGCAC |
| | | GCAAUGCUAGCUGCCCCUUUCCCGUCCUGGGUACCCCGAGUCUCCCCCGACCUCGGGUCCCAGGUAUGCUCCCAC |
| | | CUCCACCUGCCCCACUCACCACCUCUGCUAGUUCCAGACACCUCCCAAGCACGCAGCAAUGCAGCUCAAAACGCUU |
| | | AGCCUAGCCACACCCCCACGGGAAACAGCAGUGAUUAACCUUUAGCAAUAAACGAAAGUUUAACUAAGCUAUACU |
| | | AACCCCAGGGUUGGUCAAUUUCGUGCCAGCCACACCCUGGAGCUAGCAAAAAAAAAAAAAAAAAAAAAAAAAAAA |
| | | AAGCAUAUGACUAAAAAAAAAAAAAAAAAAAAAAAAAAAAAAAAAAAAAAAAAAAAAAAAAAAAAAAAAAAAAAAA |
| | | AAAAAA |

pST1-OPAL-LysPA26-A30LA70

| | | |
|---|---|---|
| 8 | nucleotide | AUGGCGGAUGUGUGACAUACACGACGCCAAAAGAUUUUGUUCCAGCUCCUGCCACCUCCGCUACGCGAGAGAUU |
| | sequence | AACCACCCACGAUGGCCGCCAAAGUGCAUGUUGAUAUUGAGGCUGACAGCCCAUUCAUCAAGUCUUUGCAGAAG |
| | | GCAUUUCCGUCGUUCGAGGUGGAGUCAUUGCAGGUCACACCAAAUGACCAUGCAAAUGCCAGAGCAUUUUCGCA |
| | | CCUGGCUACCAAAUUGAUCGAGCAGGAGACUGACAAAGACACACUCAUCUUGGAUAUCGGCAGUGCGCCUUCCAG |
| | | GAGAAUGAUGUCUACGCACAAAUACCACUGCGUAUGCCCUAUGCGCAGCGCAGAAGACCCCGAAAGGCUCGUAUG |
| | | CUACGCAAAGAAACUGGCAGCGGCCUCCGGGAAGGUGCUGGAUAGAGAGAUCGCAGGAAAAAUCACCGACCUGCA |
| | | GACCGUCAUGGCUACGCCAGACGCUGAAUCUCCUACCUUUUGCCUGCAUACAGACGUCACGUGUCGUACGGCAGC |
| | | CGAAGUGGCCGUAUACCAGGACGUGUAUGCUGUACAUGCACCAACAUCGCUGUACCAUCAGGCGAUGAAAGGUG |
| | | UCAGAACGGCGUAUUGGAUUGGGUUUGACACCACCCCGUUUAUGUUUGACGCGCUAGCAGGCGCGUAUCCAACC |
| | | UACGCCACAAACUGGGCCGACGAGCAGGUGUUACAGGCCAGGAACAUAGGACUGUGUGCAGCAUCCUUGACUGA |
| | | GGGAAGACUCGGCAAACUGUCCAUUCUCCGCAAGAAGCAAUUGAAACCUUGCGACACAGUCAUGUUCUCGGUAG |
| | | GAUCUACAUUGUACACUGAGAGCAGAAAGCUACUGAGGAGCUGGCACUUACCCUCCGUAUUCCACCUGAAAGGU |
| | | AAACAAUCCUUUACCUGUAGGUGCGAUACCAUCGUAUCAUGUGAAGGGUACGUAGUUAAGAAAAUCACUAUGUG |
| | | CCCCGGCCUGUACGGUAAAACGGUAGGGUACGCCGUGACGUAUCACGCGGAGGGAUUCCUAGUGUGCAAGACCA |
| | | CAGACACUGUCAAAGGAGAAAGAGUCUCAUUCCCUGUAUGCACCUACGUCCCCUCAACCAUCUGUGAUCAAAUGA |
| | | CUGGCAUACUAGCGACCGACGUCACACCGGAGGACGCACAGAAGUUGUUAGUGGGAUUGAAUCAGAGGAUAGUU |
| | | GUGAACGGAAGAACACAGCGAAACACUAACACGAUGAAGAACUAUCUGCUUCCGAUUGUGGCCGUCGCAUUUAG |
| | | CAAGUGGGCGAGGGAAUACAAGGCAGACCUUGAUGAUGAAAAACCUCUGGGUGUCCGAGAGAGGUCACUUACU |
| | | UGCUGCUGCUUGUGGGCAUUUAAAACGAGGAAGAUGCACACCAUGUACAAGAAACCAGACACCCAGACAAUAGU |
| | | GAAGGUGCCUUCAGAGUUUAACUCGUUCGUCAUCCCGAGCCUAUGGUCUACAGGCCUCGCAAUCCCAGUCAGAU |
| | | CACGCAUUAAGAUGCUUUUGGCCAAGAAGACCAAGCGAGAGUUAAUACCUGUUCUCGACGCGUCGUCAGCCAGG |
| | | GAUGCUGAACAAGAGGAGAAGGAGAGGUUGGAGGCCGAGCUGACUAGAGAAGCCUUACCACCCCUCGUCCCCAU |
| | | CGCGCCGGCGGAGACGGGAGUCGUCGACGUCGACGUUGAAGAACUAGAGUAUCACGCAGGUGCAGGGGUCGUGG |
| | | AAACACCUCGCAGCGCGUUGAAAGUCACCGCACAGCCGAACGACGUACUACUAGGAAAUUACGUAGUUCUGUCCC |
| | | CGCAGACCGUGCUCAAGAGCUCCAAGUUGGCCCCCGUGCACCCUCUAGCAGAGCAGGUGAAAAUAAUAACACAUA |
| | | ACGGGAGGGCCGGCCGUUACCAGGUCGACGGAUAUGACGGCAGGGUCCUACUACCAUGUGGAUCGGCCAUUCCG |
| | | GUCCCUGAGUUUCAGGCUUUGAGCGAGAGCGCCACUAUGGUGUACAACGAAAGGGAGUUCGUCAACAGGAAACU |
| | | AUACCAUAUUGCCGUUCACGGACCCUCGCUGAACACCGACGAGGAGAACUACGAGAAAGUCAGAGCUGAAAGAAC |
| | | UGACGCCGAGUACGUGUUCGACGUAGAUAAAAAAUGCUGCGUCAAGAGAGAGGAAGCGUCGGGUUUGGUGUUG |
| | | GUGGGAGAGCUAACCAACCCCCCGUUCCAUGAAUUCGCCUACGAAGGGCUGAAGAUCAGGCCGUCGGCACCAUAU |
| | | AAGACUACAGUAGUAGGAGUCUUUGGGGUUCCGGGAUCAGGCAAGUCUGCUAUUAUUAAGAGCCUCGUGACCA |
| | | AACACGAUCUGGUCACCAGCGGCAAGAAGGAGAACUGCCAGGAAAUAGUCAACGACGUGAAGAAGCACCGCGGAC |
| | | UGGACAUCCAGGCAAAAACAGUGGACUCCAUCCUGCUAAACGGGUGUCGUCGUGCCGUGGACAUCCUAUAUGUG |
| | | GACGAGGCUUUCGCUUGCCAUUCCGGUACUCUGCUAGCCCUAAUUGCUCUUGUUAAACCUCGGAGCAAAGUGGU |
| | | GUUAUGCGGAGACCCCAAGCAAUGCGGAUUCUUCAAUAUGAUGCAGCUUAAGGUGAACUUCAACCACAACAUCU |
| | | GCACUGAAGUAUGUCAUAAAAGUAUAUCCAGACGUUGCACGCGUCCAGUCACGGCCAUCGUGUCUACGUUGCAC |
| | | UACGGAGGCAAGAUGCGCACGACCAACCCGUGCAACAAACCCAUAAUCAUAGACACCACAGGACAGACCAAGCCCA |
| | | AGCCAGGAGACAUCGUGUUAACAUGCUUCCGAGGCUGGGUAAAGCAGCUGCAGUUGGACUACCGUGGACACGAA |
| | | GUCAUGACAGCAGCAGCAUCUCAGGGCCUCACCCGCAAAGGGGUAUACGCCGUAAGGCAGAAGGUGAAUGAAAA |
| | | UCCCUUGUAUGCCCCUGCGUCGGAGCACGUGAAUGUACUGCUGACGCGCACUGAGGAUAGGCUGGUGUGGAAA |
| | | ACGCUGGCCGGCGAUCCCUGGAUUAAGGUCCUAUCAAACAUUCCACAGGGUAACUUUACGGCCACAUUGGAAGA |
| | | AUGGCAAGAAGAACACGACAAAAUAAUGAAGGUGAUUGAAGGACCGGCUGCGCCUGUGGACGCGUUCCAGAACA |
| | | AAGCGAACGUGUGUUGGGCGAAAAGCCUGGUGCCUGUCCUGGACACUGCCGGAAUCAGAUUGACAGCAGAGGA |
| | | GUGGAGCACCAUAAUUACAGCAUUUAAGGAGGACAGAGCUUACUCUCCAGUGGUGGCCUUGAAUGAAAUUUGC |
| | | ACCAAGUACUAUGGAGUUGACCUGGACAGUGGCCUGUUUUCUGCCCCGAAGGUGUCCCUGUAUUACGAGAACAA |
| | | CCACUGGGAUAACAGACCUGGUGGAAGGAUGUAUGGAUUCAAUGCCGCAACAGCUGCCAGGCUGGAAGCUAGAC |
| | | AUACCUUCCUGAAGGGGCAGUGGCAUACGGGCAAGCAGGCAGUUAUCGCAGAAAGAAAAAUCCAACCGCUUUCU |
| | | GUGCUGGACAAUGUAAUUCCUAUCAACCGCAGGCUGCCGCACGCCCUGGUGGCUGAGUACAAGACGGUUAAAGG |
| | | CAGUAGGGUUGAGUGGCUGGUCAAUAAAGUAAGAGGGUACCACGUCCUGCUGGUGAGUGAGUACAACCUGGCU |
| | | UUGCCUCGACGCAGGGUCACUUGGUUGUCACCGCUGAAUGUCACAGGCGCCGAUAGGUGCUACGACCUAAGUUU |
| | | AGGACUGCCGGCUGACGCCGGCAGGUUCGACUUGGUCUUUGUGAACAUUCACACGGAAUUCAGAAUCCACCACU |
| | | ACCAGCAGUGUGUCGACCACGCCAUGAAGCUGCAGAUGCUUGGGGGAGAUGCGCUACGACUGCUAAAACCCGGC |
| | | GGCAGCCUCUUGAUGAGAGCUUACGGAUACGCCGAUAAAAUCAGCGAAGCCGUUGUUUCCUCCUUAAGCAGAAA |
| | | GUUCUCGUCUGCAAGAGUGUUGCGCCCGGAUUGUGUCACCAGCAAUACAGAAGUGUUCUUGCUGUUCUCCAAC |
| | | UUUGACAACGGAAAGAGACCCUCUACGCUACACCAGAUGAAUACCAAGCUGAGUGCCGUGUAUGCCGGAGAAGCC |
| | | AUGCACACGGCCGGGUGUGCACCAUCCUACAGAGUUAAGAGAGCAGACAUAGCCACGUGCACAGAAGCGGCUGU |
| | | GGUUAACGCAGCUAACGCCCGUGGAACUGUAGGGGAUGGCGUAUGCAGGGCCGUGGCGAAGAAAUGGCCGUCAG |
| | | CCUUUAAGGGAGAAGCAACACCAGUGGGCACAAUUAAAACAGUCAUGUGCGGCUCGUACCCCGUCAUCCACGCUG |
| | | UAGCGCCUAAUUUCUCUGCCACGACUGAAGCGGAAGGGGACCGCGAAUUGGCCGCUGUCUACCGGGCAGUGGCC |
| | | GCCGAAGUAAACAGACUGUCACUGAGCAGCGUAGCCAUCCCGCUGCUGUCCACAGGAGUGUUCAGCGGCGGUAG |
| | | AGAUAGGCUGCAGCAAUCCCUCAACCAUCUAUUCACAGCAAUGGACGCCACGGACGCUGACGUGACCAUCUACUG |
| | | CAGAGACAAAAGUUGGGAGAAGAAAAUCCAGGAAGCCAUAGACAUGAGGACGGCUGUGGAGUUGCUCAAUGAU |
| | | GACGUGGAGCUGACCACAGACUUGGUGAGAGUGCACCCGGACAGCAGCCUGGUGGGUCGUAAGGGCUACAGUAC |
| | | CACUGACGGGUCGCUGUACUCGUACUUUGAAGGUACGAAAUUCAACCAGGCUGCUAUUGAUAUGGCAGAGAUAC |
| | | UGACGUUGUGGCCCAGACUGCAAGAGGCAAACGAACAGAUAUGCCUAUACGCGCUGGGCGAAACAAUGGACAACA |
| | | UCAGAUCCAAAUGUCCGGUGAACGAUUCCGAUUCAUCAACACCUCCCAGGACAGUGCCCUGCCUGUGCCGCUACG |
| | | CAAUGACAGCAGAACGGAUCGCCCGCCUUAGGUCACACCAAGUUAAAAGCAUGGUGGUUUGCUCAUCUUUUCCC |
| | | CUCCCGAAAUACCAUGUAGAUGGGGUGCAGAAGGUAAAGUGCGAGAAGGUUCUCCUGUUCGACCCGACGGUACC |
| | | UUCAGUGGUUAGUCCGCGGAAGUAUGCCGCAUCUACGACGGACCACUCAGAUCGGUCGUUACGAGGGUUUGACU |
| | | UGGACUGGACCACCGACUCGUCUUCCACUGCCAGCGAUACCAUGUCGCUACCCAGUUUGCAGUCGUGUGACAUCG |
| | | ACUCGAUCUACGAGCCAAUGGCUCCCAUAGUAGUGACGGCUGACGUACACCCUGAACCCGCAGGCAUCGCGGACC |
| | | UGGCGGCAGAUGUGCAUCCUGAACCCGCAGACCAUGUGGACCUGGAGAACCCGAUUCCUCCACCGCGCCCAAAGA |
| | | GAGCUGCAUACCUUGCCUCCCGCGCGGCGGAGCGACCGGUGCCGGCGCCGAGAAAGCCGACGCCUGCCCCAAGGA |
| | | CUGCGUUUAGGAACAAGCUGCCUUUGACGUUCGGCGACUUUGACGAGCACGAGGUCGAUGCGUUGGCCUCCGG |
| | | GAUUACUUUCGGAGACUUCGACGACGUCCUGCGACUAGGCCGCGCGGGUGCAUAUAUUUUCUCCUCGGACACUG |
| | | GCAGCGGACAUUUACAACAAAAAUCCGUUAGGCAGCACAAUCUCCAGUGCGCACAACUGGAUGCGGUCGAGGAG |
| | | GAGAAAAUGUACCCGCCAAAAUUGGAUACUGAGAGGGAGAAGCUGUUGCUGCUGAAAAUGCAGAUGCACCCAUC |
| | | GGAGGCUAAUAAGAGUCGAUACCAGUCUCGCAAAGUGGAGAACAUGAAAGCCACGGUGGUGGACAGGCUCACAU |
| | | CGGGGGCCAGAUUGUACACGGGAGCGGACGUAGGCCGCAUACCAACAUACGCGGUUCGGUACCCCCGCCCCGUGU |
| | | ACUCCCCUACCGUGAUCGAAAGAUUCUCAAGCCCCGAUGUAGCAAUCGCAGCGUGCAACGAAUACCUAUCCAGAA |
| | | AUUACCCAACAGUGGCGUCGUACCAGAUAACAGAUGAAUACGACGCAUACUUGGACAUGGUUGACGGGUCGGAU |
| | | AGUUGCUUGGACAGAGCGACAUUCUGCCCGGCGAAGCUCCGGUGCUACCCGAAACAUCAUGCGUACCACCAGCCG |
| | | ACUGUACGCAGUGCCGUCCCGUCACCCUUUCAGAACACACUACAGAACGUGCUAGCGGCUGCCACCAAGAGAAAC |
| | | UGCAACGUCACGCAAAUGCGAGAACUACCCACCAUGGACUCGGCAGUGUUCAACGUGGAGUGCUUCAAGCGCUA |
| | | UGCCUGCUCCGGAGAAUAUUGGGAAGAAUAUGCUAAACAACCUAUCCGGAUAACCACUGAGAACAUCACUACCUA |
| | | UGUGACCAAAUUGAAAGGCCCGAAAGCUGCUGCCUUGUUCGCUAAGACCCACAACUUGGUUCCGCUGCAGGAGG |
| | | UUCCCAUGGACAGAUUCACGGUCGACAUGAAACGAGAUGUCAAAGUCACUCCAGGGACGAAACACACAGAGGAAA |
| | | GACCCAAAGUCCAGGUAAUUCAAGCAGCGGAGCCAUUGGCGACCGCUUACCUGUGCGGCAUCCACAGGGAAUUAG |
| | | UAAGGAGACUAAAUGCUGUGUUACGCCCUAACGUGCACACAUUGUUUGAUAUGUCGGCCGAAGACUUUGACGCG |
| | | AUCAUCGCCUCUCACUUCCACCCAGGAGACCCGGUUCUAGAGACGGACAUUGCAUCAUUCGACAAAAGCCAGGAC |
| | | GACUCCUUGGCUCUUACAGGUUUAAUGAUCCUCGAAGAUCUAGGGGUGGAUCAGUACCUGCUGGACUUGAUCG |
| | | AGGCAGCCUUUGGGGAAAUAUCCAGCUGUCACCUACCAACUGGCACGCGCUUCAAGUUCGGAGCUAUGAUGAAA |
| | | UCGGGCAUGUUUCUGACUUUGUUUAUUAACACUGUUUUGAACAUCACCAUAGCAAGCAGGGUACUGGAGCAGA |
| | | GACUCACUGACUCCGCCUGUGCGGCCUUCAUCGGCGACGACAACAUCGUUCACGGAGUGAUCUCCGACAAGCUGA |
| | | UGGCGGAGAGGUGCGCGUCGUGGGUCAACAUGGAGGUGAAGAUCAUUGACGCUGUCAUGGGCGAAAAACCCCC |
| | | AUAUUUUUGUGGGGGAUUCAUAGUUUUUGACAGCGUCACACAGACCGCCUGCCGUGUUUCAGACCCACUUAAGC |
| | | GCCUGUUCAAGUUGGGUAAGCCGCUAACAGCUGAAGACAAGCAGGACGAAGACAGGCGACGAGCACUGAGUGAC |
| | | GAGGUUAGCAAGUGGUUCCGGACAGGCUUGGGGGCCGAACUGGAGGUGGCACUAACAUCUAGGUAUGAGGUAG |
| | | AGGGCUGCAAAAGUAUCCUCAUAGCCAUGGCCACCUUGGCGAGGGACAUUAAGGCGUUUAAGAAAUUGAGAGGA |
| | | CCUGUUAUACACCUCUACGGCGGUCCUAGAUUGGUGCGUUAAUACACAGAAUUCUGAUUAUAGCGCACUAUUAU |
| | | AGCACCACUAGU |
| | | AUGAGAACCAACAACAUCGAUGCCAUCAAAGAACAUGAAGGACUGAGACUGGUGGCUUACCUGGAUUCUGUGGG |
| | | AGUGUGGACAAUUGGAUAUGGAGACACAGGACCUGACGUGGUGAAGGGACUGACAAUCACAAAGGAAGAAGCCG |
| | | AAAAGAGACUGAGAAAGAGACUGGUGGAAUUUGAAGGAUAUGUGAACACCUAUGUGAAAGUGCCUCUGAAACA |
| | | GCACCAGUUUGAUGCUCUGGUGAGCCUGGUGUACAACAUUGGACCCACAAACUUCAAGACAAGCACACUGCUGA |
| | | AGAAGCUGAAUGCUGGAGAUUACAUUGGAGCUGCUGAUCAGUUCCUGGUGUGGAACAAAGGCAGAGUGGAUGG |
| | | CAAACUGGUGGUGAUCAAAGGCCUGGCCAACAGGAGAGCCAAAGAAAGAAAACAGUUCCUGGGAGAAUGAUGAC |
| | | UCGAGGCGGCCGCAGGAGCUUAAUUCGACGAAUAAUUGGAUUUUUAUUUUAUUUUGCAAUUGGUUUUUAAUA |
| | | UUUCCAAAAAAAAAAAAAAAAAAAAAAAAAAAAAAGCAUAUGACUAAAAAAAAAAAAAAAAAAAAAAAAAAAAAA |
| | | AAAAAAAAAAAAAAAAAAAAAAAAAAAAAAAAAAAAAAAA |

pST1-OPAL-husec(opt)-LysPA26-A30LA70

| | | |
|---|---|---|
| 9 | nucleotide | AUGGCGGAUGUGUGACAUACACGACGCCAAAAGAUUUUGUUCCAGCUCCUGCCACCUCCGCUACGCGAGAGAUU |
| | sequence | AACCACCCACGAUGGCCGCCAAAGUGCAUGUUGAUAUUGAGGCUGACAGCCCAUUCAUCAAGUCUUUGCAGAAG |
| | | GCAUUUCCGUCGUUCGAGGUGGAGUCAUUGCAGGUCACACCAAAUGACCAUGCAAAUGCCAGAGCAUUUUCGCA |
| | | CCUGGCUACCAAAUUGAUCGAGCAGGAGACUGACAAAGACACACUCAUCUUGGAUAUCGGCAGUGCGCCUUCCAG |
| | | GAGAAUGAUGUCUACGCACAAAUACCACUGCGUAUGCCCUAUGCGCAGCGCAGAAGACCCCGAAAGGCUCGUAUG |
| | | CUACGCAAAGAAACUGGCAGCGGCCUCCGGGAAGGUGCUGGAUAGAGAGAUCGCAGGAAAAAUCACCGACCUGCA |
| | | GACCGUCAUGGCUACGCCAGACGCUGAAUCUCCUACCUUUUGCCUGCAUACAGACGUCACGUGUCGUACGGCAGC |
| | | CGAAGUGGCCGUAUACCAGGACGUGUAUGCUGUACAUGCACCAACAUCGCUGUACCAUCAGGCGAUGAAAGGUG |
| | | UCAGAACGGCGUAUUGGAUUGGGUUUGACACCACCCCGUUUAUGUUUGACGCGCUAGCAGGCGCGUAUCCAACC |
| | | UACGCCACAAACUGGGCCGACGAGCAGGUGUUACAGGCCAGGAACAUAGGACUGUGUGCAGCAUCCUUGACUGA |
| | | GGGAAGACUCGGCAAACUGUCCAUUCUCCGCAAGAAGCAAUUGAAACCUUGCGACACAGUCAUGUUCUCGGUAG |
| | | GAUCUACAUUGUACACUGAGAGCAGAAAGCUACUGAGGAGCUGGCACUUACCCUCCGUAUUCCACCUGAAAGGU |
| | | AAACAAUCCUUUACCUGUAGGUGCGAUACCAUCGUAUCAUGUGAAGGGUACGUAGUUAAGAAAAUCACUAUGUG |
| | | CAGACACUGUCAAAGGAGAAAGAGUCUCAUUCCCUGUAUGCACCUACGUCCCCUCAACCAUCUGUGAUCAAAUGA |
| | | CCCCGGCCUGUACGGUAAAACGGUAGGGUACGCCGUGACGUAUCACGCGGAGGGAUUCCUAGUGUGCAAGACCA |
| | | CUGGCAUACUAGCGACCGACGUCACACCGGAGGACGCACAGAAGUUGUUAGUGGGAUUGAAUCAGAGGAUAGUU |
| | | GUGAACGGAAGAACACAGCGAAACACUAACACGAUGAAGAACUAUCUGCUUCCGAUUGUGGCCGUCGCAUUUAG |
| | | CAAGUGGGCGAGGGAAUACAAGGCAGACCUUGAUGAUGAAAAACCUCUGGGUGUCCGAGAGAGGUCACUUACU |
| | | UGCUGCUGCUUGUGGGCAUUUAAAACGAGGAAGAUGCACACCAUGUACAAGAAACCAGACACCCAGACAAUAGU |
| | | GAAGGUGCCUUCAGAGUUUAACUCGUUCGUCAUCCCGAGCCUAUGGUCUACAGGCCUCGCAAUCCCAGUCAGAU |
| | | CACGCAUUAAGAUGCUUUUGGCCAAGAAGACCAAGCGAGAGUUAAUACCUGUUCUCGACGCGUCGUCAGCCAGG |
| | | GAUGCUGAACAAGAGGAGAAGGAGAGGUUGGAGGCCGAGCUGACUAGAGAAGCCUUACCACCCCUCGUCCCCAU |
| | | CGCGCCGGCGGAGACGGGAGUCGUCGACGUCGACGUUGAAGAACUAGAGUAUCACGCAGGUGCAGGGGUCGUGG |
| | | AAACACCUCGCAGCGCGUUGAAAGUCACCGCACAGCCGAACGACGUACUACUAGGAAAUUACGUAGUUCUGUCCC |
| | | CGCAGACCGUGCUCAAGAGCUCCAAGUUGGCCCCCGUGCACCCUCUAGCAGAGCAGGUGAAAAUAAUAACACAUA |
| | | ACGGGAGGGCCGGCCGUUACCAGGUCGACGGAUAUGACGGCAGGGUCCUACUACCAUGUGGAUCGGCCAUUCCG |
| | | GUCCCUGAGUUUCAGGCUUUGAGCGAGAGCGCCACUAUGGUGUACAACGAAAGGGAGUUCGUCAACAGGAAACU |
| | | AUACCAUAUUGCCGUUCACGGACCCUCGCUGAACACCGACGAGGAGAACUACGAGAAAGUCAGAGCUGAAAGAAC |
| | | UGACGCCGAGUACGUGUUCGACGUAGAUAAAAAAUGCUGCGUCAAGAGAGAGGAAGCGUCGGGUUUGGUGUUG |
| | | GUGGGAGAGCUAACCAACCCCCCGUUCCAUGAAUUCGCCUACGAAGGGCUGAAGAUCAGGCCGUCGGCACCAUAU |
| | | AAGACUACAGUAGUAGGAGUCUUUGGGGUUCCGGGAUCAGGCAAGUCUGCUAUUAUUAAGAGCCUCGUGACCA |
| | | AACACGAUCUGGUCACCAGCGGCAAGAAGGAGAACUGCCAGGAAAUAGUCAACGACGUGAAGAAGCACCGCGGAC |
| | | UGGACAUCCAGGCAAAAACAGUGGACUCCAUCCUGCUAAACGGGUGUCGUCGUGCCGUGGACAUCCUAUAUGUG |
| | | GACGAGGCUUUCGCUUGCCAUUCCGGUACUCUGCUAGCCCUAAUUGCUCUUGUUAAACCUCGGAGCAAAGUGGU |
| | | GUUAUGCGGAGACCCCAAGCAAUGCGGAUUCUUCAAUAUGAUGCAGCUUAAGGUGAACUUCAACCACAACAUCU |
| | | GCACUGAAGUAUGUCAUAAAAGUAUAUCCAGACGUUGCACGCGUCCAGUCACGGCCAUCGUGUCUACGUUGCAC |
| | | UACGGAGGCAAGAUGCGCACGACCAACCCGUGCAACAAACCCAUAAUCAUAGACACCACAGGACAGACCAAGCCCA |
| | | AGCCAGGAGACAUCGUGUUAACAUGCUUCCGAGGCUGGGUAAAGCAGCUGCAGUUGGACUACCGUGGACACGAA |
| | | GUCAUGACAGCAGCAGCAUCUCAGGGCCUCACCCGCAAAGGGGUAUACGCCGUAAGGCAGAAGGUGAAUGAAAA |
| | | UCCCUUGUAUGCCCCUGCGUCGGAGCACGUGAAUGUACUGCUGACGCGCACUGAGGAUAGGCUGGUGUGGAAA |
| | | ACGCUGGCCGGCGAUCCCUGGAUUAAGGUCCUAUCAAACAUUCCACAGGGUAACUUUACGGCCACAUUGGAAGA |
| | | AUGGCAAGAAGAACACGACAAAAUAAUGAAGGUGAUUGAAGGACCGGCUGCGCCUGUGGACGCGUUCCAGAACA |
| | | AAGCGAACGUGUGUUGGGCGAAAAGCCUGGUGCCUGUCCUGGACACUGCCGGAAUCAGAUUGACAGCAGAGGA |
| | | GUGGAGCACCAUAAUUACAGCAUUUAAGGAGGACAGAGCUUACUCUCCAGUGGUGGCCUUGAAUGAAAUUUGC |
| | | ACCAAGUACUAUGGAGUUGACCUGGACAGUGGCCUGUUUUCUGCCCCGAAGGUGUCCCUGUAUUACGAGAACAA |
| | | CCACUGGGAUAACAGACCUGGUGGAAGGAUGUAUGGAUUCAAUGCCGCAACAGCUGCCAGGCUGGAAGCUAGAC |
| | | AUACCUUCCUGAAGGGGCAGUGGCAUACGGGCAAGCAGGCAGUUAUCGCAGAAAGAAAAAUCCAACCGCUUUCU |
| | | GUGCUGGACAAUGUAAUUCCUAUCAACCGCAGGCUGCCGCACGCCCUGGUGGCUGAGUACAAGACGGUUAAAGG |
| | | CAGUAGGGUUGAGUGGCUGGUCAAUAAAGUAAGAGGGUACCACGUCCUGCUGGUGAGUGAGUACAACCUGGCU |
| | | UUGCCUCGACGCAGGGUCACUUGGUUGUCACCGCUGAAUGUCACAGGCGCCGAUAGGUGCUACGACCUAAGUUU |
| | | AGGACUGCCGGCUGACGCCGGCAGGUUCGACUUGGUCUUUGUGAACAUUCACACGGAAUUCAGAAUCCACCACU |
| | | ACCAGCAGUGUGUCGACCACGCCAUGAAGCUGCAGAUGCUUGGGGGAGAUGCGCUACGACUGCUAAAACCCGGC |
| | | GGCAGCCUCUUGAUGAGAGCUUACGGAUACGCCGAUAAAAUCAGCGAAGCCGUUGUUUCCUCCUUAAGCAGAAA |
| | | GUUCUCGUCUGCAAGAGUGUUGCGCCCGGAUUGUGUCACCAGCAAUACAGAAGUGUUCUUGCUGUUCUCCAAC |
| | | UUUGACAACGGAAAGAGACCCUCUACGCUACACCAGAUGAAUACCAAGCUGAGUGCCGUGUAUGCCGGAGAAGCC |
| | | AUGCACACGGCCGGGUGUGCACCAUCCUACAGAGUUAAGAGAGCAGACAUAGCCACGUGCACAGAAGCGGCUGU |
| | | GGUUAACGCAGCUAACGCCCGUGGAACUGUAGGGGAUGGCGUAUGCAGGGCCGUGGCGAAGAAAUGGCCGUCAG |
| | | CCUUUAAGGGAGAAGCAACACCAGUGGGCACAAUUAAAACAGUCAUGUGCGGCUCGUACCCCGUCAUCCACGCUG |
| | | UAGCGCCUAAUUUCUCUGCCACGACUGAAGCGGAAGGGGACCGCGAAUUGGCCGCUGUCUACCGGGCAGUGGCC |
| | | GCCGAAGUAAACAGACUGUCACUGAGCAGCGUAGCCAUCCCGCUGCUGUCCACAGGAGUGUUCAGCGGCGGUAG |
| | | AGAUAGGCUGCAGCAAUCCCUCAACCAUCUAUUCACAGCAAUGGACGCCACGGACGCUGACGUGACCAUCUACUG |
| | | CAGAGACAAAAGUUGGGAGAAGAAAAUCCAGGAAGCCAUAGACAUGAGGACGGCUGUGGAGUUGCUCAAUGAU |
| | | GACGUGGAGCUGACCACAGACUUGGUGAGAGUGCACCCGGACAGCAGCCUGGUGGGUCGUAAGGGCUACAGUAC |
| | | CACUGACGGGUCGCUGUACUCGUACUUUGAAGGUACGAAAUUCAACCAGGCUGCUAUUGAUAUGGCAGAGAUAC |
| | | UGACGUUGUGGCCCAGACUGCAAGAGGCAAACGAACAGAUAUGCCUAUACGCGCUGGGCGAAACAAUGGACAACA |
| | | UCAGAUCCAAAUGUCCGGUGAACGAUUCCGAUUCAUCAACACCUCCCAGGACAGUGCCCUGCCUGUGCCGCUACG |
| | | CAAUGACAGCAGAACGGAUCGCCCGCCUUAGGUCACACCAAGUUAAAAGCAUGGUGGUUUGCUCAUCUUUUCCC |
| | | CUCCCGAAAUACCAUGUAGAUGGGGUGCAGAAGGUAAAGUGCGAGAAGGUUCUCCUGUUCGACCCGACGGUACC |
| | | UUCAGUGGUUAGUCCGCGGAAGUAUGCCGCAUCUACGACGGACCACUCAGAUCGGUCGUUACGAGGGUUUGACU |
| | | UGGACUGGACCACCGACUCGUCUUCCACUGCCAGCGAUACCAUGUCGCUACCCAGUUUGCAGUCGUGUGACAUCG |
| | | ACUCGAUCUACGAGCCAAUGGCUCCCAUAGUAGUGACGGCUGACGUACACCCUGAACCCGCAGGCAUCGCGGACC |
| | | UGGCGGCAGAUGUGCAUCCUGAACCCGCAGACCAUGUGGACCUGGAGAACCCGAUUCCUCCACCGCGCCCAAAGA |
| | | GAGCUGCAUACCUUGCCUCCCGCGCGGCGGAGCGACCGGUGCCGGCGCCGAGAAAGCCGACGCCUGCCCCAAGGA |
| | | CUGCGUUUAGGAACAAGCUGCCUUUGACGUUCGGCGACUUUGACGAGCACGAGGUCGAUGCGUUGGCCUCCGG |
| | | GAUUACUUUCGGAGACUUCGACGACGUCCUGCGACUAGGCCGCGCGGGUGCAUAUAUUUUCUCCUCGGACACUG |
| | | GCAGCGGACAUUUACAACAAAAAUCCGUUAGGCAGCACAAUCUCCAGUGCGCACAACUGGAUGCGGUCGAGGAG |
| | | GAGAAAAUGUACCCGCCAAAAUUGGAUACUGAGAGGGAGAAGCUGUUGCUGCUGAAAAUGCAGAUGCACCCAUC |
| | | GGAGGCUAAUAAGAGUCGAUACCAGUCUCGCAAAGUGGAGAACAUGAAAGCCACGGUGGUGGACAGGCUCACAU |
| | | CGGGGGCCAGAUUGUACACGGGAGCGGACGUAGGCCGCAUACCAACAUACGCGGUUCGGUACCCCCGCCCCGUGU |
| | | ACUCCCCUACCGUGAUCGAAAGAUUCUCAAGCCCCGAUGUAGCAAUCGCAGCGUGCAACGAAUACCUAUCCAGAA |
| | | AUUACCCAACAGUGGCGUCGUACCAGAUAACAGAUGAAUACGACGCAUACUUGGACAUGGUUGACGGGUCGGAU |
| | | AGUUGCUUGGACAGAGCGACAUUCUGCCCGGCGAAGCUCCGGUGCUACCCGAAACAUCAUGCGUACCACCAGCCG |
| | | ACUGUACGCAGUGCCGUCCCGUCACCCUUUCAGAACACACUACAGAACGUGCUAGCGGCUGCCACCAAGAGAAAC |
| | | UGCAACGUCACGCAAAUGCGAGAACUACCCACCAUGGACUCGGCAGUGUUCAACGUGGAGUGCUUCAAGCGCUA |
| | | UGCCUGCUCCGGAGAAUAUUGGGAAGAAUAUGCUAAACAACCUAUCCGGAUAACCACUGAGAACAUCACUACCUA |
| | | UGUGACCAAAUUGAAAGGCCCGAAAGCUGCUGCCUUGUUCGCUAAGACCCACAACUUGGUUCCGCUGCAGGAGG |
| | | UUCCCAUGGACAGAUUCACGGUCGACAUGAAACGAGAUGUCAAAGUCACUCCAGGGACGAAACACACAGAGGAAA |
| | | GACCCAAAGUCCAGGUAAUUCAAGCAGCGGAGCCAUUGGCGACCGCUUACCUGUGCGGCAUCCACAGGGAAUUAG |
| | | UAAGGAGACUAAAUGCUGUGUUACGCCCUAACGUGCACACAUUGUUUGAUAUGUCGGCCGAAGACUUUGACGCG |
| | | AUCAUCGCCUCUCACUUCCACCCAGGAGACCCGGUUCUAGAGACGGACAUUGCAUCAUUCGACAAAAGCCAGGAC |
| | | GACUCCUUGGCUCUUACAGGUUUAAUGAUCCUCGAAGAUCUAGGGGUGGAUCAGUACCUGCUGGACUUGAUCG |
| | | AGGCAGCCUUUGGGGAAAUAUCCAGCUGUCACCUACCAACUGGCACGCGCUUCAAGUUCGGAGCUAUGAUGAAA |
| | | UCGGGCAUGUUUCUGACUUUGUUUAUUAACACUGUUUUGAACAUCACCAUAGCAAGCAGGGUACUGGAGCAGA |
| | | GACUCACUGACUCCGCCUGUGCGGCCUUCAUCGGCGACGACAACAUCGUUCACGGAGUGAUCUCCGACAAGCUGA |
| | | UGGCGGAGAGGUGCGCGUCGUGGGUCAACAUGGAGGUGAAGAUCAUUGACGCUGUCAUGGGCGAAAAACCCCC |
| | | AUAUUUUUGUGGGGGAUUCAUAGUUUUUGACAGCGUCACACAGACCGCCUGCCGUGUUUCAGACCCACUUAAGC |
| | | GCCUGUUCAAGUUGGGUAAGCCGCUAACAGCUGAAGACAAGCAGGACGAAGACAGGCGACGAGCACUGAGUGAC |
| | | GAGGUUAGCAAGUGGUUCCGGACAGGCUUGGGGGCCGAACUGGAGGUGGCACUAACAUCUAGGUAUGAGGUAG |
| | | AGGGCUGCAAAAGUAUCCUCAUAGCCAUGGCCACCUUGGCGAGGGACAUUAAGGCGUUUAAGAAAUUGAGAGGA |
| | | CCUGUUAUACACCUCUACGGCGGUCCUAGAUUGGUGCGUUAAUACACAGAAUUCUGAUUAUAGCGCACUAUUAU |
| | | AGCACCACUAGU |
| | | AUGAGAGUGAUGGCCCCCAGAACCCUGAUCCUGCUGCUGUCUGGCGCCCUGGCCCUGACAGAGACAUGGGCCGG |
| | | AAGCAGAACCAACAACAUCGAUGCCAUCAAAGAACAUGAAGGACUGAGACUGGUGGCUUACCUGGAUUCUGUGG |
| | | GAGUGUGGACAAUUGGAUAUGGAGACACAGGACCUGACGUGGUGAAGGGACUGACAAUCACAAAGGAAGAAGCC |
| | | GAAAAGAGACUGAGAAAGAGACUGGUGGAAUUUGAAGGAUAUGUGAACACCUAUGUGAAAGUGCCUCUGAAAC |
| | | AGCACCAGUUUGAUGCUCUGGUGAGCCUGGUGUACAACAUUGGACCCACAAACUUCAAGACAAGCACACUGCUG |
| | | AAGAAGCUGAAUGCUGGAGAUUACAUUGGAGCUGCUGAUCAGUUCCUGGUGUGGAACAAAGGCAGAGUGGAUG |
| | | GCAAACUGGUGGUGAUCAAAGGCCUGGCCAACAGGAGAGCCAAAGAAAGAAAACAGUUCCUGGGAGAAUGAUGA |
| | | CUCGAGGCGGCCGCAGGAGCUUAAUUCGACGAAUAAUUGGAUUUUUAUUUUAUUUUGCAAUUGGUUUUUAAU |
| | | AUUUCCAAAAAAAAAAAAAAAAAAAAAAAAAAAAAAGCAUAUGACUAAAAAAAAAAAAAAAAAAAAAAAAAAAAA |
| | | AAAAAAAAAAAAAAAAAAAAAAAAAAAAAAAAAAAAAAAAA |

Secretion signal

| | | |
|---|---|---|
| 10 | Sec (amino | MRVMAPRTLILLLSGALALTETWAGS |
| | acid) | |

5′-UTR (hAg-Kozak)

| | | |
|---|---|---|
| 11 | 5′-UTR | AUUCUUCUGGUCCCCACAGACUCAGAGAGAACCCGCCACC |

3′-UTR (FI element)

| | | |
|---|---|---|
| 12 | 3′-UTR | CUGGUACUGCAUGCACGCAAUGCUAGCUGCCCCUUUCCCGUCCUGGGUACCCCGAGUCUCCCCCGACCUCGGGUC |
| | | CCAGGUAUGCUCCCACCUCCACCUGCCCCACUCACCACCUCUGCUAGUUCCAGACACCUCCCAAGCACGCAGCAAU |
| | | GCAGCUCAAAACGCUUAGCCUAGCCACACCCCCACGGGAAACAGCAGUGAUUAACCUUUAGCAAUAAACGAAAGU |
| | | UUAACUAAGCUAUACUAACCCCAGGGUUGGUCAAUUUCGUGCCAGCCACACC |

A30L70

| | | |
|---|---|---|
| 13 | A30L70 | AAAAAAAAAAAAAAAAAAAAAAAAAAAAAAGCAUAUGACUAAAAAAAAAAAAAAAAAAAAAAAAAAAAAAAAAAA |
| | | AAAAAAAAAAAAAAAAAAAAAAAAAAAAAAAAAAA |

Lysin L0473

| | | |
|---|---|---|
| 14 | amino acid | MAKTKKQIEQRLQAYAKGTVDSPYRITKPTSYDPSFGVMEVGAIDADRYYHAQCQDLITDYVLWLTDNKVRPWGNAKDQI |
| | sequence | NQSYGTGFKIHKNLPSTVAKTGWIAVFTSGTYSQYGHIGIVYSPGDTNKFHIIEQNWNGWANKKPLTRWDNYYGLTHFIEV |
| | | PYAKETITTKKTAPKAATSKPAKKISVSHNNINLQSNFAKRGYNPKGVVIHNDAGRSTAQQYENSLKGANQNRLNNGIAHAY |
| | | ADSSHIWYAIEPERIAYHTGDGVKKGTGNYDYFGIEVCQSMSASDKEFMANEQVVLQEVARLLKKWKLPANRNTVRLHLEF |
| | | SATSCPHRSLTMRTGFNPVTQGRPSDAIIKKLKDIWIKEIRAYMEGKTPSIKTVANKPGSASTPATKSNSDGWKVNAYNTYYK |
| | | TERASFTVGSTRIAVRSTGPFTTCPFTGWLNPGVTITYDEVMVQNGFVWLGYDSYNGRRYVPIRTAQGTPGSSNYVLGPLW |
| | | GTIS |
| 15 | nucleotide | AUGGCUAAGACUAAGAAACAAAUCGAGCAAAGAUUGCAAGCUUAUGCUAAAGGUACUGUUGAUUCUCCUUACA |
| | sequence | GAAUUACUAAGCCAACUUCUUAUGAUCCUUCUUUCGGUGUUAUGGAAGUUGGUGCUAUUGAUGCUGAUAGAU |
| | | ACUAUCAUGCUCAAUGUCAAGAUUUGAUCACUGAUUACGUUUUGUGGUUGACUGAUAACAAAGUUAGACCAUG |
| | | GGGUAACGCUAAGGAUCAAAUUAAUCAAUCUUACGGUACUGGUUUUAAGAUCCACAAGAAUUUGCCUUCUACU |
| | | GUUGCUAAAACUGGUUGGAUUGCUGUUUUCACUUCUGGUACUUACUCUCAAUAUGGUCAUAUUGGUAUUGUU |
| | | UACUCUCCAGGAGAUACUAACAAGUUCCACAUCAUCGAACAAAACUGGAACGGUUGGGCUAACAAGAAACCAUU |
| | | GACUAGAUGGGAUAACUACUACGGUUUGACUCAUUUCAUCGAAGUUCCUUACGCUAAGGAGACUAUCACUACU |
| | | AAGAAAACUGCUCCAAAGGCUGCUACUUCUAAACCUGCUAAGAAAAUUUCUGUUUCUCAUAACAACAUUAACUU |
| | | GCAAUCUAAUUUUGCUAAAAGAGGUUACAACCCAAAGGGUGUUGUUAUUCACAAUGAUGCUGGUAGAUCUACU |
| | | GCUCAACAAUACGAAAAUUCUUUGAAGGGUGCUAACCAAAAUAGAUUGAACAAUGGUAUUGCUCAUGCUUACG |
| | | CUGAUUCUUCUCACAUUUGGUAUGCUAUUGAACCUGAGAGAAUUGCUUACCAUACUGGAGAUGGUGUUAAGAA |
| | | AGGUACUGGUAACUACGAUUACUUCGGUAUCGAAGUUUGUCAAUCUAUGUCUGCUUCUGAUAAAGAGUUUAU |
| | | GGCUAAUGAGCAAGUUGUUUUGCAAGAGGUUGCUAGAUUGUUGAAGAAAUGGAAGUUGCCAGCUAACAGAAA |
| | | CACUGUUAGAUUGCAUUUGGAAUUUUCUGCUACUUCUUGUCCUCACAGAUCUUUGACUAUGAGAACUGGUUU |
| | | CAAUCCAGUUACUCAAGGUAGACCUUCUGAUGCUAUUAUUAAGAAAUUGAAGGAUAUCUGGAUCAAGGAAAUC |
| | | AGAGCUUACAUGGAGGGUAAAACUCCAUCUAUUAAAACUGUUGCUAACAAGCCAGGUUCUGCUUCUACUCCUGC |
| | | UACUAAAUCUAAUUCUGAUGGUUGGAAGGUUAACGCUUACAACACUUACUACAAGACUGAGAGAGCUUCUUUU |
| | | ACUGUUGGUUCUACUAGAAUUGCUGUUAGAUCUACUGGUCCAUUCACUACUUGUCCUUUCACUGGUUGGUUG |
| | | AACCCAGGUGUUACUAUCACUUACGAUGAAGUUAUGGUUCAAAACGGUUUCGUUUGGUUGGGUUACGAUUCU |
| | | UAUAAUGGUAGAAGAUACGUUCCAAUUAGAACUGCUCAAGGUACUCCUGGUUCUUCUAAUUAUGUUUUGGGU |
| | | CCUUUGUGGGGUACUAUUUCUUAA |
| 16 | nucleotide | AUGGCCAAAACCAAGAAACAGAUAGAACAGAGACUGCAAGCUUACGCAAAGGGGACCGUGGAUUCCCCUUACCGA |
| | sequence | AUCACCAAGCCAACCAGCUACGACCCCUCCUUCGGUGUCAUGGAGGUUGGCGCUAUAGAUGCGGAUCGCUACUA |
| | (opt) | UCAUGCCCAGUGUCAAGAUCUGAUAACUGACUAUGUACUUUGGCUCACUGACAACAAAGUAAGACCUUGGGGU |
| | | AAUGCCAAGGACCAGAUUAACCAGAGCUAUGGGACUGGUUUCAAAAUCCACAAGAAUCUGCCCAGUACCGUGGC |
| | | UAAAACAGGAUGGAUUGCCGUGUUUACAUCCGGGACCUACUCACAGUACGGCCACAUCGGCAUCGUGUACAGUC |
| | | CAGGAGAUACCAACAAAUUCCAUAUCAUCGAGCAGAAUUGGAAUGGAUGGGCAAACAAGAAACCUCUGACACGU |
| | | UGGGACAACUACUACGGGCUGACCCACUUUAUCGAGGUCCCAUAUGCGAAAGAGACAAUCACAACGAAGAAAAC |
| | | UGCACCUAAAGCAGCUACCUCAAAACCGGCUAAGAAGAUCUCAGUUUCUCACAACAACAUCAACCUUCAGAGCAA |
| | | CUUUGCAAAGAGAGGGUACAACCCAAAGGGAGUGGUGAUUCACAAUGACGCCGGACGCUCUACUGCGCAACAGU |
| | | AUGAAAACAGCCUGAAGGGCGCUAAUCAGAACAGGCUGAAUAACGGCAUUGCCCAUGCAUAUGCUGACAGCAGC |
| | | CACAUUUGGUAUGCUAUCGAACCCGAGAGAAUCGCCUAUCACACAGGCGAUGGCGUUAAGAAGGGAACAGGCAA |
| | | UUACGACUACUUCGGUAUUGAAGUAUGCCAGAGCAUGAGCGCAAGUGACAAAGAGUUCAUGGCCAACGAACAG |
| | | GUGGUACUGCAGGAGGUGGCUAGGUUGCUCAAGAAAUGGAAGCUUCCCGCCAAUCGGAAUACCGUGCGGCUCC |
| | | AUCUGGAAUUCUCUGCCACAUCUUGCCCUCAUAGGAGCCUCACAAUGCGGACCGGUUUCAAUCCGGUGACUCAG |
| | | GGUAGACCCUCAGACGCCAUCAUCAAGAAGUUGAAGGAUAUCUGGAUAAAAGAGAUUCGCGCAUAUAUGGAGG |
| | | GUAAAACCCCAUCCAUUAAGACUGUCGCCAAUAAGCCCGGAUCAGCCAGUACCCCUGCAACCAAAUCCAACUCCGA |
| | | UGGGUGGAAGGUCAAUGCCUACAAUACCUACUACAAAACGGAACGGGCCUCCUUUACAGUGGGCUCUACUCGGA |
| | | UUGCUGUGCGUAGUACAGGACCCUUUACUACAUGCCCGUUUACAGGGUGGCUGAACCCCGGAGUCACGAUAACC |
| | | UAUGACGAGGUUAUGGUGCAGAACGGGUUUGUGUGGUUGGGCUAUGAUAGCUACAAUGGGAGGAGGUAUGU |
| | | CCCCAUUCGCACUGCCCAAGGAACUCCUGGCAGCUCCAAUUAUGUGCUUGGCCCUCUGUGGGGCACGAUUUCUU |
| | | GA |

Lysin L0475

| | | |
|---|---|---|
| 17 | amino acid | MQAKLTKKEFIEWLKTSEGKQFNADLWYGFQCFDYANAAWKVLFGLLLKGLGAKDIPFANNFDGLATVYQNTPDFLAQPG |
| | sequence | DMVVFGSNYGAGYGHVAWVIEATLDYIIVYEQNWLGGGWTDGIEQPGWGWEKVTRRQHAYDFPMWFIRPNFKSEIAPR |
| | | SVQSPTQAPKKETAKPQPKAVELKIIKDVVKGYDLPKRGSNPKGIVIHNDAGSKGATAEAYRNGLVNAPLSRLEAGIAHSYVS |
| | | GNTVWQALDESQVGWHTANQLGNKYYYGIEVCQSMGADNATFLKNEQATFQECARLLKKWGLPANRNTIRLHNEFTSTS |
| | | CPHRSSVLHTGFDPVTRGLLPEDKRLQLKDYFIKQIRAYMDGKIPVATVSNDSSASSNTVKPVASAWKRNKYGTYYMEESAR |
| | | FTNGNQPITVRKVGPFLYCPVGYQFQPGGYCDYTEVMLQDGHVWVGYTWEGQRYYLPIRTWNGSAPPNQILGDLWGEIS |
| 18 | nucleotide | AUGCAGGCUAAGUUGACUAAGAAAGAGUUUAUUGAGUGGUUGAAGACUUCUGAAGGUAAACAAUUCAACGCUG |
| | sequence | AUUUGUGGUAUGGUUUUCAAUGUUUCGAUUACGCUAAUGCUGCUUGGAAAGUUUUGUUUGGUUUGUUGUUG |
| | | AAGGGUUUGGGUGCUAAGGAUAUCCCUUUCGCUAACAACUUCGAUGGUUUGGCUACUGUUUACCAAAACACUC |
| | | CAGAUUUCUUGGCUCAACCUGGAGAUAUGGUUGUUUUCGGUUCUAAUUAUGGUGCUGGUUACGGUCAUGUUG |
| | | CUUGGGUUAUUGAAGCUACUUUGGAUUACAUCAUCGUUUACGAGCAAAACUGGUUGGGUGGUGGUUGGACUG |
| | | AUGGUAUUGAACAACCAGGUUGGGGUUGGGAGAAGGUUACUAGAAGACAACACGCUUAUGAUUUUCCAAUGU |
| | | GGUUCAUCAGACCUAACUUCAAGUCUGAAAUCGCUCCUAGAUCUGUUCAAUCUCCAACUCAAGCUCCUAAGAAA |
| | | GAAACUGCUAAGCCACAACCUAAAGCUGUUGAGUUGAAGAUUAUUAAGGAUGUUGUUAAGGGUUACGAUUUGC |
| | | CAAAGAGAGGUUCUAACCCUAAGGGUAUCGUUAUCCAUAACGAUGCUGGUUCUAAGGGUGCUACUGCUGAAGC |
| | | UUAUAGAAACGGUUUGGUUAAUGCUCCAUUGUCCAGAUUGGAAGCUGGUAUUGCUCAUUCUUACGUUUCUGG |
| | | UAACACUGUUUGGCAAGCUUUGGAUGAAUCUCAAGUUGGUUGGCACACUGCUAACCAAUUGGGUAAUAAGUAC |
| | | UAUUACGGUAUUGAGGUUUGUCAAUCUAUGGGUGCUGAUAACGCUACUUUCUUGAAGAACGAACAAGCUACUU |
| | | UCCAAGAGUGUGCUAGAUUGUUGAAGAAAUGGGGUUUGCCAGCUAACAGAAACACUAUCAGAUUGCACAACGA |
| | | GUUUACUUCUACUUCUUGUCCUCAUAGAUCUUCUGUUUUGCACACUGGUUUCGAUCCAGUUACUAGAGGUUU |
| | | GUUGCCUGAGGAUAAGAGAUUGCAAUUGAAGGAUUACUUCAUCAAGCAAAUCAGAGCUUACAUGGAUGGUAAA |
| | | AUUCCAGUUGCUACUGUUUCUAACGAUUCUUCUGCUUCUUCUAAUACUGUUAAACCUGUUGCUUCUGCUUGGA |
| | | AGAGAAACAAGUAUGGUACUUAUUACAUGGAAGAGUCUGCUAGAUUCACUAACGGUAAUCAACCAAUUACUGU |
| | | UAGAAAGGUUGGUCCAUUUUUGUAUUGUCCUGUUGGUUACCAAUUCCAACCUGGUGGUUAUUGUGAUUACAC |
| | | UGAAGUUAUGUUGCAAGAUGGUCAUGUUUGGGUUGGUUACACUUGGGAGGGUCAAAGAUAUUACUUGCCAAU |
| | | UAGAACUUGGAACGGUUCUGCUCCACCUAAUCAAAUUUUGGGAGAUUUGUGGGGUGAAAUUUCUUAA |
| 19 | nucleotide | AUGCAAGCGAAACUGACCAAGAAGGAGUUUAUAGAGUGGCUGAAAACCUCUGAGGGUAAACAGUUCAACGCCGA |
| | sequence | UCUGUGGUAUGGUUUCCAGUGCUUUGAUUACGCAAACGCUGCUUGGAAAGUGCUGUUUGGCCUGCUCCUGAA |
| | (opt) | GGGACUCGGUGCCAAAGACAUCCCCUUUGCCAACAAUUUCGACGGGCUGGCAACCGUCUACCAGAAUACUCCUG |
| | | ACUUUCUCGCUCAGCCAGGGGACAUGGUCGUGUUCGGCUCCAAUUAUGGGGCAGGCUAUGGACACGUAGCAUG |
| | | GGUGAUAGAAGCCACUCUGGACUACAUAAUCGUCUACGAGCAAAAUUGGCUUGGCGGUGGGUGGACCGAUGGC |
| | | AUUGAGCAGCCUGGCUGGGGAUGGGAGAAGGUUACUCGUAGACAGCAUGCUUACGACUUUCCCAUGUGGUUCA |
| | | UCCGUCCCAACUUCAAGUCCGAGAUUGCGCCCAGAAGCGUCCAGUCUCCAACCCAAGCUCCGAAGAAGGAAACAG |
| | | CCAAGCCACAGCCUAAGGCCGUUGAACUGAAGAUUAUUAAGGAUGUGGUGAAGGGAUAUGACCUCCCGAAAAGA |
| | | GGGAGCAAUCCCAAAGGGAUUGUGAUCCACAACGAUGCCGGGUCUAAAGGGGCAACAGCAGAAGCGUAUAGGAA |
| | | CGGGUUGGUGAAUGCCCCUCUCAGUCGCUUGGAGGCCGGAAUUGCACACUCAUAUGUGUCCGGAAAUACGGUU |
| | | UGGCAAGCUCUCGACGAAAGCCAAGUAGGCUGGCAUACAGCCAACCAGCUGGGCAACAAAUACUACUAUGGCAU |
| | | CGAGGUGUGCCAGAGUAUGGGCGCAGAUAAUGCCACGUUCCUGAAGAACGAGCAGGCUACCUUUCAAGAGUGU |
| | | GCGCGGUUGCUGAAGAAGUGGGGACUGCCAGCUAAUCGCAACACAAUCAGGUUGCACAACGAGUUUACGAGUAC |
| | | UAGCUGUCCACACAGAAGUAGCGUGCUGCACACAGGAUUCGAUCCUGUGACAAGGGGACUGCUGCCCGAAGAUA |
| | | AGCGGCUGCAGCUGAAAGACUACUUCAUCAAACAGAUUCGGGCCUAUAUGGACGGAAAGAUUCCCGUCGCUACU |
| | | GUUAGCAAUGACAGCUCUGCCUCAUCAAAUACCGUGAAACCGGUAGCAAGCGCCUGGAAAAGGAACAAGUAUGG |
| | | CACUUAUUACAUGGAGGAAUCCGCCCGAUUUACCAACGGUAAUCAGCCCAUCACAGUCCGCAAGGUCGGGCCAU |
| | | UCCUGUACUGUCCCGUAGGCUACCAGUUUCAGCCUGGUGGGUACUGCGACUAUACCGAGGUGAUGCUGCAAGA |
| | | UGGCCAUGUCUGGGUUGGCUAUACCUGGGAAGGGCAGCGCUACUACUUGCCAAUACGGACUUGGAAUGGUUCU |
| | | GCCCCACCCAACCAGAUCCUCGGAGAUCUGUGGGGCGAAAUCUCCUGA |

Lysin L0479

| | | |
|---|---|---|
| 20 | amino acid | MQAKLTKKEFIEWLKTSEGKQFNVDLWYGFQCFDYANAGWKVLFGLLLKGLGAKDIPFANNFDGLATVYQNTPDFLAKPG |
| | sequence | DMVVFGSNYGAGYGHVAWVIEATLDYIIVYEQNWLGGGWTDRIEQPGWGWEKVTRRQHALRFPYVVYPSYPKSETAPRS |
| | | IQSPTQASKKETAKPQPKAVELKIIKDVVKGYDLPKRGGNPKGIVIHNDAGSKGATAEAYRNGLVNAPLSRLEAGIAHSYVSG |
| | | NTVWQALDESQVGWHTANQLGNKYYYGIEVCQSMGADNATFLKNEQATFQECARLLKKWGLPANRNTIRLHNEFTSTSC |
| | | PHRSSVLHTGFDPVTRGLLPEDKRLQLKDYFIKQIRAYMDGKIPVATVSNESSASSNTVKPVASAWKRNKYGTYYMEESARF |
| | | TNGNQPITVRKVGPFLSCPVGYQFQPGGYCDYTEVMLQDGHVWVGYTWEGQRYYLPIRTWNGSAPPNQILGDLWGEIS |
| 21 | nucleotide | AUGCAGGCUAAGUUGACUAAGAAAGAGUUUAUUGAGUGGUUGAAGACUUCUGAAGGUAAACAAUUCAACGUU |
| | sequence | GAUUUGUGGUACGGUUUUCAAUGUUUCGAUUAUGCUAAUGCCGGUUGGAAAGUUUUGUUUGGUUUGUUGUU |
| | | GAAGGGUUUGGGUGCUAAGGAUAUCCCUUUCGCUAACAACUUCGAUGGUUUGGCUACUGUUUACCAAAACACU |
| | | CCAGAUUUCUUGGCUAAGCCUGGAGAUAUGGUUGUUUUCGGUUCUAAUUACGGUGCUGGUUAUGGUCAUGUU |
| | | GCUUGGGUUAUUGAAGCUACUUUGGAUUACAUCAUCGUUUACGAGCAAAAUUGGUUGGGUGGUGGUUGGACU |
| | | GAUAGAAUUGAACAACCAGGUUGGGGUUGGGAGAAGGUUACUAGAAGACAACACGCUUUGAGAUUCCCUUAUG |
| | | UUGUUUACCCAUCUUAUCCUAAAUCUGAAACUGCUCCAAGAUCUAUUCAAUCUCCUACUCAAGCUUCUAAGAAA |
| | | GAAACUGCUAAGCCACAACCUAAAGCUGUUGAGUUGAAGAUUAUUAAGGAUGUUGUUAAGGGUUACGAUUUGC |
| | | CAAAGAGAGGUGGUAACCCUAAGGGUAUCGUUAUCCAUAACGAUGCUGGUUCUAAAGGUGCUACUGCUGAAGC |
| | | UAACACUGUUUGGCAAGCUUUGGAUGAAUCUCAAGUUGGUUGGCACACUGCUAACCAAUUGGGUAAUAAGUAC |
| | | UUACAGAAACGGUUUGGUUAAUGCUCCAUUGUCCAGAUUGGAAGCUGGUAUUGCUCAUUCUUAUGUUUCUGG |
| | | UAUUACGGUAUUGAGGUUUGUCAAUCUAUGGGUGCUGAUAACGCUACUUUCUUGAAGAACGAACAAGCUACUU |
| | | UCCAAGAGUGUGCUAGAUUGUUGAAGAAAUGGGGUUUGCCAGCUAACAGAAACACUAUCAGAUUGCACAACGA |
| | | GUUUACUUCUACUUCUUGUCCUCAUAGAUCUUCUGUUUUGCACACUGGUUUCGAUCCAGUUACUAGAGGUUU |
| | | GUUGCCUGAGGAUAAGAGAUUGCAAUUGAAGGAUUACUUCAUCAAGCAAAUCAGAGCUUACAUGGAUGGUAAA |
| | | AUCCCAGUUGCUACUGUUUCUAACGAAUCUUCUGCUUCUUCUAAUACUGUUAAACCUGUUGCUUCUGCUUGGA |
| | | AGAGAAACAAAUACGGUACUUACUACAUGGAAGAGUCUGCUAGAUUCACUAACGGUAAUCAACCAAUUACUGUU |
| | | AGAAAGGUUGGUCCAUUUUUGUCUUGUCCUGUUGGUUACCAAUUCCAACCUGGUGGUUACUGUGAUUAUACU |
| | | GAAGUUAUGUUGCAAGAUGGUCAUGUUUGGGUUGGUUAUACUUGGGAGGGUCAAAGAUAUUACUUGCCAAUU |
| | | AGAACUUGGAACGGUUCUGCUCCACCUAAUCAAAUUUUGGGAGAUUUGUGGGGUGAAAUUUCUUAA |
| 22 | nucleotide | AUGCAGGCCAAACUGACCAAGAAGGAGUUCAUCGAAUGGCUCAAGACUUCCGAGGGCAAGCAGUUCAAUGUGGA |
| | sequence | CCUCUGGUAUGGCUUUCAAUGCUUUGAUUAUGCCAAUGCUGGGUGGAAGGUCCUGUUUGGCCUUCUCCUGAA |
| | (opt) | AGGACUGGGUGCAAAAGACAUCCCCUUCGCCAACAACUUCGAUGGACUGGCUACCGUUUACCAGAAUACCCCGG |
| | | ACUUUCUGGCCAAGCCUGGAGAUAUGGUGGUGUUCGGAAGCAACUAUGGGGCAGGAUAUGGCCAUGUAGCCUG |
| | | GGUGAUAGAAGCUACGUUGGACUACAUCAUCGUGUACGAGCAAAAUUGGCUGGGCGGCGGCUGGACUGACCGA |
| | | AUUGAGCAACCUGGGUGGGGAUGGGAGAAAGUGACCAGACGUCAGCACGCUCUGCGCUUCCCCUAUGUAGUGU |
| | | ACCCCUCUUACCCGAAGUCUGAGACAGCUCCCCGGUCCAUUCAGAGCCCAACACAGGCGAGCAAGAAAGAGACUG |
| | | CCAAACCACAACCCAAAGCGGUUGAACUGAAGAUCAUCAAAGACGUCGUGAAAGGGUAUGAUCUGCCCAAGAGA |
| | | GGCGGAAAUCCAAAGGGGAUUGUGAUCCACAAUGACGCCGGGAGCAAAGGUGCAACUGCUGAAGCUUACCGCAA |
| | | UGGGCUGGUCAACGCACCUUUGUCCCGACUGGAAGCCGGCAUUGCUCAUAGCUAUGUGUCUGGCAACACUGUU |
| | | UGGCAAGCACUGGACGAAUCACAGGUCGGCUGGCAUACUGCCAAUCAGCUCGGAAACAAGUACUACUACGGGAU |
| | | AGAGGUCUGUCAGUCCAUGGGCGCCGAUAACGCGACUUUCCUGAAGAAUGAGCAGGCCACAUUCCAGGAAUGCG |
| | | CUCGGUUGUUGAAGAAAUGGGGCCUUCCUGCAAACAGGAACACAAUCAGGCUGCACAAUGAGUUCACAUCCACG |
| | | UCUUGUCCACACAGGUCUAGCGUGCUGCACACAGGUUUUGAUCCCGUGACGAGAGGGCUUCUCCCCGAAGAUAA |
| | | ACGUCUGCAGCUCAAGGAUUACUUCAUCAAGCAGAUAAGGGCCUAUAUGGACGGGAAAAUUCCGGUAGCUACCG |
| | | UCAGCAACGAGAGUAGCGCAUCAUCCAACACCGUUAAGCCAGUGGCCUCAGCCUGGAAGCGCAACAAAUACGGUA |
| | | CCUAUUACAUGGAGGAAUCUGCAAGAUUCACAAAUGGUAAUCAGCCUAUUACCGUGCGGAAAGUCGGCCCUUU |
| | | UCUCAGCUGUCCUGUUGGAUAUCAGUUUCAGCCAGGCGGUUACUGCGACUAUACCGAAGUAAUGCUGCAAGAU |
| | | GGGCAUGUGUGGGUUGGGUACACAUGGGAAGGCCAACGGUACUAUCUGCCCAUUCGCACCUGGAACGGCAGUG |
| | | CCCCACCCAACCAGAUCCUUGGAGAUCUGUGGGGUGAGAUUUCAUGA |

Lysin L0480

| | | |
|---|---|---|
| 23 | amino acid | MAKTQAEINKRLDAYAKGTVDSPYRVKKATSYDPSFGVMEAGAIDADGYYHAQCQDLITDYVLWLTDNKVRTWGNAKDQ |
| | sequence | IKQSYGTGFKIHENKPSTVPKKGWIAVFTSGSYEQWGHIGIVYDGGNTSTFTILEQNWNGYANKKPTKRVDNYYGLTHFIEIP |
| | | VKAGTTVKKETAKKSASKTPAPKKKATLKVSKNHINYTMDKRGKKPEGMVIHNDAGRSSGQQYENSLANAGYARYANGIA |
| | | HYYGSEGYVWEAIDAKNQIAWHTGDGTGANSGNFRFAGIEVCQSMSASDAQFLKNEQAVFQFTAEKFKEWGLTPNRKTV |
| | | RLHMEFVPTACPHRSMVLHTGFNPVTQGRPSQAIMNKLKDYFIKQIKNYMDKGTSSSTVVKDGKTSSASTPATRPVTGSW |
| | | KKNQYGTWYKPENATFVNGNQPIVTRIGSPFLNAPVGGNLPAGATIVYDEVCIQAGHIWIGYNAYNGNRVYCPVRTCQGV |
| | | PPNQIPGVAWGVFK |
| | nucleotide | AUGGCUAAGACUCAAGCUGAGAUUAAUAAGAGAUUGGAUGCUUACGCUAAGGGUACUGUUGAUUCUCCAUACA |
| | sequence | GAGUUAAGAAAGCUACUUCUUAUGAUCCUUCUUUCGGUGUUAUGGAAGCUGGUGCUAUUGAUGCUGAUGGUU |
| | | ACUAUCAUGCUCAAUGUCAAGAUUUGAUCACUGAUUACGUUUUGUGGUUGACUGAUAACAAGGUUAGAACUU |
| | | GGGGUAACGCUAAGGAUCAAAUUAAGCAAUCUUACGGUACUGGUUUUAAAAUCCAUGAAAACAAGCCAUCUACU |
| | | GUUCCUAAGAAAGGUUGGAUUGCUGUUUUCACUUCUGGUUCUUACGAGCAAUGGGGUCACAUUGGUAUUGUU |
| | | UACGAUGGUGGUAACACUUCUACUUUUACUAUUUUGGAACAAAACUGGAAUGGUUACGCUAACAAGAAACCAA |
| | | CUAAGAGAGUUGAUAACUACUACGGUUUGACUCACUUCAUUGAAAUUCCUGUUAAGGCUGGUACUACUGUUAA |
| | | GAAAGAGACUGCUAAGAAAUCUGCUUCUAAAACUCCAGCUCCUAAGAAAAAGGCUACUUUGAAAGUUUCUAAGA |
| | | ACCAUAUCAACUACACUAUGGAUAAGAGAGGUAAAAAGCCAGAGGGUAUGGUUAUUCACAACGAUGCUGGUAG |
| | | AUCUUCUGGUCAACAAUACGAGAACUCUUUGGCUAAUGCUGGUUACGCUAGAUAUGCUAACGGUAUUGCUCAU |
| 24 | | UACUAUGGUUCUGAAGGUUAUGUUUGGGAGGCUAUUGAUGCUAAAAAUCAAAUUGCUUGGCACACUGGAGAU |
| | | GGUACUGGUGCUAACUCUGGUAAUUUUAGAUUCGCUGGUAUUGAAGUUUGUCAAUCUAUGUCUGCUUCUGAU |
| | | GCUCAAUUUUUGAAGAACGAGCAAGCUGUUUUCCAAUUCACUGCUGAAAAAUUCAAGGAGUGGGGUUUGACUC |
| | | CUAACAGAAAGACUGUUAGAUUGCAUAUGGAAUUUGUUCCAACUGCUUGUCCUCAUAGAUCUAUGGUUUUGCA |
| | | CACUGGUUUCAAUCCAGUUACUCAAGGUAGACCUUCUCAAGCUAUCAUGAACAAAUUGAAGGAUUACUUCAUCA |
| | | AGCAAAUUAAGAAUUACAUGGAUAAGGGUACUUCUUCUUCUACUGUUGUUAAAGAUGGUAAAACUUCUUCUGC |
| | | UUCUACUCCAGCUACUAGACCUGUUACUGGUUCUUGGAAAAAGAACCAAUACGGUACUUGGUAUAAACCAGAGA |
| | | AUGCUACUUUUGUUAACGGUAAUCAACCUAUUGUUACUAGAAUUGGUUCUCCAUUCUUGAACGCUCCUGUUGG |
| | | UGGUAAUUUGCCAGCUGGUGCUACUAUCGUUUACGAUGAAGUUUGUAUCCAAGCUGGUCAUAUUUGGAUUGG |
| | | UUACAACGCUUAUAACGGUAAUAGAGUUUAUUGUCCAGUUAGAACUUGUCAAGGUGUUCCACCUAAUCAAAUU |
| | | CCUGGUGUUGCUUGGGGUGUUUUCAAGUAA |
| 25 | nucleotide | AUGGCCAAGACACAGGCCGAGAUCAACAAGCGGCUGGACGCCUAUGCCAAGGGCACAGUGGAUAGCCCCUACAGA |
| | sequence | GUGAAGAAGGCCACCAGCUACGACCCCAGCUUCGGCGUUAUGGAAGCCGGCGCUAUUGACGCCGACGGCUAUUA |
| | (opt) | UCAUGCCCAGUGCCAGGACCUGAUCACCGACUAUGUGCUGUGGCUGACCGACAACAAAGUGCGGACAUGGGGCA |
| | | ACGCCAAGGACCAGAUCAAGCAGAGCUACGGCACCGGCUUCAAGAUCCACGAGAACAAGCCUAGCACCGUGCCUA |
| | | AGAAAGGCUGGAUCGCCGUGUUUACCAGCGGCAGCUAUGAGCAGUGGGGCCACAUCGGCAUCGUGUACGAUGG |
| | | CGGCAAUACCAGCACCUUCACCAUCCUGGAACAGAACUGGAACGGCUACGCCAACAAGAAACCCACCAAGAGAGU |
| | | GGACAACUACUACGGCCUGACACACUUCAUCGAGAUCCCCGUGAAGGCCGGCACCACCGUGAAGAAAGAGACAGC |
| | | CAAGAAGUCCGCCAGCAAGACCCCUGCUCCUAAGAAGAAAGCCACACUGAAGGUGUCCAAGAACCACAUCAACUA |
| | | CACCAUGGACAAGCGGGGCAAAAAGCCCGAAGGCAUGGUCAUCCACAACGACGCCGGCAGAUCUAGCGGCCAGCA |
| | | GUACGAAAACAGCCUGGCCAAUGCCGGCUAUGCCAGAUACGCCAAUGGAAUCGCCCACUACUAUGGCAGCGAGG |
| | | GCUAUGUGUGGGAAGCCAUCGACGCCAAGAAUCAGAUCGCCUGGCAUACAGGCGACGGAACCGGCGCCAAUUCC |
| | | GGCAAUUUCAGAUUCGCCGGCAUCGAAGUGUGCCAGAGCAUGUCUGCCAGCGACGCCCAGUUCCUGAAGAAUGA |
| | | GCAGGCCGUGUUCCAGUUUACCGCCGAGAAGUUCAAAGAGUGGGGACUGACCCCUAACAGAAAGACCGUGCGGC |
| | | UGCACAUGGAAUUCGUGCCUACCGCAUGUCCCCACCGCUCUAUGGUGCUGCACACCGGCUUUAACCCCGUGACAC |
| | | AGGGCAGACCUAGCCAGGCCAUCAUGAACAAGCUGAAGGACUACUUCAUUAAGCAGAUCAAGAACUACAUGGAU |
| | | AAGGGCACCAGCAGCAGCACCGUGGUCAAGGAUGGCAAGACCAGCAGCGCCUCUACACCAGCCACAAGACCUGUG |
| | | ACAGGCAGCUGGAAGAAGAACCAGUACGGCACCUGGUACAAGCCCGAGAACGCCACCUUCGUGAACGGCAACCAG |
| | | CCUAUCGUGACCAGAAUCGGCAGCCCCUUCCUGAAUGCCCCUGUCGGAGGAAAUCUUCCUGCUGGCGCCACCAUC |
| | | GUGUAUGACGAAGUCUGUAUUCAGGCCGGCCACAUCUGGAUCGGCUACAACGCCUACAACGGCAACAGAGUGUA |
| | | CUGCCCCGUGCGGACUUGUCAGGGCGUUCCACCUAAUCAGAUUCCAGGCGUGGCCUGGGGCGUGUUCAAAUGA |

Lysin L0482

| | | |
|---|---|---|
| 26 | amino acid | REAPKTQIYTVKKGDTLSAIALKYKTTVSNIQNTNNIANPNLIFIGQKLKVPMTPLVEPKPKTVSSNNKSNSNSSTLNYLKTLEN |
| | sequence | RGWDFDGSYGWQCFDLVNVYWNHLYGHGLKGYGAKDIPYANNFNSEAKIYHNTPTFKAEPGDLVVFSGRFGGGYGHTAI |
| | | VLNGDYDGKLMKFQSLDQNWNNGGWRKAEVAHKVVHNYENDMIFIRPFKKA |
| 27 | nucleotide | AGAGAGGCUCCUAAGACUCAAAUCUACACUGUUAAGAAAGGAGAUACUUUGUCUGCUAUCGCUUUGAAGUACA |
| | sequence | AGACUACUGUUUCUAACAUCCAAAACACUAACAACAUCGCUAACCCAAAUUUGAUUUUCAUUGGUCAAAAGUUG |
| | | AAAGUUCCAAUGACUCCUUUGGUUGAACCAAAGCCUAAGACUGUUUCUUCUAACAAUAAGUCUAACUCUAAUUC |
| | | UUCUACUUUGAACUACUUGAAGACUUUGGAGAAUAGAGGUUGGGAUUUUGAUGGUUCUUAUGGUUGGCAAU |
| | | GUUUCGAUUUGGUUAACGUUUACUGGAAUCAUUUGUAUGGUCACGGUUUGAAGGGUUACGGUGCUAAGGAUA |
| | | UCCCUUACGCUAACAACUUCAACUCUGAAGCUAAGAUCUACCAUAAUACUCCAACUUUCAAAGCUGAGCCUGGAG |
| | | AUUUGGUUGUUUUCUCUGGUAGAUUCGGUGGUGGUUACGGUCACACUGCUAUUGUUUUGAACGGAGAUUAC |
| | | GAUGGUAAAUUGAUGAAGUUCCAAUCUUUGGAUCAAAAUUGGAACAAUGGUGGUUGGAGAAAGGCUGAAGUU |
| | | GCUCAUAAGGUUGUUCACAACUACGAGAACGAUAUGAUUUUCAUUAGACCAUUCAAGAAAGCUUAA |
| 28 | nucleotide | AGAGAGGCUCCCAAAACACAGAUUUACACCGUCAAGAAGGGAGAUACGCUUAGCGCCAUUGCACUGAAAUACAA |
| | sequence | GACCACCGUAUCAAACAUCCAGAACACCAAUAACAUAGCCAAUCCCAAUCUCAUCUUCAUUGGCCAGAAGCUCAA |
| | (opt) | GGUUCCCAUGACUCCACUGGUCGAACCGAAACCUAAGACAGUGUCCUCCAAUAACAAGAGUAACAGCAACUCCUC |
| | | UACACUGAACUAUCUCAAGACGUUGGAGAAUCGCGGUUGGGACUUUGACGGCAGUUACGGCUGGCAAUGCUUC |
| | | GACUUGGUGAAUGUGUACUGGAAUCACCUGUAUGGGCAUGGACUGAAAGGCUAUGGGGCCAAAGACAUACCUU |
| | | AUGCUAACAAUUUCAACUCUGAGGCCAAGAUCUACCACAACACUCCUACCUUCAAGGCAGAACCAGGAGAUCUGG |
| | | UUGUGUUUAGCGGACGGUUUGGCGGAGGCUAUGGGCAUACUGCCAUUGUGCUUAAUGGGGAUUACGAUGGCA |
| | | AACUGAUGAAAUUCCAGUCACUGGACCAGAACUGGAACAAUGGUGGUUGGCGAAAAGCGGAAGUAGCCCACAAG |
| | | GUCGUCCACAAUUACGAGAACGACAUGAUCUUCAUCCGUCCCUUUAAGAAAGCGUGA |

Lysin L0483

| | | |
|---|---|---|
| 29 | amino acid | AATHEHSAQWLNNYKKGYGYGPYPLGINGGMHYGVDFFMNIGTPVKAISSGKIVEAGWSNYGGGNQIGLIENDGVHRQ |
| | sequence | WYMHLSKYNVKVGDYVKAGQIIGWSGSTGYSTAPHLHFQRMVNSFSNSTAQDPMPFLKSAGYGKAGGTVTPTPNTGWK |
| | | TNKYGTLYKSESASFTPNTDIITRTTGPFRSMPQSGVLKAGQTIHYDEVMKQDGHVWVGYTGNSGQRIYLPVRTWNKSTNT |
| | | LGVLWGTIK |
| 30 | nucleotide | GCUGCUACUCAUGAGCACUCUGCUCAAUGGUUGAACAAUUACAAGAAAGGUUACGGUUAUGGUCCAUAUCCUU |
| | sequence | UGGGUAUUAACGGUGGUAUGCAUUACGGUGUUGAUUUCUUUAUGAACAUCGGUACUCCUGUUAAGGCUAUUU |
| | | CUUCUGGUAAAAUUGUUGAAGCUGGUUGGUCUAACUAUGGUGGUGGUAAUCAAAUUGGUUUGAUUGAGAACG |
| | | AUGGUGUUCAUAGACAAUGGUACAUGCACUUGUCUAAGUACAACGUUAAGGUUGGAGAUUACGUUAAGGCUG |
| | | GUCAAAUUAUUGGUUGGUCUGGUUCUACUGGUUAUUCUACUGCUCCACAUUUGCACUUCCAAAGAAUGGUUAA |
| | | CUCUUUCUCUAAUUCUACUGCUCAAGAUCCAAUGCCUUUCUUGAAGUCUGCUGGUUACGGUAAAGCUGGUGGU |
| | | ACUGUUACUCCAACUCCUAACACUGGUUGGAAGACUAACAAGUACGGUACUUUGUACAAGUCUGAAUCUGCUUC |
| | | UUUUACUCCAAACACUGAUAUCAUCACUAGAACUACUGGUCCAUUCAGAUCUAUGCCUCAAUCUGGUGUUUUG |
| | | AAGGCUGGUCAAACUAUCCAUUACGAUGAGGUUAUGAAGCAAGAUGGUCACGUUUGGGUUGGUUACACUGGU |
| | | AAUUCUGGUCAAAGAAUCUACUUGCCUGUUAGAACUUGGAACAAGUCUACUAAUACUUUGGGUGUUUUGUGG |
| | | GGUACUAUUAAAUAA |
| 31 | nucleotide | GCAGCAACGCAUGAGCAUUCCGCUCAAUGGCUCAACAACUACAAGAAGGGUUACGGCUAUGGCCCCUAUCCUCU |
| | sequence | GGGGAUAAACGGAGGGAUGCACUAUGGAGUCGAUUUCUUCAUGAAUAUUGGCACUCCAGUGAAAGCGAUUUCC |
| | (opt) | AGCGGGAAGAUUGUGGAAGCUGGGUGGAGCAACUAUGGUGGUGGCAAUCAGAUCGGACUGAUUGAGAAUGAC |
| | | GGCGUGCACAGACAGUGGUAUAUGCAUCUGAGUAAGUACAACGUCAAGGUGGGAGACUACGUCAAAGCCGGGC |
| | | AGAUCAUAGGAUGGUCCGGCUCUACAGGUUACUCAACAGCGCCACAUCUCCACUUUCAGCGCAUGGUGAAUAGC |
| | | UUCAGCAAUUCAACCGCCCAAGAUCCGAUGCCCUUCCUGAAGUCUGCCGGCUAUGGGAAAGCUGGAGGAACAGU |
| | | GACACCUACUCCCAAUACCGGAUGGAAAACGAACAAGUACGGCACUCUGUACAAAUCCGAAUCUGCCAGUUUCAC |
| | | ACCCAAUACCGACAUCAUCACCCGAACCACUGGACCUUUUCGGUCAAUGCCGCAGUCUGGAGUACUGAAAGCAGG |
| | | GCAAACUAUCCACUAUGACGAGGUGAUGAAGCAGGAUGGCCACGUAUGGGUGGGGUACACAGGCAAUAGCGGC |
| | | CAGAGGAUUUACUUGCCAGUCCGUACCUGGAACAAAAGCACGAACACUCUUGGCGUUUUGUGGGGCACCAUCAA |
| | | GUGA |

Lysin L0484

| | | |
|---|---|---|
| 32 | amino acid | DQFLDEKEGNHTTAYRDGSGIWTICRGATMVDGKPVIPGMRLSKEKCDQVNAIERDKALAWVERNIKVPLTEPQKAGIASF |
| | sequence | CPYNIGPGKCFPSTFYKRLNAGDRKGACEAIRWWIKDGGRDCRIRSNNCYGQVIRRDQESALACWGIDQ |
| 33 | nucleotide | GAUCAAUUCUUGGAUGAAAAAGAGGGUAAUCAUACUACUGCUUAUAGAGAUGGUUCUGGUAUUUGGACUAUU |
| | sequence | UGUAGAGGUGCUACUAUGGUUGAUGGUAAACCAGUUAUUCCUGGUAUGAGAUUGUCUAAGGAAAAGUGUGAU |
| | | CAAGUUAACGCUAUUGAGAGAGAUAAGGCUUUGGCUUGGGUUGAAAGAAAUAUUAAGGUUCCAUUGACUGAG |
| | | CCUCAAAAAGCUGGUAUUGCUUCUUUUUGUCCAUACAACAUUGGUCCUGGUAAAUGUUUCCCAUCUACUUUCU |
| | | ACAAGAGAUUGAAUGCUGGAGAUAGAAAGGGUGCUUGUGAAGCUAUUAGAUGGUGGAUUAAGGAUGGUGGUA |
| | | GAGAUUGUAGAAUUAGAUCUAACAACUGUUACGGUCAAGUUAUUAGAAGAGAUCAAGAAUCUGCUUUGGCUU |
| | | GUUGGGGUAUUGAUCAAUAA |
| 34 | nucleotide | GAUCAGUUUCUCGACGAGAAGGAAGGCAAUCACACCACUGCGUAUAGGGAUGGCUCUGGGAUAUGGACCAUCU |
| | sequence | GUAGAGGCGCCACAAUGGUCGAUGGCAAACCCGUGAUACCCGGAAUGCGUCUGUCAAAGGAGAAGUGCGAUCAG |
| | (opt) | GUGAAUGCCAUCGAACGCGACAAAGCUCUGGCCUGGGUUGAGCGGAACAUCAAGGUUCCACUGACGGAACCACA |
| | | GAAAGCCGGAAUUGCGUCCUUCUGUCCGUACAACAUUGGCCCUGGAAAGUGCUUUCCCUCCACUUUCUACAAGC |
| | | GCCUGAAUGCAGGGGACAGAAAAGGAGCCUGUGAGGCUAUCAGGUGGUGGAUCAAAGAUGGAGGUAGGGACUG |
| | | UCGAAUCCGGAGCAACAACUGCUAUGGGCAAGUGAUUCGGAGAGAUCAGGAGAGUGCUCUGGCUUGCUGGGGU |
| | | AUUGACCAGUGA |

Lysin L0487

| | | |
|---|---|---|
| 35 | amino acid | MRYIPAAHHSAGSNKPVNRVVIHATCPDVGFPSASRKGRAVSTANYFASPSSGGSAHYVCDIGETVQCLSESTIGWHAPPN |
| | sequence | PHSLGIEICADGGSHASFRVPGHAYTREQWLDPRVWPAVERAAVLCRQLCDKHGVPKRKLSVADLKAGRRGICGHTDVTD |
| | | AWHQSDHDDPGPWFPWDKFMAVVNGHGGGSSSEELTVADVKALHNQIKQLSAQLSGSVNKLHHDVGVVQVQNGDLS |
| | | KRVDALSWVKNPVTGKLWRTKDALWSVWYYVLECRSRIDRLESAVNGLKK |
| 36 | nucleotide | AUGAGAUAUAUUCCUGCUGCUCAUCACUCUGCUGGUUCUAACAAGCCAGUUAAUAGAGUUGUUAUUCAUGCUA |
| | sequence | CUUGUCCAGAUGUUGGUUUUCCUUCUGCUUCCAGAAAAGGUAGAGCUGUUUCUACUGCUAACUACUUCGCUUC |
| | | UCCUUCUUCUGGUGGUUCUGCUCAUUAUGUUUGUGAUAUUGGUGAAACUGUUCAAUGUUUGUCUGAGUCUAC |
| | | UAUUGGUUGGCAUGCUCCACCUAAUCCACACUCUUUGGGUAUUGAAAUUUGUGCUGAUGGUGGUUCUCAUGC |
| | | UUCUUUCAGAGUUCCUGGUCACGCUUACACUAGAGAACAAUGGUUGGAUCCAAGAGUUUGGCCUGCUGUUGAG |
| | | AGAGCUGCUGUUUUGUGUAGACAAUUGUGUGAUAAGCAUGGUGUUCCAAAGAGAAAAUUGUCUGUUGCUGAU |
| | | UUGAAAGCUGGUAGAAGAGGUAUUUGUGGUCACACUGAUGUUACUGAUGCUUGGCAUCAAUCUGAUCACGAU |
| | | GAUCCAGGUCCUUGGUUUCCUUGGGAUAAGUUCAUGGCUGUUGUUAACGGUCAUGGUGGUGGUUCUUCUUCU |
| | | GAAGAGUUGACUGUUGCUGAUGUUAAGGCUUUGCACAACCAAAUUAAACAAUUGUCUGCUCAAUUGUCUGGUU |
| | | CCGUUAACAAAUUGCAUCACGAUGUUGGUGUUGUUCAAGUUCAAAAUGGAGAUUUGUCUAAGAGAGUUGAUG |
| | | CUUUGUCUUGGGUUAAAAACCCAGUUACUGGUAAAUUGUGGAGAACUAAAGAUGCUUUGUGGUCUGUUUGGU |
| | | ACUAUGUUUUGGAAUGUAGAUCCAGAAUUGAUAGAUUGGAGUCUGCUGUUAAUGGUUUGAAGAAAUAA |
| 37 | nucleotide | AUGCGCUAUAUUCCUGCUGCACACCAUUCUGCAGGGAGCAACAAGCCCGUGAAUCGCGUUGUCAUCCAUGCCAC |
| | sequence | UUGUCCCGAUGUGGGCUUUCCCUCCGCCUCACGGAAAGGAAGGGCAGUGAGCACUGCCAACUACUUCGCCAGUC |
| | (opt) | CGUCCUCAGGCGGGUCAGCCCACUAUGUGUGCGACAUAGGGGAGACAGUACAGUGUCUCAGCGAAAGCACGAUU |
| | | GGGUGGCAUGCCCCACCGAAUCCACACUCUUUGGGGAUCGAGAUUUGCGCAGAUGGAGGUUCUCAUGCCUCUU |
| | | UUCGCGUGCCAGGCCAUGCAUACACCAGAGAGCAGUGGCUCGAUCCACGAGUUUGGCCCGCAGUUGAACGAGCU |
| | | GCUGUGCUGUGCCGGCAAUUGUGCGACAAGCAUGGCGUACCCAAACGGAAACUGAGCGUUGCGGACCUGAAAGC |
| | | CGGGAGGAGGGGUAUAUGCGGACAUACCGACGUGACUGACGCGUGGCACCAGAGUGACCACGAUGACCCUGGGC |
| | | CUUGGUUUCCUUGGGACAAGUUCAUGGCGGUGGUAAACGGACAUGGUGGCGGCAGUAGUAGCGAGGAACUGA |
| | | CCGUUGCCGAUGUCAAGGCCCUGCACAACCAGAUCAAGCAGCUGUCUGCUCAACUCUCCGGAAGCGUGAACAAGC |
| | | UGCACCACGAUGUGGGAGUCGUCCAAGUGCAGAAUGGCGACCUCUCCAAGAGAGUGGAUGCCUUGAGCUGGGU |
| | | CAAGAAUCCCGUUACAGGCAAACUUUGGCGUACCAAAGAUGCCCUGUGGAGCGUCUGGUACUAUGUACUGGAG |
| | | UGUCGGUCCAGGAUCGAUAGACUGGAGUCAGCUGUCAAUGGUCUGAAGAAGUGA |

Lysin L0489

| | | |
|---|---|---|
| 38 | amino acid | MLMTKNQAEKWFDNSLGKQFNPDGWYGFQCYDYANMFFMLATGERLQGLYAYNIPFDNKAKIEKYGQIIKNYDSFLPQK |
| | sequence | LDIVVFPSKYGGGAGHVEIVESANLNTFTSFGQNWNGKGWTNGVAQPGWGPETVTRHVHYYDNPMYFIRLNFPNNLSV |
| | | GNKAKGIIKQATTKKEAVIKPKKIMLVAGHGYNDPGAVGNGTNERDFIRKYITPNIAKYLRHAGHEVALYGGSSQSQDMYQ |
| | | DTAYGVNVGNKKDYGLYWVKSQGYDIVLEIHLDAAGESASGGHVIISSQFNADTIDKSIQDVIKNNLGQIRGVTPRNDLLNV |
| | | NVSAEININYRLSELGFITNKNDMDWIKKNYDLYSKLIAGAIHGKPIGGLVAGNVKTSAKNKKNPPVPAGYTLDKNNVPYKKE |
| | | QGNYTVANVKGNNVRDGYSTNSRITGVLPNNTTITYDGAYCINGYRWITYIANSGQRRYIATGEVDKAGNRISSFGKFSTI |
| 39 | nucleotide | AUGUUGAUGACUAAGAACCAAGCUGAAAAAUGGUUCGAUAACUCUUUGGGUAAACAAUUCAAUCCUGAUGGUU |
| | sequence | GGUACGGUUUCCAAUGUUACGAUUACGCUAACAUGUUUUUCAUGUUGGCUACUGGUGAAAGAUUGCAAGGUU |
| | | UGUACGCUUACAACAUCCCAUUCGAUAAUAAGGCUAAGAUCGAGAAGUACGGUCAAAUUAUUAAGAACUACGAU |
| | | UCUUUCUUGCCACAAAAGUUGGAUAUCGUUGUUUUCCCUUCUAAAUACGGUGGUGGUGCUGGUCAUGUUGAA |
| | | AUUGUUGAGUCUGCUAACUUGAAUACUUUUACUUCUUUCGGUCAAAACUGGAAUGGUAAAGGUUGGACUAAC |
| | | GGUGUUGCUCAACCAGGUUGGGGUCCUGAAACUGUUACUAGACAUGUUCACUACUACGAUAACCCAAUGUACU |
| | | UCAUCAGAUUGAAUUUCCCUAACAAUUUGUCUGUUGGUAAUAAGGCUAAGGGUAUUAUUAAGCAAGCUACUAC |
| | | UAAGAAAGAGGCUGUUAUUAAACCAAAGAAAAUUAUGUUGGUUGCUGGUCACGGUUAUAACGAUCCUGGUGCU |
| | | GUUGGUAACGGUACUAACGAAAGAGAUUUCAUUAGAAAGUACAUCACUCCUAACAUCGCUAAGUAUUUGAGAC |
| | | AUGCUGGUCACGAGGUUGCUUUGUACGGUGGUUCUUCUCAAUCUCAAGAUAUGUACCAAGAUACUGCUUAUG |
| | | GUGUUAACGUUGGUAAUAAGAAAGAUUACGGUUUGUACUGGGUUAAGUCUCAAGGUUACGAUAUCGUUUUGG |
| | | AAAUCCAUUUGGAUGCUGCUGGUGAAUCUGCUUCUGGUGGUCACGUUAUCAUCUCUUCUCAAUUCAACGCUGA |
| | | UACUAUCGAUAAGUCUAUCCAAGAUGUUAUUAAAAACAAUUUGGGUCAAAUUAGAGGUGUUACUCCAAGAAAC |
| | | GAUUUGUUGAACGUUAACGUUUCUGCUGAAAUUAACAUCAACUACAGAUUGUCUGAGUUGGGUUUUAUUACU |
| | | AACAAAAACGAUAUGGAUUGGAUUAAGAAAAACUACGAUUUGUAUUCUAAGUUGAUUGCUGGUGCUAUUCAU |
| | | GGUAAACCUAUUGGUGGUUUGGUUGCUGGUAACGUUAAGACUUCUGCUAAAAACAAGAAAAAUCCACCUGUUC |
| | | CAGCUGGUUACACUUUGGAUAAGAACAAUGUUCCUUACAAGAAAGAACAGGGUAACUACACUGUUGCUAACGU |
| | | UAAGGGUAACAACGUUAGAGAUGGUUACUCUACUAACUCCAGAAUUACUGGUGUUUUGCCAAACAACACUACU |
| | | AUCACUUACGAUGGUGCUUACUGUAUCAACGGUUACAGAUGGAUCACUUACAUCGCUAAUUCUGGUCAAAGAA |
| | | GAUAUAUUGCUACUGGUGAAGUUGAUAAGGCUGGUAAUAGAAUUUCUUCUUUUGGUAAAUUCUCUACUAUCU |
| | | AA |
| 40 | nucleotide | AUGCUGAUGACCAAGAACCAGGCCGAGAAGUGGUUCGACAACAGCCUGGGCAAGCAGUUCAACCCCGAUGGAUG |
| | sequence | GUACGGCUUCCAGUGCUACGACUACGCCAACAUGUUCUUCAUGCUGGCCACCGGCGAGAGACUGCAGGGCCUGU |
| | (opt) | ACGCCUACAACAUCCCCUUCGAUAACAAGGCCAAGAUCGAGAAGUACGGCCAGAUCAUCAAGAACUACGACAGCU |
| | | UCCUGCCUCAGAAACUGGACAUCGUGGUGUUCCCCAGCAAAUAUGGCGGCGGAGCCGGACACGUGGAAAUCGUG |
| | | GAAAGCGCCAACCUGAACACCUUCACCAGCUUCGGCCAGAACUGGAACGGCAAAGGCUGGACCAAUGGCGUGGCA |
| | | CAACCUGGCUGGGGACCUGAGACAGUGACAAGACACGUGCACUACUACGACAACCCCAUGUACUUCAUCCGGCUG |
| | | AACUUCCCCAACAACCUGAGCGUGGGCAACAAAGCCAAGGGCAUCAUCAAGCAGGCCACCACCAAGAAAGAAGCCG |
| | | UGAUCAAGCCCAAGAAAAUCAUGCUGGUGGCUGGCCACGGCUACAACGAUCCUGGCGCUGUUGGCAACGGCACC |
| | | AACGAGAGAGACUUCAUCAGAAAGUACAUCACGCCCAAUAUCGCCAAGUACCUGAGGCACGCCGGACAUGAGGU |
| | | UGCCCUGUAUGGCGGAUCUAGCCAGAGCCAGGACAUGUACCAGGAUACCGCCUACGGCGUGAACGUGGGAAACA |
| | | AGAAGGACUACGGCCUGUACUGGGUCAAGAGCCAGGGCUACGACAUCGUGCUGGAAAUCCACCUGGAUGCCGCU |
| | | GGCGAAUCUGCCUCUGGCGGACAUGUGAUCAUCAGCAGCCAGUUCAAUGCCGACACCAUCGACAAGAGCAUCCA |
| | | GGAUGUGAUCAAGAACAACCUGGGCCAGAUCCGGGGCGUGACCCCUAGAAACGAUCUGCUGAAUGUGAACGUG |
| | | UCCGCCGAGAUCAACAUCAACUACCGGCUGAGCGAGCUGGGCUUCAUCACCAACAAGAACGACAUGGACUGGAUC |
| | | AAGAAGAAUUACGACCUCUACAGCAAGCUGAUCGCUGGCGCCAUCCACGGCAAGCCUAUUGGAGGACUGGUGGC |
| | | CGGCAACGUGAAAACCAGCGCCAAGAACAAGAAAAACCCUCCUGUGCCUGCCGGCUACACCCUGGACAAGAACAA |
| | | UGUGCCCUACAAGAAAGAGCAGGGCAACUACACCGUGGCCAACGUGAAGGGAAACAACGUGCGCGACGGCUACA |
| | | GCACCAACAGCAGAAUUACAGGCGUGCUGCCCAACAACACCACAAUCACCUACGACGGCGCCUACUGCAUCAACGG |
| | | CUAUCGGUGGAUCACCUACAUUGCCAACUCCGGCCAGCGGCGGUAUAUUGCCACAGGCGAAGUGGACAAGGCCG |
| | | GCAAUAGAAUCAGCUCCUUCGGCAAGUUCAGCACCAUCUAA |

Lysin L0499

| | | |
|---|---|---|
| 41 | amino acid | TQNYVVKKGDYLGAIAKKFNTTVSKIQKLNNIKNPNFILIGQVLKVPFISSNNSSTNTNRRNNSSSTLNYLNSLAYKGWDFDG |
| | sequence | AYGNQCFDLVNVYWNHLYGHGLRGYGAKDIPFANNFRGEATVYRNTPTFKAELGDVAVFSGRYGGGFGHTAIVLNGNYD |
| | | GKLMKFQSLDQNWYGGDSRRADIAHKVVHNYDYDMYFIRPYKKA |
| 42 | nucleotide | ACUCAAAACUACGUUGUUAAGAAAGGAGAUUAUUUGGGUGCUAUCGCUAAGAAAUUCAAUACUACUGUUUCUA |
| | sequence | AGAUCCAAAAGUUGAACAACAUUAAGAACCCAAACUUCAUCUUGAUCGGUCAAGUUUUGAAAGUUCCUUUCAUU |
| | | UCUUCUAACAACUCUUCUACUAACACUAACAGAAGAAACAACUCUUCUUCUACUUUGAACUACUUGAACUCUUU |
| | | GGCUUAUAAGGGUUGGGAUUUUGAUGGUGCUUACGGUAACCAAUGUUUCGAUUUGGUUAACGUUUACUGGAA |
| | | UCAUUUGUAUGGUCACGGUUUGAGAGGUUAUGGUGCUAAGGAUAUUCCAUUUGCUAACAAUUUCAGAGGUGA |
| | | AGCUACUGUUUACAGAAACACUCCUACUUUUAAAGCUGAGUUGGGAGAUGUUGCUGUUUUCUCUGGUAGAUA |
| | | CGGUGGUGGUUUCGGUCAUACUGCUAUCGUUUUGAACGGUAACUACGAUGGUAAAUUGAUGAAGUUCCAAUC |
| | | UUUGGAUCAAAACUGGUACGGUGGAGAUUCUCGUAGAGCUGAUAUCGCUCAUAAGGUUGUUCACAACUACGAU |
| | | UACGAUAUGUACUUCAUCAGACCUUACAAGAAAGCUUAA |
| 43 | nucleotide | ACACAGAAUUACGUGGUGAAGAAGGGCGAUUACCUGGGUGCGAUCGCCAAGAAGUUCAACACCACAGUGAGCAA |
| | sequence | GAUCCAGAAACUCAACAACAUCAAGAAUCCCAACUUCAUCCUGAUUGGCCAAGUGCUGAAAGUACCGUUCAUAA |
| | (opt) | GCUCUAACAACUCAAGCACCAAUACGAACCGCCGGAACAACUCCAGUAGCACACUGAACUACCUGAAUUCCCUCGC |
| | | AUACAAAGGGUGGGAUUUCGAUGGAGCCUAUGGCAAUCAGUGUUUUGACCUCGUCAACGUGUACUGGAAUCAC |
| | | CUCUAUGGUCAUGGGCUGAGAGGGUACGGUGCUAAGGACAUACCUUUUGCCAAUAAUUUUCGCGGCGAAGCUA |
| | | CUGUGUAUCGGAAUACCCCAACCUUCAAAGCAGAGCUUGGAGAUGUUGCUGUUUUCUCAGGGAGGUAUGGAGG |
| | | AGGCUUUGGCCAUACUGCCAUUGUCCUGAAUGGCAACUAUGACGGGAAACUGAUGAAAUUCCAGUCUUUGGAC |
| | | CAGAAUUGGUAUGGCGGGGAUAGUAGACGUGCCGAUAUUGCGCACAAGGUUGUCCACAACUAUGACUACGACA |
| | | UGUACUUUAUCCGACCUUACAAGAAGGCCUGA |

Lysin L0502

| | | |
|---|---|---|
| 44 | amino acid | MNKVDAWLSKHDLKNLTTLIVVGAFILFLILLFMLMNHSDQGSNRITVSENAELRTDPNAAYPVIYQVEKGDTFTRLSKSGK |
| | sequence | WIEVESRDGSEKGWIAGWHTSLNIEADSNKKVNPLKNKVIVLDPGHGGNDQGASSNTSKKSLEKNYTLKTAKELRRALEKA |
| | | GATVKLTRSDDTYVSLDDRKLNGDAYISIHNDSLDSPNANGATVYWYKNNQEALAETLNTNIQKKAMLSNRGTRQENFQVL |
| | | RQTNKPAVLLELGYISNPTDEVMIRNQIHRQVVEEAVVDGLKQYFAS |
| 45 | nucleotide | AUGAACAAGGUUGAUGCUUGGUUGUCUAAGCAUGAUUUGAAAAAUUUGACUACUUUGAUCGUUGUUGGUGCU |
| | sequence | UUUAUUUUGUUCUUGAUCUUGUUGUUUAUGUUGAUGAACCACUCUGAUCAAGGUUCUAACAGAAUUACUGUU |
| | | UCUGAAAAUGCUGAGUUGAGAACUGAUCCAAAUGCUGCUUACCCAGUUAUCUACCAAGUUGAAAAGGGAGAUA |
| | | CUUUCACUAGAUUGUCUAAGUCUGGUAAAUGGAUUGAAGUUGAGUCCAGAGAUGGUUCUGAAAAAGGUUGGA |
| | | UUGCUGGUUGGCAUACUUCUUUGAACAUUGAGGCUGAUUCUAACAAGAAAGUUAACCCAUUGAAGAACAAAGU |
| | | UAUUGUUUUGGAUCCUGGUCACGGUGGUAACGAUCAAGGUGCUUCUUCUAAUACUUCUAAGAAGUCUUUGGA |
| | | AAAGAACUACACUUUGAAGACUGCUAAAGAAUUGCGUAGAGCUUUGGAAAAGGCUGGUGCUACUGUUAAGUUG |
| | | ACUAGAUCUGAUGAUACUUACGUUUCUUUGGAUGAUAGAAAGUUGAACGGAGAUGCUUACAUCUCUAUCCAUA |
| | | ACGAUUCUUUGGAUUCUCCUAACGCUAAUGGUGCUACUGUUUACUGGUACAAGAACAACCAAGAAGCUUUGGC |
| | | UGAGACUUUGAACACUAACAUCCAAAAGAAAGCUAUGUUGUCUAACAGAGGUACUAGACAAGAAAACUUCCAAG |
| | | UUUUGAGACAAACUAACAAGCCAGCUGUUUUGUUGGAAUUGGGUUACAUUUCUAACCCUACUGAUGAGGUUAU |
| | | GAUUAGAAAUCAAAUCCACAGACAAGUUGUUGAAGAGGCUGUUGUUGAUGGUUUGAAACAAUAUUUCGCUUC |
| | | UUAA |
| 46 | nucleotide | AUGAAUAAGGUGGAUGCUUGGCUCAGCAAACACGACCUUAAGAACCUCACUACCUUGAUCGUUGUAGGAGCCU |
| | sequence | UCAUUCUGUUCCUGAUACUGCUGUUCAUGCUGAUGAAUCACUCUGAUCAGGGGUCAAACCGCAUAACUGUUUC |
| | (opt) | CGAGAAUGCCGAACUUCGGACAGAUCCAAACGCCGCCUAUCCGGUCAUCUAUCAGGUCGAGAAAGGGGACACUU |
| | | UCACCAGACUGUCUAAAAGCGGCAAAUGGAUUGAGGUCGAGAGUAGAGAUGGGAGUGAGAAGGGGUGGAUAGC |
| | | AGGAUGGCACACAUCCCUGAACAUUGAAGCCGAUAGCAACAAGAAAGUGAACCCUCUCAAGAACAAAGUGAUCG |
| | | UGCUGGAUCCUGGUCAUGGUGGCAACGAUCAGGGCGCUUCAAGCAAUACCAGCAAGAAGUCACUGGAGAAGAA |
| | | UUACACGCUGAAAACAGCCAAGGAACUGCGACGGGCUCUUGAGAAAGCAGGAGCUACGGUGAAACUGACACGGU |
| | | CUGACGACACCUAUGUGAGUCUGGACGACAGGAAGCUGAAUGGCGACGCGUAUAUCUCCAUUCACAAUGACAGC |
| | | CUGGAUUCUCCCAAUGCAAAUGGGGCUACCGUCUACUGGUACAAGAACAACCAAGAGGCCCUGGCAGAAACCCUC |
| | | AAUACCAACAUCCAGAAGAAGGCGAUGUUGUCCAAUAGGGGUACUAGGCAGGAAAACUUUCAGGUGCUUAGAC |
| | | AGACAAACAAACCCGCAGUUCUGUUGGAACUCGGCUACAUCUCCAACCCAACAGACGAGGUGAUGAUCCGCAAUC |
| | | AGAUUCACCGUCAAGUAGUGGAAGAAGCAGUGGUCGAUGGCCUGAAACAGUACUUUGCCAGUUGA |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The practice of the present disclosure will employ, unless otherwise indicated, conventional chemistry, biochemistry, cell biology, immunology, and recombinant DNA techniques which are explained in the literature in the field.

Throughout this specification and the claims which follow, unless the context requires otherwise, the word “comprise”, and variations such as “comprises” and “comprising”, will be understood to imply the inclusion of a stated feature, element, member, integer or step or group of features, elements, members, integers or steps but not the exclusion of any other feature, element, member, integer or step or group of features, elements, members, integers or steps. The term “consisting essentially of” limits the scope of a claim or disclosure to the specified features, elements, members, integers, or steps and those that do not materially affect the basic and novel characteristic(s) of the claim or disclosure. The term “consisting of” limits the scope of a claim or disclosure to the specified features, elements, members, integers, or steps. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

The term “optional” or “optionally” as used herein means that the subsequently described event, circumstance or condition may or may not occur, and that the description includes instances where said event, circumstance, or condition occurs and instances in which it does not occur.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by +10%, +5%, +4%, +3%, +2%, +1%, +0.9%, +0.8%, +0.7%, +0.6%, +0.5%, +0.4%, +0.3%, +0.2%, +0.1%, +0.05%, and for example+0.01%. In some embodiments, “about” indicates deviation from the indicated numerical value by +10%. In some embodiments, “about” indicates deviation from the indicated numerical value by +5%. In some embodiments, “about” indicates deviation from the indicated numerical value by +4%. In some embodiments, “about” indicates deviation from the indicated numerical value by +3%. In some embodiments, “about” indicates deviation from the indicated numerical value by +2%. In some embodiments, “about” indicates deviation from the indicated numerical value by +1%. In some embodiments, “about” indicates deviation from the indicated numerical value by +0.9%. In some embodiments, “about” indicates deviation from the indicated numerical value by +0.8%.

In some embodiments, “about” indicates deviation from the indicated numerical value by +0.7%. In some embodiments, “about” indicates deviation from the indicated numerical value by +0.6%. In some embodiments, “about” indicates deviation from the indicated numerical value by +0.5%. In some embodiments, “about” indicates deviation from the indicated numerical value by +0.4%. In some embodiments, “about” indicates deviation from the indicated numerical value by +0.3%. In some embodiments, “about” indicates deviation from the indicated numerical value by +0.2%. In some embodiments, “about” indicates deviation from the indicated numerical value by +0.1%. In some embodiments, “about” indicates deviation from the indicated numerical value by +0.05%. In some embodiments, “about” indicates deviation from the indicated numerical value by +0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “enhance” as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level.

“Physiological pH” as used herein refers to a pH of about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material.

The term “ionic strength” refers to the mathematical relationship between the number of different kinds of ionic species in a particular solution and their respective charges. Thus, ionic strength I is represented mathematically by the formula:

$$I = \frac{1}{2} \cdot \sum {z}_{i}^{2} \cdot {c}_{i}$$

in which c is the molar concentration of a particular ionic species and z the absolute value of its charge. The sum 2 is taken over all the different kinds of ions (i) in solution.

According to the disclosure, the term “ionic strength” in some embodiments relates to the presence of monovalent ions. Regarding the presence of divalent ions, in particular divalent cations, their concentration or effective concentration (presence of free ions) due to the presence of chelating agents is, in some embodiments, sufficiently low so as to prevent degradation of the nucleic acid. In some embodiments, the concentration or effective concentration of divalent ions is below the catalytic level for hydrolysis of the phosphodiester bonds between nucleotides such as RNA nucleotides. In some embodiments, the concentration of free divalent ions is 20 UM or less. In some embodiments, there are no or essentially no free divalent ions.

“Osmolality” refers to the concentration of a particular solute expressed as the number of osmoles of solute per kilogram of solvent.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., e.g., from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C.

The term “EDTA” refers to ethylenediaminetetraacetic acid disodium salt. All concentrations are given with respect to the EDTA disodium salt.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

According to the present disclosure, the term “peptide” refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds.

The term “polypeptide” refers to large peptides, in particular peptides having at least about 151 amino acids. “Peptides” and “polypeptides” are both protein molecules, although the terms “protein” and “polypeptide” are used herein usually as synonyms.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may be any portion from 0.1% to 99.9% (such as 0.1%, 0.5%, 1%, 5%, 10%, 50%, 90%, or 99%) of said composition.

“Fragment”, with reference to an amino acid sequence (peptide or polypeptide), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence comprises, e.g., at least 6, in particular at least 8, at least 10, at least 12, at least 15, at least 20, at least 30, at least 50, or at least 100 consecutive amino acids from an amino acid sequence. A fragment of an amino acid sequence comprises, e.g., a sequence of up to 8, in particular up to 10, up to 12, up to 15, up to 20, up to 30 or up to 55, consecutive amino acids of the amino acid sequence.

“Variant,” as used herein and with reference to an amino acid sequence (peptide or polypeptide), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, such as from 1 to about 10 or from 1 to about 5 amino acid differences compared to the parent.

By “wild type” or “WT” or “native” herein is meant an amino acid sequence that is found in nature, including allelic variations. A wild type amino acid sequence, peptide or polypeptide has an amino acid sequence that has not been intentionally modified.

For the purposes of the present disclosure, “variants” of an amino acid sequence (peptide or polypeptide) may comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous peptides or polypeptides and/or to replacing amino acids with other ones having similar properties. In some embodiments, amino acid changes in peptide and polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

In some embodiments the degree of similarity, such as identity between a given amino acid sequence and an amino acid sequence which is a variant of said given amino acid sequence, will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the degree of similarity or identity is given for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given, e.g., for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, such as sequence identity, can be done with art known tools, such as using the best sequence alignment, for example, using Align, using standard settings, preferably EMBOSS::needle, Matrix: Blosum62, Gap Open 10.0, Gap Extend 0.5. “Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence. Homologous amino acid sequences exhibit according to the disclosure at least 40%, in particular at least 50%, at least 60%, at least 70%, at least 80%, at least 90% and, e.g., at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or polypeptides having substitutions, additions, insertions or deletions, is described in detail in Molecular Cloning: A Laboratory Manual, 4^{th} Edition, M. R. Green and J. Sambrook eds., Cold Spring Harbor Laboratory Press, Cold Spring Harbor 2012, for example. Furthermore, the peptides, polypeptides and amino acid variants described herein may be readily prepared with the aid of known peptide synthesis techniques such as, for example, by solid phase synthesis and similar methods.

In some embodiments, a fragment or variant of an amino acid sequence (peptide or polypeptide) is a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to peptidoglycan hydrolases, one particular function is the activity to degrade peptidoglycan displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., degrading peptidoglycan. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., function of the functional fragment or functional variant may be at least 50%, at least 60%, at least 70%, at least 80%, or at least 90% of the parent molecule or sequence. However, in other embodiments, function of the functional fragment or functional variant may be enhanced compared to the parent molecule or sequence.

An amino acid sequence (peptide or polypeptide) “derived from” a designated amino acid sequence (peptide or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the peptidoglycan hydrolases suitable for use herein may be altered such that they vary in sequence from the naturally occurring or native sequences from which they were derived, while retaining the desirable activity of the native sequences.

In some embodiments, “isolated” means removed (e.g., purified) from the natural state or from an artificial composition, such as a composition from a production process. For example, a nucleic acid, peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell.

The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient, or the cell may be in vitro, e.g., outside of a patient. Thus, according to the present disclosure, a cell for transfection of a nucleic acid described herein can be present in vitro or in vivo, e.g. the cell can form part of an organ, a tissue and/or the body of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection, for example. Generally, nucleic acid encoding antigen is transiently transfected into cells. RNA can be transfected into cells to transiently express its coded protein.

The disclosure includes analogs of a peptide or polypeptide. According to the present disclosure, an analog of a peptide or polypeptide is a modified form of said peptide or polypeptide from which it has been derived and has at least one functional property of said peptide or polypeptide. E.g., a pharmacological active analog of a peptide or polypeptide has at least one of the pharmacological activities of the peptide or polypeptide from which the analog has been derived. Such modifications include any chemical modification and comprise single or multiple substitutions, deletions and/or additions of any molecules associated with the peptide or polypeptide, such as carbohydrates, lipids and/or peptides or polypeptides. In some embodiments, “analogs” of peptides or polypeptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said peptides and polypeptides.

As used herein, the terms “linked”, “fused”, or “fusion” are used interchangeably. These terms refer to the joining together of two or more elements or components or domains.

As used herein “endogenous” refers to any material from or produced inside an organism, cell, tissue or system.

As used herein, the term “exogenous” refers to any material introduced from or produced outside an organism, cell, tissue or system.

According to various embodiments of the present disclosure, a nucleic acid such as RNA encoding a peptide or polypeptide is taken up by or introduced, i.e. transfected or transduced, into a cell which cell may be present in vitro or in a subject, resulting in expression of said peptide or polypeptide. The cell may, e.g., express the encoded peptide or polypeptide intracellularly (e.g. in the cytoplasm and/or in the nucleus), may secrete the encoded peptide or polypeptide, and/or may express it on the surface. In some embodiments, the cell secretes the encoded peptide or polypeptide.

According to the present disclosure, terms such as “nucleic acid expressing” and “nucleic acid encoding” or similar terms are used interchangeably herein and with respect to a particular peptide or polypeptide mean that the nucleic acid, if present in the appropriate environment, e.g. within a cell, can be expressed to produce said peptide or polypeptide.

The term “expression” as used herein includes the transcription and/or translation of a particular nucleotide sequence.

In the context of the present disclosure, the term “transcription” relates to a process, wherein the genetic code in a DNA sequence is transcribed into RNA (especially mRNA). Subsequently, the RNA may be translated into peptide or polypeptide.

With respect to RNA, the term “expression” or “translation” relates to the process in the ribosomes of a cell by which a strand of mRNA directs the assembly of a sequence of amino acids to make a peptide or polypeptide.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

In the present specification, a structural formula of a compound may represent a certain isomer of said compound. It is to be understood, however, that the present invention includes all isomers such as geometrical isomers, optical isomers based on an asymmetrical carbon, stereoisomers, tautomers and the like which occur structurally and isomer mixtures and is not limited to the description of the formula.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (+). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimensional forms of (hetero) cyclic rings, such as chair, half-chair, boat, and twist-boat forms of cyclohexane.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is may be calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses m; (i=1, 2, 3, . . . , n), located at fixed distances sᵢ from the center of mass, R_{g} is the square-root of the mass average of sᵢ² over all mass elements and can be calculated as follows:

$${R}_{q} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors \vec{q} the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; T is the temperature; η is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles contained in a sample or control composition as disclosed herein or the hydrodynamic radius of a particle peak obtained from subjecting such a sample or control composition to field-flow fractionation) is to measure the DLS signal of said particle or population of particles (such as DLS signal of particles contained in a sample or control composition as disclosed herein or the DLS signal of a particle peak obtained from subjecting such a sample or control composition to field-flow fractionation).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In certain embodiments, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{K * c} = {M}_{w} P ( θ ) - 2 {A}_{2} c {M}_{w}^{2} {P}^{2} ( θ )$$

wherein c is the mass concentration of the particles in the solvent (g/mL); A₂ is the second virial coefficient (mol·mL/g²); P(ϑ) is a form factor relating to the dependence of scattered light intensity on angle; R_{ϑ} is the excess Rayleigh ratio (cm⁻¹); and K* is an optical constant that is equal to 4π²η₀(dn/dc)²λ₀⁻⁴N_{A}⁻¹, where no is the refractive index of the solvent at the incident radiation (vacuum) wavelength, λ₀ is the incident radiation (vacuum) wavelength (nm), N_{A} is Avogadro's number (mol⁻¹), and dn/dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated the following term:

$$\sqrt{\frac{{R}_{θ}}{K * c}}$$

wherein c, R_{ϑ} and K* are as defined above. Preferably, the Debye plot is calculated the following term:

$$\frac{K * c}{{R}_{θ}}$$

wherein c, R_{ϑ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

### Nucleic Acids

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In some embodiments, a nucleic acid is DNA. In some embodiments, a nucleic acid is RNA. In some embodiments, a nucleic acid is a mixture of DNA and RNA. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In

RNA, they would be represented as A, C and G, whereas in DNA they would be represented as dA, dC and dG.

A modified purine (A or G) or pyrimidine (C, T, or U) base moiety is, in some embodiments, modified by one or more alkyl groups, e.g., one or more C₁₋₄ alkyl groups, e.g., one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷—C₁₋₄ alkyl-guanine, N⁶—C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N6-methyl-adenine, 5-methyl-cytosine, 5-methyl-uracil, and N(1)-methyl-uracil.

Herein, the term “DNA” relates to a nucleic acid molecule which includes deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

The term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). “RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA).

In some embodiments, “RNA” refers to mRNA.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

### RNA

According to the present disclosure, the term “RNA” includes “mRNA”. According to the present disclosure, the term “mRNA” means “messenger-RNA” and includes a “transcript” which may be generated by using a DNA template. Generally, mRNA encodes a peptide or polypeptide. mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide or polypeptide.

In some embodiments, the mRNA which preferably encodes a peptide or polypeptide has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

As established in the art, mRNA generally contains a 5′ untranslated region (5′-UTR), a peptide/polypeptide coding region and a 3′ untranslated region (3′-UTR). In some embodiments, the mRNA is produced by in vitro transcription or chemical synthesis. In some embodiments, the mRNA is produced by in vitro transcription using a DNA template. The in vitro transcription methodology is known to the skilled person; cf., e.g., Molecular Cloning: A Laboratory Manual, 4^{th} Edition, M. R. Green and J. Sambrook eds., Cold Spring Harbor Laboratory Press, Cold Spring Harbor 2012. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified mRNA, correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, RNA is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA is “replicon RNA” or simply a “replicon”, in particular “self-replicating RNA” or “self-amplifying RNA”. In certain embodiments, the replicon or self-replicating RNA is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication (trans-amplification) systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the RNA contains one or more modifications, e.g., in order to increase its stability and/or increase translation efficiency and/or decrease immunogenicity and/or decrease cytotoxicity. For example, in order to increase expression of the RNA, it may be modified within the coding region, i.e., the sequence encoding the expressed peptide or polypeptide, preferably without altering the sequence of the expressed peptide or polypeptide. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the GC content of the RNA).

In some embodiments, the RNA comprises a 5′-cap structure. In some embodiments, the RNA does not have uncapped 5′-triphosphates. In some embodiments, the RNA may comprise a conventional 5′-cap and/or a 5′-cap analog. The term “conventional 5′-cap” refers to a cap structure found on the 5′-end of an RNA molecule and generally comprises a guanosine 5′-triphosphate (Gppp) which is connected via its triphosphate moiety to the 5′-end of the next nucleotide of the RNA (i.e., the guanosine is connected via a 5′ to 5′ triphosphate linkage to the rest of the RNA). The guanosine may be methylated at position N7 (resulting in the cap structure m⁷Gppp). The term “5′-cap analog” includes a 5′-cap which is based on a conventional 5′-cap but which has been modified at either the 2′- or 3′-position of the m⁷guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂^{7,2′O}G(5′)ppSp(5′) G (referred to as beta-S-ARCA or β-S-ARCA)), as described in PCT/EP2019/056502. Providing an RNA with a 5′-cap structure as described herein may be achieved by in vitro transcription of a DNA template in presence of a corresponding 5′-cap compound, wherein said 5′-cap structure is co-transcriptionally incorporated into the generated RNA strand, or the RNA may be generated, for example, by in vitro transcription, and the 5′-cap structure may be attached to the RNA post-transcriptionally using capping enzymes, for example, capping enzymes of vaccinia virus.

In some embodiments, the RNA comprises a 5′-cap structure selected from the group consisting of m₂^{7,2′O}G(5′)ppSp(5′) G (in particular its D1 diastereomer), m₂^{7,3′O}G(5′)ppp(5′)G, and m₂^{7,3′-O}Gppp(m₁^{2′-O})ApG.

In some embodiments, the RNA comprises a cap0, cap1, or cap2, preferably cap1 or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷GpppNmNm”, wherein each Nm is independently any nucleoside bearing an OCH₃ moiety at position 2′.

The 5′-cap analog beta-S-ARCA (β-S-ARCA) has the following structure:

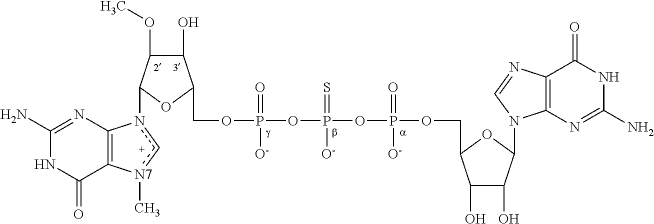

The “D1 diastereomer of beta-S-ARCA” or “beta-S-ARCA (D1)” is the diastereomer of beta-S-ARCA which elutes first on an HPLC column compared to the D2 diastereomer of beta-S-ARCA (beta-S-ARCA (D2)) and thus exhibits a shorter retention time. The HPLC preferably is an analytical HPLC. In some embodiments, a Supelcosil LC-18-T RP column, preferably of the format: 5 μm, 4.6×250 mm is used for separation, whereby a flow rate of 1.3 ml/min can be applied. In some embodiments, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O}Gppp(m₁^{2′-O})ApG (also referred to as m₂^{7,3′O}G(5′)ppp(5′)m^{2′-O}ApG) which is a building block of a cap1 has the following structure:

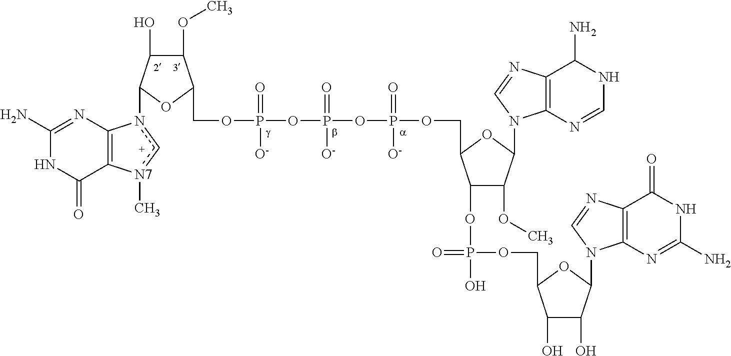

An exemplary cap0 mRNA comprising β-S-ARCA and mRNA has the following structure:

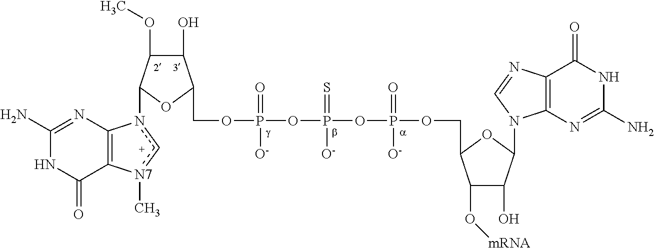

An exemplary cap0 mRNA comprising m₂^{7,3′O}G(5′)ppp(5′) G and mRNA has the following structure:

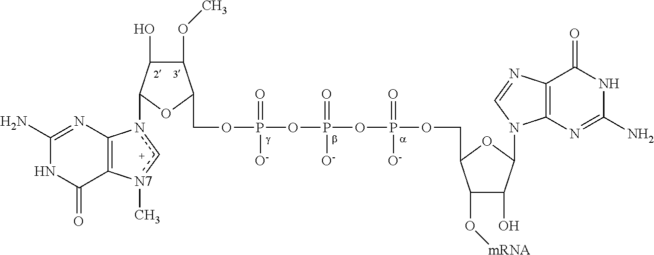

An exemplary cap1 mRNA comprising m₂^{7,3″O}Gppp(m₁^{2′-O})ApG and mRNA has the following structure:

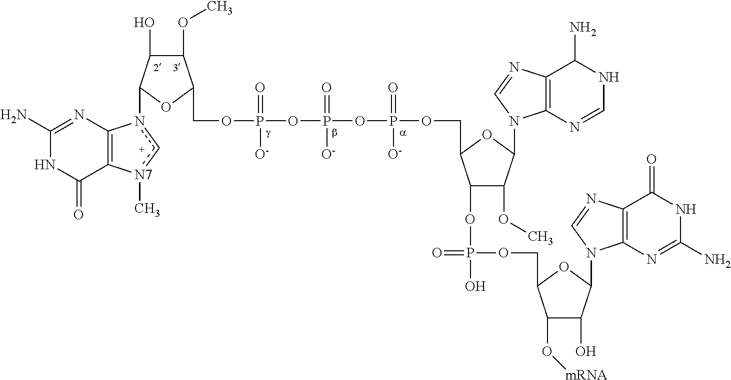

As used herein, the term “poly-A tail” or “poly-A sequence” refers to an uninterrupted or interrupted sequence of adenylate residues which is typically located at the 3′-end of an RNA molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises the poly-A tail shown in SEQ ID NO: 13. In some embodiments, the poly-A tail comprises at least 100 nucleotides. In some embodiments, the poly-A tail comprises about 150 nucleotides. In some embodiments, the poly-A tail comprises about 120 nucleotides.

In some embodiments, RNA used in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does generally not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (e.g., mRNA) into which they are introduced. In certain embodiments, the 3′-UTR is derived from a globin gene or mRNA, such as a gene or mRNA of alpha2-globin, alpha1-globin, or beta-globin, e.g., beta-globin, e.g., human beta-globin. For example, the RNA (e.g., mRNA) may be modified by the replacement of the existing 3′-UTR with or the insertion of one or more, e.g., two copies of a 3′-UTR derived from a globin gene, such as alpha2-globin, alpha1-globin, beta-globin, e.g., beta-globin, e.g., human beta-globin.

A particularly preferred 5′-UTR comprises the nucleotide sequence of SEQ ID NO: 11. A particularly preferred 3′-UTR comprises the nucleotide sequence of SEQ ID NO: 12.

In some embodiments, RNA comprises a 5′-UTR comprising the nucleotide sequence of SEQ ID NO: 11, or a nucleotide sequence having at least 99%, 98%, 97%, 96%, 95%, 90%, 85%, or 80% identity to the nucleotide sequence of SEQ ID NO: 11.

In some embodiments, RNA comprises a 3′-UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

The RNA may have modified ribonucleotides in order to increase its stability and/or decrease immunogenicity and/or decrease cytotoxicity. For example, in some embodiments, uridine in the RNA described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), N1-methyl-pseudouridine (m1ψ), 5-methyl-uridine (m5U), and combinations thereof.

In some embodiments, the modified nucleoside replacing (partially or completely, preferably completely) uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 ψ) 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art.

An RNA (preferably mRNA) which is modified by pseudouridine (replacing partially or completely, preferably completely, uridine) is referred to herein as “Ψ-modified”, whereas the term “m1Ψ-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such Ψ- or m1Ψ- or m5U-modified RNAs usually exhibit decreased immunogenicity compared to their unmodified forms and, thus, are preferred in applications where the induction of an immune response is to be avoided or minimized. In some embodiments, the RNA (preferably mRNA) contains N(1)-methylpseudouridine replacing completely uridine

The codons of the RNA used in the present disclosure may further be optimized, e.g., to increase the GC content of the RNA and/or to replace codons which are rare in the cell (or subject) in which the peptide or polypeptide of interest is to be expressed by codons which are synonymous frequent codons in said cell (or subject). In some embodiments, the amino acid sequence encoded by the RNA used in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In some embodiments, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions may be codon-optimized for optimal expression in a subject to be treated using the RNA described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments, the guanosine/cytosine (G/C) content of the coding region of the RNA described herein is increased compared to the G/C content of the corresponding coding sequence of the wild type RNA, wherein the amino acid sequence encoded by the RNA is preferably not modified compared to the amino acid sequence encoded by the wild type RNA.

This modification of the RNA sequence is based on the fact that the sequence of any RNA region to be translated is important for efficient translation of that RNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m5U) for uridine), and codon optimization, has a synergistic influence on the stability of RNA (preferably mRNA) and increase in translation efficiency. Thus, in some embodiments, the RNA used in the present disclosure contains a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m5U) for uridine), and (v) codon optimization.

Some aspects of the disclosure involve the targeted delivery of the RNA disclosed herein to certain cells or tissues.

Lipid-based RNA delivery systems have an inherent preference to the liver. Liver accumulation is caused by the discontinuous nature of the hepatic vasculature or the lipid metabolism (liposomes and lipid or cholesterol conjugates). In some embodiments, the target organ is liver and the target tissue is liver tissue. The delivery to such target tissue is preferred, in particular, if presence of RNA or of the encoded peptide or polypeptide in this organ or tissue is desired and/or if it is desired to express large amounts of the encoded peptide or polypeptide and/or if systemic presence of the encoded peptide or polypeptide, in particular in significant amounts, is desired or required.

In some embodiments, after administration of the RNA described herein, at least a portion of the RNA is delivered to a target cell or target organ. In some embodiments, at least a portion of the RNA is delivered to the cytosol of the target cell. In some embodiments, the RNA is RNA encoding a peptide or polypeptide and the RNA is translated by the target cell to produce the peptide or polypeptide. In some embodiments, the target cell is a cell in the liver. In some embodiments, the target cell is a muscle cell. In some embodiments, the target cell is a cell in the lymph nodes. In some embodiments, the target cell is a cell in the lung. In some embodiments, the target cell is a cell in the skin. Thus, RNA particles described herein may be used for delivering RNA to such target cell.

Peptidoglycan hydrolases/Endolysins and RNA encoding Peptidoglycan hydrolases/Endolysins

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In some embodiments, RNA such as mRNA used in the present disclosure comprises a nucleic acid sequence encoding a peptidoglycan hydrolase such as an endolysin.

In some embodiments, RNA such as mRNA used in the present disclosure comprises a nucleic acid sequence encoding a peptidoglycan hydrolase such as an endolysin, and is capable of expressing said peptidoglycan hydrolase such as endolysin, in particular if transferred into a cell or subject, preferably a human cell or subject. Thus, in some embodiments, the RNA used in the present disclosure contains a coding region (open reading frame (ORF)) encoding a peptidoglycan hydrolase such as an endolysin. In this respect, an “open reading frame” or “ORF” is a continuous stretch of codons beginning with a start codon and ending with a stop codon. In some embodiments, RNA such as mRNA used in the present disclosure comprises a nucleic acid sequence encoding more than one peptidoglycan hydrolase such as endolysin, e.g., two, three, four or more peptidoglycan hydrolases such as endolysins. In some embodiments, the one or more peptidoglycan hydrolases such as endolysins encoded by the RNA may comprise or consist of naturally occurring sequences, may comprise or consist of variants of naturally occurring sequences, or may comprise or consist of sequences which are not naturally occurring, e.g., recombinant sequences. In some embodiments, the peptide or polypeptide encoded by the RNA described herein may consist of the one or more peptidoglycan hydrolases such as endolysins, or may comprise the one or more peptidoglycan hydrolases such as endolysins and may comprise additional sequences such as secretions signals, extended-PK groups, tags and any other sequences. In some embodiments, the additional sequences are fused to the one or more peptidoglycan hydrolases such as endolysins, in some embodiments, separated by a linker. In these embodiments, the one or more peptidoglycan hydrolases such as endolysins may be considered the pharmaceutically active peptide or polypeptide even if additional sequences support the function or effect of the one or more peptidoglycan hydrolases such as endolysins.

According to the present disclosure, the term “pharmaceutically active peptide or polypeptide” means a peptide or polypeptide that can be used in the treatment of an individual where the expression of a peptide or polypeptide would be of benefit, e.g., in ameliorating the symptoms of a disease. Preferably, a pharmaceutically active peptide or polypeptide has curative or palliative properties and may be administered to ameliorate, relieve, alleviate, reverse, delay onset of or lessen the severity of one or more symptoms of a disease. In some embodiments, a pharmaceutically active peptide or polypeptide has a positive or advantageous effect on the condition or disease state of an individual when administered to the individual in a therapeutically effective amount. A pharmaceutically active peptide or polypeptide may have prophylactic properties and may be used to delay the onset of a disease or to lessen the severity of such disease. The term “pharmaceutically active peptide or polypeptide” includes entire peptides or polypeptides, and can also refer to pharmaceutically active fragments thereof. It can also include pharmaceutically active variants and/or analogs of a peptide or polypeptide.

Peptidoglycan hydrolases such as endolysins described herein can be prepared as fusion or chimeric polypeptides that include a peptidoglycan hydrolase portion such as endolysin portion and a heterologous polypeptide (i.e., a polypeptide that is not a peptidoglycan hydrolase such as an endolysin). The peptidoglycan hydrolase such as endolysin may be fused to an extended-PK group, which increases circulation half-life. Non-limiting examples of extended-PK groups are described infra. It should be understood that other PK groups that increase the circulation half-life of peptides or polypeptides such as peptidoglycan hydrolases such as endolysins, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16 (7): 903-15 which is herein incorporated by reference in its entirety. As used herein, an “extended-PK” peptidoglycan hydrolase such as “extended-PK” endolysin refers to a peptidoglycan hydrolase moiety such as endolysin moiety in combination with an extended-PK group. In some embodiments, the extended-PK peptidoglycan hydrolase such as extended-PK endolysin is a fusion protein in which a peptidoglycan hydrolase moiety such as endolysin moiety is linked or fused to an extended-PK group.

In certain embodiments, the serum half-life of an extended-PK peptidoglycan hydrolase such as extended-PK endolysin is increased relative to the peptidoglycan hydrolase such as endolysin alone (i.e., the peptidoglycan hydrolase such as endolysin not fused to an extended-PK group). In certain embodiments, the serum half-life of the extended-PK peptidoglycan hydrolase such as extended-PK endolysin is at least 20, 40, 60, 80, 100, 120, 150, 180, 200, 400, 600, 800, or 1000% longer relative to the serum half-life of the peptidoglycan hydrolase such as endolysin alone. In certain embodiments, the serum half-life of the extended-PK peptidoglycan hydrolase such as extended-PK endolysin is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the peptidoglycan hydrolase such as endolysin alone. In certain embodiments, the serum half-life of the extended-PK peptidoglycan hydrolase such as extended-PK endolysin is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or polypeptide to reduce by 50%, in vivo, for example due to degradation and/or clearance or sequestration by natural mechanisms. An extended-PK peptidoglycan hydrolase such as extended-PK endolysin suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular a peptidoglycan hydrolase such as an endolysin. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In some embodiments, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used. In other embodiments, the albumin fusion protein has a therapeutic protein fused to both the N-terminus and the C-terminus of albumin. In a preferred embodiment, the therapeutic proteins fused at the N- and C-termini are the same therapeutic proteins. In another preferred embodiment, the therapeutic proteins fused at the N- and C-termini are different therapeutic proteins. In some embodiments, the different therapeutic proteins are both peptidoglycan hydrolases such as endolysins.

In some embodiments, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof.

In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3 (HSA), i.e., a Fn3 protein that binds to human serum albumin. In certain aspects, the extended-PK peptidoglycan hydrolase such as extended-PK endolysin, suitable for use according to the disclosure, can employ one or more peptide linkers. As used herein, the term “peptide linker” refers to a peptide or polypeptide sequence which connects two or more domains (e.g., the extended-PK moiety and a peptidoglycan hydrolase moiety such as an endolysin moiety) in a linear amino acid sequence of a polypeptide chain. For example, peptide linkers may be used to connect a peptidoglycan hydrolase moiety such as an endolysin moiety to a HSA domain.

Linkers suitable for fusing the extended-PK group to e.g. a peptidoglycan hydrolase such as an endolysin are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In some embodiments, the RNA encoding a peptidoglycan hydrolase such as an endolysin is a single-stranded, 5′ capped RNA that is translated into the respective protein upon entering cells of a subject being administered the RNA, e.g., liver cells, or cells of a tissue affected by a bacterial infection. Preferably, the RNA contains structural elements optimized for maximal efficacy of the RNA with respect to stability and translational efficiency (5′ cap, 5′ UTR, 3′ UTR, poly(A) sequence).

In some embodiments, beta-S-ARCA (D1) is utilized as specific capping structure at the 5′-end of the RNA. In some embodiments, m₂^{7,3′-O}Gppp(m₁^{2′-O})ApG is utilized as specific capping structure at the 5′-end of the RNA. In some embodiments, the 5′-UTR comprises the nucleotide sequence of SEQ ID NO: 11, or a nucleotide sequence having at least 99%, 98%, 97%, 96%, 95%, 90%, 85%, or 80% identity to the nucleotide sequence of SEQ ID NO: 11. In some embodiments, the 3′-UTR comprises the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12. In some embodiments, the poly(A) sequence is 110 nucleotides in length and consists of a stretch of 30 adenosine residues, followed by a 10 nucleotide linker sequence and another 70 adenosine residues. This poly(A) sequence was designed to enhance RNA stability and translational efficiency in dendritic cells. In some embodiments, the poly(A) sequence comprises the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In some embodiments, RNA described herein has the structure: Cap-5′ UTR-Peptidoglycan hydrolase-3′ UTR-PolyA. In some embodiments, RNA described herein has the structure: Cap-5′ UTR-SP-Peptidoglycan hydrolase-3′ UTR-PolyA, wherein SP comprises a signal peptide encoding sequence.

In some embodiments, the RNA encoding a peptidoglycan hydrolase such as an endolysin is expressed in cells of the subject to provide the peptidoglycan hydrolase such as endolysin. In some embodiments, the peptidoglycan hydrolase such as endolysin is secreted. In some embodiments, the RNA encoding the peptidoglycan hydrolase such as endolysin is transiently expressed in cells of the subject. In some embodiments, the RNA encoding the peptidoglycan hydrolase such as endolysin is administered systemically. In some embodiments, after systemic administration of the RNA encoding the peptidoglycan hydrolase such as endolysin, expression of the RNA encoding the peptidoglycan hydrolase such as endolysin in liver occurs.

In some embodiments, after systemic administration of the RNA encoding the peptidoglycan hydrolase such as endolysin, expression of the RNA encoding the peptidoglycan hydrolase such as endolysin in cells of a tissue affected by a bacterial infection, e.g., lung cells occurs.

The term “peptidoglycan hydrolase”, as used herein, refers to any polypeptide which is capable of hydrolyzing the peptidoglycan of bacteria, such as Gram-negative bacteria. The term is not restricted to a specific enzymatic cleavage mechanism. Three main classes of bacterial peptidoglycan hydrolases are glycosidases that cleave the backbone of glycan, the amidases that cleave the side-chain peptide and peptidases (endopeptidases and carboxypeptidases) that cleave within the peptide side-chain. The term encompasses naturally occurring peptidoglycan hydrolases, such as peptidoglycan hydrolases of eukaryotic, prokaryotic or viral (in particular bacteriophage) origin. The term encompasses for example lysozymes, endolysins, tail-spike depolymerases, Virion-associated peptidoglycan hydrolases (VAPGH), bacteriocins (e.g. lysostaphin) and autolysins. The “peptidoglycan hydrolase” may also be a synthetic or modified polypeptide (e.g., recombinant polypeptide) capable of hydrolyzing the peptidoglycan of bacteria. For example, enzymatically active shuffled endolysins in which domains of two or more endolysins have been swapped/exchanged qualify as “peptidoglycan hydrolase” just as truncated endolysins, in which only the enzymatic active domain remains. Peptidoglycan hydrolase activity can be measured by assays well known in the art, e.g., by antibacterial assays, e.g., by assays which are e.g. described in Briers et al. (J. Biochem. Biophys Methods; 2007; 70:531-533) or Donovan et al. (J. FEMS Microbiol Lett. 2006 December; 265 (1).

Endolysins include bacteriophage-encoded enzymes, which act by hydrolyzing the host cell wall and subsequently allow the release of bacteriophage progenies. The lytic activity of endolysins is classified into different types: (i) acetylmuramidases, (ii) transglycosylases, (iii) glucosaminidases, (iv) amidases, and (v) endopeptidases.

Generally, endolysins have a molecular weight ranging from 15 to 40 kDa. The majority of endolysins have a modular configuration. Modular endolysins are often characterized by the presence of one or two (multi-domain) N-terminal enzymatically active domains (EADs) linked by a short, flexible linker region to a C-terminal cell wall-binding domain (CBD). The enzymatically active domain (EAD) of modular endolysins functions to cleave various specific peptidoglycan bonds in the murein layer of the host bacterium, while the cell wall-binding domain (CBD) recognizes and binds to different epitopes in the cell wall for proper fixation of the catalytic effect of the EAD.

Generally, endolysins acting on Gram-negative bacteria have a simple globular configuration of the EAD without a CBD. Recent studies also show the occurrence of Gram-negative phage endolysins with globular configuration, having one or two CBDs at the N-terminus while the EAD module is at the C-terminus.

EADs may be categorized into three classes of six distinct enzymatic activities based on the mode of action and individual enzymatic specificities:
(i) Glycosidases generally cleave the β-1,4 glycosidic bonds linking alternating polymeric structures of N-acetylmuramic acids (MurNAc) and N-acetylglucosamines (GlcNAc) in the peptidoglycan layer;
(ii) Amidases catalyze the cleavage of amide bonds between the MurNAc and the first amino acid in the peptide stem moiety, L-alanine;
(iii) Endopeptidases cleave bonds between two amino acids of the stem peptide. Bond cleavage can either occur within interpeptide bridge or stem peptide-interpeptide bridge.

As used herein, the term “endolysin” includes peptidoglycan hydrolases encoded by bacteriophages (or bacterial viruses) and includes any peptidoglycan hydrolase which is suitable to hydrolyse bacterial cell walls. Such peptidoglycan hydrolases may be naturally occurring enzymes of bacteriophages, modified enzymes of bacteriophages and non-naturally occurring enzymes, e.g., recombinant enzymes, such as enzymes having a non-naturally occurring arrangement of domains, which domains may be derived from naturally occurring enzymes. In some embodiments, endolysins are enzymes which have been engineered to alter their host range and efficiency. In some embodiments, endolysins comprise endolysins comprising naturally occurring domains and naturally occurring endolysins, e.g., endolysins comprising an N-terminal naturally occurring catalytic domain, and a C-terminal naturally occurring cell wall binding domain, chimeric lysins characterized by shuffling of natural domains to create desirable properties, artilysins characterized by addition of an LPS disruptor to disrupt Gram-negative outer membrane, and truncated lysine, e.g., wherein the C-terminal cell wall binding domain has been removed to increase activity.

In some embodiments, an endolysin is derived from a bacteriophage (bacterial virus). In some embodiments, an endolysin may be a β-(1,4)-glycosylase (lysozyme), transglycosylase, amidase or endopeptidases. In some embodiments, “endolysins” comprise at least one “enzymatically active domain” (EAD) having at least one of the following activities: endopeptidase, N-acetyl-muramoyl-L-alanine-amidase (amidase), N-acetyl-muramidase, N-acetyl-glucosaminidase (lysozyme) or transglycosylases. In some embodiments, endolysins may contain also regions which are enzymatically inactive, and bind to the cell wall of the host bacteria, the so-called CBDs (cell wall binding domains). Endolysins may contain one or more, e.g., two or more CBDs. However, the term “endolysin” as used herein also includes enzymes having at least one EAD but no CBDs. Generally, the cell wall binding domain is able to bind different components on the surface of bacteria. Preferably, the cell wall binding domain is a peptidoglycan binding domain and binds to the bacteria's peptidoglycan structure. The different domains of an endolysin can be connected by a domain linker.

In gram-positive bacteria, the cytoplasmic membrane is surrounded by a peptidoglycan layer. The main purpose of the cell wall of Gram-positive bacteria is to maintain the shape of the bacteria and counteract the pressure inside the bacterial cells. Peptidoglycan or murein is a polymer composed of sugar and amino acid. The sugar component is composed of N-acetylglucosamine residues and a N-acetylmuramic acid residues that are β-(1,4) linked. A peptide chain consisting of 3 to 5 amino acids is bound to N-acetylmuramic acid. Peptide chains can be cross-linked to peptide chains of other chains to form a 3D mesh-like layer. The peptide chain can contain D- and L-amino acid residues, and its composition can vary depending on the type of bacteria.

In contrast to gram-positive bacteria, gram-negative bacteria have an outer membrane with a characteristic asymmetric bilayer. The outer membrane bilayer consists of an inner monolayer containing phospholipids (primarily phosphatidylethanolamine) and an outer monolayer composed primarily of lipopolysaccharide (LPS).

The term “cell wall” as used herein refers to all components that form the outer cell enclosure of bacteria and thus guarantee their integrity. In particular, the term “cell wall” as used herein refers to peptidoglycan, the outer membrane of the Gram-negative bacteria with the lipopolysaccharide, the bacterial cell membrane, but also to additional layers deposited on the peptidoglycan as e.g. capsules, outer protein layers or slimes.

The term “EAD” as used herein refers to the enzymatically active domain of an endolysin. The EAD is responsible for hydrolysing bacterial peptidoglycans. It exhibits at least one enzymatic activity of an endolysin. The EAD can also be composed of more than one enzymatically active module. The term “EAD” is used herein synonymously with the term “catalytic domain”. Most gram-negative bacteria and many gram-positive bacteria form bacterial biofilms. A biofilm is defined as an aggregate or aggregate of microorganisms attached to a surface. Adherent bacteria are often surrounded and protected by extracellular polymeric substances produced by Gram-negative and Gram-positive bacteria. Bacteria are more resistant to antibacterials such as antibiotics, disinfectants and cell wall degrading enzymes through biofilms. Furthermore, currently, biofilm treatment is not feasible because extracellular polymeric substances protect themselves against degradation by antibacterials, disinfectants or biofilm degradants.

In some embodiments, the endolysin is or is derived from a Gram-negative phage endolysin.

In some embodiments, the endolysin is or is derived from a Pseudomonas phage endolysin.

In some embodiments, the endolysin is or is derived from a Pseudomonas aeruginosa phage endolysin. In some embodiments, the endolysin is or is derived from endolysin LysPA26 having the following sequence:

(SEQ ID NO: 1)
MRTNNIDAIKEHEGLRLVAYLDSVGVWTIGYGDTGPDVVKGLTITKEEA
EKRLRKRLVEFEGYVNTYVKVPLKQHQFDALVSLVYNIGPTNFKTSTLL
KKLNAGDYIGAADQFLVWNKGRVDGKLVVIKGLANRRAKERKQFLGE

For purposes of the present disclosure, the above sequence is considered the wildtype LysPA26 amino acid sequence. Position numberings in LysPA26 protein given herein are in relation to the amino acid sequence according to SEQ ID NO: 1 and corresponding positions in LysPA26 protein variants.

In some embodiments, the encoded amino acid sequence comprises, consists essentially of or consists of an endolysin such as LysPA26.

In some embodiments, the encoded amino acid sequence comprises the amino acid sequence of amino acids 6 to 138 of SEQ ID NO: 1, an amino acid sequence having at least 99%, 98%, 97%, 96%, 95%, 90%, 85%, or 80% identity to the amino acid sequence of amino acids 6 to 138 of SEQ ID NO: 1, or a functional fragment of the amino acid sequence of amino acids 6 to 138 of SEQ ID NO: 1, or the amino acid sequence having at least 99%, 98%, 97%, 96%, 95%, 90%, 85%, or 80% identity to the amino acid sequence of amino acids 6 to 138 of SEQ ID NO: 1. In some embodiments, the encoded amino acid sequence comprises the amino acid sequence of amino acids 6 to 138 of SEQ ID NO: 1.

In some embodiments, the encoded amino acid sequence comprises the amino acid sequence of amino acids 2 to 145 of SEQ ID NO: 1, an amino acid sequence having at least 99%, 98%, 97%, 96%, 95%, 90%, 85%, or 80% identity to the amino acid sequence of amino acids 2 to 145 of SEQ ID NO: 1, or a functional fragment of the amino acid sequence of amino acids 2 to 145 of SEQ ID NO: 1, or the amino acid sequence having at least 99%, 98%, 97%, 96%, 95%, 90%, 85%, or 80% identity to the amino acid sequence of amino acids 2 to 145 of SEQ ID NO: 1. In some embodiments, the encoded amino acid sequence comprises the amino acid sequence of amino acids 2 to 145 of SEQ ID NO: 1.

In some embodiments, the encoded amino acid sequence comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In some embodiments, the encoded amino acid sequence comprises the amino acid sequence of SEQ ID NO: 1.

In some embodiments, RNA comprises the nucleotide sequence of SEQ ID NO: 2, a nucleotide sequence having at least 99%, 98%, 97%, 96%, 95%, 90%, 85%, or 80% identity to the nucleotide sequence of SEQ ID NO: 2, or a fragment of the nucleotide sequence of SEQ ID NO: 2, or the nucleotide sequence having at least 99%, 98%, 97%, 96%, 95%, 90%, 85%, or 80% identity to the nucleotide sequence of SEQ ID NO: 2. In some embodiments, RNA comprises the nucleotide sequence of SEQ ID NO: 2.

In some embodiments, RNA (i) comprises the nucleotide sequence of SEQ ID NO: 2, a nucleotide sequence having at least 99%, 98%, 97%, 96%, 95%, 90%, 85%, or 80% identity to the nucleotide sequence of SEQ ID NO: 2, or a fragment of the nucleotide sequence of SEQ ID NO: 2, or the nucleotide sequence having at least 99%, 98%, 97%, 96%, 95%, 90%, 85%, or 80% identity to the nucleotide sequence of SEQ ID NO: 2; and/or (ii) encodes an amino acid sequence comprising the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In some embodiments, RNA (i) comprises the nucleotide sequence of SEQ ID NO: 2; and/or (ii) encodes an amino acid sequence comprising the amino acid sequence of SEQ ID NO: 1.

In some embodiments, RNA comprises the nucleotide sequence of SEQ ID NO: 3, a nucleotide sequence having at least 99%, 98%, 97%, 96%, 95%, 90%, 85%, or 80% identity to the nucleotide sequence of SEQ ID NO: 3, or a fragment of the nucleotide sequence of SEQ ID NO: 3, or the nucleotide sequence having at least 99%, 98%, 97%, 96%, 95%, 90%, 85%, or 80% identity to the nucleotide sequence of SEQ ID NO: 3. In some embodiments, RNA comprises the nucleotide sequence of SEQ ID NO: 3.

In some embodiments, RNA (i) comprises the nucleotide sequence of SEQ ID NO: 3, a nucleotide sequence having at least 99%, 98%, 97%, 96%, 95%, 90%, 85%, or 80% identity to the nucleotide sequence of SEQ ID NO: 3, or a fragment of the nucleotide sequence of SEQ ID NO: 3, or the nucleotide sequence having at least 99%, 98%, 97%, 96%, 95%, 90%, 85%, or 80% identity to the nucleotide sequence of SEQ ID NO: 3; and/or (ii) encodes an amino acid sequence comprising the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In some embodiments, RNA (i) comprises the nucleotide sequence of SEQ ID NO: 3; and/or (ii) encodes an amino acid sequence comprising the amino acid sequence of SEQ ID NO: 1.

In some embodiments, RNA comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO:

6, or the nucleotide sequence having at least 99%, 98%, 97%, 96%, 95%, 90%, 85%, or 80% identity to the nucleotide sequence of SEQ ID NO: 6. In some embodiments, RNA comprises the nucleotide sequence of SEQ ID NO: 6.

In some embodiments, RNA (i) comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6, or the nucleotide sequence having at least 99%, 98%, 97%, 96%, 95%, 90%, 85%, or 80% identity to the nucleotide sequence of SEQ ID NO: 6; and/or (ii) encodes an amino acid sequence comprising the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In some embodiments, RNA (i) comprises the nucleotide sequence of SEQ ID NO: 6; and/or (ii) encodes an amino acid sequence comprising the amino acid sequence of SEQ ID NO: 1.

In some embodiments, RNA comprises the nucleotide sequence of SEQ ID NO: 8, a nucleotide sequence having at least 99%, 98%, 97%, 96%, 95%, 90%, 85%, or 80% identity to the nucleotide sequence of SEQ ID NO: 8, or a fragment of the nucleotide sequence of SEQ ID NO: 8, or the nucleotide sequence having at least 99%, 98%, 97%, 96%, 95%, 90%, 85%, or 80% identity to the nucleotide sequence of SEQ ID NO: 8. In some embodiments, RNA comprises the nucleotide sequence of SEQ ID NO: 8.

In some embodiments, RNA (i) comprises the nucleotide sequence of SEQ ID NO: 8, a nucleotide sequence having at least 99%, 98%, 97%, 96%, 95%, 90%, 85%, or 80% identity to the nucleotide sequence of SEQ ID NO: 8, or a fragment of the nucleotide sequence of SEQ ID NO: 8, or the nucleotide sequence having at least 99%, 98%, 97%, 96%, 95%, 90%, 85%, or 80% identity to the nucleotide sequence of SEQ ID NO: 8; and/or (ii) encodes an amino acid sequence comprising the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In some embodiments, RNA (i) comprises the nucleotide sequence of SEQ ID NO: 8; and/or (ii) encodes an amino acid sequence comprising the amino acid sequence of SEQ ID NO: 1.

According to certain embodiments, a signal peptide (or signal sequence) is fused, either directly or through a linker, to a peptidoglycan hydrolase, e.g., an endolysin. Accordingly, in some embodiments, a signal peptide is fused to the above described amino acid sequences derived from LysPA26 endolysin or functional fragments thereof comprised by the encoded amino acid sequences described above.

In some embodiments, an open reading frame of the RNA described herein encodes a polypeptide that includes a signal sequence, e.g., that is functional in mammalian cells.

In some embodiments, a utilized signal sequence is “intrinsic” in that it is, in nature, associated with (e.g., linked to) the encoded polypeptide.

In some embodiments, a utilized signal sequence is heterologous to the encoded polypeptide—e.g., is not naturally part of a polypeptide (e.g., protein) whose sequences are included in the encoded polypeptide.

In some embodiments, signal peptides are sequences, which are typically characterized by a length of about 15 to 30 amino acids.

In many embodiments, signal peptides are positioned at the N-terminus of an encoded polypeptide as described herein, without being limited thereto. In some embodiments, signal peptides preferably allow the transport of the polypeptide encoded by RNAs of the present disclosure with which they are associated into a defined cellular compartment, preferably the cell surface, the endoplasmic reticulum (ER) or the endosomal-lysosomal compartment.

In some embodiments, an RNA sequence encodes a peptidoglycan hydrolase, e.g., an endolysin, that may comprise or otherwise be linked to a signal sequence (e.g., secretory sequence), such as those listed in Table 2, or a sequence having 1, 2, 3, 4, or 5 amino acid differences relative thereto. In some embodiments, a signal sequence such as MRVMAPRTLILLLSGALALTETWAGS, or a sequence having 1, 2, 3, 4, or at the most 5 amino acid differences relative thereto is utilized. In some embodiments, a sequence such as MRVMAPRTLILLLSGALALTETWAGS, or a sequence having 1, 2, 3, 4, or at the most 5 amino acid differences relative thereto, is utilized.

In some embodiments, a signal sequence is selected from those included in the Table 2 below:

TABLE 2
Signal sequences

| Signal | Sequence (Amino Acid) |
|---|---|
| HSV-1 gD SP | MGGAAARLGAVILFVVIVGLHGVRSKY |
| HSV-2 gD SP | MGRLTSGVGTAALLVVAVGLRVVCA |
| HSV-2 | MGRLTSGVGTAALLVVAVGLRVVCAKYA |
| SARS-CoV-2-S | MFVFLVLLPLVSSQCVNLT |
| human Ig | MDWIWRILFLVGAATGAHSQM |
| heavy chain | |
| signal peptide | |
| human Ig | MDWTWRVFCLLAVAPGAHS |
| heavy chain | |
| signal peptide | |
| HuIgGk signal | METPAQLLFLLLLWLPDTTG |
| peptide | |
| IgE heavy | MDWTWILFLVAAATRVHS |
| chain epsilon- | |
| 1signal | |
| peptide | |
| Japanese | MLGSNSGQRVVFTILLLLVAPAYS |
| encephalitis | |
| PRM signal | |
| sequence | |
| VSVg protein | MKCLLYLAFLFIGVNCA |
| signal | |
| sequence | |
| | MDWTWILFLVAAATRVHS |
| | ETPAQLLFLLLLWLPDTTG |
| | MLGSNSGQRVVFTILLLLVAPAYS |
| | MKCLLYLAFLFIGVNCA |
| | MWLVSLAIVTACAGA |
| | MFVFLVLLPLVSSQC |

In some embodiments, the encoded amino acid sequence comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In some embodiments, the encoded amino acid sequence comprises the amino acid sequence of SEQ ID NO: 4.

In some embodiments, RNA comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a fragment of the nucleotide sequence of SEQ ID NO: 5, or the nucleotide sequence having at least 99%, 98%, 97%, 96%, 95%, 90%, 85%, or 80% identity to the nucleotide sequence of SEQ ID NO: 5. In some embodiments, RNA comprises the nucleotide sequence of SEQ ID NO: 5.

In some embodiments, RNA (i) comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a fragment of the nucleotide sequence of SEQ ID NO: 5, or the nucleotide sequence having at least 99%, 98%, 97%, 96%, 95%, 90%, 85%, or 80% identity to the nucleotide sequence of SEQ ID NO: 5; and/or (ii) encodes an amino acid sequence comprising the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In some embodiments, RNA (i) comprises the nucleotide sequence of SEQ ID NO: 5; and/or (ii) encodes an amino acid sequence comprising the amino acid sequence of SEQ ID NO: 4.

In some embodiments, RNA comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a fragment of the nucleotide sequence of SEQ ID NO:

7, or the nucleotide sequence having at least 99%, 98%, 97%, 96%, 95%, 90%, 85%, or 80% identity to the nucleotide sequence of SEQ ID NO: 7. In some embodiments, RNA comprises the nucleotide sequence of SEQ ID NO: 7.

In some embodiments, RNA (i) comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a fragment of the nucleotide sequence of SEQ ID NO: 7, or the nucleotide sequence having at least 99%, 98%, 97%, 96%, 95%, 90%, 85%, or 80% identity to the nucleotide sequence of SEQ ID NO: 7; and/or (ii) encodes an amino acid sequence comprising the amino acid sequence of SEQ ID NO: 4, an amino acid sequence having at least 99%, 98%, 97%, 96%, 95%, 90%, 85%, or 80% identity to the amino acid sequence of

SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In some embodiments, RNA (i) comprises the nucleotide sequence of SEQ ID NO: 7; and/or (ii) encodes an amino acid sequence comprising the amino acid sequence of SEQ ID NO: 4.

In some embodiments, RNA comprises the nucleotide sequence of SEQ ID NO: 9, a nucleotide sequence having at least 99%, 98%, 97%, 96%, 95%, 90%, 85%, or 80% identity to the nucleotide sequence of SEQ ID NO: 9, or a fragment of the nucleotide sequence of SEQ ID NO: 9, or the nucleotide sequence having at least 99%, 98%, 97%, 96%, 95%, 90%, 85%, or 80% identity to the nucleotide sequence of SEQ ID NO: 9. In some embodiments, RNA comprises the nucleotide sequence of SEQ ID NO: 9.

In some embodiments, RNA (i) comprises the nucleotide sequence of SEQ ID NO: 9, a nucleotide sequence having at least 99%, 98%, 97%, 96%, 95%, 90%, 85%, or 80% identity to the nucleotide sequence of SEQ ID NO: 9, or a fragment of the nucleotide sequence of SEQ ID NO: 9, or the nucleotide sequence having at least 99%, 98%, 97%, 96%, 95%, 90%, 85%, or 80% identity to the nucleotide sequence of SEQ ID NO: 9; and/or (ii) encodes an amino acid sequence comprising the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In some embodiments, RNA (i) comprises the nucleotide sequence of SEQ ID NO: 9; and/or (ii) encodes an amino acid sequence comprising the amino acid sequence of SEQ ID NO: 4.

In some embodiments, the encoded amino acid sequence comprises the amino acid sequence of SEQ ID NO: 14, an amino acid sequence having at least 99%, 98%, 97%, 96%, 95%, 90%, 85%, or 80% identity to the amino acid sequence of SEQ ID NO: 14, or a functional fragment of the amino acid sequence of SEQ ID NO: 14, or the amino acid sequence having at least 99%, 98%, 97%, 96%, 95%, 90%, 85%, or 80% identity to the amino acid sequence of SEQ ID NO: 14.

In some embodiments, the encoded amino acid sequence comprises the amino acid sequence of SEQ ID NO: 14.

In some embodiments, RNA comprises the nucleotide sequence of SEQ ID NO: 15 or 16, a nucleotide sequence having at least 99%, 98%, 97%, 96%, 95%, 90%, 85%, or 80% identity to the nucleotide sequence of SEQ ID NO: 15 or 16, or a fragment of the nucleotide sequence of SEQ ID NO: 15 or 16, or the nucleotide sequence having at least 99%, 98%, 97%, 96%, 95%, 90%, 85%, or 80% identity to the nucleotide sequence of SEQ ID NO: 15 or 16. In some embodiments, RNA comprises the nucleotide sequence of SEQ ID NO: 15 or 16.

In some embodiments, RNA (i) comprises the nucleotide sequence of SEQ ID NO: 15 or 16, a nucleotide sequence having at least 99%, 98%, 97%, 96%, 95%, 90%, 85%, or 80% identity to the nucleotide sequence of SEQ ID NO: 15 or 16, or a fragment of the nucleotide sequence of SEQ ID NO: 15 or 16, or the nucleotide sequence having at least 99%, 98%, 97%, 96%, 95%, 90%, 85%, or 80% identity to the nucleotide sequence of SEQ ID NO: 15 or 16; and/or (ii) encodes an amino acid sequence comprising the amino acid sequence of SEQ ID NO: 14, an amino acid sequence having at least 99%, 98%, 97%, 96%, 95%, 90%, 85%, or 80% identity to the amino acid sequence of SEQ ID NO: 14, or a functional fragment of the amino acid sequence of SEQ ID NO: 14, or the amino acid sequence having at least 99%, 98%, 97%, 96%, 95%, 90%, 85%, or 80% identity to the amino acid sequence of SEQ ID NO: 14. In some embodiments, RNA (i) comprises the nucleotide sequence of SEQ ID NO: 15 or 16; and/or (ii) encodes an amino acid sequence comprising the amino acid sequence of SEQ ID NO: 14.

In some embodiments, the encoded amino acid sequence comprises the amino acid sequence of SEQ ID NO: 17, an amino acid sequence having at least 99%, 98%, 97%, 96%, 95%, 90%, 85%, or 80% identity to the amino acid sequence of SEQ ID NO: 17, or a functional fragment of the amino acid sequence of SEQ ID NO: 17, or the amino acid sequence having at least 99%, 98%, 97%, 96%, 95%, 90%, 85%, or 80% identity to the amino acid sequence of SEQ ID NO: 17.

In some embodiments, the encoded amino acid sequence comprises the amino acid sequence of SEQ ID NO: 17.

In some embodiments, RNA comprises the nucleotide sequence of SEQ ID NO: 18 or 19, a nucleotide sequence having at least 99%, 98%, 97%, 96%, 95%, 90%, 85%, or 80% identity to the nucleotide sequence of SEQ ID NO: 18 or 19, or a fragment of the nucleotide sequence of SEQ ID NO: 18 or 19, or the nucleotide sequence having at least 99%, 98%, 97%, 96%, 95%, 90%, 85%, or 80% identity to the nucleotide sequence of SEQ ID NO: 18 or 19. In some embodiments, RNA comprises the nucleotide sequence of SEQ ID NO: 18 or 19.

In some embodiments, RNA (i) comprises the nucleotide sequence of SEQ ID NO: 18 or 19, a nucleotide sequence having at least 99%, 98%, 97%, 96%, 95%, 90%, 85%, or 80% identity to the nucleotide sequence of SEQ ID NO: 18 or 19, or a fragment of the nucleotide sequence of SEQ ID NO: 18 or 19, or the nucleotide sequence having at least 99%, 98%, 97%, 96%, 95%, 90%, 85%, or 80% identity to the nucleotide sequence of SEQ ID NO: 18 or 19; and/or (ii) encodes an amino acid sequence comprising the amino acid sequence of SEQ ID NO: 17, an amino acid sequence having at least 99%, 98%, 97%, 96%, 95%, 90%, 85%, or 80% identity to the amino acid sequence of SEQ ID NO: 17, or a functional fragment of the amino acid sequence of SEQ ID NO: 17, or the amino acid sequence having at least 99%, 98%, 97%, 96%, 95%, 90%, 85%, or 80% identity to the amino acid sequence of SEQ ID NO: 17. In some embodiments, RNA (i) comprises the nucleotide sequence of SEQ ID NO: 18 or 19; and/or (ii) encodes an amino acid sequence comprising the amino acid sequence of SEQ ID NO: 17.

In some embodiments, the encoded amino acid sequence comprises the amino acid sequence of SEQ ID NO: 20, an amino acid sequence having at least 99%, 98%, 97%, 96%, 95%, 90%, 85%, or 80% identity to the amino acid sequence of SEQ ID NO: 20, or a functional fragment of the amino acid sequence of SEQ ID NO: 20, or the amino acid sequence having at least 99%, 98%, 97%, 96%, 95%, 90%, 85%, or 80% identity to the amino acid sequence of SEQ ID NO: 20.

In some embodiments, the encoded amino acid sequence comprises the amino acid sequence of SEQ ID NO: 20.

In some embodiments, RNA comprises the nucleotide sequence of SEQ ID NO: 21 or 22, a nucleotide sequence having at least 99%, 98%, 97%, 96%, 95%, 90%, 85%, or 80% identity to the nucleotide sequence of SEQ ID NO: 21 or 22, or a fragment of the nucleotide sequence of SEQ ID NO: 21 or 22, or the nucleotide sequence having at least 99%, 98%, 97%, 96%, 95%, 90%, 85%, or 80% identity to the nucleotide sequence of SEQ ID NO: 21 or 22. In some embodiments, RNA comprises the nucleotide sequence of SEQ ID NO: 21 or 22.

In some embodiments, RNA (i) comprises the nucleotide sequence of SEQ ID NO: 21 or 22, a nucleotide sequence having at least 99%, 98%, 97%, 96%, 95%, 90%, 85%, or 80% identity to the nucleotide sequence of SEQ ID NO: 21 or 22, or a fragment of the nucleotide sequence of SEQ ID NO: 21 or 22, or the nucleotide sequence having at least 99%, 98%, 97%, 96%, 95%, 90%, 85%, or 80% identity to the nucleotide sequence of SEQ ID NO: 21 or 22; and/or (ii) encodes an amino acid sequence comprising the amino acid sequence of SEQ ID NO: 20, an amino acid sequence having at least 99%, 98%, 97%, 96%, 95%, 90%, 85%, or 80% identity to the amino acid sequence of SEQ ID NO: 20, or a functional fragment of the amino acid sequence of SEQ ID NO: 20, or the amino acid sequence having at least 99%, 98%, 97%, 96%, 95%, 90%, 85%, or 80% identity to the amino acid sequence of SEQ ID NO: 20. In some embodiments, RNA (i) comprises the nucleotide sequence of SEQ ID NO: 21 or 22; and/or (ii) encodes an amino acid sequence comprising the amino acid sequence of SEQ ID NO: 20.

In some embodiments, the encoded amino acid sequence comprises the amino acid sequence of SEQ ID NO: 23, an amino acid sequence having at least 99%, 98%, 97%, 96%, 95%, 90%, 85%, or 80% identity to the amino acid sequence of SEQ ID NO: 23, or a functional fragment of the amino acid sequence of SEQ ID NO: 23, or the amino acid sequence having at least 99%, 98%, 97%, 96%, 95%, 90%, 85%, or 80% identity to the amino acid sequence of SEQ ID NO: 23.

In some embodiments, the encoded amino acid sequence comprises the amino acid sequence of SEQ ID NO: 23.

In some embodiments, RNA comprises the nucleotide sequence of SEQ ID NO: 24 or 25, a nucleotide sequence having at least 99%, 98%, 97%, 96%, 95%, 90%, 85%, or 80% identity to the nucleotide sequence of SEQ ID NO: 24 or 25, or a fragment of the nucleotide sequence of SEQ ID NO: 24 or 25, or the nucleotide sequence having at least 99%, 98%, 97%, 96%, 95%, 90%, 85%, or 80% identity to the nucleotide sequence of SEQ ID NO: 24 or 25. In some embodiments, RNA comprises the nucleotide sequence of SEQ ID NO: 24 or 25.

In some embodiments, RNA (i) comprises the nucleotide sequence of SEQ ID NO: 24 or 25, a nucleotide sequence having at least 99%, 98%, 97%, 96%, 95%, 90%, 85%, or 80% identity to the nucleotide sequence of SEQ ID NO: 24 or 25, or a fragment of the nucleotide sequence of SEQ ID NO: 24 or 25, or the nucleotide sequence having at least 99%, 98%, 97%, 96%, 95%, 90%, 85%, or 80% identity to the nucleotide sequence of SEQ ID NO: 24 or 25; and/or (ii) encodes an amino acid sequence comprising the amino acid sequence of SEQ ID NO: 23, an amino acid sequence having at least 99%, 98%, 97%, 96%, 95%, 90%, 85%, or 80% identity to the amino acid sequence of SEQ ID NO: 23, or a functional fragment of the amino acid sequence of SEQ ID NO: 23, or the amino acid sequence having at least 99%, 98%, 97%, 96%, 95%, 90%, 85%, or 80% identity to the amino acid sequence of SEQ ID NO: 23. In some embodiments, RNA (i) comprises the nucleotide sequence of SEQ ID NO: 24 or 25; and/or (ii) encodes an amino acid sequence comprising the amino acid sequence of SEQ ID NO: 23.

In some embodiments, the encoded amino acid sequence comprises the amino acid sequence of SEQ ID NO: 26, an amino acid sequence having at least 99%, 98%, 97%, 96%, 95%, 90%, 85%, or 80% identity to the amino acid sequence of SEQ ID NO: 26, or a functional fragment of the amino acid sequence of SEQ ID NO: 26, or the amino acid sequence having at least 99%, 98%, 97%, 96%, 95%, 90%, 85%, or 80% identity to the amino acid sequence of SEQ ID NO: 26.

In some embodiments, the encoded amino acid sequence comprises the amino acid sequence of SEQ ID NO: 26.

In some embodiments, RNA comprises the nucleotide sequence of SEQ ID NO: 27 or 28, a nucleotide sequence having at least 99%, 98%, 97%, 96%, 95%, 90%, 85%, or 80% identity to the nucleotide sequence of SEQ ID NO: 27 or 28, or a fragment of the nucleotide sequence of

SEQ ID NO: 27 or 28, or the nucleotide sequence having at least 99%, 98%, 97%, 96%, 95%, 90%, 85%, or 80% identity to the nucleotide sequence of SEQ ID NO: 27 or 28. In some embodiments, RNA comprises the nucleotide sequence of SEQ ID NO: 27 or 28.

In some embodiments, RNA (i) comprises the nucleotide sequence of SEQ ID NO: 27 or 28, a nucleotide sequence having at least 99%, 98%, 97%, 96%, 95%, 90%, 85%, or 80% identity to the nucleotide sequence of SEQ ID NO: 27 or 28, or a fragment of the nucleotide sequence of SEQ ID NO: 27 or 28, or the nucleotide sequence having at least 99%, 98%, 97%, 96%, 95%, 90%, 85%, or 80% identity to the nucleotide sequence of SEQ ID NO: 27 or 28; and/or (ii) encodes an amino acid sequence comprising the amino acid sequence of SEQ ID NO: 26, an amino acid sequence having at least 99%, 98%, 97%, 96%, 95%, 90%, 85%, or 80% identity to the amino acid sequence of SEQ ID NO: 26, or a functional fragment of the amino acid sequence of SEQ ID NO: 26, or the amino acid sequence having at least 99%, 98%, 97%, 96%, 95%, 90%, 85%, or 80% identity to the amino acid sequence of SEQ ID NO: 26. In some embodiments, RNA (i) comprises the nucleotide sequence of SEQ ID NO: 27 or 28; and/or (ii) encodes an amino acid sequence comprising the amino acid sequence of SEQ ID NO: 26.

In some embodiments, the encoded amino acid sequence comprises the amino acid sequence of SEQ ID NO: 29, an amino acid sequence having at least 99%, 98%, 97%, 96%, 95%, 90%, 85%, or 80% identity to the amino acid sequence of SEQ ID NO: 29, or a functional fragment of the amino acid sequence of SEQ ID NO: 29, or the amino acid sequence having at least 99%, 98%, 97%, 96%, 95%, 90%, 85%, or 80% identity to the amino acid sequence of SEQ ID NO: 29.

In some embodiments, the encoded amino acid sequence comprises the amino acid sequence of SEQ ID NO: 29.

In some embodiments, RNA comprises the nucleotide sequence of SEQ ID NO: 30 or 31, a nucleotide sequence having at least 99%, 98%, 97%, 96%, 95%, 90%, 85%, or 80% identity to the nucleotide sequence of SEQ ID NO: 30 or 31, or a fragment of the nucleotide sequence of SEQ ID NO: 30 or 31, or the nucleotide sequence having at least 99%, 98%, 97%, 96%, 95%, 90%, 85%, or 80% identity to the nucleotide sequence of SEQ ID NO: 30 or 31. In some embodiments, RNA comprises the nucleotide sequence of SEQ ID NO: 30 or 31.

In some embodiments, RNA (i) comprises the nucleotide sequence of SEQ ID NO: 30 or 31, a nucleotide sequence having at least 99%, 98%, 97%, 96%, 95%, 90%, 85%, or 80% identity to the nucleotide sequence of SEQ ID NO: 30 or 31, or a fragment of the nucleotide sequence of

SEQ ID NO: 30 or 31, or the nucleotide sequence having at least 99%, 98%, 97%, 96%, 95%, 90%, 85%, or 80% identity to the nucleotide sequence of SEQ ID NO: 30 or 31; and/or (ii) encodes an amino acid sequence comprising the amino acid sequence of SEQ ID NO: 29, an amino acid sequence having at least 99%, 98%, 97%, 96%, 95%, 90%, 85%, or 80% identity to the amino acid sequence of SEQ ID NO: 29, or a functional fragment of the amino acid sequence of SEQ ID NO: 29, or the amino acid sequence having at least 99%, 98%, 97%, 96%, 95%, 90%, 85%, or 80% identity to the amino acid sequence of SEQ ID NO: 29. In some embodiments, RNA (i) comprises the nucleotide sequence of SEQ ID NO: 30 or 31; and/or (ii) encodes an amino acid sequence comprising the amino acid sequence of SEQ ID NO: 29.

In some embodiments, the encoded amino acid sequence comprises the amino acid sequence of SEQ ID NO: 32, an amino acid sequence having at least 99%, 98%, 97%, 96%, 95%, 90%, 85%, or 80% identity to the amino acid sequence of SEQ ID NO: 32, or a functional fragment of the amino acid sequence of SEQ ID NO: 32, or the amino acid sequence having at least 99%, 98%, 97%, 96%, 95%, 90%, 85%, or 80% identity to the amino acid sequence of SEQ ID NO: 32.

In some embodiments, the encoded amino acid sequence comprises the amino acid sequence of SEQ ID NO: 32.

In some embodiments, RNA comprises the nucleotide sequence of SEQ ID NO: 33 or 34, a nucleotide sequence having at least 99%, 98%, 97%, 96%, 95%, 90%, 85%, or 80% identity to the nucleotide sequence of SEQ ID NO: 33 or 34, or a fragment of the nucleotide sequence of SEQ ID NO: 33 or 34, or the nucleotide sequence having at least 99%, 98%, 97%, 96%, 95%, 90%, 85%, or 80% identity to the nucleotide sequence of SEQ ID NO: 33 or 34. In some embodiments, RNA comprises the nucleotide sequence of SEQ ID NO: 33 or 34.

In some embodiments, RNA (i) comprises the nucleotide sequence of SEQ ID NO: 33 or 34, a nucleotide sequence having at least 99%, 98%, 97%, 96%, 95%, 90%, 85%, or 80% identity to the nucleotide sequence of SEQ ID NO: 33 or 34, or a fragment of the nucleotide sequence of SEQ ID NO: 33 or 34, or the nucleotide sequence having at least 99%, 98%, 97%, 96%, 95%, 90%, 85%, or 80% identity to the nucleotide sequence of SEQ ID NO: 33 or 34; and/or (ii) encodes an amino acid sequence comprising the amino acid sequence of SEQ ID NO: 32, an amino acid sequence having at least 99%, 98%, 97%, 96%, 95%, 90%, 85%, or 80% identity to the amino acid sequence of SEQ ID NO: 32, or a functional fragment of the amino acid sequence of SEQ ID NO: 32, or the amino acid sequence having at least 99%, 98%, 97%, 96%, 95%, 90%, 85%, or 80% identity to the amino acid sequence of SEQ ID NO: 32. In some embodiments, RNA (i) comprises the nucleotide sequence of SEQ ID NO: 33 or 34; and/or (ii) encodes an amino acid sequence comprising the amino acid sequence of SEQ ID NO: 32.

In some embodiments, the encoded amino acid sequence comprises the amino acid sequence of SEQ ID NO: 35, an amino acid sequence having at least 99%, 98%, 97%, 96%, 95%, 90%, 85%, or 80% identity to the amino acid sequence of SEQ ID NO: 35, or a functional fragment of the amino acid sequence of SEQ ID NO: 35, or the amino acid sequence having at least 99%, 98%, 97%, 96%, 95%, 90%, 85%, or 80% identity to the amino acid sequence of SEQ ID NO: 35.

In some embodiments, the encoded amino acid sequence comprises the amino acid sequence of SEQ ID NO: 35.

In some embodiments, RNA comprises the nucleotide sequence of SEQ ID NO: 36 or 37, a nucleotide sequence having at least 99%, 98%, 97%, 96%, 95%, 90%, 85%, or 80% identity to the nucleotide sequence of SEQ ID NO: 36 or 37, or a fragment of the nucleotide sequence of SEQ ID NO: 36 or 37, or the nucleotide sequence having at least 99%, 98%, 97%, 96%, 95%, 90%, 85%, or 80% identity to the nucleotide sequence of SEQ ID NO: 36 or 37. In some embodiments, RNA comprises the nucleotide sequence of SEQ ID NO: 36 or 37.

In some embodiments, RNA (i) comprises the nucleotide sequence of SEQ ID NO: 36 or 37, a nucleotide sequence having at least 99%, 98%, 97%, 96%, 95%, 90%, 85%, or 80% identity to the nucleotide sequence of SEQ ID NO: 36 or 37, or a fragment of the nucleotide sequence of SEQ ID NO: 36 or 37, or the nucleotide sequence having at least 99%, 98%, 97%, 96%, 95%, 90%, 85%, or 80% identity to the nucleotide sequence of SEQ ID NO: 36 or 37; and/or (ii) encodes an amino acid sequence comprising the amino acid sequence of SEQ ID NO: 35, an amino acid sequence having at least 99%, 98%, 97%, 96%, 95%, 90%, 85%, or 80% identity to the amino acid sequence of SEQ ID NO: 35, or a functional fragment of the amino acid sequence of SEQ ID NO: 35, or the amino acid sequence having at least 99%, 98%, 97%, 96%, 95%, 90%, 85%, or 80% identity to the amino acid sequence of SEQ ID NO: 35. In some embodiments, RNA (i) comprises the nucleotide sequence of SEQ ID NO: 36 or 37; and/or (ii) encodes an amino acid sequence comprising the amino acid sequence of SEQ ID NO: 35.

In some embodiments, the encoded amino acid sequence comprises the amino acid sequence of SEQ ID NO: 38, an amino acid sequence having at least 99%, 98%, 97%, 96%, 95%, 90%, 85%, or 80% identity to the amino acid sequence of SEQ ID NO: 38, or a functional fragment of the amino acid sequence of SEQ ID NO: 38, or the amino acid sequence having at least 99%, 98%, 97%, 96%, 95%, 90%, 85%, or 80% identity to the amino acid sequence of SEQ ID NO: 38.

In some embodiments, the encoded amino acid sequence comprises the amino acid sequence of SEQ ID NO: 38.

In some embodiments, RNA comprises the nucleotide sequence of SEQ ID NO: 39 or 40, a nucleotide sequence having at least 99%, 98%, 97%, 96%, 95%, 90%, 85%, or 80% identity to the nucleotide sequence of SEQ ID NO: 39 or 40, or a fragment of the nucleotide sequence of SEQ ID NO: 39 or 40, or the nucleotide sequence having at least 99%, 98%, 97%, 96%, 95%, 90%, 85%, or 80% identity to the nucleotide sequence of SEQ ID NO: 39 or 40. In some embodiments, RNA comprises the nucleotide sequence of SEQ ID NO: 39 or 40.

In some embodiments, RNA (i) comprises the nucleotide sequence of SEQ ID NO: 39 or 40, a nucleotide sequence having at least 99%, 98%, 97%, 96%, 95%, 90%, 85%, or 80% identity to the nucleotide sequence of SEQ ID NO: 39 or 40, or a fragment of the nucleotide sequence of SEQ ID NO: 39 or 40, or the nucleotide sequence having at least 99%, 98%, 97%, 96%, 95%, 90%, 85%, or 80% identity to the nucleotide sequence of SEQ ID NO: 39 or 40; and/or (ii) encodes an amino acid sequence comprising the amino acid sequence of SEQ ID NO: 38, an amino acid sequence having at least 99%, 98%, 97%, 96%, 95%, 90%, 85%, or 80% identity to the amino acid sequence of SEQ ID NO: 38, or a functional fragment of the amino acid sequence of SEQ ID NO: 38, or the amino acid sequence having at least 99%, 98%, 97%, 96%, 95%, 90%, 85%, or 80% identity to the amino acid sequence of SEQ ID NO: 38. In some embodiments, RNA (i) comprises the nucleotide sequence of SEQ ID NO: 39 or 40; and/or (ii) encodes an amino acid sequence comprising the amino acid sequence of SEQ ID NO: 38.

In some embodiments, the encoded amino acid sequence comprises the amino acid sequence of SEQ ID NO: 41, an amino acid sequence having at least 99%, 98%, 97%, 96%, 95%, 90%, 85%, or 80% identity to the amino acid sequence of SEQ ID NO: 41, or a functional fragment of the amino acid sequence of SEQ ID NO: 41, or the amino acid sequence having at least 99%, 98%, 97%, 96%, 95%, 90%, 85%, or 80% identity to the amino acid sequence of SEQ ID NO: 41.

In some embodiments, the encoded amino acid sequence comprises the amino acid sequence of SEQ ID NO: 41.

In some embodiments, RNA comprises the nucleotide sequence of SEQ ID NO: 42 or 43, a nucleotide sequence having at least 99%, 98%, 97%, 96%, 95%, 90%, 85%, or 80% identity to the nucleotide sequence of SEQ ID NO: 42 or 43, or a fragment of the nucleotide sequence of SEQ ID NO: 42 or 43, or the nucleotide sequence having at least 99%, 98%, 97%, 96%, 95%, 90%, 85%, or 80% identity to the nucleotide sequence of SEQ ID NO: 42 or 43. In some embodiments, RNA comprises the nucleotide sequence of SEQ ID NO: 42 or 43.

In some embodiments, RNA (i) comprises the nucleotide sequence of SEQ ID NO: 42 or 43, a nucleotide sequence having at least 99%, 98%, 97%, 96%, 95%, 90%, 85%, or 80% identity to the nucleotide sequence of SEQ ID NO: 42 or 43, or a fragment of the nucleotide sequence of SEQ ID NO: 42 or 43, or the nucleotide sequence having at least 99%, 98%, 97%, 96%, 95%, 90%, 85%, or 80% identity to the nucleotide sequence of SEQ ID NO: 42 or 43; and/or (ii) encodes an amino acid sequence comprising the amino acid sequence of SEQ ID NO: 41, an amino acid sequence having at least 99%, 98%, 97%, 96%, 95%, 90%, 85%, or 80% identity to the amino acid sequence of SEQ ID NO: 41, or a functional fragment of the amino acid sequence of SEQ ID NO: 41, or the amino acid sequence having at least 99%, 98%, 97%, 96%, 95%, 90%, 85%, or 80% identity to the amino acid sequence of SEQ ID NO: 41. In some embodiments, RNA (i) comprises the nucleotide sequence of SEQ ID NO: 42 or 43; and/or (ii) encodes an amino acid sequence comprising the amino acid sequence of SEQ ID NO: 41.

In some embodiments, the encoded amino acid sequence comprises the amino acid sequence of SEQ ID NO: 44, an amino acid sequence having at least 99%, 98%, 97%, 96%, 95%, 90%, 85%, or 80% identity to the amino acid sequence of SEQ ID NO: 44, or a functional fragment of the amino acid sequence of SEQ ID NO: 44, or the amino acid sequence having at least 99%, 98%, 97%, 96%, 95%, 90%, 85%, or 80% identity to the amino acid sequence of SEQ ID NO: 44.

In some embodiments, the encoded amino acid sequence comprises the amino acid sequence of SEQ ID NO: 44.

In some embodiments, RNA comprises the nucleotide sequence of SEQ ID NO: 45 or 46, a nucleotide sequence having at least 99%, 98%, 97%, 96%, 95%, 90%, 85%, or 80% identity to the nucleotide sequence of SEQ ID NO: 45 or 46, or a fragment of the nucleotide sequence of

SEQ ID NO: 45 or 46, or the nucleotide sequence having at least 99%, 98%, 97%, 96%, 95%, 90%, 85%, or 80% identity to the nucleotide sequence of SEQ ID NO: 45 or 46. In some embodiments, RNA comprises the nucleotide sequence of SEQ ID NO: 45 or 46.

In some embodiments, RNA (i) comprises the nucleotide sequence of SEQ ID NO: 45 or 46, a nucleotide sequence having at least 99%, 98%, 97%, 96%, 95%, 90%, 85%, or 80% identity to the nucleotide sequence of SEQ ID NO: 45 or 46, or a fragment of the nucleotide sequence of SEQ ID NO: 45 or 46, or the nucleotide sequence having at least 99%, 98%, 97%, 96%, 95%, 90%, 85%, or 80% identity to the nucleotide sequence of SEQ ID NO: 45 or 46; and/or (ii) encodes an amino acid sequence comprising the amino acid sequence of SEQ ID NO: 44, an amino acid sequence having at least 99%, 98%, 97%, 96%, 95%, 90%, 85%, or 80% identity to the amino acid sequence of SEQ ID NO: 44, or a functional fragment of the amino acid sequence of SEQ ID NO: 44, or the amino acid sequence having at least 99%, 98%, 97%, 96%, 95%, 90%, 85%, or 80% identity to the amino acid sequence of SEQ ID NO: 44. In some embodiments, RNA (i) comprises the nucleotide sequence of SEQ ID NO: 45 or 46; and/or (ii) encodes an amino acid sequence comprising the amino acid sequence of SEQ ID NO: 44.

### Embodiments of RNA Described Herein

In the following, embodiments of RNA for use herein are described, wherein certain terms used when describing elements thereof have the following meanings:
hAg-Kozak: 5′-UTR sequence of the human alpha-globin mRNA with an optimized ‘Kozak sequence’ to increase translational efficiency.
SP: Signal peptide.
Endolysin: Sequences encoding the respective endolysin or fragment.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues.

In some embodiments, RNA described herein has the structure:
Cap-hAg-Kozak-Endolysin-FI-A30L70

In some embodiments, RNA described herein has the structure:
Cap-hAg-Kozak-SP-Endolysin-FI-A30L70

In some embodiments, encoded amino acid sequences described herein have the structure:
SP-Endolysin

In some embodiments, hAg-Kozak comprises the nucleotide sequence of SEQ ID NO: 11. In some embodiments, SP comprises the amino acid sequence of SEQ ID NO: 10. In some embodiments, FI comprises the nucleotide sequence of SEQ ID NO: 12. In some embodiments, A30L70 comprises the nucleotide sequence of SEQ ID NO: 13.

In some embodiments, the RNA used in the present disclosure is non-immunogenic.

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In certain embodiments, non-immunogenic RNA, which is also termed modified RNA (modRNA) herein, is rendered non-immunogenic by incorporating modified nucleosides suppressing RNA-mediated activation of innate immune receptors into the RNA and/or limiting the amount of double-stranded RNA (dsRNA), e.g., by limiting the formation of double-stranded RNA (dsRNA), e.g., during in vitro transcription, and/or by removing double-stranded RNA (dsRNA), e.g., following in vitro transcription. In certain embodiments, non-immunogenic RNA is rendered non-immunogenic by incorporating modified nucleosides suppressing RNA-mediated activation of innate immune receptors into the RNA and/or by removing double-stranded RNA (dsRNA), e.g., following in vitro transcription.

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In certain embodiments, the nucleoside comprising a modified nucleobase is pseudouridine (ψ), N1-methyl-pseudouridine (m1ψ) or 5-methyl-uridine (m5U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of RNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. Formation of dsRNA can be limited during synthesis of RNA by in vitro transcription (IVT), for example, by limiting the amount of uridine triphosphate (UTP) during synthesis. Optionally, UTP may be added once or several times during synthesis of RNA. Also, dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the amount of double-stranded RNA (dsRNA) is limited, e.g., dsRNA (especially mRNA) is removed from non-immunogenic RNA, such that less than 10%, less than 5%, less than 4%, less than 3%, less than 2%, less than 1%, less than 0.5%, less than 0.3%, less than 0.1%, less than 0.05%, less than 0.03%, less than 0.01%, less than 0.005%, less than 0.004%, less than 0.003%, less than 0.002%, less than 0.001%, or less than 0.0005% of the RNA in the non-immunogenic RNA composition is dsRNA. In some embodiments, the non-immunogenic RNA (especially mRNA) is free or essentially free of dsRNA. In some embodiments, the non-immunogenic RNA (especially mRNA) composition comprises a purified preparation of single-stranded nucleoside modified RNA. In some embodiments, the non-immunogenic RNA (especially mRNA) composition comprises single-stranded nucleoside modified RNA (especially mRNA) and is substantially free of double stranded RNA (dsRNA). In some embodiments, the non-immunogenic RNA (especially mRNA) composition comprises at least 90%, at least 91%, at least 92%, at least 93%, at least 94%, at least 95%, at least 96%, at least 97%, at least 98%, at least 99%, at least 99.5%, at least 99.9%, at least 99.99%, at least 99.991%, at least 99.992%, at least 99.993%, at least 99.994%, at least 99.995%, at least 99.996%, at least 99.997%, or at least 99.998% single stranded nucleoside modified RNA, relative to all other nucleic acid molecules (DNA, dsRNA, etc.).

Various methods can be used to determine the amount of dsRNA. For example, a sample may be contacted with dsRNA-specific antibody and the amount of antibody binding to RNA may be taken as a measure for the amount of dsRNA in the sample. A sample containing a known amount of dsRNA may be used as a reference.

For example, RNA may be spotted onto a membrane, e.g., nylon blotting membrane. The membrane may be blocked, e.g., in TBS-T buffer (20 mM TRIS pH 7.4, 137 mM NaCl, 0.1% (v/v) TWEEN-20) containing 5% (w/v) skim milk powder. For detection of dsRNA, the membrane may be incubated with dsRNA-specific antibody, e.g., dsRNA-specific mouse mAb (English & Scientific Consulting, Szirák, Hungary). After washing, e.g., with TBS-T, the membrane may be incubated with a secondary antibody, e.g., HRP-conjugated donkey anti-mouse IgG (Jackson ImmunoResearch, Cat #715-035-150), and the signal provided by the secondary antibody may be detected.

In some embodiments, the non-immunogenic RNA (especially mRNA) is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor.

In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA (especially mRNA) exhibits significantly less innate immunogenicity than standard RNA with the same sequence. In some embodiments, the non-immunogenic RNA (especially mRNA) exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor.

In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA (especially mRNA) can be administered without triggering a detectable innate immune response. In some embodiments, the term refers to a decrease such that the non-immunogenic RNA (especially mRNA) can be repeatedly administered without eliciting an innate immune response sufficient to detectably reduce production of the protein encoded by the non-immunogenic RNA. In some embodiments, the decrease is such that the non-immunogenic RNA (especially mRNA) can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

In some embodiments, an RNA described herein is a single-stranded RNA. In some embodiments, an RNA described herein is a linear RNA. In some embodiments, a single-stranded RNA is a coding RNA in that its nucleotide sequence includes an open reading frame.

In some embodiments, a single-stranded RNA has a nucleotide sequence that encodes a polypeptide or a plurality of polypeptides of the present disclosure.

In many embodiments, a relevant RNA is an mRNA.

In some embodiments, an RNA includes unmodified uridine residues; an RNA that includes only unmodified uridine residues may be referred to as a “uRNA”. In some embodiments, an RNA includes one or more modified uridine residues; in some embodiments, such an RNA (e.g., an RNA including entirely modified uridine residues) is referred to as a “modRNA”. In some embodiments, an RNA may be a self-amplifying RNA (saRNA). In some embodiments, an RNA may be a trans-amplifying RNA (see, for example, WO2017/162461).

In some embodiments, RNA (e.g., a single stranded RNA) described herein has a length of at least 500 ribonucleotides (such as, e.g., at least 600 ribonucleotides, at least 700 ribonucleotides, at least 800 ribonucleotides, at least 900 ribonucleotides, at least 1000 ribonucleotides, at least 1250 ribonucleotides, at least 1500 ribonucleotides, at least 1750 ribonucleotides, at least 2000 ribonucleotides, at least 2500 ribonucleotides, at least 3000 ribonucleotides, at least 3500 ribonucleotides, at least 4000 ribonucleotides, at least 4500 ribonucleotides, at least 5000 ribonucleotides, or longer). In some embodiments, RNA described herein is single-stranded RNA having a length of about 800 ribonucleotides to 5000 ribonucleotides.

In some embodiments, a relevant RNA includes a polypeptide-encoding portion or a plurality of polypeptide-encoding portions. In some particular embodiments, such a portion or portions encode at least polypeptide or polypeptides that is or comprises a peptidoglycan hydrolase, e.g., endolysin. In certain embodiments, an encoded peptidoglycan hydrolase, e.g., endolysin, may be a variant of a wild type polypeptide.

In some embodiments, a single-stranded RNA (e.g., mRNA) may comprise a secretion signal-encoding region (e.g., a secretion signal-encoding region that allows an encoded target entity or entities (comprising a peptidoglycan hydrolase, e.g., endolysin) to be secreted upon translation by cells). In some embodiments, such a secretion signal-encoding region may be or comprise a non-human secretion signal. In some embodiments, such a secretion signal-encoding region may be or comprise a human secretion signal.

In some embodiments, a single-stranded RNA (e.g., mRNA) may comprise at least one non-coding sequence element (e.g., to enhance RNA stability and/or translation efficiency). Examples of non-coding sequence elements include but are not limited to a 3′ untranslated region (UTR), a 5′ UTR, a cap structure, a poly adenine (polyA) tail, and any combinations thereof.

At least four formats useful for RNA pharmaceutical compositions may be used herein, namely non-modified uridine containing mRNA (uRNA), nucleoside modified mRNA (modRNA), self-amplifying mRNA (saRNA), and trans-amplifying RNAs.

Features of modified uridine (e.g., pseudouridine) platform may include reduced adjuvant effect, blunted immune innate immune sensor activating capacity and thus augmented polypeptide (e.g., protein) expression.

Features of self-amplifying platform may include, for example, long duration of polypeptide (e.g., protein) expression, good tolerability and safety, higher likelihood for efficacy with very low RNA dose.

In some embodiments, a self-amplifying platform (e.g., RNA) comprises two nucleic acid molecules, wherein one nucleic acid molecule encodes a replicase (e.g., a viral replicase) and the other nucleic acid molecule is capable of being replicated (e.g., a replicon) by said replicase in trans (trans-replication system). In some embodiments, a self-amplifying platform (e.g., RNA) comprises a plurality of nucleic acid molecules, wherein said nucleic acids encode a plurality of replicases and/or replicons.

In some embodiments, a trans-replication system comprises the presence of both nucleic acid molecules in a single host cell.

In some such embodiments, a nucleic acid encoding a replicase (e.g., a viral replicase) is not capable of self-replication in a target cell and/or target organism. In some such embodiments, a nucleic acid encoding a replicase (e.g., a viral replicase) lacks at least one conserved sequence element important for (−) strand synthesis based on a (+) strand template and/or for (+) strand synthesis based on a (−) strand template.

In some embodiments, a self-amplifying RNA comprises a 3′ untranslated region (UTR), a 5′ UTR, a cap structure, a poly adenine (polyA) tail, and any combinations thereof.

In some embodiments, a self-amplifying platform does not require propagation of virus particles (e.g., is not associated with undesired virus-particle formation). In some embodiments, a self-amplifying platform is not capable of forming virus particles.

### Particles

RNA, in particular mRNA, described herein may be present in particles comprising (i) the RNA, and (ii) at least one cationic or cationically ionizable compound such as a polymer or lipid complexing the RNA. Electrostatic interactions between positively charged molecules such as polymers and lipids and negatively charged RNA are involved in particle formation. This results in complexation and spontaneous formation of RNA particles.

Different types of RNA containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” includes nanoparticles.

An “RNA particle” can be used to deliver RNA to a target site of interest (e.g., cell, tissue, organ, and the like). An RNA particle may be formed from lipids comprising at least one cationic or cationically ionizable lipid or lipid-like material. Without intending to be bound by any theory, it is believed that the cationic or cationically ionizable lipid or lipid-like material combines together with the RNA to form aggregates, and this aggregation results in colloidally stable particles.

RNA particles described herein include lipid nanoparticle (LNP)-based and lipoplex (LPX)-based formulations.

In general, a lipoplex (LPX) is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of RNA are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with RNAs. In some embodiments, formed lipoplexes possess distinct internal arrangements of molecules that arise due to the transformation from liposomal structure into compact RNA-lipoplexes. In some embodiments, these formulations are characterized by their poor encapsulation of the RNA and incomplete entrapment of the RNA.

In some embodiments, an LPX particle comprises an amphiphilic lipid, in particular cationic or cationically ionizable amphiphilic lipid, and RNA (especially mRNA) as described herein. In some embodiments, electrostatic interactions between positively charged liposomes (made from one or more amphiphilic lipids, in particular cationic or cationically ionizable amphiphilic lipids) and negatively charged RNA (especially mRNA) results in complexation and spontaneous formation of RNA lipoplex particles. Positively charged liposomes may be generally synthesized using a cationic or cationically ionizable amphiphilic lipid, such as DOTMA and/or DODMA, and additional lipids, such as DOPE. In some embodiments, an RNA (especially mRNA) lipoplex particle is a nanoparticle.

In general, a lipid nanoparticle (LNP) is obtainable from direct mixing of RNA in an aqueous phase with lipids in a phase comprising an organic solvent, such as ethanol. In that case, lipids or lipid mixtures can be used for particle formation, which do not form lamellar (bilayer) phases in water.

In some embodiments, LNPs comprise or consist of a cationic/ionizable lipid and helper lipids such as phospholipids, cholesterol, and/or polyethylene glycol (PEG) lipids. In some embodiments, in the RNA LNPs described herein the RNA is bound by ionizable lipid that occupies the central core of the LNP. In some embodiments, PEG lipid forms the surface of the LNP, along with phospholipids. In some embodiments, the surface comprises a bilayer. In some embodiments, cholesterol and ionizable lipid in charged and uncharged forms can be distributed throughout the LNP.

In some embodiments, RNA (e.g., mRNA) may be noncovalently associated with a particle as described herein. In embodiments, the RNA (especially mRNA) may be adhered to the outer surface of the particle (surface RNA (especially surface mRNA)) and/or may be contained in the particle (encapsulated RNA (especially encapsulated mRNA)).

In some embodiments, the particles (e.g., LNPs and LPXs) described herein have a size (such as a diameter) in the range of about 10 to about 2000 nm, such as at least about 15 nm (e.g., at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most 1900 nm (e.g., at most about 1900 nm, at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), such as in the range of about 20 to about 1500 nm, such as about 30 to about 1200 nm, about 40 to about 1100 nm, about 50 to about 1000 nm, about 60 to about 900 nm, about 70 to 800 nm, about 80 to 700 nm, about 90 to 600 nm, or about 50 to 500 nm or about 100 to 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, or 70 to 150 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm.

In some embodiments, the particles described herein are nanoparticles. The term “nanoparticle” relates to a nano-sized particle comprising nucleic acid (especially mRNA) as described herein and at least one cationic or cationically ionizable lipid, wherein all three external dimensions of the particle are in the nanoscale, i.e., at least about 1 nm and below about 1000 nm. Preferably, the size of a particle is its diameter.

RNA particles described herein (especially mRNA particles) may exhibit a polydispersity index

(PDI) less than about 0.5, less than about 0.4, less than about 0.3, less than about 0.2, less than about 0.1, or less than about 0.05. By way of example, the RNA particles can exhibit a polydispersity index in a range of about 0.01 to about 0.4 or about 0.1 to about 0.3. The N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

RNA particles (especially mRNA particles) described herein can be prepared using a wide range of methods that may involve obtaining a colloid from at least one cationic or cationically ionizable lipid and mixing the colloid with RNA to obtain RNA particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA (especially mRNA) lipoplex particles described herein are obtainable by adding RNA (especially mRNA) to a colloidal liposome dispersion. Using the ethanol injection technique, such colloidal liposome dispersion is, in some embodiments, formed as follows: an ethanol solution comprising lipids, such as cationic or cationically ionizable lipids like DOTMA and/or DODMA and additional lipids, is injected into an aqueous solution under stirring. In some embodiments, the RNA (especially mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some embodiments, LNPs comprise four components: ionizable cationic lipids, neutral lipids such as phospholipids, a steroid such as cholesterol, and a polymer conjugated lipid. In some embodiments, LNPs may be prepared by mixing lipids dissolved in ethanol rapidly with RNA in an aqueous buffer. While RNA particles described herein may comprise polymer conjugated lipids such as PEG lipids, provided herein are also RNA particles which do not comprise polymer conjugated lipids such as PEG lipids.

In some embodiments, the LNPs comprising RNA and at least one cationic or cationically ionizable lipid described herein are prepared by (a) preparing an RNA solution containing water and a buffering system; (b) preparing an ethanolic solution comprising the cationic or cationically ionizable lipid and, if present, one or more additional lipids; and (c) mixing the RNA solution prepared under (a) with the ethanolic solution prepared under (b), thereby preparing the formulation comprising LNPs. After step (c) one or more steps selected from diluting and filtrating, such as tangential flow filtrating, can follow.

In some embodiments, the LNPs comprising RNA and at least one cationic or cationically ionizable lipid described herein are prepared by (a′) preparing liposomes or a colloidal preparation of the cationic or cationically ionizable lipid and, if present, one or more additional lipids in an aqueous phase; and (b′) preparing an RNA solution containing water and a buffering system; and (c′) mixing the liposomes or colloidal preparation prepared under (a′) with the RNA solution prepared under (b′). After step (c′) one or more steps selected from diluting and filtrating, such as tangential flow filtrating, can follow.

The present disclosure describes particles comprising RNA (especially mRNA) and at least one cationic or cationically ionizable lipid which associates with the RNA to form RNA particles and compositions comprising such particles. The RNA particles may comprise RNA which is complexed in different forms by non-covalent interactions to the particle. The particles described herein are not viral particles, in particular infectious viral particles, i.e., they are not able to virally infect cells.

Suitable cationic or cationically ionizable lipids are those that form RNA particles and are included by the term “particle forming components” or “particle forming agents”. The term “particle forming components” or “particle forming agents” relates to any components which associate with RNA to form RNA particles. Such components include any component which can be part of RNA particles.

In some embodiments, RNA particles (especially mRNA particles) comprise more than one type of RNA molecules, where the molecular parameters of the RNA molecules may be similar or different from each other, like with respect to molar mass or fundamental structural elements such as molecular architecture, capping, coding regions or other features.

In particulate formulation, it is possible that each RNA species is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, followed by a step of mixing of the individual particulate formulations. By the step of mixing, a formulation comprising a mixed population of RNA-containing particles is obtainable. Individual particulate populations may be together in one container, comprising a mixed population of individual particulate formulations.

Alternatively, it is possible that all RNA species of the pharmaceutical composition are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Polymers

Given their high degree of chemical flexibility, polymers are commonly used materials for nanoparticle-based delivery. Typically, cationic polymers are used to electrostatically condense the negatively charged RNA into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly (β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75.102 to 107 Da, preferably 1000 to 105 Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment.

Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes alkyl, alkenyl, or alkynyl as defined herein. It should be appreciated that one or more of the hydrogen in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have six to thirty, typically six to twenty, often six to eighteen carbon atoms. Exemplary nonpolar alkyl groups include, but are not limited to, hexyl, decyl, dodecyl, tetradecyl, hexadecyl, octadecyl, and the like.

The term “alkenyl” refers to a linear or branched monovalent hydrocarbon moiety having at least one carbon carbon double bond in which the total carbon atoms may be six to thirty, typically six to twenty often six to eighteen.

The term “alkynyl” refers to a linear or branched monovalent hydrocarbon moiety having at least one carbon carbon triple bond in which the total carbon atoms may be six to thirty, typically six to twenty, often six to eighteen. Alkynyl groups can optionally have one or more carbon carbon double bonds.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids are generally cylindrical. The area occupied by the two alkyl chains is similar to the area occupied by the polar head group. Lipids have low solubility as monomers and tend to aggregate into planar bilayers that are water insoluble. Traditional surfactant monomers are generally cone shaped. The hydrophilic head groups tend to occupy more molecular space than the linear alkyl chains. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides. Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic/Cationically Ionizable Lipids

The RNA particles described herein comprise at least one cationic or cationically ionizable lipid as particle forming agent. Cationic or cationically ionizable lipids contemplated for use herein include any cationic or cationically ionizable lipids (including lipid-like materials) which are able to electrostatically bind nucleic acid. In some embodiments, cationic or cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” refers to a lipid or lipid-like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In some embodiments, a cationic lipid has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., which is not permanently cationic. Thus, depending on the pH of the composition in which the cationically ionizable lipid is solved, the cationically ionizable lipid is either positively charged or neutral. For purposes of the present disclosure, cationically ionizable lipids are covered by the term “cationic lipid” unless contradicted by the circumstances.

In some embodiments, the cationic or cationically ionizable lipid comprises a head group which includes at least one nitrogen atom (N) which is positive charged or capable of being protonated, e.g., under physiological conditions.

Examples of cationic or cationically ionizable lipids include, but are not limited to N,N-dimethyl-2,3-dioleyloxypropylamine (DODMA), 1,2-dioleoyl-3-trimethylammonium propane (DOTAP); 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di (tetradecoxy) propyl-(2-hydroxyethyl)-dimethylazanium (DMRIE), 1,2-dimyristoyl-sn-glycero-3-ethylphosphocholine (DMEPC), I,2-dimyristoyl-3-propane (DMTAP), 1,2-dioleyloxypropyl-3-dimethyl-hydroxyethyl trimethylammonium ammonium bromide (DORIE), and 2,3-dioleoyloxy-N-[2 (spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di (3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy) heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic or cationically ionizable lipid is DOTMA. In some embodiments, the cationic or cationically ionizable lipid is DODMA.

DOTMA is a cationic lipid with a quaternary amine headgroup. The structure of DOTMA may be represented as follows:

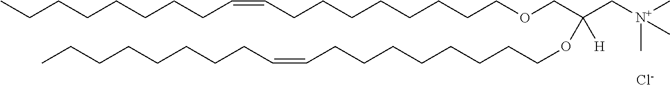 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

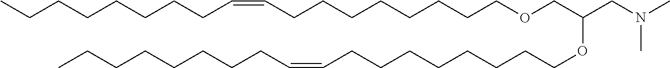 [SMILES: CCCCCCCC/C=C\CCCCCCCCOCC(CN(C)C)OCCCCCCCC/C=C\CCCCCCCC]

In some embodiments, the cationic or cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle.

### Additional Lipids

Particles described herein may also comprise lipids (including lipid-like materials) other than cationic or cationically ionizable lipids (also collectively referred to herein as cationic lipids), i.e., non-cationic lipids (including non-cationic or non-cationically ionizable lipids or lipid-like materials). Collectively, anionic and neutral lipids or lipid-like materials are referred to herein as non-cationic lipids. Optimizing the formulation of RNA particles by addition of other hydrophobic moieties, such as cholesterol and lipids, in addition to a cationic or cationically ionizable lipid may enhance particle stability and efficacy of RNA delivery.

One or more additional lipids may or may not affect the overall charge of the RNA particles.

In some embodiments, the or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In some embodiments, the RNA particles (especially the particles comprising mRNA) described herein comprise a cationic or cationically ionizable lipid and one or more additional lipids.

Without wishing to be bound by theory, the amount of the cationic or cationically ionizable lipid compared to the amount of the one or more additional lipids may affect important RNA particle characteristics, such as charge, particle size, stability, tissue selectivity, and bioactivity of the RNA. Accordingly, in some embodiments, the molar ratio of the cationic or cationically ionizable lipid to the one or more additional lipids is from about 10:0 to about 1:9, about 4:1 to about 1:2, about 4:1 to about 1:1, about 3:1 to about 1:1, or about 3:1 to about 2:1.

In some embodiments, the one or more additional lipids comprised in the RNA particles (especially in the particles comprising mRNA) described herein comprise one or more of the following: neutral lipids, steroids, and combinations thereof.

In some embodiments, the one or more additional lipids comprise a neutral lipid which is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-(18:0 Diether PC), 1-oleoyl-2-cholesterylhemisuccinoyl-sn-glycero-3-phosphocholine dipalmitoylphosphatidylcholine (DPPC), phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DOPE.

In some embodiments, the additional lipid comprises one of the following: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. Thus, in some embodiments, the RNA particles (especially the particles comprising mRNA) described herein comprise (1) a cationic or cationically ionizable lipid, and a phospholipid such as DOPE or (2) a cationic or cationically ionizable lipid and a phospholipid such as DOPE and cholesterol.

In some embodiments, the RNA particles (especially the particles comprising mRNA) described herein comprise (1) DOTMA and DOPE, (2) DOTMA, DOPE and cholesterol, (3) DODMA and DOPE or (4) DODMA, DOPE and cholesterol.

DOPE is a neutral phospholipid. The structure of DOPE may be represented as follows:

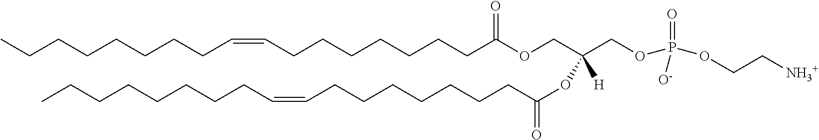 [SMILES: CCCCCCCC/C=C\CCCCCCCC(=O)OC[C@H](COP(=O)([O-])OCC[NH3+])OC(=O)CCCCCCC/C=C\CCCCCCCC]

The structure of cholesterol may be represented as follows:

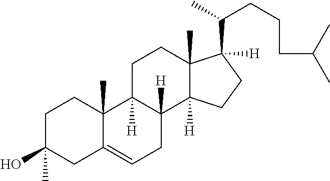

In some embodiments, particles described herein do not include a polymer conjugated lipid such as a pegylated lipid. The term “pegylated lipid” refers to a molecule comprising both a lipid portion and a polyethylene glycol portion. Pegylated lipids are known in the art.

In some embodiments, the additional lipid (e.g., one or more phospholipids and/or cholesterol) may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle. In some embodiments, the additional lipid (e.g., one or more phospholipids and/or cholesterol) comprises about 10 mol %, about 15 mol %, or about 20 mol % of the total lipid present in the particle.

In some embodiments, the additional lipid comprises a mixture of: (i) a phospholipid such as DOPE; and (ii) cholesterol or a derivative thereof. In some embodiments, the molar ratio of the phospholipid such as DOPE to the cholesterol or a derivative thereof is from about 9:0 to about 1:10, about 2:1 to about 1:4, about 1:1 to about 1:4, or about 1:1 to about 1:3.

### Polymer-Conjugated Lipids

In some embodiments, a particle may comprise at least one polymer-conjugated lipid. A polymer-conjugated lipid is typically a molecule comprising a lipid portion and a polymer portion conjugated thereto. In some embodiments, a polymer-conjugated lipid is a PEG-conjugated lipid, also referred to herein as pegylated lipid or PEG-lipid.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

### Polyethyleneglycol (PEG)-Conjugated Lipids

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di (tetradecanoyloxy) propyl-1-O-(ω-methoxy (polyethoxy)ethyl) butanedioate (PEG-S-DMG), a pegylated ceramide (PEG-cer), or a PEG dialkoxypropylcarbamate such as ω-methoxy (polyethoxy)ethyl-N-(2,3-di (tetradecanoxy) propyl) carbamate or 2,3-di (tetradecanoxy) propyl-N-(0) methoxy (polyethoxy)ethyl) carbamate, and the like.

In some embodiments, a particle may comprise one or more PEG-conjugated lipids or pegylated lipids as described in WO 2017/075531 and WO 2018/081480, the entire contents of each of which are incorporated herein by reference for the purposes described herein.

### Embodiments of Lipoplex Particles

In some embodiments of the present disclosure, the RNA described herein may be present in RNA lipoplex particles.

Lipoplexes (LPX) are electrostatic complexes which are generally formed by mixing preformed cationic lipid liposomes with anionic RNA. Formed lipoplexes possess distinct internal arrangements of molecules that arise due to the transformation from liposomal structure into compact RNA-lipoplexes. These formulations are generally characterized by their poor encapsulation of the RNA and incomplete entrapment of the RNA.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration.

### Embodiments of Lipid Nanoparticles (LNPs)

In some embodiments, RNA described herein is present in the form of lipid nanoparticles (LNPs). The LNP may comprise any lipid capable of forming a particle to which the one or more RNA molecules are attached, or in which the one or more RNA molecules are encapsulated. LNPs typically comprise four components: ionizable cationic lipids, neutral lipids such as phospholipids, a steroid such as cholesterol, and a polymer-conjugated lipid such as PEG-lipid. LNPs may be prepared by mixing lipids dissolved in ethanol with RNA in an aqueous buffer.

In some embodiments, in the RNA LNPs described herein the RNA is bound by ionizable lipid that occupies the central core of the LNP. PEG lipid forms the surface of the LNP, along with phospholipids. In some embodiments, the surface comprises a bilayer. In some embodiments, cholesterol and ionizable lipid in charged and uncharged forms can be distributed throughout the LNP.

In some embodiments, the LNP comprises one or more cationic lipids, and one or more stabilizing lipids. Stabilizing lipids include neutral lipids and pegylated lipids.

In some embodiments, the LNP comprises a cationic lipid, a neutral lipid, a steroid, a polymer-conjugated lipid; and the RNA, encapsulated within or associated with the lipid nanoparticle.

In some embodiments, the LNP comprises from 40 to 60 mol percent, 40 to 55 mol percent, from 45 to 55 mol percent, or from 45 to 50 mol percent of the cationic lipid.

In some embodiments, the neutral lipid is present in a concentration ranging from 5 to 15 mol percent, from 7 to 13 mol percent, or from 9 to 11 mol percent.

In some embodiments, the steroid is present in a concentration ranging from 30 to 50 mol percent, from 30 to 45 mol percent, from 35 to 45 mol percent or from 35 to 43 mol percent.

In some embodiments, the LNP comprises from 1 to 10 mol percent, from 1 to 5 mol percent, or from 1 to 2.5 mol percent of the polymer-conjugated lipid.

In some embodiments, the LNP comprises from 45 to 55 mol percent a cationic lipid; from 5 to 15 mol percent of a neutral lipid; from 30 to 45 mol percent of a steroid; from 1 to 5 mol percent of a polymer-conjugated lipid; and the RNA, encapsulated within or associated with the lipid nanoparticle.

In some embodiments, the mol percent is determined based on total mol of lipid present in the lipid nanoparticle. In some embodiments, the mol percent is determined based on total mol of cationic lipid, neutral lipid, steroid and polymer-conjugated lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid. In some embodiments, the pegylated lipid has the following structure:

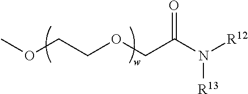

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is or comprises 2-[(Polyethylene glycol)-2000]-N,N-ditetradecylacetamide, e.g., having the following structure:

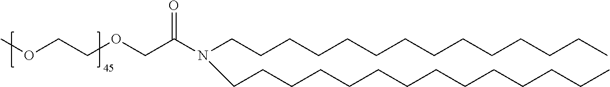

In some embodiments, a pegylated lipid is or comprises PEG2000-C-DMA, e.g., having the following structure:

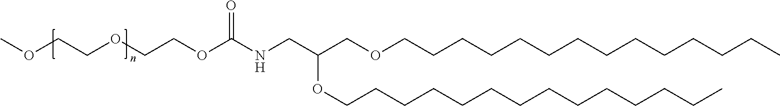

wherein n may have a mean value ranging from 30 to 60, such as about 50.

In some embodiments, the pegylated lipid is or comprises DMG-PEG 2000, e.g., having the following structure:

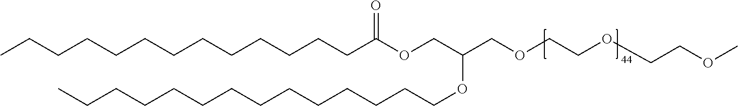

In some embodiments, the cationic lipid component of the LNPs has the structure of Formula (III):

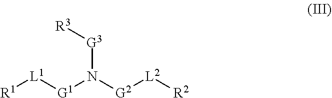

or a pharmaceutically acceptable salt, tautomer, prodrug or stereoisomer thereof, wherein: one of L¹ or L² is —O(C═O)—, —(C═O)O—, —C(═O)—, —O—, —S(O), —, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

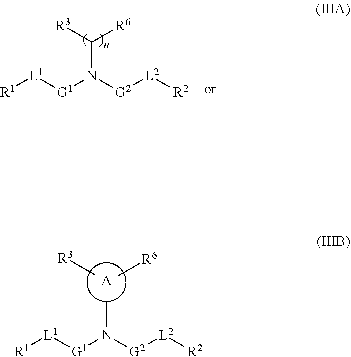

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

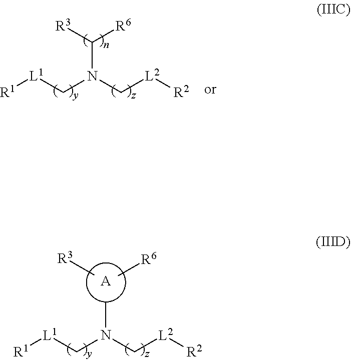

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

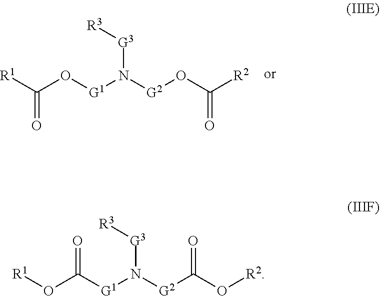

In some of the foregoing embodiments of Formula (III), the lipid has one of the following structures (IIIG), (IIIH), (IIII), or (IIIJ):

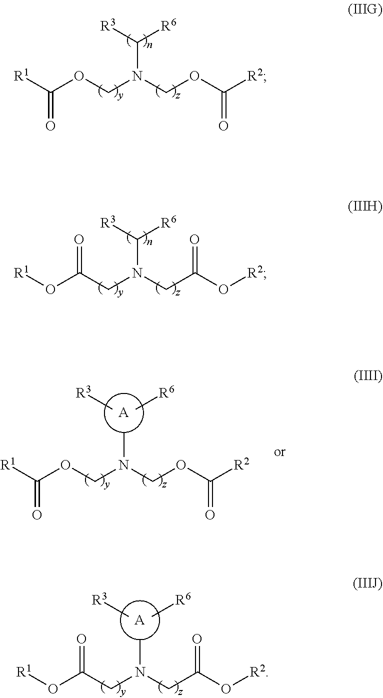

In some of the foregoing embodiments of Formula (III), n is an integer ranging from 2 to 12, for example from 2 to 8 or from 2 to 4. For example, in some embodiments, n is 3, 4, 5 or 6.

In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

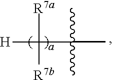

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

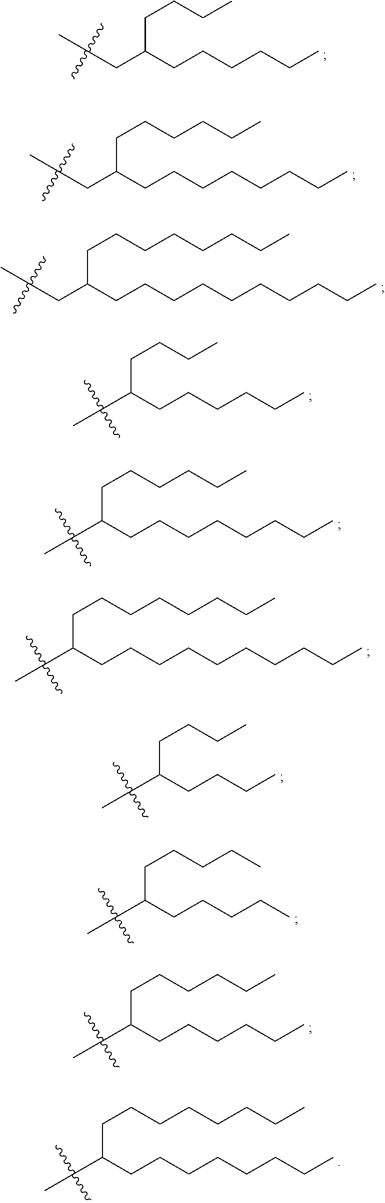

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

Representative Compounds of Formula (III).

| No. | Structure |
|---|---|
| III-1 | 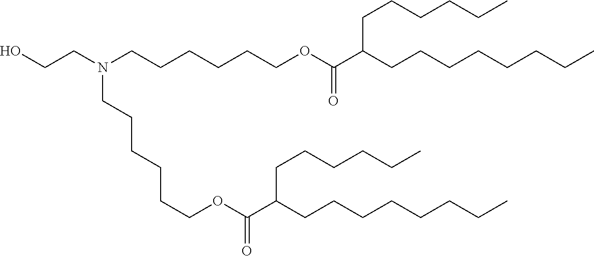 [SMILES: CCCCCCCCC(CCCCCC)C(=O)OCCCCCCN(CCO)CCCCCCOC(=O)C(CCCCCC)CCCCCCCC] |
| III-2 | 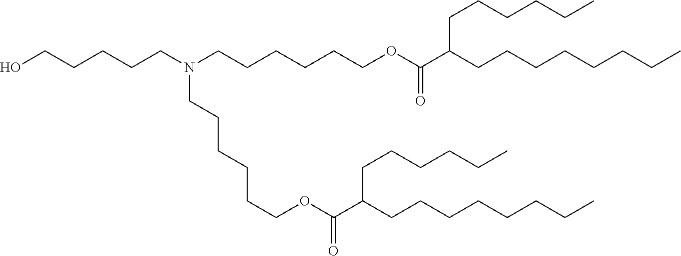 [SMILES: CCCCCCCCC(CCCCCC)C(=O)OCCCCCCN(CCCCCO)CCCCCCOC(=O)C(CCCCCC)CCCCCCCC] |
| III-3 | 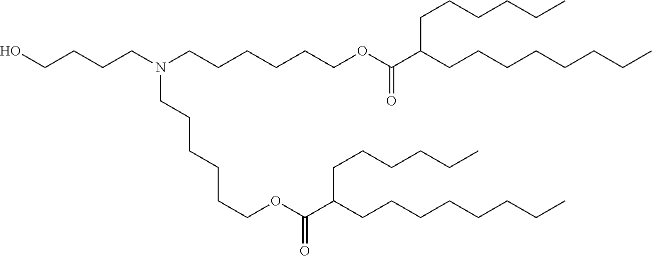 [SMILES: CCCCCCCCC(CCCCCC)C(=O)OCCCCCCN(CCCCO)CCCCCCOC(=O)C(CCCCCC)CCCCCCCC] |
| III-4 | 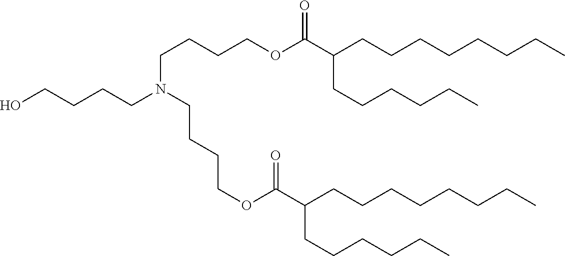 [SMILES: CCCCCCCCC(CCCCCC)C(=O)OCCCCN(CCCCO)CCCCOC(=O)C(CCCCCC)CCCCCCCC] |
| III-5 | 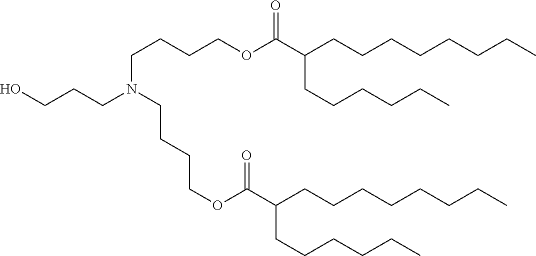 [SMILES: CCCCCCCCC(CCCCCC)C(=O)OCCCCN(CCCO)CCCCOC(=O)C(CCCCCC)CCCCCCCC] |
| III-6 | 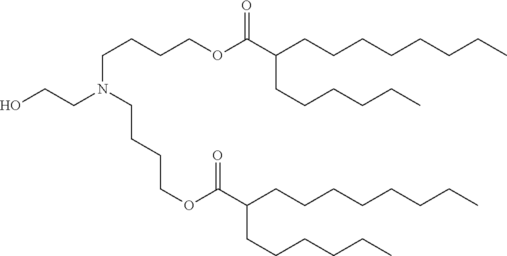 [SMILES: CCCCCCCCC(CCCCCC)C(=O)OCCCCN(CCO)CCCCOC(=O)C(CCCCCC)CCCCCCCC] |
| III-7 | 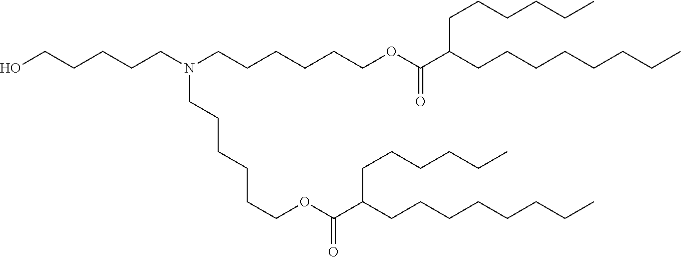 [SMILES: CCCCCCCCC(CCCCCC)C(=O)OCCCCCCN(CCCCCO)CCCCCCOC(=O)C(CCCCCC)CCCCCCCC] |
| III-8 | 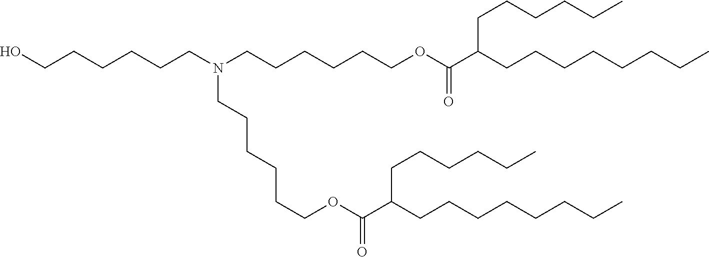 [SMILES: CCCCCCCCC(CCCCCC)C(=O)OCCCCCCN(CCCCCCO)CCCCCCOC(=O)C(CCCCCC)CCCCCCCC] |
| III-9 | 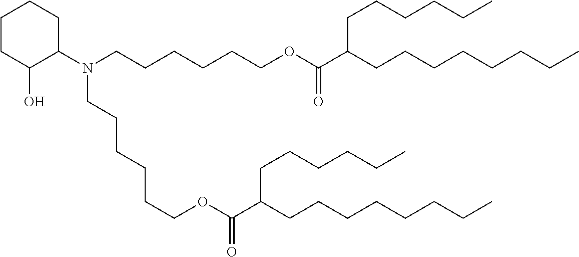 [SMILES: CCCCCCCCC(CCCCCC)C(=O)OCCCCCCN(CCCCCCOC(=O)C(CCCCCC)CCCCCCCC)C1CCCCC1O] |
| III-10 | 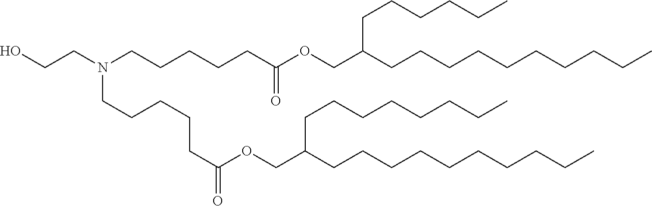 [SMILES: CCCCCCCCCCC(CCCCCC)COC(=O)CCCCCN(CCO)CCCCCC(=O)OCC(CCCCCCCC)CCCCCCCCCC] |
| III-11 | 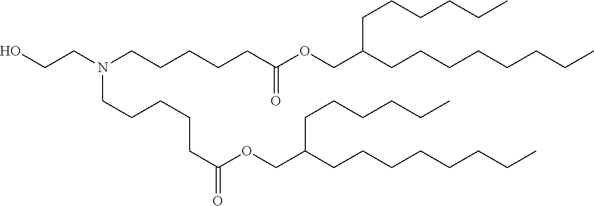 [SMILES: CCCCCCCCC(CCCCCC)COC(=O)CCCCCN(CCO)CCCCCC(=O)OCC(CCCCCC)CCCCCCCC] |
| III-12 | 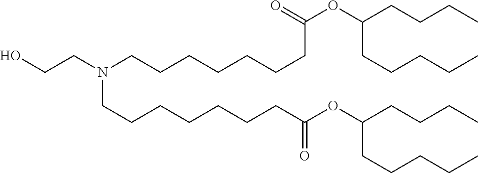 [SMILES: CCCCCC(CCCC)OC(=O)CCCCCCCN(CCO)CCCCCCCC(=O)OC(CCCC)CCCCC] |
| III-13 | 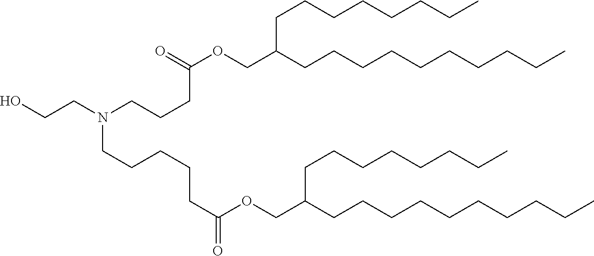 [SMILES: CCCCCCCCCCC(CCCCCCCC)COC(=O)CCCCCN(CCO)CCCC(=O)OCC(CCCCCCCC)CCCCCCCCCC] |
| III-14 | 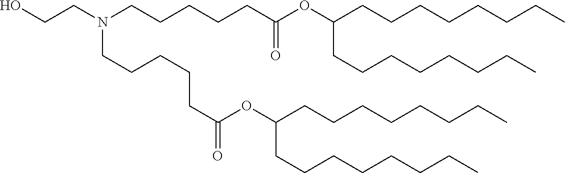 [SMILES: CCCCCCCCC(CCCCCCCC)OC(=O)CCCCCN(CCO)CCCCCC(=O)OC(CCCCCCCC)CCCCCCCC] |
| III-15 | 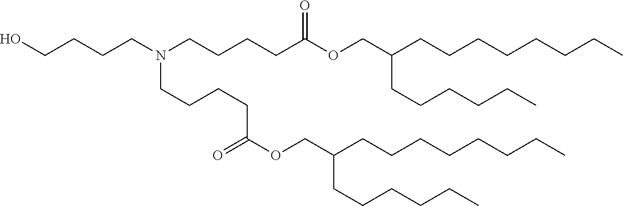 [SMILES: CCCCCCCCC(CCCCCC)COC(=O)CCCCN(CCCCO)CCCCC(=O)OCC(CCCCCC)CCCCCCCC] |
| III-16 | 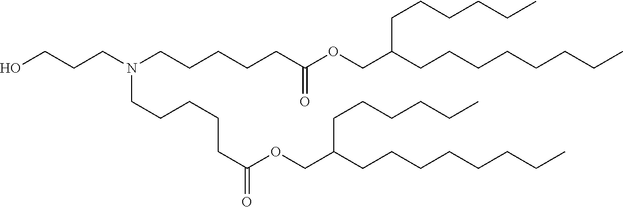 [SMILES: CCCCCCCCC(CCCCCC)COC(=O)CCCCCN(CCCO)CCCCCC(=O)OCC(CCCCCC)CCCCCCCC] |
| III-17 | 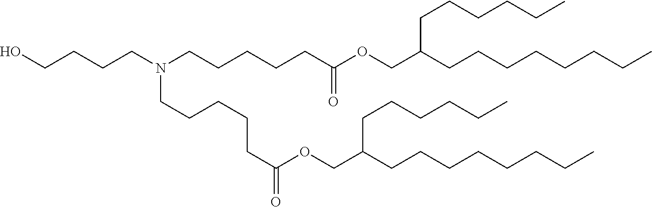 [SMILES: CCCCCCCCC(CCCCCC)COC(=O)CCCCCN(CCCCO)CCCCCC(=O)OCC(CCCCCC)CCCCCCCC] |
| III-18 | 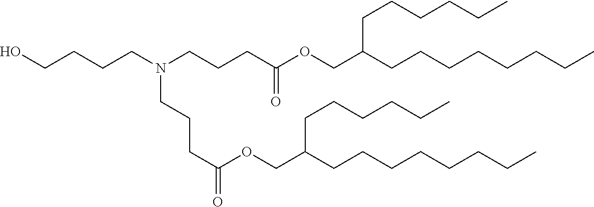 [SMILES: CCCCCCCCC(CCCCCC)COC(=O)CCCN(CCCCO)CCCC(=O)OCC(CCCCCC)CCCCCCCC] |
| III-19 | 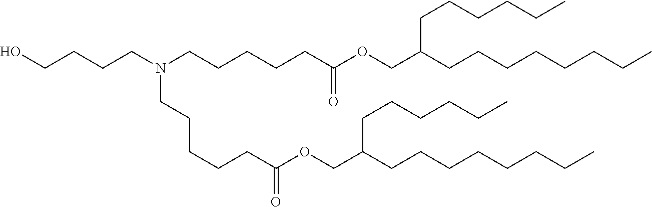 [SMILES: CCCCCCCCC(CCCCCC)COC(=O)CCCCCN(CCCCO)CCCCCC(=O)OCC(CCCCCC)CCCCCCCC] |
| III-20 | 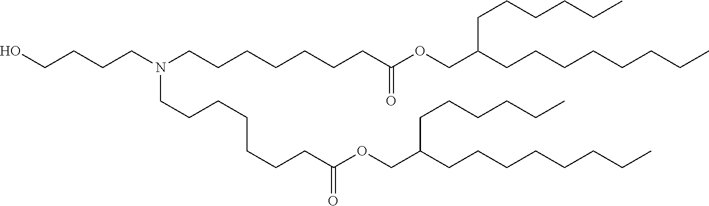 [SMILES: CCCCCCCCC(CCCCCC)COC(=O)CCCCCCCN(CCCCO)CCCCCCCC(=O)OCC(CCCCCC)CCCCCCCC] |
| III-21 | 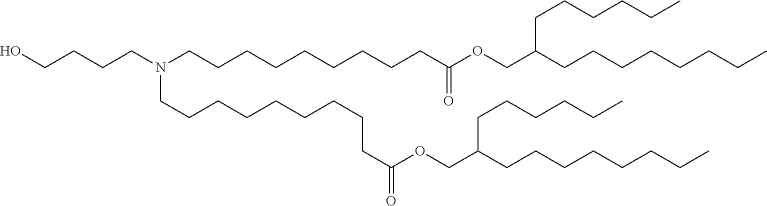 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 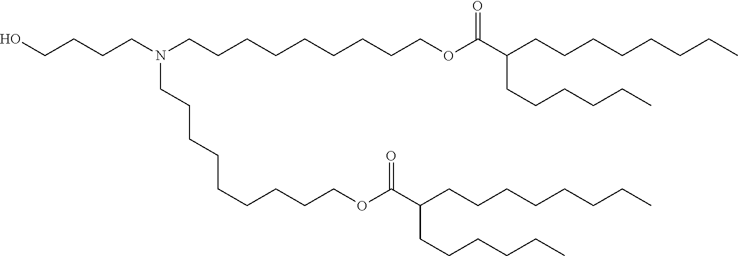 [SMILES: CCCCCCCCC(CCCCCC)C(=O)OCCCCCCCCCN(CCCCO)CCCCCCCCCOC(=O)C(CCCCCC)CCCCCCCC] |
| III-23 | 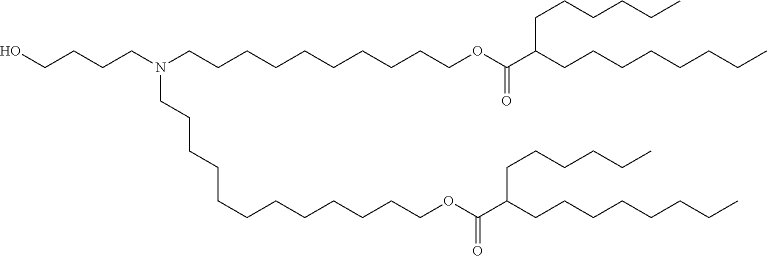 [SMILES: CCCCCCCCC(CCCCCC)C(=O)OCCCCCCCCCCCCN(CCCCO)CCCCCCCCCCOC(=O)C(CCCCCC)CCCCCCCC] |
| III-24 | 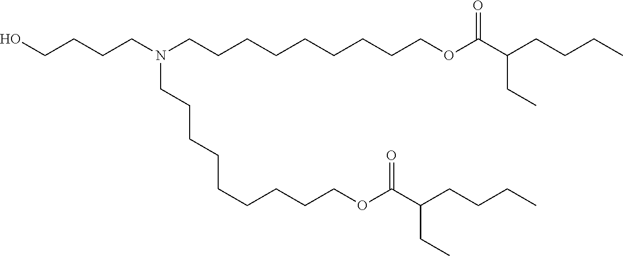 [SMILES: CCCCC(CC)C(=O)OCCCCCCCCCN(CCCCO)CCCCCCCCCOC(=O)C(CC)CCCC] |
| III-25 | 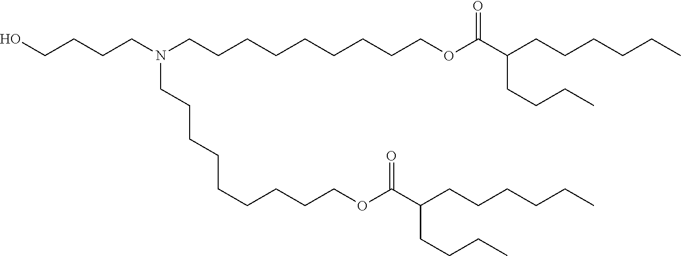 [SMILES: CCCCCCC(CCCC)C(=O)OCCCCCCCCCN(CCCCO)CCCCCCCCCOC(=O)C(CCCC)CCCCCC] |
| III-26 | 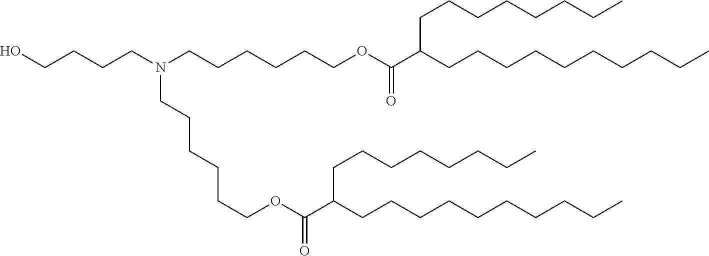 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 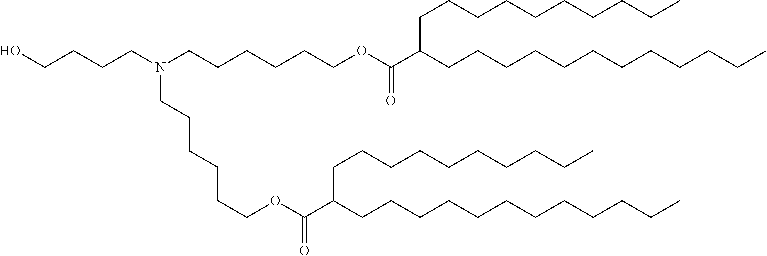 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 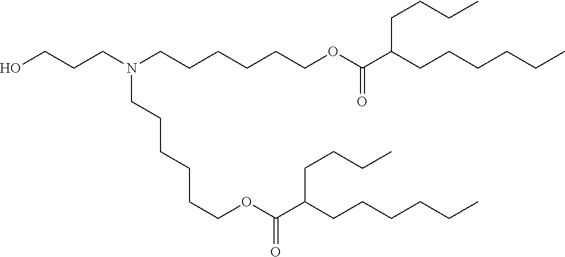 [SMILES: CCCCCCC(CCCC)C(=O)OCCCCCCN(CCCO)CCCCCCOC(=O)C(CCCC)CCCCCC] |
| III-29 | 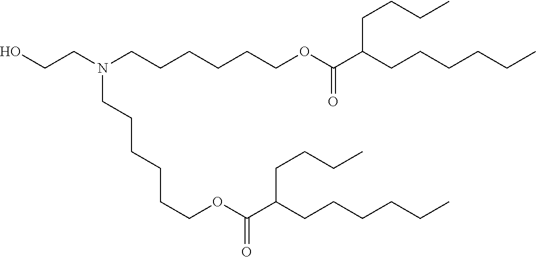 [SMILES: CCCCCCC(CCCC)C(=O)OCCCCCCN(CCO)CCCCCCOC(=O)C(CCCC)CCCCCC] |
| III-30 | 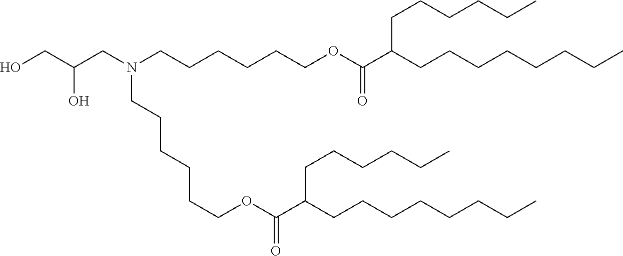 [SMILES: CCCCCCCCC(CCCCCC)C(=O)OCCCCCCN(CCCCCCOC(=O)C(CCCCCC)CCCCCCCC)CC(O)CO] |
| III-31 | 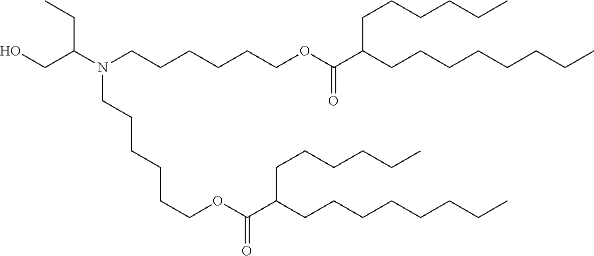 [SMILES: CCCCCCCCC(CCCCCC)C(=O)OCCCCCCN(CCCCCCOC(=O)C(CCCCCC)CCCCCCCC)C(CC)CO] |
| III-32 | 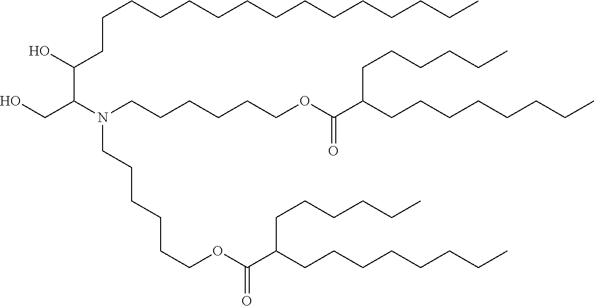 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 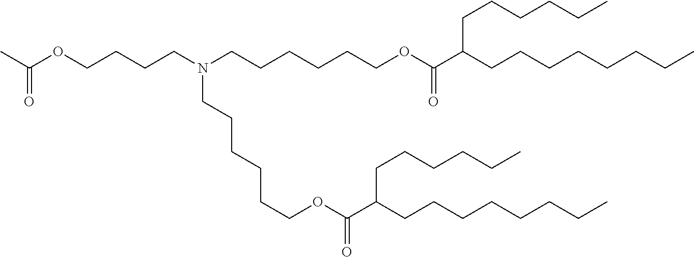 [SMILES: CCCCCCCCC(CCCCCC)C(=O)OCCCCCCN(CCCCCCOC(=O)C(CCCCCC)CCCCCCCC)CCCCOC(C)=O] |
| III-34 | 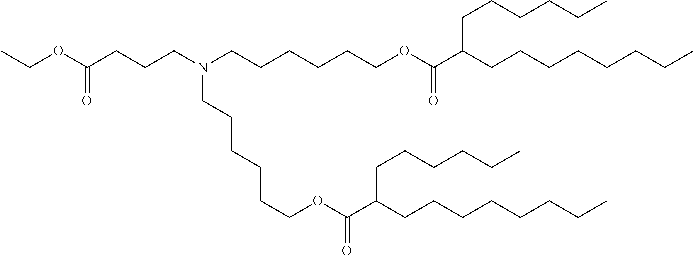 [SMILES: CCCCCCCCC(CCCCCC)C(=O)OCCCCCCN(CCCCCCOC(=O)C(CCCCCC)CCCCCCCC)CCCC(=O)OCC] |
| III-35 | 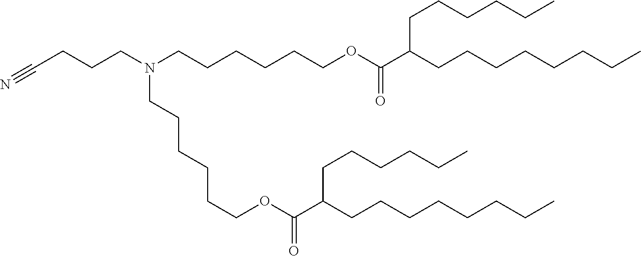 [SMILES: CCCCCCCCC(CCCCCC)C(=O)OCCCCCCN(CCCC#N)CCCCCCOC(=O)C(CCCCCC)CCCCCCCC] |
| III-36 | 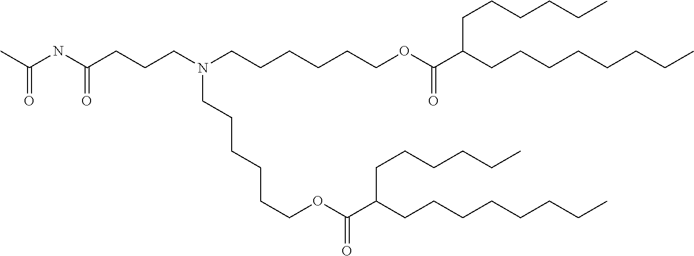 [SMILES: CCCCCCCCC(CCCCCC)C(=O)OCCCCCCN(CCCCCCOC(=O)C(CCCCCC)CCCCCCCC)CCCC(=O)NC(C)=O] |
| III-37 | 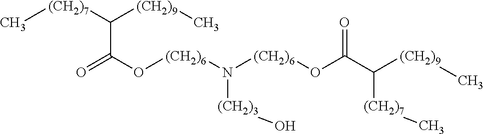 |
| III-38 | 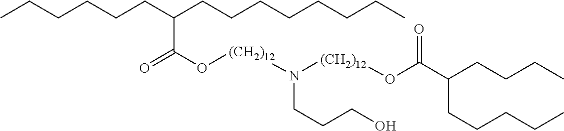 |
| III-39 | 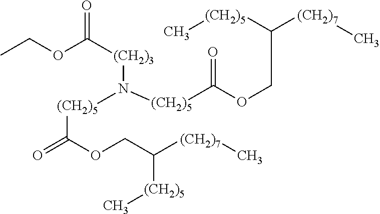 |
| III-40 | 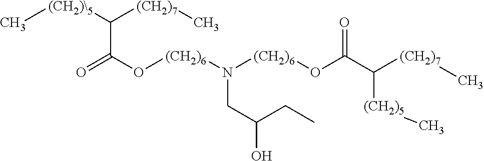 |
| III-41 | 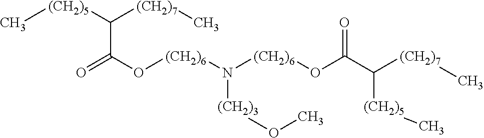 |
| III-42 | 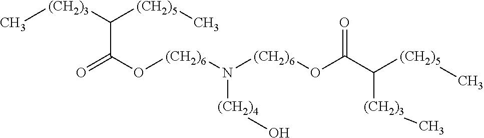 |
| III-43 | 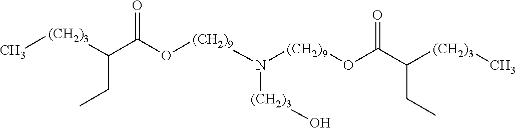 |
| III-44 | 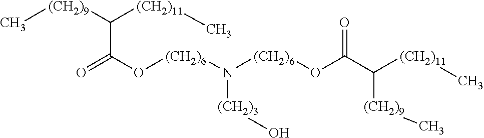 |
| III-45 | 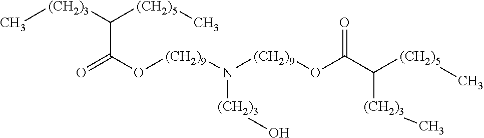 |
| III-46 | 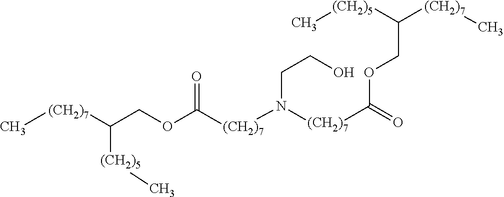 |
| III-47 | 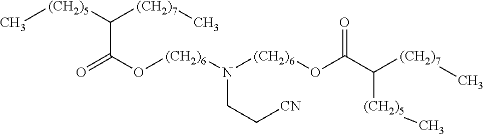 |
| III-48 | 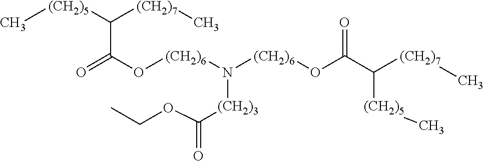 |
| III-49 | 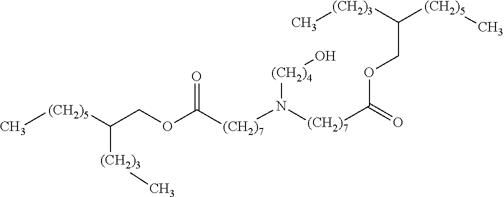 |

Further representative cationic lipids are as follows:

| No. | Structure |
|---|---|
| A | 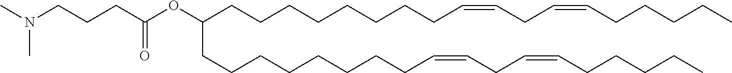 [SMILES: CCCCC/C=C\C/C=C\CCCCCCCCC(CCCCCCCC/C=C\C/C=C\CCCCC)OC(=O)CCCN(C)C] |
| B | 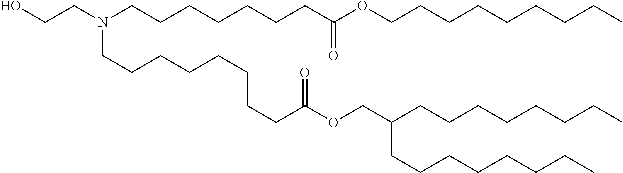 [SMILES: CCCCCCCCCOC(=O)CCCCCCCN(CCO)CCCCCCCCC(=O)OCC(CCCCCCCC)CCCCCCCC] |
| C | 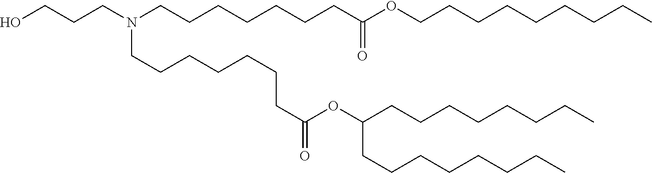 [SMILES: CCCCCCCCCOC(=O)CCCCCCCN(CCCO)CCCCCCCC(=O)OC(CCCCCCCC)CCCCCCCC] |
| D | 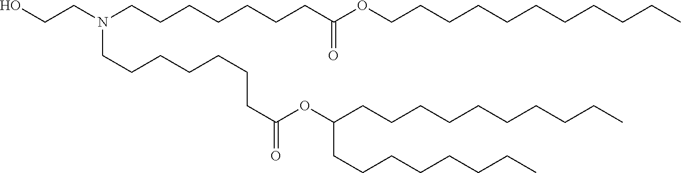 [SMILES: CCCCCCCCCCCOC(=O)CCCCCCCN(CCO)CCCCCCCC(=O)OC(CCCCCCCC)CCCCCCCCCC] |
| E | 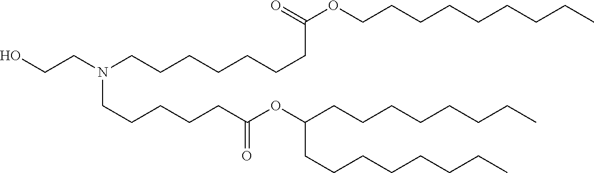 [SMILES: CCCCCCCCCOC(=O)CCCCCCCN(CCO)CCCCCC(=O)OC(CCCCCCCC)CCCCCCCC] |
| F | 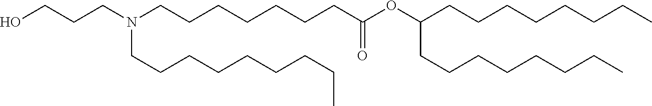 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC.

In some embodiments, the steroid is cholesterol. In some embodiments, the pegylated lipid is DMG-PEG 2000.

Various lipids (including, e.g., cationic lipids, neutral lipids, and polymer-conjugated lipids) are known in the art and can be used herein to form lipid nanoparticles, e.g., lipid nanoparticles targeting a specific cell type (e.g., liver cells). In some embodiments, a neutral lipid may be or comprise a phospholipid or derivative thereof (e.g., 1,2-Distearoyl-sn-glycero-3-phosphocholine (DPSC)) and/or cholesterol. In some embodiments, a polymer-conjugated lipid may be a PEG-conjugated lipid (e.g., 2-[(polyethylene glycol)-2000]-N,N-ditetradecylacetamide or a derivative thereof).

In some embodiments, the LNP comprises a lipid of Formula (III), RNA, a neutral lipid, a steroid and a pegylated lipid. In some embodiments, the neutral lipid is DSPC. In some embodiments, the steroid is cholesterol. In some embodiments, the pegylated lipid is ALC-0159.

### ALC-0159:

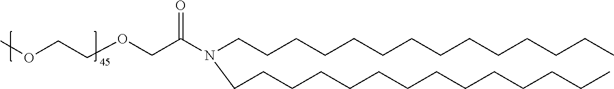

In some embodiments, RNA described herein is formulated in a composition comprising a cationic lipid, a neutral lipid, a steroid, a polymer conjugated lipid; and the RNA.

In some embodiments, the composition comprises a lipid of Formula (III), RNA, a neutral lipid, a steroid and a pegylated lipid. In some embodiments, the neutral lipid is DSPC. In some embodiments, the steroid is cholesterol. In some embodiments, the pegylated lipid is ALC-0159. In some embodiments, the cationic lipid is a lipid of Formula (III), the neutral lipid is DSPC, the steroid is cholesterol, and the pegylated lipid is ALC-0159.

In some embodiments, the composition comprises ALC-0315, RNA, a neutral lipid, a steroid and a pegylated lipid. In some embodiments, the neutral lipid is DSPC. In some embodiments, the steroid is cholesterol. In some embodiments, the pegylated lipid is ALC-0159. In some embodiments, the cationic lipid is ALC-0315, the neutral lipid is DSPC, the steroid is cholesterol, and the pegylated lipid is ALC-0159.

ALC-0315=((4-hydroxybutyl) azanediyl)bis(hexane-6,1-diyl)bis(2-hexyldecanoate)/6-[N-6-(2-hexyldecanoyloxy) hexyl-N-(4-hydroxybutyl)amino]hexyl 2-hexyldecanoate

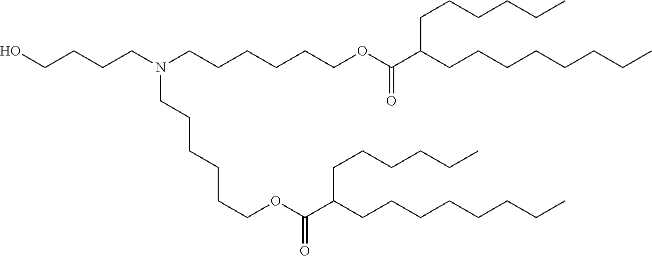 [SMILES: CCCCCCCCC(CCCCCC)C(=O)OCCCCCCN(CCCCO)CCCCCCOC(=O)C(CCCCCC)CCCCCCCC]

ALC-0159=2-[(polyethylene glycol)-2000]-N,N-ditetradecylacetamide/2-[2-(ω-methoxy (polyethyleneglycol2000) ethoxy]-N,N-ditetradecylacetamide

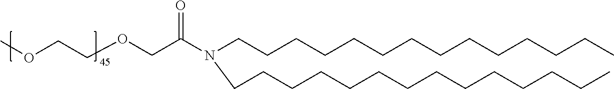

DSPC=1,2-Distearoyl-sn-glycero-3-phosphocholine

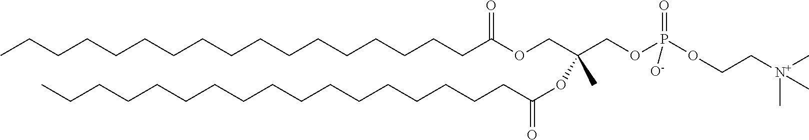 [SMILES: CCCCCCCCCCCCCCCCCC(=O)OC[C@](C)(COP(=O)([O-])OCC[N+](C)(C)C)OC(=O)CCCCCCCCCCCCCCCCC]

### Cholesterol:

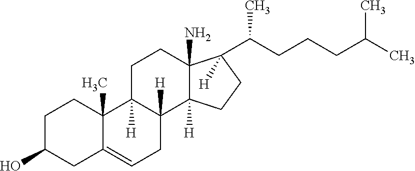

In some embodiments, the composition comprises ALC-0366, RNA, a neutral lipid, a steroid and a pegylated lipid. In some embodiments, the neutral lipid is DSPC. In some embodiments, the steroid is cholesterol. In some embodiments, the pegylated lipid is ALC-0159. In some embodiments, the cationic lipid is ALC-0366, the neutral lipid is DSPC, the steroid is cholesterol, and the pegylated lipid is ALC-0159.

ALC-0366=((3-hydroxypropyl) azanediyl)bis(nonane-9,1-diyl)bis(2-butyloctanoate)

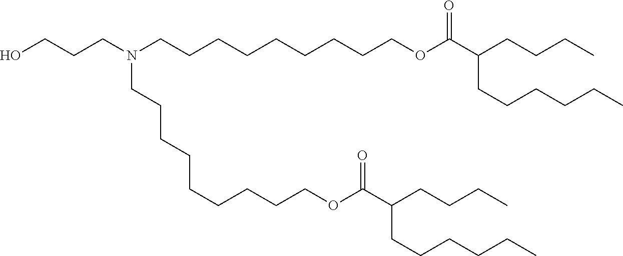 [SMILES: CCCCCCC(CCCC)C(=O)OCCCCCCCCCN(CCCO)CCCCCCCCCOC(=O)C(CCCC)CCCCCC]

In some embodiments, the composition comprises 3D-P-DMA, RNA, a neutral lipid, a steroid and a pegylated lipid. In some embodiments, the neutral lipid is DSPC. In some embodiments, the steroid is cholesterol. In some embodiments, the pegylated lipid is PEG2000-C-DMA. In some embodiments, the cationic lipid is 3D-P-DMA, the neutral lipid is DSPC, the steroid is cholesterol, and the pegylated lipid is PEG2000-C-DMA.

3D-P-DMA=(6Z,16Z)-12-((Z)-dec-4-en-1-yl) docosa-6,16-dien-11-yl 5-(dimethylamino) pentanoate

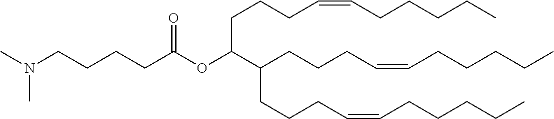 [SMILES: CCCCC/C=C\CCCC(CCC/C=C\CCCCC)C(CCC/C=C\CCCCC)OC(=O)CCCCN(C)C]

“PEG₂₀₀₀-C-DMA”: 3-N-[(ω-Methoxy polyethylene glycol)2000) carbamoyl]-1,2-dimyristyloxy-propylamine (MPEG-(2 kDa)-C-DMA or Methoxy-polyethylene glycol-2,3-bis(tetradecyloxy) propylcarbamate (2000))

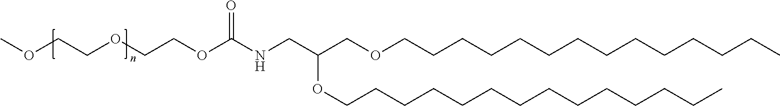

wherein n has a mean value ranging from 30 to 60, such as about 50.

The N/P value is preferably at least about 4. In some embodiments, the N/P value ranges from 4 to 20, 4 to 12, 4 to 10, 4 to 8, or 5 to 7. In some embodiments, the N/P value is about 6.

### Compositions Comprising RNA

A composition comprising one or more RNAs described herein, e.g., in the form of RNA particles, may comprise salts, buffers, or other components as further described below.

In some embodiments, a salt for use in the compositions described herein comprises sodium chloride. Without wishing to be bound by theory, sodium chloride functions as an ionic osmolality agent for preconditioning RNA prior to mixing with lipids. In some embodiments, the compositions described herein may comprise alternative organic or inorganic salts. Alternative salts include, without limitation, potassium chloride, dipotassium phosphate, monopotassium phosphate, potassium acetate, potassium bicarbonate, potassium sulfate, disodium phosphate, monosodium phosphate, sodium acetate, sodium bicarbonate, sodium sulfate, lithium chloride, magnesium chloride, magnesium phosphate, calcium chloride, and sodium salts of ethylenediaminetetraacetic acid (EDTA).

Generally, compositions for storing RNA particles such as for freezing RNA particles comprise low sodium chloride concentrations, or comprises a low ionic strength. In some embodiments, the sodium chloride is at a concentration from 0 mM to about 50 mM, from 0 mM to about 40 mM, or from about 10 mM to about 50 mM.

According to the present disclosure, the RNA particle compositions described herein have a pH suitable for the stability of the RNA particles and, in particular, for the stability of the RNA. Without wishing to be bound by theory, the use of a buffer system maintains the pH of the particle compositions described herein during manufacturing, storage and use of the compositions. In some embodiments of the present disclosure, the buffer system may comprise a solvent (in particular, water, such as deionized water, in particular water for injection) and a buffering substance. The buffering substance may be selected from 2-[4-(2-hydroxyethyl) piperazin-1-yl]ethanesulfonic acid (HEPES), 2-amino-2-(hydroxymethyl) propane-1,3-diol (Tris), acetate, and histidine. A preferred buffering substance is HEPES.

Compositions described herein may also comprise a cyroprotectant and/or a surfactant as stabilizer to avoid substantial loss of the product quality and, in particular, substantial loss of RNA activity during storage, freezing, and/or lyophilization, for example to reduce or prevent aggregation, particle collapse, RNA degradation and/or other types of damage.

In an embodiment, the cryoprotectant is a carbohydrate. The term “carbohydrate”, as used herein, refers to and encompasses monosaccharides, disaccharides, trisaccharides, oligosaccharides and polysaccharides.

In an embodiment, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In an embodiment, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like.

The term “trisaccharide” means three sugars linked together to form one molecule. Examples of a trisaccharides include raffinose and melezitose.

In an embodiment, the cryoprotectant is an oligosaccharide. The term “oligosaccharide”, as used herein refers to a compound or a chemical moiety formed by 3 to about 15, such as 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced. In an embodiment, the cryoprotectant is a cyclic oligosaccharide. The term “cyclic oligosaccharide”, as used herein refers to a compound or a chemical moiety formed by 3 to about 15, such as 6, 7, 8, 9, or 10 monosaccharide units that are bonded together through glycosidic linkages, for example through 1-4 linkages or 1-6 linkages, to form a cyclic structure. Exemplary cyclic oligosaccharide cryoprotectants include cyclic oligosaccharides that are discrete compounds, such as a cyclodextrin, β cyclodextrin, or γcyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an a, B, or γcyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-β-cyclodextrin, e.g., partially etherified cyclodextrins (e.g., partially etherified β cyclodextrins).

An exemplary cryoprotectant is a polysaccharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In some embodiments, RNA particle compositions may include sucrose. Without wishing to be bound by theory, sucrose functions to promote cryoprotection of the compositions, thereby preventing RNA (especially mRNA) particle aggregation and maintaining chemical and physical stability of the composition. In some embodiments, RNA particle compositions may include alternative cryoprotectants to sucrose. Alternative stabilizers include, without limitation, trehalose and glucose. In a specific embodiment, an alternative stabilizer to sucrose is trehalose or a mixture of sucrose and trehalose.

A preferred cryoprotectant is selected from the group consisting of sucrose, trehalose, glucose, and a combination thereof, such as a combination of sucrose and trehalose. In a preferred embodiment, the cryoprotectant is sucrose.

Some embodiments of the present disclosure contemplate the use of a chelating agent in an RNA composition described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In some embodiments, the chelating agent is EDTA or a salt of EDTA. In an exemplary embodiment, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In an alternative embodiment, the RNA particle compositions described herein do not comprise a chelating agent.

### Pharmaceutical Compositions

The agents described herein may be administered in pharmaceutical compositions or medicaments and may be administered in the form of any suitable pharmaceutical composition. In some embodiments, the pharmaceutical composition is for therapeutic or prophylactic treatments, e.g., for use in treating or preventing a disease such as bacterial infection.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease by administration of said pharmaceutical composition to a subject.

The pharmaceutical compositions of the present disclosure may be in a storable form (e.g., in a frozen or lyophilized/freeze-dried form) or in a “ready-to-use form” (i.e., in a form which can be immediately administered to a subject, e.g., without any processing such as diluting). Thus, prior to administration of a storable form of a pharmaceutical composition, this storable form has to be processed or transferred into a ready-to-use or administrable form. E.g., a frozen pharmaceutical composition has to be thawed, or a freeze-dried pharmaceutical composition has to be reconstituted, e.g. by using a suitable solvent (e.g., deionized water, such as water for injection) or liquid (e.g., an aqueous solution).

The pharmaceutical compositions according to the present disclosure are generally applied in a “pharmaceutically effective amount” and in “a pharmaceutically acceptable preparation”. The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In some embodiments relating to the treatment of a particular disease, the desired reaction may relate to inhibition of the course of the disease. This comprises slowing down the progress of the disease and, in some embodiments, interrupting or reversing the progress of the disease. The desired reaction in a treatment of a disease may also be delay of the onset or a prevention of the onset of said disease or said condition. An effective amount of the pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the pharmaceutical compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In some embodiments, the pharmaceutical compositions described herein may be administered intravenously, intraarterially, subcutaneously, intradermally, dermally, intranodally, or intramuscularly. In some embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the pharmaceutical compositions are formulated for systemic administration. In some embodiments, the systemic administration is by intravenous administration. In some embodiments, the pharmaceutical compositions are formulated for respiratory/pulmonary administration route and/or administered by respiratory/pulmonary administration route, e.g., by inhalation. In this manner, the RNA described herein may be locally/regionally or systemically delivered to lungs and/or the respiratory tract. The lungs may also be used as a portal of entry to the body, enabling delivery of the RNA via the airways into the bloodstream. Thus, inhaled formulations may be used for systemic delivery.

### Use of Compositions

Compositions described herein may be used in the therapeutic or prophylactic treatment of various diseases, in particular diseases caused by or related to bacterial infection.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories.

Diseases usually affect individuals not only physically, but also emotionally, as contracting and living with many diseases can alter one's perspective on life, and one's personality.

The term “infectious disease” or “infection” refers to any disease which can be transmitted from individual to individual or from organism to organism, and is caused by a microbial agent. Infectious diseases are known in the art and include, for example, a viral disease, a bacterial disease, or a parasitic disease, which diseases are caused by a virus, a bacterium, and a parasite, respectively.

In the present context, the term “treatment”, “treating” or “therapeutic intervention” relates to the management and care of a subject for the purpose of combating a condition such as a disease. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease (e.g., cancer, infectious diseases) but may or may not have the disease, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In some embodiments of the present disclosure, the “individual” or “subject” is a “patient”.

The term “patient” means an individual or subject for treatment, in particular a diseased individual or subject.

The agents and methods described herein find application in the treatment of a bacterial infection in a subject. In some embodiments, the agents and methods described herein are useful for treating a bacterial infection caused by Gram-positive bacteria and/or Gram-negative bacteria and/or atypical bacteria.

The term “bacterial infection” refers to the invasion of a subject by pathogenic bacteria. This includes the excessive growth of bacteria which are normally present in or on the body of the subject, but more generally, a bacterial infection can be any situation in which the presence of a bacterial population(s) is damaging to a host organism. Thus, for example, a subject suffers from a bacterial population when excessive numbers of a bacterial population are present in or on the subject's body, or when the effects of the presence of a bacterial population(s) is damaging to the cells, tissue, or organs of the subject.

The agents and methods described herein are applicable to a variety of bacterial infections caused by different bacterial strains, in particular bacteria which are a major cause of morbidity and mortality in hospital-based infections, e.g., Staphylococcus aureus, Streptococcus pneumoniae, various Enterococci, and Pseudomonas aeruginosa. However, the approach described below is clearly applicable to any human bacterial pathogens including but not restricted to Mycobacterium tuberculosis, Neisseria gonorrhoeae, Haemophilus influenza, Acinobacter, Escherichia coli, Shigella dysenteria, Streptococcus pyogenes, Helicobacter pylori, and Mycoplasma species. In some embodiments, the peptidoglycan hydrolase, e.g., endolysin, used is effective in breaking down peptidoglycan in bacterial cell wall of the bacterium causing the infection to be treated. In some embodiments, the peptidoglycan hydrolase, e.g., endolysin, used is derived from a bacteriophage of the bacterium causing the infection to be treated.

In some embodiments, the bacterial infection is caused by at least one bacterium selected from the genera Enterococcus, Staphylococcus, Streptococcus, Bacillus, Acinetobacter, Burkholderia, Coxiella, Francisella, Yersina, Klebsiella, Escherichia, Enterobacter and Pseudomonas.

In some embodiments, the bacterial infection is caused by at least one bacterium selected from the genera Enterococcus, Staphylococcus, Acinetobacter, Burkholderia, Klebsiella, Escherichia, Enterobacter and Pseudomonas.

In some embodiments, the bacterial infection is caused by at least one bacterium selected from Enterococcus faeculis, Enterococcus faecium, Staphylococcus aureus, Streptococcus pyogenes, Streptococcus pneumoniae, Streptococcus agalactiae, Bacillus anthracis, Bacillus cereus, Bacillus subtilis, Haemophilus influenzae, Acinetobacter baumannii, Burkholderia multivorans, Burkholderia cenocepacia, Burkholderia cepacia, Burkholderia mallei, Burkholderia pseudomallei, Coxiella burnetii, Citrobacter freundii, Escherichia coli, Enterobacter cloacae, Enterobacter aerogenes, Francisella tularensis, Yersina pestis, Klebsiella pneumoniae, Serratia marcesens, Salmonella typhi, Salmonella typhimurum, Stenotrophomonas maltophilia, Pseudomonas aeruginosa and Neisseria gonorrhoeae. In some embodiments, the bacterial infection is caused by at least one bacterium selected from Enterococcus faeculis, Enterococcus faecium, Staphylococcus aureus, Acinetobacter baumannii, Burkholderia multivorans, Burkholderia cenocepacia, Burkholderia cepacia, Klebsiella pneumonia and Pseudomonas aeruginosa.

In some embodiments, the bacterial infection is caused by Gram-positive bacteria selected from Enterococcus faeculis, Enterococcus faecium, Staphylococcus aureus, Streptococcus pyogenes, Streptococcus pneumoniae, Streptococcus agalactiae, Bacillus anthracis, Bacillus cereus and Bacillus subtilis.

In some embodiments, the infection is caused by Gram-negative bacteria, such as Haemophilus influenzae, Acinetobacter baumannii, Burkholderia multivorans, Burkholderia cenocepacia, Burkholderia cepacia, Burkholderia mallei, Burkholderia pseudomallei, Coxiella burnetii, Citrobacter freundii, Escherichia coli, Enterobacter cloacae, Enterobacter aerogenes, Francisella tularensis, Yersina pestis, Klebsiella pneumoniae, Pseudomonas aeruginosa and Neisseria gonorrhoeae.

In some embodiments, the bacterial infection is caused by drug-resistant bacteria. Such drug-resistant bacteria are bacteria that are resistant to one or more antibacterials other than the agents described herein. The term “resistance”, “antibacterial resistance”, or “drug-resistant” refers to bacteria that are able to survive exposure to one or more antibacterial drugs. In some embodiments, the drug-resistant bacteria include Streptococcus pyogenes, Streptococcus agalactiae, Streptococcus pneumoniae (including penicillin-resistant Streptococcus pneumoniae), Staphylococcus aureus (including vancomycin-resistant Staphylococcus aureus (VRSA)), methicillin-resistant Staphylococcus aureus (MRSA) (including hospital-acquired MRSA, community acquired MRSA and coagulase negative staphylocci), Acinetobacter baumannii, Burkholderia multivorans, Burkholderia cenocepacia, Burkholderia cepacia, Klebsiella pneumoniae Pseudomonas aeruginosa and Neisseria gonorrhoeae (including penicillin-resistant Neisseria gonorrhoeae).

In some embodiments, the drug-resistant bacteria is a multiple drug resistant bacteria. The language “multiple drug resistant bacteria” includes bacteria that is resistant to two or more drugs, e.g., antibiotics, typically used for the treatment of such bacterial infections, for example, tetracycline, penicillin, cephalosporins (e.g., ceftriazone or cefixime), glycopeptides (e.g. vancomycin), quinolones (e.g., norfloxacin, ciprofloxacin or ofloxacin), co-trimoxazole, sulfonamides, aminoglycosides (e.g., kanamycin or gentamicin) and macrolides (e.g., azithromycin).

In some embodiments, the invention provides a method for treating bacterial skin infections in a subject using the agents and methods described herein.

In some embodiments, the bacterial skin infections are caused by Streptococcus pyogenes, Streptococcus agalactiae, or Staphylococcus aureus, including MRSA and/or VRSA.

In some embodiments, the invention provides a method for treating pneumonia in a subject using the agents and methods described herein.

The term “pneumonia” refers to an inflammatory condition of the lungs caused by a bacterial infection. In some embodiments, the pneumonia is caused by a Klebsiella pneumoniae, Streptococcus pneumoniae or Staphylococcus aureus infection. In some embodiments, the pneumonia is nocosomial pneumonia (e.g., hospital-acquired pneumonia) or community-acquired pneumonia. In some embodiments, the pneumonia is caused by penicillin-resistant Klebsiella pneumoniae.

In some embodiments, the invention provides a method for treating a condition selected from urinary tract infections, sepsis and bacteremia in a subject using the agents and methods described herein.

In some embodiments, administration of RNA may be performed by single administration or by multiple administrations.

In some embodiments, an amount the RNA described herein from 0.1 μg to 300 μg, 0.5 μg to 200 μg, or 1 μg to 100 μg, such as about 1 μg, about 3 μg, about 10 μg, about 30 μg, about 50 μg, or about 100 μg may be administered per dose.

In some embodiments, a regimen described herein includes at least one dose. In some embodiments, a regimen includes a first dose and at least one subsequent dose. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a regimen comprises two doses. In some embodiments, a provided regimen consists of two doses.

In some embodiments, a regimen administered to a subject may comprise a plurality of doses (e.g., at least two doses, at least three doses, or more). In some embodiments, a regimen administered to a subject may comprise a first dose and a second dose, which are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, doses may be separated by a period of about 7 to about 60 days, such as for example about 14 to about 48 days, etc. In some embodiments, a minimum number of days between doses may be about 1, 2, 3, 4, 5, 6, 7, 8, 9, 10, 11, 12, 13, 14, 15, 16, 17, 18, 19, 20, 21 or more. In some embodiments, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In some embodiments, doses may be about 21 to about 28 days apart. In some embodiments, doses may be about 19 to about 42 days apart.

In some embodiments, doses may be about 7 to about 28 days apart. In some embodiments, doses may be about 14 to about 24 days. In some embodiments, doses may be about 21 to about 42 days.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, 5 techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, 10 but are to be accorded the scope consistent with the claims.

### EXAMPLES

### Example 1: Bactericidal Activity of Lysins Secreted from P. pastoris Cells

P. pastoris is a well established eukaryotic expression system for the high level production of recombinant proteins. Proteins in P. pastoris are typically translated, folded and processed by a similar set of factors as in higher eukaryotes, such as human cells. In particular, expression of proteins containing N-terminal signal sequences results in their targeting to the secretory pathway and secretion into the extracellular space. The steps occurring during this process are highly conserved between eukaryotes and involve the translocation of the protein across the membrane of the endoplasmic reticulum (ER), its glycosylation and its trafficking to the plasma membrane. The use of human cells for the delivery of lysins by mRNA technology necessitates that lysins are able to pass through the secretory pathway in an active state. Since the majority of lysins are derived from phages and have evolved to be synthesized in a bacterial cytoplasm, it has been unclear if different lysins can be expressed and secreted in a eukaryotic cell. Using P. pastoris as an expression system, the inventors established here that a set of 11 model lysins can be trafficked through the eukaryotic secretory pathway and at the same time maintain their bactericidal activity.

For this purpose, the amino acid sequences (excluding the start methionine) of 11 lysins with predicted activity against S. aureus were modified with the N-terminal pre- and pro sequences of S. cerevisiae alpha mating factor, followed by a Kex2/Ste13 signal peptidase site, a hexa-histidine tag and a 3C protease cleavage site (see Appendix 1). The signal sequences target the lysin constructs to the secretory pathway and are cleaved off at the signal peptidase sites after translocation into the ER. The hexa-histidine tag and 3C sites were further introduced to facilitate lysin purification by affinity capture and removal of the hexa-histidine tag after proteolytic cleavage, respectively. The constructs were codon optimized for expression in P. pastoris and ordered from Genescript in plasmids conferring antibiotic resistance and control of lysin expression by an AOX1 methanol-inducible promoter. Plasmids were linearized by restriction and transformed into a wild type (mut+) P. pastoris strain, followed by selection of transformants on YPD plates containing 100 μg/ml Zeocin. Multiple colonies per construct were transferred each into 3 ml BMGY medium in 24 well plates and grown shaking at 30° C. After 24 hours, protein expression was induced by exchanging the medium to BMMY (containing 1% methanol). The medium was supplemented with 1% methanol every 24 hours and culture supernatants were harvested after 72 hours of expression, flash frozen in liquid nitrogen and stored at −80° C.

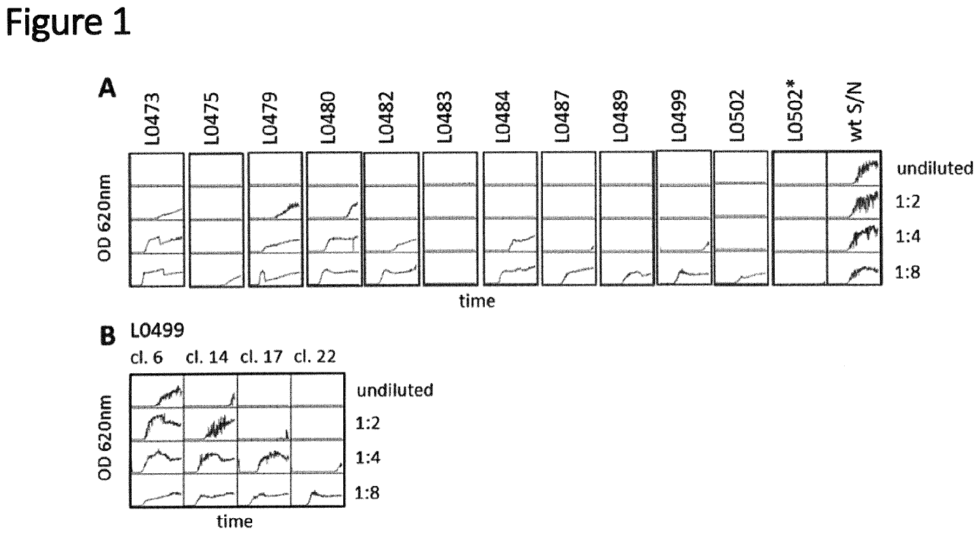

For each transformant, lysin secretion into culture supernatants was estimated by SDS-PAGE and Coomassie staining. Supernatants with highest apparent levels of secretion were serially diluted with lysin buffer (20 mM NaPO₄ PH 6.0, 100 mM NaCl) and tested for bactericidal activity. For this purpose, 30 μl of supernatants (and dilutions thereof) were mixed with S. aureus cells in 70 μl BHI medium such that the reaction contained 5×10⁵ cfu/ml. The optical density of these solutions (OD) at 620 nm was monitored in a plate reader over the course of 24 hours to quantify bacterial growth. In these experiments, S. aureus cells were able to grow in the presence of supernatants from a parental P. pastoris strain, showing that the culture medium itself does not have lytic activity. In contrast, S. aureus failed to grow in presence of supernatants from strains expressing each of the lysin constructs. These experiments show that the level of active lysins in undiluted supernatants was sufficiently high to lyse all S. aureus cells in the culture (FIG. 1A). Depending on the construct and clone, mixing of supernatants and buffer resulted in bacterial growth at different folds of dilution. For example, bacterial growth started to appear after a one-fold dilution of the L0475 supernatant, whereas growth only appeared at an eight-fold dilution of the L0502 supernatant, suggesting that the L0502 supernatant had a higher concentration and/or lytic activity compared to the L0475 supernatant. Similarly, different clones of one lysin construct varied in the fold dilutions at which bacterial growth started to appear (FIG. 1B), suggesting differences in the levels of expression and secretion between different transformants. Taken together, the data demonstrate that lysins can be channeled through the secretory pathway and maintain their activity after secretion. Surprisingly, all of the tested lysins were expressible in an active form in P. pastoris.

### Example 2: Comparison of Lysin Activity after Expression in E. coli and P. pastoris

Expression of the lysins in P. pastoris was compared to their expression in E. coli. Constructs containing N-terminal hexa-histidine tags and 3C protease cleavage sites (but lacking the export signal sequences) were codon optimized for expression in E. coli followed by DNA synthesis at TWIST Biosciences. The constructs were subsequently cloned into an IPTG-inducible pET21 vector conferring resistance to Kanamycin. After transformation into BL21 cells, lysins were expressed in 2 ml cultures of TB+1.5% lactose for 24 hours at 25° C. Bacterial pellets were lysed in 1.5 ml lysis buffer (50 mM Hepes pH 7.5, 300 mM NaCl, 20 mM Imidazole, 0.5 mM TCEP, 2 μl Benzonase per ml, 1× FastBreak) and centrifuged at 21130 rcf at 4° C. for 15 min. Cleared lysates were applied to 100 μl Nickel affinity matrix, washed with 15 column volumes each of wash buffer I (50 mM Hepes pH 7.5, 300 mM NaCl, 20 mM Imidazole, 0.5 mM TCEP) and wash buffer II (50 mM Hepes pH 7.5, 300 mM NaCl, 40 mM Imidazole, 0.5 mM TCEP) and eluted in 4 column volumes elution buffer (50 mM Hepes pH 7.5, 300 mM NaCl, 500 mM Imidazole, 0.5 mM TCEP). Eluates were buffer exchanged into 20 mM NaH₂PO₄/Na₂HPO₄ PH 6, 100 mM NaCl using a CentriPure P96 Gelfiltration column array, snap frozen in liquid nitrogen and stored at −80° C.

SDS-PAGE of eluates followed by Coomassie staining showed that three of the 11 constructs could not be purified in a soluble form. Surprisingly, S. aureus killing assays (as performed in Example 1) showed that several of these eluates had no lytic activity. Of the constructs that could be purified in a soluble form, a large fraction was also inactive (7 out of 11). In contrast, all lysin constructs showed lytic activity against S. aureus cells after expression and secretion in P. pastoris (Table 3, FIG. 1A). Taken together, this data indicates that the use of P. pastoris might facilitate the expression of active lysins, possibly due to the increased folding capacity of eukaryotic cells.

TABLE 3
Comparison of the bactericidal activity of lysins purified in E. coli and lysins secreted
in P. pastoris. Domain architecture, internal name and NCBI identifiers are indicated. Lysin
constructs that were found active in P. pastoris supernatants but could not be purified from
E. coli in an active form are shaded in grey. soluble(Ec): classification if protein could be
purified in a soluble form from E. coli. active(Ec): classification if the lysin purified
from E. coli was active in S. aureus killing assays. active(Pp): classification if the lysin
secreted in P. pastoris was active in preventing S. aureus growth. y: yes. n: no.

| domain | Internal | | soluble | active | active |
|---|---|---|---|---|---|
| architecture | name | NCBI ID | (Ec) | (Ec) | (Pp) |
| Ami2 | L0487 | WP_115921155 | n | n | y |
| CHAP-Ami2-SH3 | L0489 | ABF71642 | y | n | y |
| | L0479 | AWH13065 | y | n | y |
| | L0480 | YP_024461 | y | y | y |
| | L0473 | WP_195722665 | y | y | y |
| | L0475 | YP_009268702 | y | n | y |
| LysM-CHAP | L0499 | WP_070648624 | n | n | y |
| | L0482 | SWP_143119924 | y | y | y |
| PepM23-SH3 | L0483 | Lysostaphin | y | y | y |
| phage lysozyme | L0484 | PTF44644 | y | n | y |
| SH3-Ami3 | L0502 | WP_053025743 | n | n | y |

APPENDIX 1
Amino acid and DNA sequences of 11 lysins for expression in P.pastoris

>L0473_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMAKTKKQIEQRLQAYAKGTVDSPYRITKPTSYDPSFGVMEVGAIDADRYYHAQ
CQDLITDYVLWLTDNKVRPWGNAKDQINQSYGTGFKIHKNLPSTVAKTGWIAVFTSGTYSQYGHIGIVYSPGDTNK
FHIIEQNWNGWANKKPLTRWDNYYGLTHFIEVPYAKETITTKKTAPKAATSKPAKKISVSHNNINLQSNFAKRGYNP
KGVVIHNDAGRSTAQQYENSLKGANQNRLNNGIAHAYADSSHIWYAIEPERIAYHTGDGVKKGTGNYDYFGIEVC
QSMSASDKEFMANEQVVLQEVARLLKKWKLPANRNTVRLHLEFSATSCPHRSLTMRTGFNPVTQGRPSDAIIKKLK
DIWIKEIRAYMEGKTPSIKTVANKPGSASTPATKSNSDGWKVNAYNTYYKTERASFTVGSTRIAVRSTGPFTTCPFTG
WLNPGVTITYDEVMVQNGFVWLGYDSYNGRRYVPIRTAQGTPGSSNYVLGPLWGTIS*
>L0473_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCTTTCTCTAACTCTACTAACAACGGTTTGTTGTTTATTAACACTACTATCGCTTCTATTGCTGCTAAA
GAAGAGGGTGTTTCTTTGGAAAAGAGAGAAGCTGAGGCTCATCACCATCACCATCACTTGGAGGTTTTGTTCC
AAGGTCCAATGGCTAAGACTAAGAAACAAATCGAGCAAAGATTGCAAGCTTATGCTAAAGGTACTGTTGATTC
TCCTTACAGAATTACTAAGCCAACTTCTTATGATCCTTCTTTCGGTGTTATGGAAGTTGGTGCTATTGATGCTGA
TAGATACTATCATGCTCAATGTCAAGATTTGATCACTGATTACGTTTTGTGGTTGACTGATAACAAAGTTAGACC
ATGGGGTAACGCTAAGGATCAAATTAATCAATCTTACGGTACTGGTTTTAAGATCCACAAGAATTTGCCTTCTA
CTGTTGCTAAAACTGGTTGGATTGCTGTTTTCACTTCTGGTACTTACTCTCAATATGGTCATATTGGTATTGTTTA
CTCTCCAGGAGATACTAACAAGTTCCACATCATCGAACAAAACTGGAACGGTTGGGCTAACAAGAAACCATTG
ACTAGATGGGATAACTACTACGGTTTGACTCATTTCATCGAAGTTCCTTACGCTAAGGAGACTATCACTACTAA
GAAAACTGCTCCAAAGGCTGCTACTTCTAAACCTGCTAAGAAAATTTCTGTTTCTCATAACAACATTAACTTGCA
ATCTAATTTTGCTAAAAGAGGTTACAACCCAAAGGGTGTTGTTATTCACAATGATGCTGGTAGATCTACTGCTC
AACAATACGAAAATTCTTTGAAGGGTGCTAACCAAAATAGATTGAACAATGGTATTGCTCATGCTTACGCTGAT
TCTTCTCACATTTGGTATGCTATTGAACCTGAGAGAATTGCTTACCATACTGGAGATGGTGTTAAGAAAGGTAC
TGGTAACTACGATTACTTCGGTATCGAAGTTTGTCAATCTATGTCTGCTTCTGATAAAGAGTTTATGGCTAATGA
GCAAGTTGTTTTGCAAGAGGTTGCTAGATTGTTGAAGAAATGGAAGTTGCCAGCTAACAGAAACACTGTTAGA
TTGCATTTGGAATTTTCTGCTACTTCTTGTCCTCACAGATCTTTGACTATGAGAACTGGTTTCAATCCAGTTACTC
AAGGTAGACCTTCTGATGCTATTATTAAGAAATTGAAGGATATCTGGATCAAGGAAATCAGAGCTTACATGGA
GGGTAAAACTCCATCTATTAAAACTGTTGCTAACAAGCCAGGTTCTGCTTCTACTCCTGCTACTAAATCTAATTC
TGATGGTTGGAAGGTTAACGCTTACAACACTTACTACAAGACTGAGAGAGCTTCTTTTACTGTTGGTTCTACTA
GAATTGCTGTTAGATCTACTGGTCCATTCACTACTTGTCCTTTCACTGGTTGGTTGAACCCAGGTGTTACTATCA
CTTACGATGAAGTTATGGTTCAAAACGGTTTCGTTTGGTTGGGTTACGATTCTTATAATGGTAGAAGATACGTT
CCAATTAGAACTGCTCAAGGTACTCCTGGTTCTTCTAATTATGTTTTGGGTCCTTTGTGGGGTACTATTTCTTAA
>L0475_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMQAKLTKKEFIEWLKTSEGKQFNADLWYGFQCFDYANAAWKVLFGLLLKGLG
AKDIPFANNFDGLATVYQNTPDFLAQPGDMVVFGSNYGAGYGHVAWVIEATLDYIIVYEQNWLGGGWTDGIEQP
GWGWEKVTRRQHAYDFPMWFIRPNFKSEIAPRSVQSPTQAPKKETAKPQPKAVELKIIKDVVKGYDLPKRGSNPK
GIVIHNDAGSKGATAEAYRNGLVNAPLSRLEAGIAHSYVSGNTVWQALDESQVGWHTANQLGNKYYYGIEVCQS
MGADNATFLKNEQATFQECARLLKKWGLPANRNTIRLHNEFTSTSCPHRSSVLHTGFDPVTRGLLPEDKRLQLKDY
FIKQIRAYMDGKIPVATVSNDSSASSNTVKPVASAWKRNKYGTYYMEESARFTNGNQPITVRKVGPFLYCPVGYQF
QPGGYCDYTEVMLQDGHVWVGYTWEGQRYYLPIRTWNGSAPPNQILGDLWGEIS*
>L0475_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTATTCTGATTTGGAGGGAGATTTTGATGTTG
CTGTTTTGCCATTCTCTAACTCTACTAACAACGGTTTGTTGTTTATTAACACTACTATCGCTTCTATTGCTGCTAA
GGAAGAGGGTGTTTCTTTGGAAAAGAGAGAAGCTGAGGCTCATCACCATCACCATCACTTGGAGGTTTTGTTC
CAAGGTCCTATGCAGGCTAAGTTGACTAAGAAAGAGTTTATTGAGTGGTTGAAGACTTCTGAAGGTAAACAAT
TCAACGCTGATTTGTGGTATGGTTTTCAATGTTTCGATTACGCTAATGCTGCTTGGAAAGTTTTGTTTGGTTTGT
TGTTGAAGGGTTTGGGTGCTAAGGATATCCCTTTCGCTAACAACTTCGATGGTTTGGCTACTGTTTACCAAAAC
ACTCCAGATTTCTTGGCTCAACCTGGAGATATGGTTGTTTTCGGTTCTAATTATGGTGCTGGTTACGGTCATGTT
GCTTGGGTTATTGAAGCTACTTTGGATTACATCATCGTTTACGAGCAAAACTGGTTGGGTGGTGGTTGGACTG
ATGGTATTGAACAACCAGGTTGGGGTTGGGAGAAGGTTACTAGAAGACAACACGCTTATGATTTTCCAATGTG
GTTCATCAGACCTAACTTCAAGTCTGAAATCGCTCCTAGATCTGTTCAATCTCCAACTCAAGCTCCTAAGAAAGA
AACTGCTAAGCCACAACCTAAAGCTGTTGAGTTGAAGATTATTAAGGATGTTGTTAAGGGTTACGATTTGCCAA
AGAGAGGTTCTAACCCTAAGGGTATCGTTATCCATAACGATGCTGGTTCTAAGGGTGCTACTGCTGAAGCTTAT
AGAAACGGTTTGGTTAATGCTCCATTGTCCAGATTGGAAGCTGGTATTGCTCATTCTTACGTTTCTGGTAACACT
GTTTGGCAAGCTTTGGATGAATCTCAAGTTGGTTGGCACACTGCTAACCAATTGGGTAATAAGTACTATTACGG
TATTGAGGTTTGTCAATCTATGGGTGCTGATAACGCTACTTTCTTGAAGAACGAACAAGCTACTTTCCAAGAGT
GTGCTAGATTGTTGAAGAAATGGGGTTTGCCAGCTAACAGAAACACTATCAGATTGCACAACGAGTTTACTTCT
ACTTCTTGTCCTCATAGATCTTCTGTTTTGCACACTGGTTTCGATCCAGTTACTAGAGGTTTGTTGCCTGAGGAT
AAGAGATTGCAATTGAAGGATTACTTCATCAAGCAAATCAGAGCTTACATGGATGGTAAAATTCCAGTTGCTA
CTGTTTCTAACGATTCTTCTGCTTCTTCTAATACTGTTAAACCTGTTGCTTCTGCTTGGAAGAGAAACAAGTATG
GTACTTATTACATGGAAGAGTCTGCTAGATTCACTAACGGTAATCAACCAATTACTGTTAGAAAGGTTGGTCCA
TTTTTGTATTGTCCTGTTGGTTACCAATTCCAACCTGGTGGTTATTGTGATTACACTGAAGTTATGTTGCAAGAT
GGTCATGTTTGGGTTGGTTACACTTGGGAGGGTCAAAGATATTACTTGCCAATTAGAACTTGGAACGGTTCTG
CTCCACCTAATCAAATTTTGGGAGATTTGTGGGGTGAAATTTCTTAA
>L0479_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMQAKLTKKEFIEWLKTSEGKQFNVDLWYGFQCFDYANAGWKVLFGLLLKGLG
AKDIPFANNFDGLATVYQNTPDFLAKPGDMVVFGSNYGAGYGHVAWVIEATLDYIIVYEQNWLGGGWTDRIEQP
GWGWEKVTRRQHALRFPYVVYPSYPKSETAPRSIQSPTQASKKETAKPQPKAVELKIIKDVVKGYDLPKRGGNPKGI
VIHNDAGSKGATAEAYRNGLVNAPLSRLEAGIAHSYVSGNTVWQALDESQVGWHTANQLGNKYYYGIEVCQSM
GADNATFLKNEQATFQECARLLKKWGLPANRNTIRLHNEFTSTSCPHRSSVLHTGFDPVTRGLLPEDKRLQLKDYFIK
QIRAYMDGKIPVATVSNESSASSNTVKPVASAWKRNKYGTYYMEESARFTNGNQPITVRKVGPFLSCPVGYQFQP
GGYCDYTEVMLQDGHVWVGYTWEGQRYYLPIRTWNGSAPPNQILGDLWGEIS*
>L0479_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCATTCTCTAACTCTACTAACAACGGTTTGTTGTTTATTAACACTACTATCGCTTCTATTGCTGCTAA
GGAAGAGGGTGTTTCTTTGGAAAAGAGAGAAGCTGAGGCTCATCACCATCACCATCACTTGGAGGTTTTGTTC
CAAGGTCCTATGCAGGCTAAGTTGACTAAGAAAGAGTTTATTGAGTGGTTGAAGACTTCTGAAGGTAAACAAT
TCAACGTTGATTTGTGGTACGGTTTTCAATGTTTCGATTATGCTAATGCCGGTTGGAAAGTTTTGTTTGGTTTGT
TGTTGAAGGGTTTGGGTGCTAAGGATATCCCTTTCGCTAACAACTTCGATGGTTTGGCTACTGTTTACCAAAAC
ACTCCAGATTTCTTGGCTAAGCCTGGAGATATGGTTGTTTTCGGTTCTAATTACGGTGCTGGTTATGGTCATGTT
GCTTGGGTTATTGAAGCTACTTTGGATTACATCATCGTTTACGAGCAAAATTGGTTGGGTGGTGGTTGGACTG
ATAGAATTGAACAACCAGGTTGGGGTTGGGAGAAGGTTACTAGAAGACAACACGCTTTGAGATTCCCTTATGT
TGTTTACCCATCTTATCCTAAATCTGAAACTGCTCCAAGATCTATTCAATCTCCTACTCAAGCTTCTAAGAAAGA
AACTGCTAAGCCACAACCTAAAGCTGTTGAGTTGAAGATTATTAAGGATGTTGTTAAGGGTTACGATTTGCCAA
AGAGAGGTGGTAACCCTAAGGGTATCGTTATCCATAACGATGCTGGTTCTAAAGGTGCTACTGCTGAAGCTTA
CAGAAACGGTTTGGTTAATGCTCCATTGTCCAGATTGGAAGCTGGTATTGCTCATTCTTATGTTTCTGGTAACAC
TGTTTGGCAAGCTTTGGATGAATCTCAAGTTGGTTGGCACACTGCTAACCAATTGGGTAATAAGTACTATTACG
GTATTGAGGTTTGTCAATCTATGGGTGCTGATAACGCTACTTTCTTGAAGAACGAACAAGCTACTTTCCAAGAG
TGTGCTAGATTGTTGAAGAAATGGGGTTTGCCAGCTAACAGAAACACTATCAGATTGCACAACGAGTTTACTTC
TACTTCTTGTCCTCATAGATCTTCTGTTTTGCACACTGGTTTCGATCCAGTTACTAGAGGTTTGTTGCCTGAGGA
TAAGAGATTGCAATTGAAGGATTACTTCATCAAGCAAATCAGAGCTTACATGGATGGTAAAATCCCAGTTGCT
ACTGTTTCTAACGAATCTTCTGCTTCTTCTAATACTGTTAAACCTGTTGCTTCTGCTTGGAAGAGAAACAAATAC
GGTACTTACTACATGGAAGAGTCTGCTAGATTCACTAACGGTAATCAACCAATTACTGTTAGAAAGGTTGGTCC
ATTTTTGTCTTGTCCTGTTGGTTACCAATTCCAACCTGGTGGTTACTGTGATTATACTGAAGTTATGTTGCAAGA
TGGTCATGTTTGGGTTGGTTATACTTGGGAGGGTCAAAGATATTACTTGCCAATTAGAACTTGGAACGGTTCTG
CTCCACCTAATCAAATTTTGGGAGATTTGTGGGGTGAAATTTCTTAA
>L0480_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMAKTQAEINKRLDAYAKGTVDSPYRVKKATSYDPSFGVMEAGAIDADGYYHA
QCQDLITDYVLWLTDNKVRTWGNAKDQIKQSYGTGFKIHENKPSTVPKKGWIAVFTSGSYEQWGHIGIVYDGGNT
STFTILEQNWNGYANKKPTKRVDNYYGLTHFIEIPVKAGTTVKKETAKKSASKTPAPKKKATLKVSKNHINYTMDKR
GKKPEGMVIHNDAGRSSGQQYENSLANAGYARYANGIAHYYGSEGYVWEAIDAKNQIAWHTGDGTGANSGNFR
FAGIEVCQSMSASDAQFLKNEQAVFQFTAEKFKEWGLTPNRKTVRLHMEFVPTACPHRSMVLHTGFNPVTQGRP
SQAIMNKLKDYFIKQIKNYMDKGTSSSTVVKDGKTSSASTPATRPVTGSWKKNQYGTWYKPENATFVNGNQPIVT
RIGSPFLNAPVGGNLPAGATIVYDEVCIQAGHIWIGYNAYNGNRVYCPVRTCQGVPPNQIPGVAWGVFK*
>L0480_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCATTCTCTAACTCTACTAACAACGGTTTGTTGTTTATTAACACTACTATCGCTTCTATTGCTGCTAA
GGAAGAGGGTGTTTCTTTGGAGAAAAGAGAAGCTGAGGCTCATCACCATCACCATCACTTGGAAGTTTTGTTC
CAAGGTCCTATGGCTAAGACTCAAGCTGAGATTAATAAGAGATTGGATGCTTACGCTAAGGGTACTGTTGATT
CTCCATACAGAGTTAAGAAAGCTACTTCTTATGATCCTTCTTTCGGTGTTATGGAAGCTGGTGCTATTGATGCTG
ATGGTTACTATCATGCTCAATGTCAAGATTTGATCACTGATTACGTTTTGTGGTTGACTGATAACAAGGTTAGA
ACTTGGGGTAACGCTAAGGATCAAATTAAGCAATCTTACGGTACTGGTTTTAAAATCCATGAAAACAAGCCATC
TACTGTTCCTAAGAAAGGTTGGATTGCTGTTTTCACTTCTGGTTCTTACGAGCAATGGGGTCACATTGGTATTGT
TTACGATGGTGGTAACACTTCTACTTTTACTATTTTGGAACAAAACTGGAATGGTTACGCTAACAAGAAACCAA
CTAAGAGAGTTGATAACTACTACGGTTTGACTCACTTCATTGAAATTCCTGTTAAGGCTGGTACTACTGTTAAG
AAAGAGACTGCTAAGAAATCTGCTTCTAAAACTCCAGCTCCTAAGAAAAAGGCTACTTTGAAAGTTTCTAAGAA
CCATATCAACTACACTATGGATAAGAGAGGTAAAAAGCCAGAGGGTATGGTTATTCACAACGATGCTGGTAGA
TCTTCTGGTCAACAATACGAGAACTCTTTGGCTAATGCTGGTTACGCTAGATATGCTAACGGTATTGCTCATTAC
TATGGTTCTGAAGGTTATGTTTGGGAGGCTATTGATGCTAAAAATCAAATTGCTTGGCACACTGGAGATGGTA
CTGGTGCTAACTCTGGTAATTTTAGATTCGCTGGTATTGAAGTTTGTCAATCTATGTCTGCTTCTGATGCTCAAT
TTTTGAAGAACGAGCAAGCTGTTTTCCAATTCACTGCTGAAAAATTCAAGGAGTGGGGTTTGACTCCTAACAGA
AAGACTGTTAGATTGCATATGGAATTTGTTCCAACTGCTTGTCCTCATAGATCTATGGTTTTGCACACTGGTTTC
AATCCAGTTACTCAAGGTAGACCTTCTCAAGCTATCATGAACAAATTGAAGGATTACTTCATCAAGCAAATTAA
GAATTACATGGATAAGGGTACTTCTTCTTCTACTGTTGTTAAAGATGGTAAAACTTCTTCTGCTTCTACTCCAGC
TACTAGACCTGTTACTGGTTCTTGGAAAAAGAACCAATACGGTACTTGGTATAAACCAGAGAATGCTACTTTTG
TTAACGGTAATCAACCTATTGTTACTAGAATTGGTTCTCCATTCTTGAACGCTCCTGTTGGTGGTAATTTGCCAG
CTGGTGCTACTATCGTTTACGATGAAGTTTGTATCCAAGCTGGTCATATTTGGATTGGTTACAACGCTTATAAC
GGTAATAGAGTTTATTGTCCAGTTAGAACTTGTCAAGGTGTTCCACCTAATCAAATTCCTGGTGTTGCTTGGGG
TGTTTTCAAGTAA
>L0482_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPREAPKTQIYTVKKGDTLSAIALKYKTTVSNIQNTNNIANPNLIFIGQKLKVPMTP
LVEPKPKTVSSNNKSNSNSSTLNYLKTLENRGWDFDGSYGWQCFDLVNVYWNHLYGHGLKGYGAKDIPYANNEN
SEAKIYHNTPTFKAEPGDLVVFSGRFGGGYGHTAIVLNGDYDGKLMKFQSLDQNWNNGGWRKAEVAHKVVHNY
ENDMIFIRPFKKA*
>L0482_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCTTTCTCTAACTCTACTAACAACGGTTTGTTGTTTATTAACACTACTATCGCTTCTATTGCTGCTAA
GGAAGAGGGTGTTTCTTTGGAAAAGAGAGAAGCTGAGGCTCATCACCATCACCATCACTTGGAAGTTTTGTTC
CAAGGTCCAAGAGAGGCTCCTAAGACTCAAATCTACACTGTTAAGAAAGGAGATACTTTGTCTGCTATCGCTTT
GAAGTACAAGACTACTGTTTCTAACATCCAAAACACTAACAACATCGCTAACCCAAATTTGATTTTCATTGGTCA
AAAGTTGAAAGTTCCAATGACTCCTTTGGTTGAACCAAAGCCTAAGACTGTTTCTTCTAACAATAAGTCTAACTC
TAATTCTTCTACTTTGAACTACTTGAAGACTTTGGAGAATAGAGGTTGGGATTTTGATGGTTCTTATGGTTGGC
AATGTTTCGATTTGGTTAACGTTTACTGGAATCATTTGTATGGTCACGGTTTGAAGGGTTACGGTGCTAAGGAT
ATCCCTTACGCTAACAACTTCAACTCTGAAGCTAAGATCTACCATAATACTCCAACTTTCAAAGCTGAGCCTGGA
GATTTGGTTGTTTTCTCTGGTAGATTCGGTGGTGGTTACGGTCACACTGCTATTGTTTTGAACGGAGATTACGA
TGGTAAATTGATGAAGTTCCAATCTTTGGATCAAAATTGGAACAATGGTGGTTGGAGAAAGGCTGAAGTTGCT
CATAAGGTTGTTCACAACTACGAGAACGATATGATTTTCATTAGACCATTCAAGAAAGCTTAA
>L0483_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPAATHEHSAQWLNNYKKGYGYGPYPLGINGGMHYGVDFFMNIGTPVKAISSG
KIVEAGWSNYGGGNQIGLIENDGVHRQWYMHLSKYNVKVGDYVKAGQIIGWSGSTGYSTAPHLHFQRMVNSFS
NSTAQDPMPFLKSAGYGKAGGTVTPTPNTGWKTNKYGTLYKSESASFTPNTDIITRTTGPFRSMPQSGVLKAGQTI
HYDEVMKQDGHVWVGYTGNSGQRIYLPVRTWNKSTNTLGVLWGTIK*
>L0483_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCTTTCTCTAACTCTACTAACAACGGTTTGTTGTTTATTAACACTACTATCGCTTCTATTGCTGCTAA
GGAAGAGGGTGTTTCTTTGGAAAAGAGAGAAGCTGAGGCTCATCACCATCACCATCACTTGGAAGTTTTGTTC
CAAGGTCCAGCTGCTACTCATGAGCACTCTGCTCAATGGTTGAACAATTACAAGAAAGGTTACGGTTATGGTCC
ATATCCTTTGGGTATTAACGGTGGTATGCATTACGGTGTTGATTTCTTTATGAACATCGGTACTCCTGTTAAGGC
TATTTCTTCTGGTAAAATTGTTGAAGCTGGTTGGTCTAACTATGGTGGTGGTAATCAAATTGGTTTGATTGAGA
ACGATGGTGTTCATAGACAATGGTACATGCACTTGTCTAAGTACAACGTTAAGGTTGGAGATTACGTTAAGGC
TGGTCAAATTATTGGTTGGTCTGGTTCTACTGGTTATTCTACTGCTCCACATTTGCACTTCCAAAGAATGGTTAA
CTCTTTCTCTAATTCTACTGCTCAAGATCCAATGCCTTTCTTGAAGTCTGCTGGTTACGGTAAAGCTGGTGGTAC
TGTTACTCCAACTCCTAACACTGGTTGGAAGACTAACAAGTACGGTACTTTGTACAAGTCTGAATCTGCTTCTTT
TACTCCAAACACTGATATCATCACTAGAACTACTGGTCCATTCAGATCTATGCCTCAATCTGGTGTTTTGAAGGC
TGGTCAAACTATCCATTACGATGAGGTTATGAAGCAAGATGGTCACGTTTGGGTTGGTTACACTGGTAATTCTG
GTCAAAGAATCTACTTGCCTGTTAGAACTTGGAACAAGTCTACTAATACTTTGGGTGTTTTGTGGGGTACTATT
AAATAA
>L0484_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPDQFLDEKEGNHTTAYRDGSGIWTICRGATMVDGKPVIPGMRLSKEKCDQVN
AIERDKALAWVERNIKVPLTEPQKAGIASFCPYNIGPGKCFPSTFYKRLNAGDRKGACEAIRWWIKDGGRDCRIRSN
NCYGQVIRRDQESALACWGIDQ*
>L0484_DNA
ATGAGATTTCCTTCTATTTTCACTGCTGTTTTGTTTGCTGCTTCTTCTGCTTTGGCTGCTCCTGTTAACACTACTAC
TGAAGATGAGACTGCTCAAATTCCAGCTGAAGCTGTTATTGGTTACTCTGATTTGGAGGGAGATTTTGATGTTG
CTGTTTTGCCTTTCTCTAACTCTACTAACAACGGTTTGTTGTTCATTAACACTACTATCGCTTCTATCGCTGCTAA
GGAAGAGGGTGTTTCTTTGGAAAAGAGAGAAGCTGAGGCTCATCACCATCACCATCACTTGGAGGTTTTGTTT
CAAGGTCCAGATCAATTCTTGGATGAAAAAGAGGGTAATCATACTACTGCTTATAGAGATGGTTCTGGTATTTG
GACTATTTGTAGAGGTGCTACTATGGTTGATGGTAAACCAGTTATTCCTGGTATGAGATTGTCTAAGGAAAAGT
GTGATCAAGTTAACGCTATTGAGAGAGATAAGGCTTTGGCTTGGGTTGAAAGAAATATTAAGGTTCCATTGAC
TGAGCCTCAAAAAGCTGGTATTGCTTCTTTTTGTCCATACAACATTGGTCCTGGTAAATGTTTCCCATCTACTTTC
TACAAGAGATTGAATGCTGGAGATAGAAAGGGTGCTTGTGAAGCTATTAGATGGTGGATTAAGGATGGTGGT
AGAGATTGTAGAATTAGATCTAACAACTGTTACGGTCAAGTTATTAGAAGAGATCAAGAATCTGCTTTGGCTTG
TTGGGGTATTGATCAATAA
>L0487_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMRYIPAAHHSAGSNKPVNRVVIHATCPDVGFPSASRKGRAVSTANYFASPSSG
GSAHYVCDIGETVQCLSESTIGWHAPPNPHSLGIEICADGGSHASFRVPGHAYTREQWLDPRVWPAVERAAVLCR
QLCDKHGVPKRKLSVADLKAGRRGICGHTDVTDAWHQSDHDDPGPWFPWDKFMAVVNGHGGGSSSEELTVAD
VKALHNQIKQLSAQLSGSVNKLHHDVGVVQVQNGDLSKRVDALSWVKNPVTGKLWRTKDALWSVWYYVLECRS
RIDRLESAVNGLKK*
>L0487_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCTTTCTCTAACTCTACTAACAACGGTTTGTTGTTTATTAACACTACTATCGCTTCTATTGCTGCTAA
GGAAGAGGGTGTTTCTTTGGAAAAGAGAGAAGCTGAGGCTCATCACCATCACCATCACTTGGAGGTTTTGTTC
CAAGGTCCAATGAGATATATTCCTGCTGCTCATCACTCTGCTGGTTCTAACAAGCCAGTTAATAGAGTTGTTATT
CATGCTACTTGTCCAGATGTTGGTTTTCCTTCTGCTTCCAGAAAAGGTAGAGCTGTTTCTACTGCTAACTACTTC
GCTTCTCCTTCTTCTGGTGGTTCTGCTCATTATGTTTGTGATATTGGTGAAACTGTTCAATGTTTGTCTGAGTCTA
CTATTGGTTGGCATGCTCCACCTAATCCACACTCTTTGGGTATTGAAATTTGTGCTGATGGTGGTTCTCATGCTT
CTTTCAGAGTTCCTGGTCACGCTTACACTAGAGAACAATGGTTGGATCCAAGAGTTTGGCCTGCTGTTGAGAG
AGCTGCTGTTTTGTGTAGACAATTGTGTGATAAGCATGGTGTTCCAAAGAGAAAATTGTCTGTTGCTGATTTGA
AAGCTGGTAGAAGAGGTATTTGTGGTCACACTGATGTTACTGATGCTTGGCATCAATCTGATCACGATGATCCA
GGTCCTTGGTTTCCTTGGGATAAGTTCATGGCTGTTGTTAACGGTCATGGTGGTGGTTCTTCTTCTGAAGAGTT
GACTGTTGCTGATGTTAAGGCTTTGCACAACCAAATTAAACAATTGTCTGCTCAATTGTCTGGTTCCGTTAACAA
ATTGCATCACGATGTTGGTGTTGTTCAAGTTCAAAATGGAGATTTGTCTAAGAGAGTTGATGCTTTGTCTTGGG
TTAAAAACCCAGTTACTGGTAAATTGTGGAGAACTAAAGATGCTTTGTGGTCTGTTTGGTACTATGTTTTGGAA
TGTAGATCCAGAATTGATAGATTGGAGTCTGCTGTTAATGGTTTGAAGAAATAA
>L0489_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMLMTKNQAEKWFDNSLGKQFNPDGWYGFQCYDYANMFFMLATGERLQGL
YAYNIPFDNKAKIEKYGQIIKNYDSFLPQKLDIVVFPSKYGGGAGHVEIVESANLNTFTSFGQNWNGKGWTNGVAQ
PGWGPETVTRHVHYYDNPMYFIRLNFPNNLSVGNKAKGIIKQATTKKEAVIKPKKIMLVAGHGYNDPGAVGNGTN
ERDFIRKYITPNIAKYLRHAGHEVALYGGSSQSQDMYQDTAYGVNVGNKKDYGLYWVKSQGYDIVLEIHLDAAGES
ASGGHVIISSQFNADTIDKSIQDVIKNNLGQIRGVTPRNDLLNVNVSAEININYRLSELGFITNKNDMDWIKKNYDLY
SKLIAGAIHGKPIGGLVAGNVKTSAKNKKNPPVPAGYTLDKNNVPYKKEQGNYTVANVKGNNVRDGYSTNSRITGV
LPNNTTITYDGAYCINGYRWITYIANSGQRRYIATGEVDKAGNRISSFGKFSTI*
>L0489_DNA
ATGAGATTTCCATCTATTTTCACTGCTGTTTTGTTCGCTGCTTCTTCTGCTTTGGCTGCTCCAGTTAACACTACTA
CTGAAGATGAGACTGCTCAAATTCCTGCTGAAGCTGTTATTGGTTACTCTGATTTGGAGGGAGATTTTGATGTT
GCTGTTTTGCCTTTCTCTAACTCTACTAACAACGGTTTGTTGTTCATTAACACTACTATCGCTTCTATCGCTGCTA
AGGAAGAGGGTGTTTCTTTGGAAAAGAGAGAAGCTGAGGCTCATCACCATCACCATCACTTGGAGGTTTTGTT
TCAAGGTCCAATGTTGATGACTAAGAACCAAGCTGAAAAATGGTTCGATAACTCTTTGGGTAAACAATTCAATC
CTGATGGTTGGTACGGTTTCCAATGTTACGATTACGCTAACATGTTTTTCATGTTGGCTACTGGTGAAAGATTG
CAAGGTTTGTACGCTTACAACATCCCATTCGATAATAAGGCTAAGATCGAGAAGTACGGTCAAATTATTAAGAA
CTACGATTCTTTCTTGCCACAAAAGTTGGATATCGTTGTTTTCCCTTCTAAATACGGTGGTGGTGCTGGTCATGT
TGAAATTGTTGAGTCTGCTAACTTGAATACTTTTACTTCTTTCGGTCAAAACTGGAATGGTAAAGGTTGGACTA
ACGGTGTTGCTCAACCAGGTTGGGGTCCTGAAACTGTTACTAGACATGTTCACTACTACGATAACCCAATGTAC
TTCATCAGATTGAATTTCCCTAACAATTTGTCTGTTGGTAATAAGGCTAAGGGTATTATTAAGCAAGCTACTACT
AAGAAAGAGGCTGTTATTAAACCAAAGAAAATTATGTTGGTTGCTGGTCACGGTTATAACGATCCTGGTGCTG
TTGGTAACGGTACTAACGAAAGAGATTTCATTAGAAAGTACATCACTCCTAACATCGCTAAGTATTTGAGACAT
GCTGGTCACGAGGTTGCTTTGTACGGTGGTTCTTCTCAATCTCAAGATATGTACCAAGATACTGCTTATGGTGT
TAACGTTGGTAATAAGAAAGATTACGGTTTGTACTGGGTTAAGTCTCAAGGTTACGATATCGTTTTGGAAATCC
ATTTGGATGCTGCTGGTGAATCTGCTTCTGGTGGTCACGTTATCATCTCTTCTCAATTCAACGCTGATACTATCG
ATAAGTCTATCCAAGATGTTATTAAAAACAATTTGGGTCAAATTAGAGGTGTTACTCCAAGAAACGATTTGTTG
AACGTTAACGTTTCTGCTGAAATTAACATCAACTACAGATTGTCTGAGTTGGGTTTTATTACTAACAAAAACGAT
ATGGATTGGATTAAGAAAAACTACGATTTGTATTCTAAGTTGATTGCTGGTGCTATTCATGGTAAACCTATTGG
TGGTTTGGTTGCTGGTAACGTTAAGACTTCTGCTAAAAACAAGAAAAATCCACCTGTTCCAGCTGGTTACACTT
TGGATAAGAACAATGTTCCTTACAAGAAAGAACAGGGTAACTACACTGTTGCTAACGTTAAGGGTAACAACGT
TAGAGATGGTTACTCTACTAACTCCAGAATTACTGGTGTTTTGCCAAACAACACTACTATCACTTACGATGGTGC
TTACTGTATCAACGGTTACAGATGGATCACTTACATCGCTAATTCTGGTCAAAGAAGATATATTGCTACTGGTG
AAGTTGATAAGGCTGGTAATAGAATTTCTTCTTTTGGTAAATTCTCTACTATCTAA
>L0499_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPTQNYVVKKGDYLGAIAKKFNTTVSKIQKLNNIKNPNFILIGQVLKVPFISSNNSST
NTNRRNNSSSTLNYLNSLAYKGWDFDGAYGNQCFDLVNVYWNHLYGHGLRGYGAKDIPFANNFRGEATVYRNTP
TFKAELGDVAVFSGRYGGGFGHTAIVLNGNYDGKLMKFQSLDQNWYGGDSRRADIAHKVVHNYDYDMYFIRPYK
KA*
>L0499_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTATTCTGATTTGGAGGGAGATTTTGATGTTG
CTGTTTTGCCATTCTCTAACTCTACTAACAACGGTTTGTTGTTCATTAACACTACTATCGCTTCTATCGCTGCTAA
GGAAGAGGGTGTTTCTTTGGAAAAGAGAGAAGCTGAGGCTCATCACCATCACCATCACTTGGAGGTTTTGTTT
CAAGGTCCTACTCAAAACTACGTTGTTAAGAAAGGAGATTATTTGGGTGCTATCGCTAAGAAATTCAATACTAC
TGTTTCTAAGATCCAAAAGTTGAACAACATTAAGAACCCAAACTTCATCTTGATCGGTCAAGTTTTGAAAGTTCC
TTTCATTTCTTCTAACAACTCTTCTACTAACACTAACAGAAGAAACAACTCTTCTTCTACTTTGAACTACTTGAAC
TCTTTGGCTTATAAGGGTTGGGATTTTGATGGTGCTTACGGTAACCAATGTTTCGATTTGGTTAACGTTTACTG
GAATCATTTGTATGGTCACGGTTTGAGAGGTTATGGTGCTAAGGATATTCCATTTGCTAACAATTTCAGAGGTG
AAGCTACTGTTTACAGAAACACTCCTACTTTTAAAGCTGAGTTGGGAGATGTTGCTGTTTTCTCTGGTAGATAC
GGTGGTGGTTTCGGTCATACTGCTATCGTTTTGAACGGTAACTACGATGGTAAATTGATGAAGTTCCAATCTTT
GGATCAAAACTGGTACGGTGGAGATTCTCGTAGAGCTGATATCGCTCATAAGGTTGTTCACAACTACGATTAC
GATATGTACTTCATCAGACCTTACAAGAAAGCTTAA
>L0502_Protein
MRFPSIFTAVLFAASSALAAPVNTTTEDETAQIPAEAVIGYSDLEGDFDVAVLPFSNSTNNGLLFINTTIASIAAKEEGV
SLEKREAEAHHHHHHLEVLFQGPMNKVDAWLSKHDLKNLTTLIVVGAFILFLILLFMLMNHSDQGSNRITVSENAEL
RTDPNAAYPVIYQVEKGDTFTRLSKSGKWIEVESRDGSEKGWIAGWHTSLNIEADSNKKVNPLKNKVIVLDPGHGG
NDQGASSNTSKKSLEKNYTLKTAKELRRALEKAGATVKLTRSDDTYVSLDDRKLNGDAYISIHNDSLDSPNANGATV
YWYKNNQEALAETLNTNIQKKAMLSNRGTRQENFQVLRQTNKPAVLLELGYISNPTDEVMIRNQIHRQVVEEAVV
DGLKQYFAS*
>L0502_DNA
ATGAGATTTCCATCTATTTTCACTGCTGTTTTGTTTGCTGCTTCTTCTGCTTTGGCTGCTCCAGTTAACACTACTAC
TGAAGATGAGACTGCTCAAATTCCTGCTGAAGCTGTTATTGGTTACTCTGATTTGGAGGGAGATTTTGATGTTG
CTGTTTTGCCATTCTCTAACTCTACTAACAACGGTTTGTTGTTTATTAACACTACTATCGCTTCTATTGCTGCTAA
GGAAGAGGGTGTTTCTTTGGAAAAGAGAGAAGCTGAGGCTCATCACCATCACCATCACTTGGAGGTTTTGTTC
CAAGGTCCTATGAACAAGGTTGATGCTTGGTTGTCTAAGCATGATTTGAAAAATTTGACTACTTTGATCGTTGT
TGGTGCTTTTATTTTGTTCTTGATCTTGTTGTTTATGTTGATGAACCACTCTGATCAAGGTTCTAACAGAATTACT
GTTTCTGAAAATGCTGAGTTGAGAACTGATCCAAATGCTGCTTACCCAGTTATCTACCAAGTTGAAAAGGGAG
ATACTTTCACTAGATTGTCTAAGTCTGGTAAATGGATTGAAGTTGAGTCCAGAGATGGTTCTGAAAAAGGTTG
GATTGCTGGTTGGCATACTTCTTTGAACATTGAGGCTGATTCTAACAAGAAAGTTAACCCATTGAAGAACAAAG
TTATTGTTTTGGATCCTGGTCACGGTGGTAACGATCAAGGTGCTTCTTCTAATACTTCTAAGAAGTCTTTGGAAA
AGAACTACACTTTGAAGACTGCTAAAGAATTGCGTAGAGCTTTGGAAAAGGCTGGTGCTACTGTTAAGTTGAC
TAGATCTGATGATACTTACGTTTCTTTGGATGATAGAAAGTTGAACGGAGATGCTTACATCTCTATCCATAACG
ATTCTTTGGATTCTCCTAACGCTAATGGTGCTACTGTTTACTGGTACAAGAACAACCAAGAAGCTTTGGCTGAG
ACTTTGAACACTAACATCCAAAAGAAAGCTATGTTGTCTAACAGAGGTACTAGACAAGAAAACTTCCAAGTTTT
GAGACAAACTAACAAGCCAGCTGTTTTGTTGGAATTGGGTTACATTTCTAACCCTACTGATGAGGTTATGATTA
GAAATCAAATCCACAGACAAGTTGTTGAAGAGGCTGTTGTTGATGGTTTGAAACAATATTTCGCTTCTTAA

### Example 3: Construct Design and mRNA Production

In vitro transcription of lysin encoding mRNAs is based on the pST1-T7-AGA-dEarl-hAg-MCS-FI-A30LA70 plasmid-backbone and derivative DNA-constructs. These plasmid constructs contain a 5′ UTR (untranslated region, a derivate of the 5′-UTR of Homo sapiens hemoglobin subunit alpha 1 (hAg)), a 3′ FI element (where F is a 136 nucleotide long 3′-UTR fragment of amino-terminal enhancer of split, mRNA and I is a 142 nucleotide long fragment of mitochondrially encoded 12S RNA both identified in Homo sapiens; WO 2017/060314) and a poly(A) tail of 100 nucleotides, with a linker after 70 nucleotides.

Lysins with a predicted activity against Staphylococcus aureus were selected and the encoding sequences originate from Staphylococcus species or their phages/viruses (Table 4). The husec signal sequence (derived from Ig heavy chain V-I region HG3; UniProtKB entry HV102_HUMAN) as well as a hexa-histidine tag followed by a 3C protease cleavage site is added N-terminally. The signal sequence targets the lysin constructs to the secretory pathway and is cleaved off at the signal peptidase site after translocation into the ER. The hexa-histidine tag and 3C sites are introduced to facilitate the quantification of translated lysin and its purification by affinity capture and removal of the hexa-histidine tag after proteolytic cleavage, respectively. The lysin sequences are codon-optimized for expression in Pichia pastoris or Homo sapiens.

TABLE 4
Origin of lysin sequences.

| Domain architecture | Internal name | NCBI ID | Species of origin |
|---|---|---|---|
| Ami2 | L0487 | WP_115921155 | Staphylococcus epidermidis |
| CHAP-Ami2-SH3 | L0489 | ABF71642 | Staphylococcus virus 80alpha |
| | L0479 | AWH13065 | Staphylococcus phage trsa10 |
| | L0480 | YP_024461 | Staphylococcus phage K |
| | L0473 | WP_195722665 | Staphylococcus kloosii |
| | L0475 | YP_009268702 | Staphylococcus virus 80 |
| LysM-CHAP | L0499 | WP_070648624 | Staphylococcus |
| | L0482 | WP_143119924 | Staphylococcus aureus |
| PepM23-SH3 | L0483 | Lysostaphin | Staphylococcus simulans |
| | | WP_013012297 | |
| Phage lysozyme | L0484 | PTF44644 | Staphylococcus epidermidis |
| SH3-Ami3 | L0502 | WP_053025743 | Staphylococcus haemolyticus |

mRNA is generated by in vitro transcription as described by Kreiter et al. (Kreiter, S. et al. Cancer Immunol. Immunother. 56, 1577-87 (2007)) with substitution of the normal nucleoside uridine by 1-methyl-pseudouridine. Resulting mRNAs are equipped with a Cap1-structure and double-stranded (dsRNA) molecules is depleted. Purified mRNA is eluted in H₂O and stored at −80° C. until further use. In vitro transcription of all described mRNA constructs is carried out at BioNTech SE. A list of all lysin constructs, which are used in subsequent experiments is shown in Table 5.

TABLE 5
Nucleotide and amino acid sequences of mRNA encoded lysins.

| | |
|---|---|
| L0473 | MDWTWRVFCLLAVAPGAHSMKHHHHHHSAGLEVLFQGPMAKTKKQIEQRLQAYAKGTY [symbol] |
| | DSPYRITKPTSYDPSFGVMEVGAIDADRYYHAQCQDLITDYVLWLTDNKVRPWGNAKDQ [symbol] |
| | NQSYGTGFKIHKNLPSTVAKTGWIAVFTSGTYSQYGHIGIVYSPGDTNKFHIIEQNWNGWA |
| | NKKPLTRWDNYYGLTHFIEVPYAKETITTKKTAPKAATSKPAKKISVSHNNINLQSNFAKRGY |
| | NPKGVVIHNDAGRSTAQQYENSLKGANQNRLNNGIAHAYADSSHIWYAIEPERIAYHTGD |
| | GVKKGTGNYDYFGIEVCQSMSASDKEFMANEQVVLQEVARLLKKWKLPANRNTVRLHLEF |
| | SATSCPHRSLTMRTGFNPVTQGRPSDAIIKKLKDIWIKEIRAYMEGKTPSIKTVANKPGSAST |
| | PATKSNSDGWKVNAYNTYYKTERASFTVGSTRIAVRSTGPFTTCPFTGWLNPGVTITYDEV |
| | MVQNGFVWLGYDSYNGRRYVPIRTAQGTPGSSNYVLGPLWGTIS* |
| | ACTAGTATTCTTCTGGTCCCCACAGACTCAGAGAGAACCCGCCACCATGGATTGGACGT |
| | GGCGAGTTTTCTGTCTGCTGGCTGTTGCCCCAGGCGCGCATTCTATGAAGCATCACCAC [symbol] |
| | ATCACCACTCAGCAGGGCTCGAAGTCCTGTTCCAAGGACCAATGGCCAAAACCAAGAAA |
| | CAGATAGAACAGAGACTGCAAGCTTACGCAAAGGGGACCGTGGATTCCCCTTACCGAA |
| | TCACCAAGCCAACCAGCTACGACCCCTCCTTCGGTGTCATGGAGGTTGGCGCTATAGAT |
| | GCGGATCGCTACTATCATGCCCAGTGTCAAGATCTGATAACTGACTATGTACTTTGGCT [symbol] |
| | ACTGACAACAAAGTAAGACCTTGGGGTAATGCCAAGGACCAGATTAACCAGAGCTATG |
| | GGACTGGTTTCAAAATCCACAAGAATCTGCCCAGTACCGTGGCTAAAACAGGATGGATT |
| | GCCGTGTTTACATCCGGGACCTACTCACAGTACGGCCACATCGGCATCGTGTACAGTCC |
| | AGGAGATACCAACAAATTCCATATCATCGAGCAGAATTGGAATGGATGGGCAAACAAG |
| | AAACCTCTGACACGTTGGGACAACTACTACGGGCTGACCCACTTTATCGAGGTCCCATA |
| | TGCGAAAGAGACAATCACAACGAAGAAAACTGCACCTAAAGCAGCTACCTCAAAACCG |
| | GCTAAGAAGATCTCAGTTTCTCACAACAACATCAACCTTCAGAGCAACTTTGCAAAGAG |
| | AGGGTACAACCCAAAGGGAGTGGTGATTCACAATGACGCCGGACGCTCTACTGCGCAA |
| | CAGTATGAAAACAGCCTGAAGGGCGCTAATCAGAACAGGCTGAATAACGGCATTGCCC |
| | ATGCATATGCTGACAGCAGCCACATTTGGTATGCTATCGAACCCGAGAGAATCGCCTAT |
| | CACACAGGCGATGGCGTTAAGAAGGGAACAGGCAATTACGACTACTTCGGTATTGAAG |
| | TATGCCAGAGCATGAGCGCAAGTGACAAAGAGTTCATGGCCAACGAACAGGTGGTACT |
| | GCAGGAGGTGGCTAGGTTGCTCAAGAAATGGAAGCTTCCCGCCAATCGGAATACCGTG |
| | CGGCTCCATCTGGAATTCTCTGCCACATCTTGCCCTCATAGGAGCCTCACAATGCGGAC [symbol] |
| | GGTTTCAATCCGGTGACTCAGGGTAGACCCTCAGACGCCATCATCAAGAAGTTGAAGGA |
| | TATCTGGATAAAAGAGATTCGCGCATATATGGAGGGTAAAACCCCATCCATTAAGACTG |
| | TCGCCAATAAGCCCGGATCAGCCAGTACCCCTGCAACCAAATCCAACTCCGATGGGTGG |
| | AAGGTCAATGCCTACAATACCTACTACAAAACGGAACGGGCCTCCTTTACAGTGGGCT [symbol] |
| | TACTCGGATTGCTGTGCGTAGTACAGGACCCTTTACTACATGCCCGTTTACAGGGTGGCT |
| | GAACCCCGGAGTCACGATAACCTATGACGAGGTTATGGTGCAGAACGGGTTTGTGTGG |
| | TTGGGCTATGATAGCTACAATGGGAGGAGGTATGTCCCCATTCGCACTGCCCAAGGAA [symbol] |
| | TCCTGGCAGCTCCAATTATGTGCTTGGCCCTCTGTGGGGCACGATTTCTTGActcgagCTG |
| | GTACTGCATGCACGCAATGCTAGCTGCCCCTTTCCCGTCCTGGGTACCCCGAGTCTCCCC |
| | CGACCTCGGGTCCCAGGTATGCTCCCACCTCCACCTGCCCCACTCACCACCTCTGCTAGT |
| | TCCAGACACCTCCCAAGCACGCAGCAATGCAGCTCAAAACGCTTAGCCTAGCCACACCC |
| | CCACGGGAAACAGCAGTGATTAACCTTTAGCAATAAACGAAAGTTTAACTAAGCTATAC |
| | TAACCCCAGGGTTGGTCAATTTCGTGCCAGCCACACCCTGGAGCTAGCAAAAAAAAAAA |
| | AAAAAAAAAAAAAAAAAAAGCATATGACTAAAAAAAAAAAAAAAAAAAAAAAAAAAA |
| | AAAAAAAAAAAAAAAAAAAAAAAAAAAAAAAAAAAAAAAAAA |
| L0475 | MDWTWRVFCLLAVAPGAHSMKHHHHHHSAGLEVLFQGPMQAKLTKKEFIEWLKTSEGK |
| | QFNADLWYGFQCFDYANAAWKVLFGLLLKGLGAKDIPFANNFDGLATVYQNTPDFLAQPG |
| | DMVVFGSNYGAGYGHVAWVIEATLDYIIVYEQNWLGGGWTDGIEQPGWGWEKVTRRQ |
| | HAYDFPMWFIRPNFKSEIAPRSVQSPTQAPKKETAKPQPKAVELKIIKDVVKGYDLPKRGSN |
| | PKGIVIHNDAGSKGATAEAYRNGLVNAPLSRLEAGIAHSYVSGNTVWQALDESQVGWHTA |
| | NQLGNKYYYGIEVCOSMGADNATFLKNEQATFQECARLLKKWGLPANRNTIRLHNEFTSTS |
| | CPHRSSVLHTGFDPVTRGLLPEDKRLOLKDYFIKQIRAYMDGKIPVATVSNDSSASSNTVKPV |
| | ASAWKRNKYGTYYMEESARFTNGNQPITVRKVGPFLYCPVGYQFQPGGYCDYTEVMLQD |
| | GHVWVGYTWEGQRYYLPIRTWNGSAPPNQILGDLWGEIS* |
| | ACTAGTATTCTTCTGGTCCCCACAGACTCAGAGAGAACCCGCCACCATGGATTGGACAT |
| | GGCGAGTGTTCTGCCTTCTTGCTGTTGCACCTGGTGCCCATTCCATGAAACATCACCACC |
| | ATCACCATTCAGCTGGCCTTGAAGTGCTTTTCCAGGGACCTATGCAAGCGAAACTGACC |
| | AAGAAGGAGTTTATAGAGTGGCTGAAAACCTCTGAGGGTAAACAGTTCAACGCCGATC |
| | TGTGGTATGGTTTCCAGTGCTTTGATTACGCAAACGCTGCTTGGAAAGTGCTGTTTGGCC |
| | TGCTCCTGAAGGGACTCGGTGCCAAAGACATCCCCTTTGCCAACAATTTCGACGGGCTG |
| | GCAACCGTCTACCAGAATACTCCTGACTTTCTCGCTCAGCCAGGGGACATGGTCGTGTTC |
| | GGCTCCAATTATGGGGCAGGCTATGGACACGTAGCATGGGTGATAGAAGCCACTCTGG |
| | ACTACATAATCGTCTACGAGCAAAATTGGCTTGGCGGTGGGTGGACCGATGGCATTGA |
| | GCAGCCTGGCTGGGGATGGGAGAAGGTTACTCGTAGACAGCATGCTTACGACTTTCCC |
| | ATGTGGTTCATCCGTCCCAACTTCAAGTCCGAGATTGCGCCCAGAAGCGTCCAGTCTCCA |
| | ACCCAAGCTCCGAAGAAGGAAACAGCCAAGCCACAGCCTAAGGCCGTTGAACTGAAGA |
| | TTATTAAGGATGTGGTGAAGGGATATGACCTCCCGAAAAGAGGGAGCAATCCCAAAGG |
| | GATTGTGATCCACAACGATGCCGGGTCTAAAGGGGCAACAGCAGAAGCGTATAGGAAC |
| | GGGTTGGTGAATGCCCCTCTCAGTCGCTTGGAGGCCGGAATTGCACACTCATATGTGTC |
| | CGGAAATACGGTTTGGCAAGCTCTCGACGAAAGCCAAGTAGGCTGGCATACAGCCAAC |
| | CAGCTGGGCAACAAATACTACTATGGCATCGAGGTGTGCCAGAGTATGGGCGCAGATA |
| | ATGCCACGTTCCTGAAGAACGAGCAGGCTACCTTTCAAGAGTGTGCGCGGTTGCTGAA |
| | GAAGTGGGGACTGCCAGCTAATCGCAACACAATCAGGTTGCACAACGAGTTTACGAGT |
| | ACTAGCTGTCCACACAGAAGTAGCGTGCTGCACACAGGATTCGATCCTGTGACAAGGG |
| | GACTGCTGCCCGAAGATAAGCGGCTGCAGCTGAAAGACTACTTCATCAAACAGATTCGG |
| | GCCTATATGGACGGAAAGATTCCCGTCGCTACTGTTAGCAATGACAGCTCTGCCTCATC |
| | AAATACCGTGAAACCGGTAGCAAGCGCCTGGAAAAGGAACAAGTATGGCACTTATTAC |
| | ATGGAGGAATCCGCCCGATTTACCAACGGTAATCAGCCCATCACAGTCCGCAAGGTCGG |
| | GCCATTCCTGTACTGTCCCGTAGGCTACCAGTTTCAGCCTGGTGGGTACTGCGACTATAC |
| | CGAGGTGATGCTGCAAGATGGCCATGTCTGGGTTGGCTATACCTGGGAAGGGCAGCGC |
| | TACTACTTGCCAATACGGACTTGGAATGGTTCTGCCCCACCCAACCAGATCCTCGGAGAT |
| | CTGTGGGGCGAAATCTCCTGActcgagCTGGTACTGCATGCACGCAATGCTAGCTGCCCC |
| | TTTCCCGTCCTGGGTACCCCGAGTCTCCCCCGACCTCGGGTCCCAGGTATGCTCCCACCT |
| | CCACCTGCCCCACTCACCACCTCTGCTAGTTCCAGACACCTCCCAAGCACGCAGCAATGC |
| | AGCTCAAAACGCTTAGCCTAGCCACACCCCCACGGGAAACAGCAGTGATTAACCTTTAG |
| | CAATAAACGAAAGTTTAACTAAGCTATACTAACCCCAGGGTTGGTCAATTTCGTGCCAG |
| | CCACACCCTGGAGCTAGCAAAAAAAAAAAAAAAAAAAAAAAAAAAAAAGCATATGACT |
| | AAAAAAAAAAAAAAAAAAAAAAAAAAAAAAAAAAAAAAAAAAAAAAAAAAAAAAAAA |
| | AAAAAAAAAAAAA |
| L0479 | MDWTWRVFCLLAVAPGAHSMKHHHHHHSAGLEVLFQGPMQAKLTKKEFIEWLKTSEGK |
| | QFNVDLWYGFQCFDYANAGWKVLFGLLLKGLGAKDIPFANNFDGLATVYQNTPDFLAKPG |
| | DMVVFGSNYGAGYGHVAWVIEATLDYIIVYEQNWLGGGWTDRIEQPGWGWEKVTRRQ |
| | HALRFPYVVYPSYPKSETAPRSIQSPTQASKKETAKPQPKAVELKIIKDVVKGYDLPKRGGNP |
| | KGIVIHNDAGSKGATAEAYRNGLVNAPLSRLEAGIAHSYVSGNTVWQALDESQVGWHTAN |
| | QLGNKYYYGIEVCOSMGADNATFLKNEQATFQECARLLKKWGLPANRNTIRLHNEFTSTSC |
| | PHRSSVLHTGFDPVTRGLLPEDKRLQLKDYFIKQIRAYMDGKIPVATVSNESSASSNTVKPVA |
| | SAWKRNKYGTYYMEESARFTNGNQPITVRKVGPFLSCPVGYQFQPGGYCDYTEVMLQDG |
| | HVWVGYTWEGQRYYLPIRTWNGSAPPNQILGDLWGEIS* |
| | ACTAGTATTCTTCTGGTCCCCACAGACTCAGAGAGAACCCGCCACCATGGACTGGACCT |
| | GGAGAGTCTTTTGCCTCCTTGCAGTTGCTCCTGGTGCCCACAGTATGAAGCATCACCATC |
| | ATCACCACAGTGCGGGACTGGAGGTGTTGTTTCAGGGACCAATGCAGGCCAAACTGAC |
| | CAAGAAGGAGTTCATCGAATGGCTCAAGACTTCCGAGGGCAAGCAGTTCAATGTGGAC |
| | CTCTGGTATGGCTTTCAATGCTTTGATTATGCCAATGCTGGGTGGAAGGTCCTGTTTGGC |
| | CTTCTCCTGAAAGGACTGGGTGCAAAAGACATCCCCTTCGCCAACAACTTCGATGGACT |
| | GGCTACCGTTTACCAGAATACCCCGGACTTTCTGGCCAAGCCTGGAGATATGGTGGTGT |
| | TCGGAAGCAACTATGGGGCAGGATATGGCCATGTAGCCTGGGTGATAGAAGCTACGTT |
| | GGACTACATCATCGTGTACGAGCAAAATTGGCTGGGCGGCGGCTGGACTGACCGAATT |
| | GAGCAACCTGGGTGGGGATGGGAGAAAGTGACCAGACGTCAGCACGCTCTGCGCTTCC |
| | CCTATGTAGTGTACCCCTCTTACCCGAAGTCTGAGACAGCTCCCCGGTCCATTCAGAGCC |
| | CAACACAGGCGAGCAAGAAAGAGACTGCCAAACCACAACCCAAAGCGGTTGAACTGAA |
| | GATCATCAAAGACGTCGTGAAAGGGTATGATCTGCCCAAGAGAGGCGGAAATCCAAAG |
| | GGGATTGTGATCCACAATGACGCCGGGAGCAAAGGTGCAACTGCTGAAGCTTACCGCA |
| | ATGGGCTGGTCAACGCACCTTTGTCCCGACTGGAAGCCGGCATTGCTCATAGCTATGTG |
| | TCTGGCAACACTGTTTGGCAAGCACTGGACGAATCACAGGTCGGCTGGCATACTGCCAA |
| | TCAGCTCGGAAACAAGTACTACTACGGGATAGAGGTCTGTCAGTCCATGGGCGCCGAT |
| | AACGCGACTTTCCTGAAGAATGAGCAGGCCACATTCCAGGAATGCGCTCGGTTGTTGAA |
| | GAAATGGGGCCTTCCTGCAAACAGGAACACAATCAGGCTGCACAATGAGTTCACATCCA |
| | CGTCTTGTCCACACAGGTCTAGCGTGCTGCACACAGGTTTTGATCCCGTGACGAGAGGG |
| | CTTCTCCCCGAAGATAAACGTCTGCAGCTCAAGGATTACTTCATCAAGCAGATAAGGGC |
| | CTATATGGACGGGAAAATTCCGGTAGCTACCGTCAGCAACGAGAGTAGCGCATCATCCA |
| | ACACCGTTAAGCCAGTGGCCTCAGCCTGGAAGCGCAACAAATACGGTACCTATTACATG |
| | GAGGAATCTGCAAGATTCACAAATGGTAATCAGCCTATTACCGTGCGGAAAGTCGGCCC |
| | TTTTCTCAGCTGTCCTGTTGGATATCAGTTTCAGCCAGGCGGTTACTGCGACTATACCGA |
| | AGTAATGCTGCAAGATGGGCATGTGTGGGTTGGGTACACATGGGAAGGCCAACGGTAC |
| | TATCTGCCCATTCGCACCTGGAACGGCAGTGCCCCACCCAACCAGATCCTTGGAGATCT |
| | GTGGGGTGAGATTTCATGActcgagCTGGTACTGCATGCACGCAATGCTAGCTGCCCCTTT |
| | CCCGTCCTGGGTACCCCGAGTCTCCCCCGACCTCGGGTCCCAGGTATGCTCCCACCTCCA |
| | CCTGCCCCACTCACCACCTCTGCTAGTTCCAGACACCTCCCAAGCACGCAGCAATGCAGC |
| | TCAAAACGCTTAGCCTAGCCACACCCCCACGGGAAACAGCAGTGATTAACCTTTAGCAA |
| | TAAACGAAAGTTTAACTAAGCTATACTAACCCCAGGGTTGGTCAATTTCGTGCCAGCCAC |
| | ACCCTGGAGCTAGCAAAAAAAAAAAAAAAAAAAAAAAAAAAAAAGCATATGACTAAAA |
| | AAAAAAAAAAAAAAAAAAAAAAAAAAAAAAAAAAAAAAAAAAAAAAAAAAAAAAAAA |
| | AAAAAAAAA |
| L0480 | MDWTWRVFCLLAVAPGAHSMKHHHHHHSAGLEVLFQGPMAKTQAEINKRLDAYAKGTV |
| | DSPYRVKKATSYDPSFGVMEAGAIDADGYYHAQCQDLITDYVLWLTDNKVRTWGNAKDQI |
| | KQSYGTGFKIHENKPSTVPKKGWIAVFTSGSYEQWGHIGIVYDGGNTSTFTILEQNWNGYA |
| | NKKPTKRVDNYYGLTHFIEIPVKAGTTVKKETAKKSASKTPAPKKKATLKVSKNHINYTMDKR |
| | GKKPEGMVIHNDAGRSSGQQYENSLANAGYARYANGIAHYYGSEGYVWEAIDAKNQIAW |
| | HTGDGTGANSGNFRFAGIEVCQSMSASDAQFLKNEQAVFQFTAEKFKEWGLTPNRKTVRL |
| | HMEFVPTACPHRSMVLHTGFNPVTQGRPSQAIMNKLKDYFIKQIKNYMDKGTSSSTVVKD |
| | GKTSSASTPATRPVTGSWKKNQYGTWYKPENATFVNGNQPIVTRIGSPFLNAPVGGNLPA |
| | GATIVYDEVCIQAGHIWIGYNAYNGNRVYCPVRTCQGVPPNQIPGVAWGVFK* |
| | ACTAGTATTCTTCTGGTCCCCACAGACTCAGAGAGAACCCGCCACCATGGACTGGACTT |
| | GGCGGGTCTTCTGTTTGCTTGCCGTCGCCCCCGGAGCACACTCTATGAAGCACCACCAC |
| | CATCACCACTCTGCCGGCCTGGAAGTGCTGTTTCAGGGCCCTATGGCCAAGACACAGGC |
| | CGAGATCAACAAGCGGCTGGACGCCTATGCCAAGGGCACAGTGGATAGCCCCTACAGA |
| | GTGAAGAAGGCCACCAGCTACGACCCCAGCTTCGGCGTTATGGAAGCCGGCGCTATTG |
| | ACGCCGACGGCTATTATCATGCCCAGTGCCAGGACCTGATCACCGACTATGTGCTGTGG |
| | CTGACCGACAACAAAGTGCGGACATGGGGCAACGCCAAGGACCAGATCAAGCAGAGC |
| | TACGGCACCGGCTTCAAGATCCACGAGAACAAGCCTAGCACCGTGCCTAAGAAAGGCT |
| | GGATCGCCGTGTTTACCAGCGGCAGCTATGAGCAGTGGGGCCACATCGGCATCGTGTA |
| | CGATGGCGGCAATACCAGCACCTTCACCATCCTGGAACAGAACTGGAACGGCTACGCCA |
| | ACAAGAAACCCACCAAGAGAGTGGACAACTACTACGGCCTGACACACTTCATCGAGATC |
| | CCCGTGAAGGCCGGCACCACCGTGAAGAAAGAGACAGCCAAGAAGTCCGCCAGCAAG |
| | ACCCCTGCTCCTAAGAAGAAAGCCACACTGAAGGTGTCCAAGAACCACATCAACTACAC |
| | CATGGACAAGCGGGGCAAAAAGCCCGAAGGCATGGTCATCCACAACGACGCCGGCAG |
| | ATCTAGCGGCCAGCAGTACGAAAACAGCCTGGCCAATGCCGGCTATGCCAGATACGCC |
| | AATGGAATCGCCCACTACTATGGCAGCGAGGGCTATGTGTGGGAAGCCATCGACGCCA |
| | AGAATCAGATCGCCTGGCATACAGGCGACGGAACCGGCGCCAATTCCGGCAATTTCAG |
| | ATTCGCCGGCATCGAAGTGTGCCAGAGCATGTCTGCCAGCGACGCCCAGTTCCTGAAGA |
| | ATGAGCAGGCCGTGTTCCAGTTTACCGCCGAGAAGTTCAAAGAGTGGGGACTGACCCC |
| | TAACAGAAAGACCGTGCGGCTGCACATGGAATTCGTGCCTACCGCATGTCCCCACCGCT |
| | CTATGGTGCTGCACACCGGCTTTAACCCCGTGACACAGGGCAGACCTAGCCAGGCCATC |
| | ATGAACAAGCTGAAGGACTACTTCATTAAGCAGATCAAGAACTACATGGATAAGGGCA |
| | CCAGCAGCAGCACCGTGGTCAAGGATGGCAAGACCAGCAGCGCCTCTACACCAGCCAC |
| | AAGACCTGTGACAGGCAGCTGGAAGAAGAACCAGTACGGCACCTGGTACAAGCCCGA |
| | GAACGCCACCTTCGTGAACGGCAACCAGCCTATCGTGACCAGAATCGGCAGCCCCTTCC |
| | TGAATGCCCCTGTCGGAGGAAATCTTCCTGCTGGCGCCACCATCGTGTATGACGAAGTC |
| | TGTATTCAGGCCGGCCACATCTGGATCGGCTACAACGCCTACAACGGCAACAGAGTGTA |
| | CTGCCCCGTGCGGACTTGTCAGGGCGTTCCACCTAATCAGATTCCAGGCGTGGCCTGGG |
| | GCGTGTTCAAATGActcgagCTGGTACTGCATGCACGCAATGCTAGCTGCCCCTTTCCCGT |
| | CCTGGGTACCCCGAGTCTCCCCCGACCTCGGGTCCCAGGTATGCTCCCACCTCCACCTGC |
| | CCCACTCACCACCTCTGCTAGTTCCAGACACCTCCCAAGCACGCAGCAATGCAGCTCAAA |
| | ACGCTTAGCCTAGCCACACCCCCACGGGAAACAGCAGTGATTAACCTTTAGCAATAAAC |
| | GAAAGTTTAACTAAGCTATACTAACCCCAGGGTTGGTCAATTTCGTGCCAGCCACACCCT |
| | GGAGCTAGCAAAAAAAAAAAAAAAAAAAAAAAAAAAAAAGCATATGACTAAAAAAAA |
| | AAAAAAAAAAAAAAAAAAAAAAAAAAAAAAAAAAAAAAAAAAAAAAAAAAAAAAAAA |
| | AAAAA |
| L0482 | MDWTWRVFCLLAVAPGAHSMKHHHHHHSAGLEVLFQGPREAPKTQIYTVKKGDTLSAIA |
| | LKYKTTVSNIQNTNNIANPNLIFIGQKLKVPMTPLVEPKPKTVSSNNKSNSNSSTLNYLKTLEN |
| | RGWDFDGSYGWQCFDLVNVYWNHLYGHGLKGYGAKDIPYANNFNSEAKIYHNTPTFKAE |
| | PGDLVVFSGRFGGGYGHTAIVLNGDYDGKLMKFQSLDQNWNNGGWRKAEVAHKVVHN |
| | YENDMIFIRPFKKA* |
| | ACTAGTATTCTTCTGGTCCCCACAGACTCAGAGAGAACCCGCCACCATGGATTGGACAT |
| | GGAGGGTGTTTTGTCTGCTCGCAGTTGCTCCAGGTGCACATAGCATGAAACACCACCAT |
| | CACCACCATTCTGCTGGGCTTGAGGTGCTGTTTCAAGGACCTAGAGAGGCTCCCAAAAC |
| | ACAGATTTACACCGTCAAGAAGGGAGATACGCTTAGCGCCATTGCACTGAAATACAAGA |
| | CCACCGTATCAAACATCCAGAACACCAATAACATAGCCAATCCCAATCTCATCTTCATTG |
| | GCCAGAAGCTCAAGGTTCCCATGACTCCACTGGTCGAACCGAAACCTAAGACAGTGTCC |
| | TCCAATAACAAGAGTAACAGCAACTCCTCTACACTGAACTATCTCAAGACGTTGGAGAA |
| | TCGCGGTTGGGACTTTGACGGCAGTTACGGCTGGCAATGCTTCGACTTGGTGAATGTGT |
| | ACTGGAATCACCTGTATGGGCATGGACTGAAAGGCTATGGGGCCAAAGACATACCTTAT |
| | GCTAACAATTTCAACTCTGAGGCCAAGATCTACCACAACACTCCTACCTTCAAGGCAGAA |
| | CCAGGAGATCTGGTTGTGTTTAGCGGACGGTTTGGCGGAGGCTATGGGCATACTGCCA |
| | TTGTGCTTAATGGGGATTACGATGGCAAACTGATGAAATTCCAGTCACTGGACCAGAAC |
| | TGGAACAATGGTGGTTGGCGAAAAGCGGAAGTAGCCCACAAGGTCGTCCACAATTACG |
| | AGAACGACATGATCTTCATCCGTCCCTTTAAGAAAGCGTGActcgagCTGGTACTGCATGC |
| | ACGCAATGCTAGCTGCCCCTTTCCCGTCCTGGGTACCCCGAGTCTCCCCCGACCTCGGGT |
| | CCCAGGTATGCTCCCACCTCCACCTGCCCCACTCACCACCTCTGCTAGTTCCAGACACCTC |
| | CCAAGCACGCAGCAATGCAGCTCAAAACGCTTAGCCTAGCCACACCCCCACGGGAAACA |
| | GCAGTGATTAACCTTTAGCAATAAACGAAAGTTTAACTAAGCTATACTAACCCCAGGGTT |
| | GGTCAATTTCGTGCCAGCCACACCCTGGAGCTAGCAAAAAAAAAAAAAAAAAAAAAAA |
| | AAAAAAAGCATATGACTAAAAAAAAAAAAAAAAAAAAAAAAAAAAAAAAAAAAAAAA |
| | AAAAAAAAAAAAAAAAAAAAAAAAAAAAAA |
| L0483 | MDWTWRVFCLLAVAPGAHSMKHHHHHHSAGLEVLFQGPAATHEHSAQWLNNYKKGYG |
| | YGPYPLGINGGMHYGVDFFMNIGTPVKAISSGKIVEAGWSNYGGGNQIGLIENDGVHRQ |
| | WYMHLSKYNVKVGDYVKAGQIIGWSGSTGYSTAPHLHFQRMVNSFSNSTAQDPMPFLKS |
| | AGYGKAGGTVTPTPNTGWKTNKYGTLYKSESASFTPNTDIITRTTGPFRSMPQSGVLKAGQ |
| | TIHYDEVMKQDGHVWVGYTGNSGQRIYLPVRTWNKSTNTLGVLWGTIK* |
| | ACTAGTATTCTTCTGGTCCCCACAGACTCAGAGAGAACCCGCCACCATGGATTGGACCT |
| | GGAGAGTGTTTTGCCTTCTCGCTGTTGCCCCTGGTGCCCATTCCATGAAGCACCACCACC |
| | ACCATCATAGTGCAGGGCTGGAAGTTCTGTTTCAGGGTCCAGCAGCAACGCATGAGCAT |
| | TCCGCTCAATGGCTCAACAACTACAAGAAGGGTTACGGCTATGGCCCCTATCCTCTGGG |
| | GATAAACGGAGGGATGCACTATGGAGTCGATTTCTTCATGAATATTGGCACTCCAGTGA |
| | AAGCGATTTCCAGCGGGAAGATTGTGGAAGCTGGGTGGAGCAACTATGGTGGTGGCA |
| | ATCAGATCGGACTGATTGAGAATGACGGCGTGCACAGACAGTGGTATATGCATCTGAG |
| | TAAGTACAACGTCAAGGTGGGAGACTACGTCAAAGCCGGGCAGATCATAGGATGGTCC |
| | GGCTCTACAGGTTACTCAACAGCGCCACATCTCCACTTTCAGCGCATGGTGAATAGCTTC |
| | AGCAATTCAACCGCCCAAGATCCGATGCCCTTCCTGAAGTCTGCCGGCTATGGGAAAGC |
| | TGGAGGAACAGTGACACCTACTCCCAATACCGGATGGAAAACGAACAAGTACGGCACT |
| | CTGTACAAATCCGAATCTGCCAGTTTCACACCCAATACCGACATCATCACCCGAACCACT |
| | GGACCTTTTCGGTCAATGCCGCAGTCTGGAGTACTGAAAGCAGGGCAAACTATCCACTA |
| | TGACGAGGTGATGAAGCAGGATGGCCACGTATGGGTGGGGTACACAGGCAATAGCGG |
| | CCAGAGGATTTACTTGCCAGTCCGTACCTGGAACAAAAGCACGAACACTCTTGGCGTTT |
| | TGTGGGGCACCATCAAGTGActcgagCTGGTACTGCATGCACGCAATGCTAGCTGCCCCT |
| | TTCCCGTCCTGGGTACCCCGAGTCTCCCCCGACCTCGGGTCCCAGGTATGCTCCCACCTC |
| | CACCTGCCCCACTCACCACCTCTGCTAGTTCCAGACACCTCCCAAGCACGCAGCAATGCA |
| | GCTCAAAACGCTTAGCCTAGCCACACCCCCACGGGAAACAGCAGTGATTAACCTTTAGC |
| | AATAAACGAAAGTTTAACTAAGCTATACTAACCCCAGGGTTGGTCAATTTCGTGCCAGC |
| | CACACCCTGGAGCTAGCAAAAAAAAAAAAAAAAAAAAAAAAAAAAAAGCATATGACTA |
| | AAAAAAAAAAAAAAAAAAAAAAAAAAAAAAAAAAAAAAAAAAAAAAAAAAAAAAAAA |
| | AAAAAAAAAAAA |
| L0484 | MDWTWRVFCLLAVAPGAHSMKHHHHHHSAGLEVLFQGPDQFLDEKEGNHTTAYRDGS |
| | GIWTICRGATMVDGKPVIPGMRLSKEKCDQVNAIERDKALAWVERNIKVPLTEPQKAGIAS |
| | FCPYNIGPGKCFPSTFYKRLNAGDRKGACEAIRWWIKDGGRDCRIRSNNCYGQVIRRDQES |
| | ALACWGIDQ* |
| | ACTAGTATTCTTCTGGTCCCCACAGACTCAGAGAGAACCCGCCACCATGGACTGGACAT |
| | GGCGCGTGTTTTGCCTTCTCGCCGTAGCACCTGGTGCACACAGCATGAAGCATCACCAT |
| | CACCATCACTCTGCAGGCTTGGAAGTCCTGTTCCAAGGGCCTGATCAGTTTCTCGACGA |
| | GAAGGAAGGCAATCACACCACTGCGTATAGGGATGGCTCTGGGATATGGACCATCTGT |
| | AGAGGCGCCACAATGGTCGATGGCAAACCCGTGATACCCGGAATGCGTCTGTCAAAGG |
| | AGAAGTGCGATCAGGTGAATGCCATCGAACGCGACAAAGCTCTGGCCTGGGTTGAGCG |
| | GAACATCAAGGTTCCACTGACGGAACCACAGAAAGCCGGAATTGCGTCCTTCTGTCCGT |
| | ACAACATTGGCCCTGGAAAGTGCTTTCCCTCCACTTTCTACAAGCGCCTGAATGCAGGG |
| | GACAGAAAAGGAGCCTGTGAGGCTATCAGGTGGTGGATCAAAGATGGAGGTAGGGAC |
| | TGTCGAATCCGGAGCAACAACTGCTATGGGCAAGTGATTCGGAGAGATCAGGAGAGTG |
| | CTCTGGCTTGCTGGGGTATTGACCAGTGActcgagCTGGTACTGCATGCACGCAATGCTA |
| | GCTGCCCCTTTCCCGTCCTGGGTACCCCGAGTCTCCCCCGACCTCGGGTCCCAGGTATGC |
| | TCCCACCTCCACCTGCCCCACTCACCACCTCTGCTAGTTCCAGACACCTCCCAAGCACGCA |
| | GCAATGCAGCTCAAAACGCTTAGCCTAGCCACACCCCCACGGGAAACAGCAGTGATTAA |
| | CCTTTAGCAATAAACGAAAGTTTAACTAAGCTATACTAACCCCAGGGTTGGTCAATTTCG |
| | TGCCAGCCACACCCTGGAGCTAGCAAAAAAAAAAAAAAAAAAAAAAAAAAAAAAGCAT |
| | ATGACTAAAAAAAAAAAAAAAAAAAAAAAAAAAAAAAAAAAAAAAAAAAAAAAAAAA |
| | AAAAAAAAAAAAAAAAAAA |
| L0487 | MDWTWRVFCLLAVAPGAHSMKHHHHHHSAGLEVLFQGPMRYIPAAHHSAGSNKPVNR |
| | VVIHATCPDVGFPSASRKGRAVSTANYFASPSSGGSAHYVCDIGETVQCLSESTIGWHAPPN |
| | PHSLGIEICADGGSHASFRVPGHAYTREQWLDPRVWPAVERAAVLCRQLCDKHGVPKRKL |
| | SVADLKAGRRGICGHTDVTDAWHQSDHDDPGPWFPWDKFMAVVNGHGGGSSSEELTV |
| | ADVKALHNQIKQLSAQLSGSVNKLHHDVGVVQVQNGDLSKRVDALSWVKNPVTGKLWRT |
| | KDALWSVWYYVLECRSRIDRLESAVNGLKK* |
| | ACTAGTATTCTTCTGGTCCCCACAGACTCAGAGAGAACCCGCCACCATGGACTGGACAT |
| | GGAGAGTGTTCTGTCTGCTTGCTGTGGCACCAGGCGCTCACTCCATGAAACACCACCAC |
| | CATCACCATTCTGCTGGTCTGGAAGTGCTTTTCCAGGGACCTATGCGCTATATTCCTGCT |
| | GCACACCATTCTGCAGGGAGCAACAAGCCCGTGAATCGCGTTGTCATCCATGCCACTTG |
| | TCCCGATGTGGGCTTTCCCTCCGCCTCACGGAAAGGAAGGGCAGTGAGCACTGCCAACT |
| | ACTTCGCCAGTCCGTCCTCAGGCGGGTCAGCCCACTATGTGTGCGACATAGGGGAGAC |
| | AGTACAGTGTCTCAGCGAAAGCACGATTGGGTGGCATGCCCCACCGAATCCACACTCTT |
| | TGGGGATCGAGATTTGCGCAGATGGAGGTTCTCATGCCTCTTTTCGCGTGCCAGGCCAT |
| | GCATACACCAGAGAGCAGTGGCTCGATCCACGAGTTTGGCCCGCAGTTGAACGAGCTG |
| | CTGTGCTGTGCCGGCAATTGTGCGACAAGCATGGCGTACCCAAACGGAAACTGAGCGT |
| | TGCGGACCTGAAAGCCGGGAGGAGGGGTATATGCGGACATACCGACGTGACTGACGC |
| | GTGGCACCAGAGTGACCACGATGACCCTGGGCCTTGGTTTCCTTGGGACAAGTTCATGG |
| | CGGTGGTAAACGGACATGGTGGCGGCAGTAGTAGCGAGGAACTGACCGTTGCCGATG |
| | TCAAGGCCCTGCACAACCAGATCAAGCAGCTGTCTGCTCAACTCTCCGGAAGCGTGAAC |
| | AAGCTGCACCACGATGTGGGAGTCGTCCAAGTGCAGAATGGCGACCTCTCCAAGAGAG |
| | TGGATGCCTTGAGCTGGGTCAAGAATCCCGTTACAGGCAAACTTTGGCGTACCAAAGAT |
| | GCCCTGTGGAGCGTCTGGTACTATGTACTGGAGTGTCGGTCCAGGATCGATAGACTGG |
| | AGTCAGCTGTCAATGGTCTGAAGAAGTGActcgagCTGGTACTGCATGCACGCAATGCTA |
| | GCTGCCCCTTTCCCGTCCTGGGTACCCCGAGTCTCCCCCGACCTCGGGTCCCAGGTATGC |
| | TCCCACCTCCACCTGCCCCACTCACCACCTCTGCTAGTTCCAGACACCTCCCAAGCACGCA |
| | GCAATGCAGCTCAAAACGCTTAGCCTAGCCACACCCCCACGGGAAACAGCAGTGATTAA |
| | CCTTTAGCAATAAACGAAAGTTTAACTAAGCTATACTAACCCCAGGGTTGGTCAATTTCG |
| | TGCCAGCCACACCCTGGAGCTAGCAAAAAAAAAAAAAAAAAAAAAAAAAAAAAAGCAT |
| | ATGACTAAAAAAAAAAAAAAAAAAAAAAAAAAAAAAAAAAAAAAAAAAAAAAAAAAA |
| | AAAAAAAAAAAAAAAAAAA |
| L0489 | MDWTWRVFCLLAVAPGAHSMKHHHHHHSAGLEVLFQGPMLMTKNQAEKWFDNSLGK |
| | QFNPDGWYGFQCYDYANMFFMLATGERLOGLYAYNIPFDNKAKIEKYGQIIKNYDSFLPQK |
| | LDIVVFPSKYGGGAGHVEIVESANLNTFTSFGQNWNGKGWTNGVAQPGWGPETVTRHV |
| | HYYDNPMYFIRLNFPNNLSVGNKAKGIIKQATTKKEAVIKPKKIMLVAGHGYNDPGAVGNG |
| | TNERDFIRKYITPNIAKYLRHAGHEVALYGGSSQSQDMYQDTAYGVNVGNKKDYGLYWVK |
| | SQGYDIVLEIHLDAAGESASGGHVIISSQFNADTIDKSIQDVIKNNLGQIRGVTPRNDLLNVN |
| | VSAEININYRLSELGFITNKNDMDWIKKNYDLYSKLIAGAIHGKPIGGLVAGNVKTSAKNKKN |
| | PPVPAGYTLDKNNVPYKKEQGNYTVANVKGNNVRDGYSTNSRITGVLPNNTTITYDGAYCI |
| | NGYRWITYIANSGORRYIATGEVDKAGNRISSFGKFSTI* |
| | ACTAGTATTCTTCTGGTCCCCACAGACTCAGAGAGAACCCGCCACCATGGACTGGACTT |
| | GGCGGGTCTTCTGTTTGCTTGCCGTCGCCCCCGGAGCACACTCTATGAAGCACCACCAC |
| | CATCACCACTCTGCCGGCCTGGAAGTGCTGTTTCAGGGCCCTATGCTGATGACCAAGAA |
| | CCAGGCCGAGAAGTGGTTCGACAACAGCCTGGGCAAGCAGTTCAACCCCGATGGATGG |
| | TACGGCTTCCAGTGCTACGACTACGCCAACATGTTCTTCATGCTGGCCACCGGCGAGAG |
| | ACTGCAGGGCCTGTACGCCTACAACATCCCCTTCGATAACAAGGCCAAGATCGAGAAGT |
| | ACGGCCAGATCATCAAGAACTACGACAGCTTCCTGCCTCAGAAACTGGACATCGTGGTG |
| | TTCCCCAGCAAATATGGCGGCGGAGCCGGACACGTGGAAATCGTGGAAAGCGCCAACC |
| | TGAACACCTTCACCAGCTTCGGCCAGAACTGGAACGGCAAAGGCTGGACCAATGGCGT |
| | GGCACAACCTGGCTGGGGACCTGAGACAGTGACAAGACACGTGCACTACTACGACAAC |
| | CCCATGTACTTCATCCGGCTGAACTTCCCCAACAACCTGAGCGTGGGCAACAAAGCCAA |
| | GGGCATCATCAAGCAGGCCACCACCAAGAAAGAAGCCGTGATCAAGCCCAAGAAAATC |
| | ATGCTGGTGGCTGGCCACGGCTACAACGATCCTGGCGCTGTTGGCAACGGCACCAACG |
| | AGAGAGACTTCATCAGAAAGTACATCACGCCCAATATCGCCAAGTACCTGAGGCACGCC |
| | GGACATGAGGTTGCCCTGTATGGCGGATCTAGCCAGAGCCAGGACATGTACCAGGATA |
| | CCGCCTACGGCGTGAACGTGGGAAACAAGAAGGACTACGGCCTGTACTGGGTCAAGAG |
| | CCAGGGCTACGACATCGTGCTGGAAATCCACCTGGATGCCGCTGGCGAATCTGCCTCTG |
| | GCGGACATGTGATCATCAGCAGCCAGTTCAATGCCGACACCATCGACAAGAGCATCCAG |
| | GATGTGATCAAGAACAACCTGGGCCAGATCCGGGGCGTGACCCCTAGAAACGATCTGC |
| | TGAATGTGAACGTGTCCGCCGAGATCAACATCAACTACCGGCTGAGCGAGCTGGGCTTC |
| | ATCACCAACAAGAACGACATGGACTGGATCAAGAAGAATTACGACCTCTACAGCAAGCT |
| | GATCGCTGGCGCCATCCACGGCAAGCCTATTGGAGGACTGGTGGCCGGCAACGTGAAA |
| | ACCAGCGCCAAGAACAAGAAAAACCCTCCTGTGCCTGCCGGCTACACCCTGGACAAGA |
| | ACAATGTGCCCTACAAGAAAGAGCAGGGCAACTACACCGTGGCCAACGTGAAGGGAAA |
| | CAACGTGCGCGACGGCTACAGCACCAACAGCAGAATTACAGGCGTGCTGCCCAACAAC |
| | ACCACAATCACCTACGACGGCGCCTACTGCATCAACGGCTATCGGTGGATCACCTACATT |
| | GCCAACTCCGGCCAGCGGCGGTATATTGCCACAGGCGAAGTGGACAAGGCCGGCAATA |
| | GAATCAGCTCCTTCGGCAAGTTCAGCACCATCTAActcgagCTGGTACTGCATGCACGCAA |
| | TGCTAGCTGCCCCTTTCCCGTCCTGGGTACCCCGAGTCTCCCCCGACCTCGGGTCCCAGG |
| | TATGCTCCCACCTCCACCTGCCCCACTCACCACCTCTGCTAGTTCCAGACACCTCCCAAGC |
| | ACGCAGCAATGCAGCTCAAAACGCTTAGCCTAGCCACACCCCCACGGGAAACAGCAGT |
| | GATTAACCTTTAGCAATAAACGAAAGTTTAACTAAGCTATACTAACCCCAGGGTTGGTCA |
| | ATTTCGTGCCAGCCACACCCTGGAGCTAGCAAAAAAAAAAAAAAAAAAAAAAAAAAAA |
| | AAGCATATGACTAAAAAAAAAAAAAAAAAAAAAAAAAAAAAAAAAAAAAAAAAAAAA |
| | AAAAAAAAAAAAAAAAAAAAAAAAA |
| L0499 | MDWTWRVFCLLAVAPGAHSMKHHHHHHSAGLEVLFQGPTQNYVVKKGDYLGAIAKKEN |
| | TTVSKIQKLNNIKNPNFILIGQVLKVPFISSNNSSTNTNRRNNSSSTLNYLNSLAYKGWDFDG |
| | AYGNQCFDLVNVYWNHLYGHGLRGYGAKDIPFANNFRGEATVYRNTPTFKAELGDVAVFS |
| | GRYGGGFGHTAIVLNGNYDGKLMKFQSLDQNWYGGDSRRADIAHKVVHNYDYDMYFIRP |
| | YKKA* |
| | ACTAGTATTCTTCTGGTCCCCACAGACTCAGAGAGAACCCGCCACCATGGACTGGACTT |
| | GGAGGGTGTTCTGCCTGTTGGCTGTAGCACCAGGAGCACACTCCATGAAACATCACCAT |
| | CATCACCACTCTGCCGGACTTGAGGTCCTGTTTCAAGGTCCCACACAGAATTACGTGGT |
| | GAAGAAGGGCGATTACCTGGGTGCGATCGCCAAGAAGTTCAACACCACAGTGAGCAAG |
| | ATCCAGAAACTCAACAACATCAAGAATCCCAACTTCATCCTGATTGGCCAAGTGCTGAA |
| | AGTACCGTTCATAAGCTCTAACAACTCAAGCACCAATACGAACCGCCGGAACAACTCCA |
| | GTAGCACACTGAACTACCTGAATTCCCTCGCATACAAAGGGTGGGATTTCGATGGAGCC |
| | TATGGCAATCAGTGTTTTGACCTCGTCAACGTGTACTGGAATCACCTCTATGGTCATGGG |
| | CTGAGAGGGTACGGTGCTAAGGACATACCTTTTGCCAATAATTTTCGCGGCGAAGCTAC |
| | TGTGTATCGGAATACCCCAACCTTCAAAGCAGAGCTTGGAGATGTTGCTGTTTTCTCAG |
| | GGAGGTATGGAGGAGGCTTTGGCCATACTGCCATTGTCCTGAATGGCAACTATGACGG |
| | GAAACTGATGAAATTCCAGTCTTTGGACCAGAATTGGTATGGCGGGGATAGTAGACGT |
| | GCCGATATTGCGCACAAGGTTGTCCACAACTATGACTACGACATGTACTTTATCCGACCT |
| | TACAAGAAGGCCTGActcgagCTGGTACTGCATGCACGCAATGCTAGCTGCCCCTTTCCCG |
| | TCCTGGGTACCCCGAGTCTCCCCCGACCTCGGGTCCCAGGTATGCTCCCACCTCCACCTG |
| | CCCCACTCACCACCTCTGCTAGTTCCAGACACCTCCCAAGCACGCAGCAATGCAGCTCAA |
| | AACGCTTAGCCTAGCCACACCCCCACGGGAAACAGCAGTGATTAACCTTTAGCAATAAA |
| | CGAAAGTTTAACTAAGCTATACTAACCCCAGGGTTGGTCAATTTCGTGCCAGCCACACCC |
| | TGGAGCTAGCAAAAAAAAAAAAAAAAAAAAAAAAAAAAAAGCATATGACTAAAAAAAA |
| | AAAAAAAAAAAAAAAAAAAAAAAAAAAAAAAAAAAAAAAAAAAAAAAAAAAAAAAAA |
| | AAAAA |
| L0502 | MDWTWRVFCLLAVAPGAHSMKHHHHHHSAGLEVLFQGPMNKVDAWLSKHDLKNLTTLI |
| | VVGAFILFLILLFMLMNHSDQGSNRITVSENAELRTDPNAAYPVIYQVEKGDTFTRLSKSGK |
| | WIEVESRDGSEKGWIAGWHTSLNIEADSNKKVNPLKNKVIVLDPGHGGNDQGASSNTSKK |
| | SLEKNYTLKTAKELRRALEKAGATVKLTRSDDTYVSLDDRKLNGDAYISIHNDSLDSPNANGA |
| | TVYWYKNNQEALAETLNTNIQKKAMLSNRGTRQENFQVLRQTNKPAVLLELGYISNPTDEV |
| | MIRNQIHRQVVEEAVVDGLKQYFAS* |
| | ACTAGTATTCTTCTGGTCCCCACAGACTCAGAGAGAACCCGCCACCATGGACTGGACTT |
| | GGCGCGTGTTTTGCCTGCTCGCCGTAGCTCCTGGAGCCCATAGCATGAAGCACCATCAT |
| | CACCACCATTCAGCGGGATTGGAGGTTCTCTTTCAAGGCCCCATGAATAAGGTGGATGC |
| | TTGGCTCAGCAAACACGACCTTAAGAACCTCACTACCTTGATCGTTGTAGGAGCCTTCAT |
| | TCTGTTCCTGATACTGCTGTTCATGCTGATGAATCACTCTGATCAGGGGTCAAACCGCAT |
| | AACTGTTTCCGAGAATGCCGAACTTCGGACAGATCCAAACGCCGCCTATCCGGTCATCT |
| | ATCAGGTCGAGAAAGGGGACACTTTCACCAGACTGTCTAAAAGCGGCAAATGGATTGA |
| | GGTCGAGAGTAGAGATGGGAGTGAGAAGGGGTGGATAGCAGGATGGCACACATCCCT |
| | GAACATTGAAGCCGATAGCAACAAGAAAGTGAACCCTCTCAAGAACAAAGTGATCGTG |
| | CTGGATCCTGGTCATGGTGGCAACGATCAGGGCGCTTCAAGCAATACCAGCAAGAAGT |
| | CACTGGAGAAGAATTACACGCTGAAAACAGCCAAGGAACTGCGACGGGCTCTTGAGAA |
| | AGCAGGAGCTACGGTGAAACTGACACGGTCTGACGACACCTATGTGAGTCTGGACGAC |
| | AGGAAGCTGAATGGCGACGCGTATATCTCCATTCACAATGACAGCCTGGATTCTCCCAA |
| | TGCAAATGGGGCTACCGTCTACTGGTACAAGAACAACCAAGAGGCCCTGGCAGAAACC |
| | CTCAATACCAACATCCAGAAGAAGGCGATGTTGTCCAATAGGGGTACTAGGCAGGAAA |
| | ACTTTCAGGTGCTTAGACAGACAAACAAACCCGCAGTTCTGTTGGAACTCGGCTACATC |
| | TCCAACCCAACAGACGAGGTGATGATCCGCAATCAGATTCACCGTCAAGTAGTGGAAG |
| | AAGCAGTGGTCGATGGCCTGAAACAGTACTTTGCCAGTTGActcgagCTGGTACTGCATG |
| | CACGCAATGCTAGCTGCCCCTTTCCCGTCCTGGGTACCCCGAGTCTCCCCCGACCTCGGG |
| | TCCCAGGTATGCTCCCACCTCCACCTGCCCCACTCACCACCTCTGCTAGTTCCAGACACCT |
| | CCCAAGCACGCAGCAATGCAGCTCAAAACGCTTAGCCTAGCCACACCCCCACGGGAAAC |
| | AGCAGTGATTAACCTTTAGCAATAAACGAAAGTTTAACTAAGCTATACTAACCCCAGGG |
| | TTGGTCAATTTCGTGCCAGCCACACCCTGGAGCTAGCAAAAAAAAAAAAAAAAAAAAAA |
| | AAAAAAAAGCATATGACTAAAAAAAAAAAAAAAAAAAAAAAAAAAAAAAAAAAAAAA |
| | AAAAAAAAAAAAAAAAAAAAAAAAAAAAAAA |

[symbol]

indicates data missing or illegible when filed

### Example 4: In Vitro Expression of RNA-Encoded Lysin Variants

In vitro expression and secretion of the generated lysin constructs is analyzed by lipofection of the mRNA into HEK293T/17 cells and subsequent quantification of secreted His-tagged protein by Bio-layer interferometry (BLI). One day prior to lipofection, 1.2×10⁶ HEK293T/17 cells are seeded in 3 mL DMEM (Life Technologies GmbH, cat. no. 31966-021)+10% fetal bovine serum (FBS, Biochrom GmbH, cat. no. S0115) in 6-well plates. For lipofection, 3 μg mRNA is formulated under sterile and RNase-free conditions using 400 ng mRNA per μL Lipofectamine MessengerMax (Thermo Fisher Scientific, cat. No. LMRNA015) and applied per 10 cm² culture dish to the HEK293T/17 cells at approximately 80% confluence. After 20 h of expression, supernatants are collected under sterile conditions and stored at −20° C. until further use. The levels of secreted lysins in the supernatant are quantified by the label-free optical analytical technique BLI through detection of His-tagged proteins. In short, a single point measurement of supernatant using the pre-hydrated Ni-NTA biosensor FortéBio BLItz® System (FortéBio) is performed. The measured binding rate is used to calculate the lysin concentration based on a calibration curve.

### Example 5: Bactericidal Activity of Lysins Encoded in and Secreted from HEK293T/17 Cells

Supernatants are serially diluted with lysin buffer (20 mM NaPO4 PH 6.0, 100 mM NaCl) and tested for bactericidal activity. For this purpose, 30 μL of supernatants (and dilutions thereof) are mixed with S. aureus cells in 70 μL BHI medium such that the reaction contains 5×10⁵ CFU/mL. The optical density at 620 nm (OD₆₂₀) of these solutions is monitored in a plate reader over the course of 24 hours to quantify bacterial growth. The growth of S. aureus or its failure will reveal the levels of active lysin in the (diluted) supernatant. Supernatants from mock-transfected HEK293T/17 cells serve as a control to rule out that conditioned culture medium itself has a lytic activity on S. aureus cells. The failure of S. aureus cells to grow in the presence of supernatants from cells expressing and secreting lysins indicates that the level of active lysins in the supernatant is sufficiently high to lyse the S. aureus cells in the culture. The fold dilutions at which bacterial growth starts to appear further allows to identify the most potent lysin constructs, whose lytic activity depends on expression, secretion and enzymatic activity rates.

Taken together, these experiments reveal mRNA-encoded lysins which can be expressed and secreted in an active form in human cells, resulting in successful lysis of S. aureus.

### Example 6: Endolysins Expressed in HEK Cells

mRNA Encoded Endolysin is Expressed by HEK Cells

To assess expression of phage endolysins in eukaryotic cells, HEK293T cells were transfected with modRNA endolysin constructs. Specifically, constructs 9_LysPA26 and 10_LysPA26 were used which both contain a FLAG tag for detection, and 10_LysPA26 contains an additional signal sequence for protein secretion (husec).

Construct 9_LysPA26: pST5-T7-AGA-dEarI-hAg-FLAG-LysPA26-FI-A30LA70
aa sequence:
MDYKDDDDKRTNNIDAIKEHEGLRLVAYLDSVGVWTIGYGDTGPDVVKGLTITKEEAEKRLRKRLVEFEGY
VNTYVKVPLKQHQFDALVSLVYNIGPTNFKTSTLLKKLNAGDYIGAADQFLVWNKGRVDGKLVVIKGLANR
RAKERKQFLGE
nt sequence:
AGAATAAACTAGTATTCTTCTGGTCCCCACAGACTCAGAGAGAACCCGCCACCATGGACTACAAAGAC
GATGACGACAAGAGAACCAACAACATCGATGCCATCAAAGAACATGAAGGACTGAGACTGGTGGCTT
ACCTGGATTCTGTGGGAGTGTGGACAATTGGATATGGAGACACAGGACCTGACGTGGTGAAGGGACT
GACAATCACAAAGGAAGAAGCCGAAAAGAGACTGAGAAAGAGACTGGTGGAATTTGAAGGATATGT
GAACACCTATGTGAAAGTGCCTCTGAAACAGCACCAGTTTGATGCTCTGGTGAGCCTGGTGTACAACAT
TGGACCCACAAACTTCAAGACAAGCACACTGCTGAAGAAGCTGAATGCTGGAGATTACATTGGAGCTG
CTGATCAGTTCCTGGTGTGGAACAAAGGCAGAGTGGATGGCAAACTGGTGGTGATCAAAGGCCTGGC
CAACAGGAGAGCCAAAGAAAGAAAACAGTTCCTGGGAGAATGATGACTCGAGCTGGTACTGCATGCA
CGCAATGCTAGCTGCCCCTTTCCCGTCCTGGGTACCCCGAGTCTCCCCCGACCTCGGGTCCCAGGTATG
CTCCCACCTCCACCTGCCCCACTCACCACCTCTGCTAGTTCCAGACACCTCCCAAGCACGCAGCAATGC
AGCTCAAAACGCTTAGCCTAGCCACACCCCCACGGGAAACAGCAGTGATTAACCTTTAGCAATAAACG
AAAGTTTAACTAAGCTATACTAACCCCAGGGTTGGTCAATTTCGTGCCAGCCACACCCTGGAGCTAGCA
AAAAAAAAAAAAAAAAAAAAAAAAAAAAAGCATATGACTAAAAAAAAAAAAAAAAAAAAAAAAAAA
AAAAAAAAAAAAAAAAAAAAAAAAAAAAAAAAAAAAAAAAAAA
Construct 10_LysPA26: pST5-T7-AGA-dEarI-hAg-husec(opt)-FLAG-LysPA26-FI-A30LA70
aa sequence:
MRVMAPRTLILLLSGALALTETWAGSDYKDDDDKRTNNIDAIKEHEGLRLVAYLDSVGVWTIGYGDTGPD
VVKGLTITKEEAEKRLRKRLVEFEGYVNTYVKVPLKQHQFDALVSLVYNIGPTNFKTSTLLKKLNAGDYIGAA
DQFLVWNKGRVDGKLVVIKGLANRRAKERKQFLGE
nt sequence:
AGAATAAACTAGTATTCTTCTGGTCCCCACAGACTCAGAGAGAACCCGCCACCATGAGAGTGATGGCC
CCCAGAACCCTGATCCTGCTGCTGTCTGGCGCCCTGGCCCTGACAGAGACATGGGCCGGAAGCGACTA
CAAAGACGATGACGACAAGAGAACCAACAACATCGATGCCATCAAAGAACATGAAGGACTGAGACTG
GTGGCTTACCTGGATTCTGTGGGAGTGTGGACAATTGGATATGGAGACACAGGACCTGACGTGGTGA
AGGGACTGACAATCACAAAGGAAGAAGCCGAAAAGAGACTGAGAAAGAGACTGGTGGAATTTGAAG
GATATGTGAACACCTATGTGAAAGTGCCTCTGAAACAGCACCAGTTTGATGCTCTGGTGAGCCTGGTGT
ACAACATTGGACCCACAAACTTCAAGACAAGCACACTGCTGAAGAAGCTGAATGCTGGAGATTACATT
GGAGCTGCTGATCAGTTCCTGGTGTGGAACAAAGGCAGAGTGGATGGCAAACTGGTGGTGATCAAAG
GCCTGGCCAACAGGAGAGCCAAAGAAAGAAAACAGTTCCTGGGAGAATGATGACTCGAGCTGGTACT
GCATGCACGCAATGCTAGCTGCCCCTTTCCCGTCCTGGGTACCCCGAGTCTCCCCCGACCTCGGGTCCC
AGGTATGCTCCCACCTCCACCTGCCCCACTCACCACCTCTGCTAGTTCCAGACACCTCCCAAGCACGCA
GCAATGCAGCTCAAAACGCTTAGCCTAGCCACACCCCCACGGGAAACAGCAGTGATTAACCTTTAGCA
ATAAACGAAAGTTTAACTAAGCTATACTAACCCCAGGGTTGGTCAATTTCGTGCCAGCCACACCCTGGA
GCTAGCAAAAAAAAAAAAAAAAAAAAAAAAAAAAAAGCATATGACTAAAAAAAAAAAAAAAAAAAA
AAAAAAAAAAAAAAAAAAAAAAAAAAAAAAAAAAAAAAAAAAAAAAAAAA

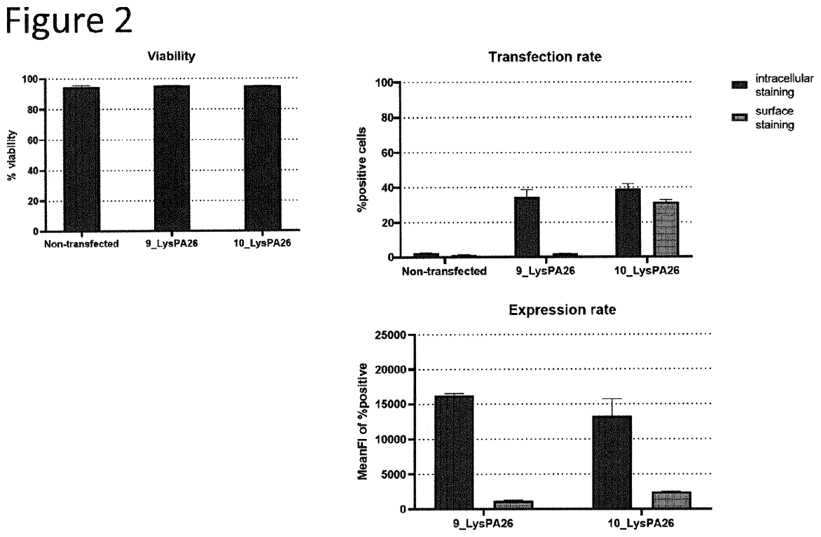

Transfected cells were assessed for viability, intracellular protein expression and surface protein expression using flow cytometry (FIG. 2). Cells were stained with a viability dye, and then to determine intracellular protein expression cells were permeabilized and stained with an anti-FLAG antibody conjugate to a fluorescent dye. Surface staining was performed without the permeabilization step. Transfection rate indicates the percentage of HEK cells positive for presence of the protein and expression rate is representative of the mean fluorescence of the positive cells. There is clear expression of protein in HEK cells, particularly evident in intracellular staining. Since the constructs do not encode for a transmembrane domain, expression on the surface was not expected, however interestingly there is some surface expression in the construct (10_LysPA26) with a secretion signal. mRNA encoded endolysin is secreted by HEK cells

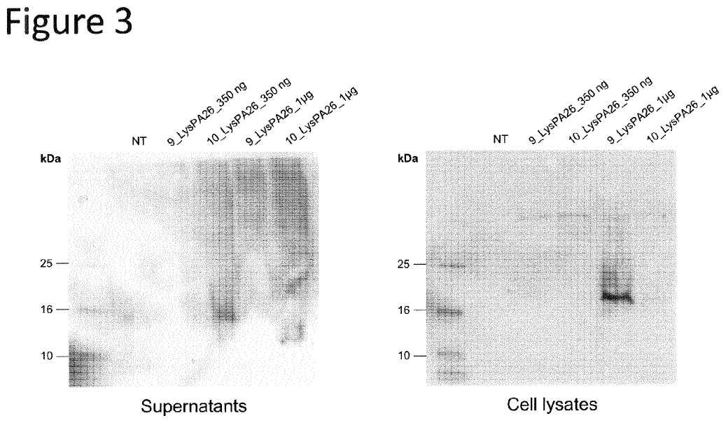

Protein secretion of the same constructs was assessed via Western blot (FIG. 3). Culture supernatants and cell lysates were collected from two separate transfection experiments, with 350 ng and 1 μg of RNA, done in parallel as a test for optimal RNA concentration for protein expression. Protein was detected using an HRP-conjugated anti-FLAG antibody. Protein expression was clearly visible at the expected size of ^{˜}17 kDa in cell lysates of cells transfected with 9_LysPA26 at the concentration of 1 μg. Protein secreted into the cell supernatant was detected with a faint band in supernatant of cells transfected with 10_LysPA26, the construct containing the secretion signal, and the band was absent in samples without the secretion signal. The results demonstrate clear protein expression, as well as secretion when a secretion signal is included in the constructs.

### Testing Secreted Endolysin for Effectiveness in Reducing Viable Cell Count

To verify the ability of the endolysin to break down bacterial cells extracellularly, log (OD₆₀₀ 0.8) and stationary phase (OD₆₀₀ 2) bacterial cells are incubated with culture supernatant of HEK cells expressing RNA encoded endolysin. XTT assay, a colorimetric assay in which metabolically active cells reduce a tetrazolium salt to a formazan dye, is used to measure bacterial cell viability. Bacterial species (Gram-positive bacteria) not susceptible to LysPA26 endolysin are used as a negative control.

### Testing Secreted Endolysin for Effectiveness in Reducing Bacterial Biofilms

To investigate the potential of the endolysin to clear bacterial biofilms, 48h old biofilms are incubated with culture supernatant of HEK cells expressing endolysin. Surface-attached bacterial biofilms are formed in 96 well plates by seeding log culture bacteria diluted to OD₆₀₀ 0.05 and static incubation at 37° C. for 48h in LB media. The media containing planktonic, not attached, cells are carefully removed, and biofilms overlayed with culture supernatant and incubated for 2 h at 37° C. Crystal violet stain is subsequently used as a stain for determining biofilm biomass and XTT colorimetric assay to determine cell viability. Bacterial biofilms incubated with culture supernatant not containing secreted endolysin are used as a control.

### Example 7: RNA Constructs for Endolysin Expression in Human Subjects

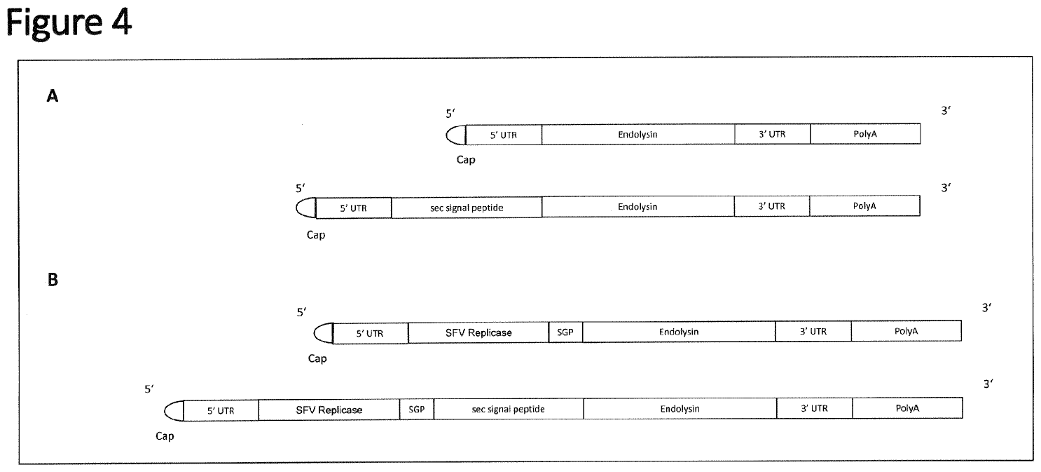

Different RNA constructs encoding endolysins as schematically shown in FIG. 4 were prepared. The different constructs either do not contain or contain a signal peptide. All constructs contain all the elements necessary for expression of the lysin LysPA26 in human cells, including a cap, a 5′ UTR, a 3′ UTR and a polyA sequence. An overview of the constructs is given in the following table:

| SEQ ID | | Signal | Length of | |
|---|---|---|---|---|
| # | Construct | peptide | RNA [nt] | Mw [Da] |
| 6 | pST5-T7-AGA-dEarl-hAg-LysPA26-FI-A30LA70 | — | 899 | 16206.5 |
| 7 | pST5-T7-AGA-dEarl-hAg-husec(opt)-LysPA26- | husec | 974 | |
| | FI-A30LA70 | | | |
| 8 | pST1-OPAL-LysPA26-A30LA70 | — | 7972 | |
| 9 | pST1-OPAL-husec(opt)-LysPA26-A30LA70 | husec | 8047 | |

RNA encoded endolysins are tested against Gram-negative bacteria, given that according to the World Health Organization the top three resistant bacterial pathogens for which novel treatment is urgently needed, carbapenem-resistant Acinetobacter baumannii, carbapenem-resistant Pseudomonas aeruginosa, and carbapenem-resistant Enterobacteriaceae, all belong to the group of Gram-negative bacteria (WHO, 2017). Following the initial testing, this treatment option is also used in other types of bacteria-including those belonging to the Gram-positive group, such as methicillin and vancomycin resistant Staphylococcus aureus and penicillin non-susceptible Streptococcus pneumoniae.

### Example 8: Endolysins Expressed in HEK Cells

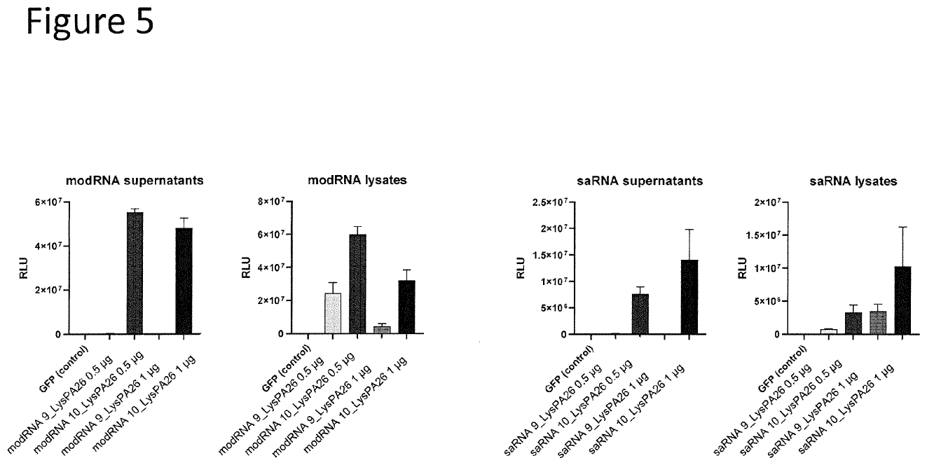

HEK293T cells were transfected with 0.5 and 1 μg of modRNA and saRNA of construct 9_LysPA26 and construct 10_LysPA26 (each containing a HiBiT tag for detection) in an 96 well plate. The nucleotide and amino acid sequences of fusion-protein nucleoside-modified RNA constructs are shown in SEQ ID NOs 111, 112 and SEQ ID NOs 113, 114, respectively. The nucleotide and amino acid sequences of fusion-protein self-amplifying RNA constructs are shown in SEQ ID NOs 115, 112 and SEQ ID NOs 116, 114, respectively. Transfection process was as previously described. After 18h, for detection of extracellular protein the Nano-Glo HiBit extracellular reagent was used. As per manufacturers instructions LgBit protein was diluted 1:100 and the Nano-Glo HiBit extracellular Substrate was diluted 1:50 in the necessary volume of the Nano-Glo HiBit Extracellular buffer. Plates were equilibrated to room temperature for 10 min, and 100 μl of the reagent was added to each well (to have equal volume of culture media and reagent). Plates were placed on an orbital shaker for 3 min at 300 rpm and luminescence measured after 10 mins using the Tecan Spark microplate reader. Data is represented in relative luminescence units (RLU). The same procedure was conducted for detecting protein in the cell lysate, except the Nano-Glow Lytic Reagents were used from the Nano-Glo Lytic Detection System kit, in which the reagents contain a cell lytic compound. Results are shown in FIG. 5. Left panels show supernatant and cell lysates for modRNA. The constructs with secretion signals (10_LysPA26) are clearly detectable at both concentrations in the supernatant, while the constructs without secretion signals (9_LysPA26) cannot be detected. The same result is seen for saRNA. This confirms that the expression and secretion of endolysins can be seen for different RNA platforms. All constructs can be detected in the cell lysates showing that the endolysins are expressed in HEK cells irrespective of the RNA platform used.

### Example 9: In Vitro Expression of DNA and RNA Encoded Lysin Variants

In vitro expression and secretion of several lysin constructs is analyzed by lipofection of DNA or mRNA into HEK293T/17 cells and subsequent quantification of secreted His-tagged protein by Western Blot (see table 6). One day prior to lipofection, 0.9×10⁶ HEK293T/17 cells are seeded in 3 ml DMEM (Life Technologies GmbH, cat. no. 31966-021)+10% fetal bovine serum (FBS, Biochrom GmbH, cat. No. S0115) per well in 6-well plates. For lipofection, 3, 5 and 7.5 μg, of mRNA or 2.5 μg of DNA are transfected. mRNA is formulated using the RiboJuice™ mRNA Transfection Kit (Sigma Aldrich, cat. no. TR-1013), DNA using Lipofectamine™ LTX Reagent with PLUS™ Reagent (Life Technologies GmbH, cat. no. 15338100) according to the manufacturer's instructions. After 24, 48 or 72 hours of expression, supernatants are collected, subjected to PNGase F digest (New England Biolabs GmbH, cat. no. P0704L) under denaturing conditions to remove potential N-glycosylation and stored at −20° C. until further use. The levels of secreted lysins are quantified by Western Blot through detection by an anti-His antibody (Abcam, ab49781 for N-terminal His-tag and Abcam, ab1187 for C-terminal His-tag) or an anti-Flag antibody (Sigma A8592). Expression levels were quantified by quantification of chemiluminescent signals using Image Lab 6. Mammalian expression of a prototype endolysin was also assessed using an mRNA formulated in lipid nanoparticles (LNP-mRNA). Here 1.5 μg of formulated mRNA were transfected into HEK293T/17 or CHO-K1 cells. Growth medium for CHO-K1 is F12-K (21127022, Thermo Fisher Scientific). Medium was changed to Opti-MEM prior to transfection for both cell lines.

TABLE 6
List of constructs tested for expression in HEK293T/17 cells. Expression from DNA as
well as from mRNA transfections was assessed

| | | | | | Predicted |
|---|---|---|---|---|---|
| | | | | | Mw [kDa] |
| | | | | | (without |
| Construct | | | | Length of | signal |
| # | RNA/DNA | Construct | Signal peptide | DNA/RNA | peptide) |
| 11 | RNA | pST4-T7-AGA-dEarI- | Ig heavy chain V-I | 1313 (T7 | 28.87 |
| | | hAg-Kozak-SS(IGHV1- | region HG3 | start to end | |
| | | 2)-His-3C-hL0483-FI- | (MDWTWRVFCLLAVAPGAHS) | of Poly-A- | |
| | | A30LA70 | | tail) | |
| 12 | DNA | pcDNA3.1(+)- | Ig heavy chain V-I | 914 (T7 | 28.87 |
| | | SS(IGHV1-2)-His-3C- | region HG3 | start to end | |
| | | hL0483 | (MDWTWRVFCLLAVAPGAHS) | of lysin) | |
| 13 | RNA | pST4-T7-AGA-dEarI- | Human insulin | 2099 (T7 | 58.05 |
| | | hAg-Kozak-SS(hINS)- | (MALWMRLLPLLALLALWGPDPAAA) | start to end | |
| | | FLAG-hL0473-His-FI- | | of Poly-A- | |
| | | A30LA70 | | tail) | |
| 14 | RNA | pST4-T7-AGA-dEarI- | Human insulin | 1268 (T7 | 27.21 |
| | | hAg-Kozak-SS(hINS)- | (MALWMRLLPLLALLALWGPDPAAA) | start to end | |
| | | FLAG-hL0482-His-FI- | | of Poly-A- | |
| | | A30LA70 | | tail) | |
| 15 | RNA | pST4-T7-AGA-dEarI- | Human insulin | 1361 (T7 | 29.93 |
| | | hAg-Kozak-SS(hINS)- | (MALWMRLLPLLALLALWGPDPAAA) | start to end | |
| | | FLAG-hL0483-His-FI- | | of Poly-A- | |
| | | A30LA70 | | tail) | |
| 16 | RNA | pST4-T7-AGA-dEarI- | Human insulin | 1073 (T7 | 19.85 |
| | | hAg-Kozak-SS(hINS)- | (MALWMRLLPLLALLALWGPDPAAA) | start to end | |
| | | FLAG-hL0484m-His-FI- | | of Poly-A- | |
| | | A30LA70 | | tail) | |
| 17 | RNA | PST4-T7-AGA-dEarI- | Human insulin | 1022 (T7 | 18.4 |
| | | hAg-Kozak-SS(hINS)- | (MALWMRLLPLLALLALWGPDPAAA) | start to end | |
| | | FLAG-hIL2-His-FI- | | of Poly-A- | |
| | | A30LA70 | | tail) | |
| 18 | RNA | pST4-T7-AGA-dEarI- | Human IL2 | 1010 (T7 | 18.4 |
| | | hAg-Kozak-SS(hIL2)- | (MYRMQLLSCIALSLALVTNS) | start to end | |
| | | FLAG-hIL2-His-FI- | | of Poly-A- | |
| | | A30LA70 | | tail) | |
| 19 | DNA | pcDNA3.1(+)-SS(hINS)- | Human insulin | 1700 (T7 | 58.05 |
| | | FLAG-hL0473-His | (MALWMRLLPLLALLALWGPDPAAA) | start to end | |
| | | | | of His tag) | |
| 20 | DNA | pcDNA3.1(+)-SS(hINS)- | Human insulin | 869 (T7 | 27.21 |
| | | FLAG-hL0482-His | (MALWMRLLPLLALLALWGPDPAAA) | start to end | |
| | | | | of His tag) | |
| 21 | DNA | pcDNA3.1(+)-SS(hINS)- | Human insulin | 962 (T7 | 29.93 |
| | | FLAG-hL0483-His | (MALWMRLLPLLALLALWGPDPAAA) | start to end | |
| | | | | of His tag) | |
| 22 | DNA | pcDNA3.1(+)-SS(hINS)- | Human insulin | 674 (T7 | 19.85 |
| | | FLAG-hL0484m-His | (MALWMRLLPLLALLALWGPDPAAA) | start to end | |
| | | | | of His tag) | |
| 23 | DNA | pcDNA3.1(+)-SS(hINS)- | Human insulin | 623 (T7 | 18.4 |
| | | FLAG-hIL2-His | (MALWMRLLPLLALLALWGPDPAAA) | start to end | |
| | | | | of His tag) | |
| 24 | DNA | pcDNA3.1(+)-SS(hIL2)- | Human IL2 | 611 (T7 | 18.4 |
| | | FLAG-hIL2-His | (MYRMQLLSCIALSLALVTNS) | start to end | |
| | | | | of His tag) | |
| 25 | RNA | pST4-T7-AGA-dEarI- | Mouse \|Gkappa | 935 (T7 | 29.1 |
| | | hAg-Kozak- | MDMRAPAGIFGFLLVLFPGYRS | start to end | |
| | | SS(IGkappa)-His-3C- | | of Poly-A- | |
| | | hL0483(PM)-FLAG-FI- | | tail | |
| | | A30LA70 | | | |

Construct 11: pST4-T7-AGA-dEarI-hAg-Kozak-SS(IGHV1-2)-His-3C-hL0483-FI-A30LA70
aa sequence:
MDWTWRVFCLLAVAPGAHSMKHHHHHHSAGLEVLFQGPAATHEHSAQWLNNYKKGYGYGPYPLGIN
GGMHYGVDFFMNIGTPVKAISSGKIVEAGWSNYGGGNQIGLIENDGVHRQWYMHLSKYNVKVGDYVK
AGQIIGWSGSTGYSTAPHLHFQRMVNSFSNSTAQDPMPFLKSAGYGKAGGTVTPTPNTGWKTNKYGTLY
KSESASFTPNTDIITRTTGPFRSMPQSGVLKAGQTIHYDEVMKQDGHVWVGYTGNSGQRIYLPVRTWNK
STNTLGVLWGTIK*
nt sequence:
AGAATAAACTAGTATTCTTCTGGTCCCCACAGACTCAGAGAGAACCCGCCACCATGGATTGGACCTG
GAGAGTGTTTTGCCTTCTCGCTGTTGCCCCTGGTGCCCATTCCATGAAGCACCACCACCACCATCATA
GTGCAGGGCTGGAAGTTCTGTTTCAGGGTCCAGCAGCAACGCATGAGCATTCCGCTCAATGGCTCAA
CAACTACAAGAAGGGTTACGGCTATGGCCCCTATCCTCTGGGGATAAACGGAGGGATGCACTATGG
AGTCGATTTCTTCATGAATATTGGCACTCCAGTGAAAGCGATTTCCAGCGGGAAGATTGTGGAAGCT
GGGTGGAGCAACTATGGTGGTGGCAATCAGATCGGACTGATTGAGAATGACGGCGTGCACAGACA
GTGGTATATGCATCTGAGTAAGTACAACGTCAAGGTGGGAGACTACGTCAAAGCCGGGCAGATCAT
AGGATGGTCCGGCTCTACAGGTTACTCAACAGCGCCACATCTCCACTTTCAGCGCATGGTGAATAGC
TTCAGCAATTCAACCGCCCAAGATCCGATGCCCTTCCTGAAGTCTGCCGGCTATGGGAAAGCTGGAG
GAACAGTGACACCTACTCCCAATACCGGATGGAAAACGAACAAGTACGGCACTCTGTACAAATCCGA
ATCTGCCAGTTTCACACCCAATACCGACATCATCACCCGAACCACTGGACCTTTTCGGTCAATGCCGC
AGTCTGGAGTACTGAAAGCAGGGCAAACTATCCACTATGACGAGGTGATGAAGCAGGATGGCCACG
TATGGGTGGGGTACACAGGCAATAGCGGCCAGAGGATTTACTTGCCAGTCCGTACCTGGAACAAAA
GCACGAACACTCTTGGCGTTTTGTGGGGCACCATCAAGTGACTCGAGCTGGTACTGCATGCACGCAA
TGCTAGCTGCCCCTTTCCCGTCCTGGGTACCCCGAGTCTCCCCCGACCTCGGGTCCCAGGTATGCTCC
CACCTCCACCTGCCCCACTCACCACCTCTGCTAGTTCCAGACACCTCCCAAGCACGCAGCAATGCAGC
TCAAAACGCTTAGCCTAGCCACACCCCCACGGGAAACAGCAGTGATTAACCTTTAGCAATAAACGAA
AGTTTAACTAAGCTATACTAACCCCAGGGTTGGTCAATTTCGTGCCAGCCACACCCTGGAGCTAGCAA
AAAAAAAAAAAAAAAAAAAAAAAAAAAAGCATATGACTAAAAAAAAAAAAAAAAAAAAAAAAAAA
AAAAAAAAAAAAAAAAAAAAAAAAAAAAAAAAAAAAAAAAAAA
Construct 12: pcDNA3.1(+)-SS(IGHV1-2)-His-3C-hL0483
aa sequence
MDWTWRVFCLLAVAPGAHSMKHHHHHHSAGLEVLFQGPAATHEHSAQWLNNYKKGYGYGPYPLGIN
GGMHYGVDFFMNIGTPVKAISSGKIVEAGWSNYGGGNQIGLIENDGVHRQWYMHLSKYNVKVGDYVK
AGQIIGWSGSTGYSTAPHLHFORMVNSFSNSTAQDPMPFLKSAGYGKAGGTVTPTPNTGWKTNKYGTL
YKSESASFTPNTDIITRTTGPFRSMPQSGVLKAGQTIHYDEVMKQDGHVWVGYTGNSGQRIYLPVRTWN
KSTNTLGVLWGTIK*
nt sequence:
GAGACCCAAGCTGGCTAGCGTTTAAACTTAAGCTTGGTACCGAGCTCGGATCCGCCACCATGGATTG
GACCTGGAGAGTGTTTTGCCTTCTCGCTGTTGCCCCTGGTGCCCATTCCATGAAGCACCACCACCACC
ATCATAGTGCAGGGCTGGAAGTTCTGTTTCAGGGTCCAGCAGCAACGCATGAGCATTCCGCTCAATG
GCTCAACAACTACAAGAAGGGTTACGGCTATGGCCCCTATCCTCTGGGGATAAACGGAGGGATGCA
CTATGGAGTCGATTTCTTCATGAATATTGGCACTCCAGTGAAAGCGATTTCCAGCGGGAAGATTGTG
GAAGCTGGGTGGAGCAACTATGGTGGTGGCAATCAGATCGGACTGATTGAGAATGACGGCGTGCA
CAGACAGTGGTATATGCATCTGAGTAAGTACAACGTCAAGGTGGGAGACTACGTCAAAGCCGGGCA
GATCATAGGATGGTCCGGCTCTACAGGTTACTCAACAGCGCCACATCTCCACTTTCAGCGCATGGTG
AATAGCTTCAGCAATTCAACCGCCCAAGATCCGATGCCCTTCCTGAAGTCTGCCGGCTATGGGAAAG
CTGGAGGAACAGTGACACCTACTCCCAATACCGGATGGAAAACGAACAAGTACGGCACTCTGTACA
AATCCGAATCTGCCAGTTTCACACCCAATACCGACATCATCACCCGAACCACTGGACCTTTTCGGTCA
ATGCCGCAGTCTGGAGTACTGAAAGCAGGGCAAACTATCCACTATGACGAGGTGATGAAGCAGGAT
GGCCACGTATGGGTGGGGTACACAGGCAATAGCGGCCAGAGGATTTACTTGCCAGTCCGTACCTGG
AACAAAAGCACGAACACTCTTGGCGTTTTGTGGGGCACCATCAAGTGA
Contruct 13: pST4-T7-AGA-dEarI-hAg-Kozak-SS(hINS)-FLAG-hL0473-His-FI-A30LA70
aa sequence:
MALWMRLLPLLALLALWGPDPAAAMKDYKDDDDKGGGGSGMAKTKKQIEQRLQAYAKGTVDSPYRIT
KPTSYDPSFGVMEVGAIDADRYYHAQCQDLITDYVLWLTDNKVRPWGNAKDQINQSYGTGFKIHKNLP
STVAKTGWIAVFTSGTYSQYGHIGIVYSPGDTNKFHIIEQNWNGWANKKPLTRWDNYYGLTHFIEVPYA
KETITTKKTAPKAATSKPAKKISVSHNNINLOSNFAKRGYNPKGVVIHNDAGRSTAQQYENSLKGANQNR
LNNGIAHAYADSSHIWYAIEPERIAYHTGDGVKKGTGNYDYFGIEVCQSMSASDKEFMANEQVVLQEVA
RLLKKWKLPANRNTVRLHLEFSATSCPHRSLTMRTGFNPVTQGRPSDAIIKKLKDIWIKEIRAYMEGKTPSI
KTVANKPGSASTPATKSNSDGWKVNAYNTYYKTERASFTVGSTRIAVRSTGPFTTCPFTGWLNPGVTITY
DEVMVQNGFVWLGYDSYNGRRYVPIRTAQGTPGSSNYVLGPLWGTISAAAGGGGSHHHHHH*
Nt sequence:
AGAATAAACTAGTATTCTTCTGGTCCCCACAGACTCAGAGAGAACCCGCCACCATGGCCCTGTGGAT
GAGACTGCTGCCTCTGCTTGCTCTGCTGGCACTGTGGGGACCTGATCCTGCTGCTGCTATGAAGGAC
TACAAAGACGATGACGACAAGGGAGGAGGCGGCTCCGGAATGGCCAAAACCAAGAAACAGATAGA
ACAGAGACTGCAAGCTTACGCAAAGGGGACCGTGGATTCCCCTTACCGAATCACCAAGCCAACCAGC
TACGACCCCTCCTTCGGTGTCATGGAGGTTGGCGCTATAGATGCGGATCGCTACTATCATGCCCAGT
GTCAAGATCTGATAACTGACTATGTACTTTGGCTCACTGACAACAAAGTAAGACCTTGGGGTAATGC
CAAGGACCAGATTAACCAGAGCTATGGGACTGGTTTCAAAATCCACAAGAATCTGCCCAGTACCGTG
GCTAAAACAGGATGGATTGCCGTGTTTACATCCGGGACCTACTCACAGTACGGCCACATCGGCATCG
TGTACAGTCCAGGAGATACCAACAAATTCCATATCATCGAGCAGAATTGGAATGGATGGGCAAACAA
GAAACCTCTGACACGTTGGGACAACTACTACGGGCTGACCCACTTTATCGAGGTCCCATATGCGAAA
GAGACAATCACAACGAAGAAAACTGCACCTAAAGCAGCTACCTCAAAACCGGCTAAGAAGATCTCA
GTTTCTCACAACAACATCAACCTTCAGAGCAACTTTGCAAAGAGAGGGTACAACCCAAAGGGAGTGG
TGATTCACAATGACGCCGGACGCTCTACTGCGCAACAGTATGAAAACAGCCTGAAGGGCGCTAATCA
GAACAGGCTGAATAACGGCATTGCCCATGCATATGCTGACAGCAGCCACATTTGGTATGCTATCGAA
CCCGAGAGAATCGCCTATCACACAGGCGATGGCGTTAAGAAGGGAACAGGCAATTACGACTACTTC
GGTATTGAAGTATGCCAGAGCATGAGCGCAAGTGACAAAGAGTTCATGGCCAACGAACAGGTGGTA
CTGCAGGAGGTGGCTAGGTTGCTCAAGAAATGGAAGCTTCCCGCCAATCGGAATACCGTGCGGCTC
CATCTGGAATTCTCTGCCACATCTTGCCCTCATAGGAGCCTCACAATGCGGACCGGTTTCAATCCGGT
GACTCAGGGTAGACCCTCAGACGCCATCATCAAGAAGTTGAAGGATATCTGGATAAAAGAGATTCG
CGCATATATGGAGGGTAAAACCCCATCCATTAAGACTGTCGCCAATAAGCCCGGATCAGCCAGTACC
CCTGCAACCAAATCCAACTCCGATGGGTGGAAGGTCAATGCCTACAATACCTACTACAAAACGGAAC
GGGCCTCCTTTACAGTGGGCTCTACTCGGATTGCTGTGCGTAGTACAGGACCCTTTACTACATGCCCG
TTTACAGGGTGGCTGAACCCCGGAGTCACGATAACCTATGACGAGGTTATGGTGCAGAACGGGTTT
GTGTGGTTGGGCTATGATAGCTACAATGGGAGGAGGTATGTCCCCATTCGCACTGCCCAAGGAACTC
CTGGCAGCTCCAATTATGTGCTTGGCCCTCTGTGGGGCACGATTTCTGCGGCCGCCGGAGGAGGCG
GCTCCCACCACCACCACCATCATTGACTCGAGCTGGTACTGCATGCACGCAATGCTAGCTGCCCCTTT
CCCGTCCTGGGTACCCCGAGTCTCCCCCGACCTCGGGTCCCAGGTATGCTCCCACCTCCACCTGCCCC
ACTCACCACCTCTGCTAGTTCCAGACACCTCCCAAGCACGCAGCAATGCAGCTCAAAACGCTTAGCCT
AGCCACACCCCCACGGGAAACAGCAGTGATTAACCTTTAGCAATAAACGAAAGTTTAACTAAGCTAT
ACTAACCCCAGGGTTGGTCAATTTCGTGCCAGCCACACCCTGGAGCTAGCAAAAAAAAAAAAAAAAA
AAAAAAAAAAAAAGCATATGACTAAAAAAAAAAAAAAAAAAAAAAAAAAAAAAAAAAAAAAAAAA
AAAAAAAAAAAAAAAAAAAAAAAAAAAA
Construct 14: pST4-T7-AGA-dEarI-hAg-Kozak-SS(hINS)-FLAG-hL0482-His-FI-A30LA70
Aa sequence
MALWMRLLPLLALLALWGPDPAAAMKDYKDDDDKGGGGSGREAPKTQIYTVKKGDTLSAIALKYKTTV
SNIQNTNNIANPNLIFIGQKLKVPMTPLVEPKPKTVSSNNKSNSNSSTLNYLKTLENRGWDFDGSYGWQC
FDLVNVYWNHLYGHGLKGYGAKDIPYANNFNSEAKIYHNTPTFKAEPGDLVVFSGRFGGGYGHTAIVLN
GDYDGKLMKFQSLDQNWNNGGWRKAEVAHKVVHNYENDMIFIRPFKKAAAAGGGGSHHHHHH*
Nt sequence
AGAATAAACTAGTATTCTTCTGGTCCCCACAGACTCAGAGAGAACCCGCCACCATGGCCCTGTGGAT
GAGACTGCTGCCTCTGCTTGCTCTGCTGGCACTGTGGGGACCTGATCCTGCTGCTGCTATGAAGGAC
TACAAAGACGATGACGACAAGGGAGGAGGCGGCTCCGGAAGAGAGGCTCCCAAAACACAGATTTA
CACCGTCAAGAAGGGAGATACGCTTAGCGCCATTGCACTGAAATACAAGACCACCGTATCAAACATC
CAGAACACCAATAACATAGCCAATCCCAATCTCATCTTCATTGGCCAGAAGCTCAAGGTTCCCATGAC
TCCACTGGTCGAACCGAAACCTAAGACAGTGTCCTCCAATAACAAGAGTAACAGCAACTCCTCTACAC
TGAACTATCTCAAGACGTTGGAGAATCGCGGTTGGGACTTTGACGGCAGTTACGGCTGGCAATGCTT
CGACTTGGTGAATGTGTACTGGAATCACCTGTATGGGCATGGACTGAAAGGCTATGGGGCCAAAGA
CATACCTTATGCTAACAATTTCAACTCTGAGGCCAAGATCTACCACAACACTCCTACCTTCAAGGCAG
AACCAGGAGATCTGGTTGTGTTTAGCGGACGGTTTGGCGGAGGCTATGGGCATACTGCCATTGTGCT
TAATGGGGATTACGATGGCAAACTGATGAAATTCCAGTCACTGGACCAGAACTGGAACAATGGTGG
TTGGCGAAAAGCGGAAGTAGCCCACAAGGTCGTCCACAATTACGAGAACGACATGATCTTCATCCGT
CCCTTTAAGAAAGCGGCGGCCGCCGGAGGAGGCGGCTCCCACCACCACCACCATCATTGACTCGAG
CTGGTACTGCATGCACGCAATGCTAGCTGCCCCTTTCCCGTCCTGGGTACCCCGAGTCTCCCCCGACC
TCGGGTCCCAGGTATGCTCCCACCTCCACCTGCCCCACTCACCACCTCTGCTAGTTCCAGACACCTCCC
AAGCACGCAGCAATGCAGCTCAAAACGCTTAGCCTAGCCACACCCCCACGGGAAACAGCAGTGATTA
ACCTTTAGCAATAAACGAAAGTTTAACTAAGCTATACTAACCCCAGGGTTGGTCAATTTCGTGCCAGC
CACACCCTGGAGCTAGCAAAAAAAAAAAAAAAAAAAAAAAAAAAAAAGCATATGACTAAAAAAAAA
AAAAAAAAAAAAAAAAAAAAAAAAAAAAAAAAAAAAAAAAAAAAAAAAAAAAAAAAAAAAA
Construct 15: pST4-T7-AGA-dEarI-hAg-Kozak-SS(hINS)-FLAG-hL0483-His-FI-A30LA70
Aa sequence
MALWMRLLPLLALLALWGPDPAAAMKDYKDDDDKGGGGSGAATHEHSAQWLNNYKKGYGYGPYPL
GINGGMHYGVDFFMNIGTPVKAISSGKIVEAGWSNYGGGNQIGLIENDGVHRQWYMHLSKYNVKVGD
YVKAGQIIGWSGSTGYSTAPHLHFQRMVNSFSNSTAQDPMPFLKSAGYGKAGGTVTPTPNTGWKTNKY
GTLYKSESASFTPNTDIITRTTGPFRSMPQSGVLKAGQTIHYDEVMKQDGHVWVGYTGNSGQRIYLPVRT
WNKSTNTLGVLWGTIKAAAGGGGSHHHHHH*
Nt sequence
AGAATAAACTAGTATTCTTCTGGTCCCCACAGACTCAGAGAGAACCCGCCACCATGGCCCTGTGGAT
GAGACTGCTGCCTCTGCTTGCTCTGCTGGCACTGTGGGGACCTGATCCTGCTGCTGCTATGAAGGAC
TACAAAGACGATGACGACAAGGGAGGAGGCGGCTCCGGAGCAGCAACGCATGAGCATTCCGCTCA
ATGGCTCAACAACTACAAGAAGGGTTACGGCTATGGCCCCTATCCTCTGGGGATAAACGGAGGGAT
GCACTATGGAGTCGATTTCTTCATGAATATTGGCACTCCAGTGAAAGCGATTTCCAGCGGGAAGATT
GTGGAAGCTGGGTGGAGCAACTATGGTGGTGGCAATCAGATCGGACTGATTGAGAATGACGGCGT
GCACAGACAGTGGTATATGCATCTGAGTAAGTACAACGTCAAGGTGGGAGACTACGTCAAAGCCGG
GCAGATCATAGGATGGTCCGGCTCTACAGGTTACTCAACAGCGCCACATCTCCACTTTCAGCGCATG
GTGAATAGCTTCAGCAATTCAACCGCCCAAGATCCGATGCCCTTCCTGAAGTCTGCCGGCTATGGGA
AAGCTGGAGGAACAGTGACACCTACTCCCAATACCGGATGGAAAACGAACAAGTACGGCACTCTGT
ACAAATCCGAATCTGCCAGTTTCACACCCAATACCGACATCATCACCCGAACCACTGGACCTTTTCGG
TCAATGCCGCAGTCTGGAGTACTGAAAGCAGGGCAAACTATCCACTATGACGAGGTGATGAAGCAG
GATGGCCACGTATGGGTGGGGTACACAGGCAATAGCGGCCAGAGGATTTACTTGCCAGTCCGTACC
TGGAACAAAAGCACGAACACTCTTGGCGTTTTGTGGGGCACCATCAAGGCGGCCGCCGGAGGAGGC
GGCTCCCACCACCACCACCATCATTGACTCGAGCTGGTACTGCATGCACGCAATGCTAGCTGCCCCTT
TCCCGTCCTGGGTACCCCGAGTCTCCCCCGACCTCGGGTCCCAGGTATGCTCCCACCTCCACCTGCCC
CACTCACCACCTCTGCTAGTTCCAGACACCTCCCAAGCACGCAGCAATGCAGCTCAAAACGCTTAGCC
TAGCCACACCCCCACGGGAAACAGCAGTGATTAACCTTTAGCAATAAACGAAAGTTTAACTAAGCTA
TACTAACCCCAGGGTTGGTCAATTTCGTGCCAGCCACACCCTGGAGCTAGCAAAAAAAAAAAAAAAA
AAAAAAAAAAAAAAGCATATGACTAAAAAAAAAAAAAAAAAAAAAAAAAAAAAAAAAAAAAAAAA
AAAAAAAAAAAAAAAAAAAAAAAAAAAAA
Construct 16: pST4-T7-AGA-dEarI-hAg-Kozak-SS(hINS)-FLAG-hL0484m-His-FI-A30LA70
MALWMRLLPLLALLALWGPDPAAAMKDYKDDDDKGGGGSGDQFLDEKEGNHTTAYRDGSGIWTICR
GATMVDGKPVIPGMRLSKEKCDQVNAIERDKALAWVERNIKVPLTEPQKAGIASFCPYNIGPGKCFPSTF
YKRLNAGDRKGACEAIRWWIKDGGRDCRIRSNNCYGQVIRRDQESALACWGIDQAAAGGGGSHHHHH
H*
Nt sequence
AGAATAAACTAGTATTCTTCTGGTCCCCACAGACTCAGAGAGAACCCGCCACCATGGCCCTGTGGAT
GAGACTGCTGCCTCTGCTTGCTCTGCTGGCACTGTGGGGACCTGATCCTGCTGCTGCTATGAAGGAC
TACAAAGACGATGACGACAAGGGAGGAGGCGGCTCCGGAGATCAGTTTCTCGACGAGAAGGAAGG
CAATCACACCACTGCGTATAGGGATGGCTCTGGGATATGGACCATCTGTAGAGGCGCCACAATGGTC
GATGGCAAACCCGTGATACCCGGAATGCGTCTGTCAAAGGAGAAGTGCGATCAGGTGAATGCCATC
GAACGCGACAAAGCTCTGGCCTGGGTTGAGCGGAACATCAAGGTTCCACTGACGGAACCACAGAAA
GCCGGAATTGCGTCCTTCTGTCCGTACAACATTGGCCCTGGAAAGTGCTTTCCCTCCACTTTCTACAA
GCGCCTGAATGCAGGGGACAGAAAAGGAGCCTGTGAGGCTATCAGGTGGTGGATCAAAGATGGAG
GTAGGGACTGTCGAATCAGGAGCAACAACTGCTATGGGCAAGTGATTCGGAGAGATCAGGAGAGT
GCTCTGGCTTGCTGGGGTATTGACCAGGCGGCCGCCGGAGGAGGCGGCTCCCACCACCACCACCAT
CATTGACTCGAGCTGGTACTGCATGCACGCAATGCTAGCTGCCCCTTTCCCGTCCTGGGTACCCCGAG
TCTCCCCCGACCTCGGGTCCCAGGTATGCTCCCACCTCCACCTGCCCCACTCACCACCTCTGCTAGTTC
CAGACACCTCCCAAGCACGCAGCAATGCAGCTCAAAACGCTTAGCCTAGCCACACCCCCACGGGAAA
CAGCAGTGATTAACCTTTAGCAATAAACGAAAGTTTAACTAAGCTATACTAACCCCAGGGTTGGTCAA
TTTCGTGCCAGCCACACCCTGGAGCTAGCAAAAAAAAAAAAAAAAAAAAAAAAAAAAAAGCATATG
ACTAAAAAAAAAAAAAAAAAAAAAAAAAAAAAAAAAAAAAAAAAAAAAAAAAAAAAAAAAAAAAA
AAAAAAAA
Construct 17: pST4-T7-AGA-dEarI-hAg-Kozak-SS(hINS)-FLAG-hIL2-His-FI-A30LA70
Aa sequence
MALWMRLLPLLALLALWGPDPAAAMKDYKDDDDKGGGGSGAPTSSSTKKTQLQLEHLLLDLQMILNGI
NNYKNPKLTRMLTFKFYMPKKATELKHLQCLEEELKPLEEVLNLAQSKNFHLRPRDLISNINVIVLELKGSET
TFMCEYADETATIVEFLNRWITFCQSIISTLTAAAGGGGSHHHHHH*
Nt sequence
AGAATAAACTAGTATTCTTCTGGTCCCCACAGACTCAGAGAGAACCCGCCACCATGGCCCTGTGGAT
GAGACTGCTGCCTCTGCTTGCTCTGCTGGCACTGTGGGGACCTGATCCTGCTGCTGCTATGAAGGAC
TACAAAGACGATGACGACAAGGGAGGAGGCGGCTCCGGAGCTCCAACATCTTCTTCAACAAAGAAA
ACACAGCTTCAGCTTGAACACCTTCTTCTTGATCTTCAGATGATTCTGAATGGAATCAACAATTACAAA
AATCCAAAACTGACAAGAATGCTGACATTTAAATTTTACATGCCAAAGAAAGCAACAGAACTGAAAC
ACCTTCAGTGCCTTGAAGAAGAACTGAAACCTCTGGAAGAAGTGCTGAATCTGGCTCAGAGCAAAAA
TTTTCACCTGAGACCAAGAGATCTGATCAGCAACATCAATGTGATTGTGCTGGAACTGAAAGGATCT
GAAACAACATTCATGTGTGAATATGCTGATGAAACAGCAACAATTGTGGAATTTCTGAACAGATGGA
TCACATTTTGCCAGTCAATCATTTCAACACTGACAGCGGCCGCCGGAGGAGGCGGCTCCCACCACCA
CCACCATCATTGACTCGAGCTGGTACTGCATGCACGCAATGCTAGCTGCCCCTTTCCCGTCCTGGGTA
CCCCGAGTCTCCCCCGACCTCGGGTCCCAGGTATGCTCCCACCTCCACCTGCCCCACTCACCACCTCTG
CTAGTTCCAGACACCTCCCAAGCACGCAGCAATGCAGCTCAAAACGCTTAGCCTAGCCACACCCCCAC
GGGAAACAGCAGTGATTAACCTTTAGCAATAAACGAAAGTTTAACTAAGCTATACTAACCCCAGGGT
TGGTCAATTTCGTGCCAGCCACACCCTGGAGCTAGCAAAAAAAAAAAAAAAAAAAAAAAAAAAAAA
GCATATGACTAAAAAAAAAAAAAAAAAAAAAAAAAAAAAAAAAAAAAAAAAAAAAAAAAAAAAAA
AAAAAAAAAAAAAAA
Construct 18: pST4-T7-AGA-dEarI-hAg-Kozak-SS(hIL2)-FLAG-hIL2-His-FI-A30LA70
Aa sequence
MYRMQLLSCIALSLALVTNSMKDYKDDDDKGGGGSGAPTSSSTKKTQLQLEHLLLDLQMILNGINNYKN
PKLTRMLTFKFYMPKKATELKHLQCLEEELKPLEEVLNLAQSKNFHLRPRDLISNINVIVLELKGSETTFMCE
YADETATIVEFLNRWITFCQSIISTLTAAAGGGGSHHHHHH*
Nt sequence
AGAATAAACTAGTATTCTTCTGGTCCCCACAGACTCAGAGAGAACCCGCCACCATGTACCGGATGCA
GCTGCTGAGCTGTATCGCCCTGTCTCTGGCCCTGGTCACCAATTCTATGAAGGACTACAAAGACGAT
GACGACAAGGGAGGAGGCGGCTCCGGAGCTCCAACATCTTCTTCAACAAAGAAAACACAGCTTCAG
CTTGAACACCTTCTTCTTGATCTTCAGATGATTCTGAATGGAATCAACAATTACAAAAATCCAAAACTG
ACAAGAATGCTGACATTTAAATTTTACATGCCAAAGAAAGCAACAGAACTGAAACACCTTCAGTGCC
TTGAAGAAGAACTGAAACCTCTGGAAGAAGTGCTGAATCTGGCTCAGAGCAAAAATTTTCACCTGAG
ACCAAGAGATCTGATCAGCAACATCAATGTGATTGTGCTGGAACTGAAAGGATCTGAAACAACATTC
ATGTGTGAATATGCTGATGAAACAGCAACAATTGTGGAATTTCTGAACAGATGGATCACATTITGCC
AGTCAATCATTTCAACACTGACAGCGGCCGCCGGAGGAGGCGGCTCCCACCACCACCACCATCATTG
ACTCGAGCTGGTACTGCATGCACGCAATGCTAGCTGCCCCTTTCCCGTCCTGGGTACCCCGAGTCTCC
CCCGACCTCGGGTCCCAGGTATGCTCCCACCTCCACCTGCCCCACTCACCACCTCTGCTAGTTCCAGAC
ACCTCCCAAGCACGCAGCAATGCAGCTCAAAACGCTTAGCCTAGCCACACCCCCACGGGAAACAGCA
GTGATTAACCTTTAGCAATAAACGAAAGTTTAACTAAGCTATACTAACCCCAGGGTTGGTCAATTTCG
TGCCAGCCACACCCTGGAGCTAGCAAAAAAAAAAAAAAAAAAAAAAAAAAAAAAGCATATGACTAA
AAAAAAAAAAAAAAAAAAAAAAAAAAAAAAAAAAAAAAAAAAAAAAAAAAAAAAAAAAAAAAAAA
AAA
Construct 19: pcDNA3.1(+)-SS(hINS)-FLAG-hL0473-His
Aa sequence
MALWMRLLPLLALLALWGPDPAAAMKDYKDDDDKGGGGSGMAKTKKQIEQRLQAYAKGTVDSPYRIT
KPTSYDPSFGVMEVGAIDADRYYHAQCQDLITDYVLWLTDNKVRPWGNAKDQINQSYGTGFKIHKNLP
STVAKTGWIAVFTSGTYSQYGHIGIVYSPGDTNKFHIIEQNWNGWANKKPLTRWDNYYGLTHFIEVPYA
KETITTKKTAPKAATSKPAKKISVSHNNINLQSNFAKRGYNPKGVVIHNDAGRSTAQQYENSLKGANQNR
LNNGIAHAYADSSHIWYAIEPERIAYHTGDGVKKGTGNYDYFGIEVCQSMSASDKEFMANEQVVLQEVA
RLLKKWKLPANRNTVRLHLEFSATSCPHRSLTMRTGFNPVTQGRPSDAIIKKLKDIWIKEIRAYMEGKTPSI
KTVANKPGSASTPATKSNSDGWKVNAYNTYYKTERASFTVGSTRIAVRSTGPFTTCPFTGWLNPGVTITY
DEVMVQNGFVWLGYDSYNGRRYVPIRTAQGTPGSSNYVLGPLWGTISAAAGGGGSHHHHHH
Nt sequence
GAGACCCAAGCTGGCTAGCGTTTAAACTTAAGCTTGGTACCGAGCTCGGATCCGCCACCATGGCCCT
GTGGATGAGACTGCTGCCTCTGCTTGCTCTGCTGGCACTGTGGGGACCTGATCCTGCTGCTGCTATG
AAGGACTACAAAGACGATGACGACAAGGGAGGAGGCGGCTCCGGAATGGCCAAAACCAAGAAACA
GATAGAACAGAGACTGCAAGCTTACGCAAAGGGGACCGTGGATTCCCCTTACCGAATCACCAAGCC
AACCAGCTACGACCCCTCCTTCGGTGTCATGGAGGTTGGCGCTATAGATGCGGATCGCTACTATCAT
GCCCAGTGTCAAGATCTGATAACTGACTATGTACTTTGGCTCACTGACAACAAAGTAAGACCTTGGG
GTAATGCCAAGGACCAGATTAACCAGAGCTATGGGACTGGTTTCAAAATCCACAAGAATCTGCCCAG
TACCGTGGCTAAAACAGGATGGATTGCCGTGTTTACATCCGGGACCTACTCACAGTACGGCCACATC
GGCATCGTGTACAGTCCAGGAGATACCAACAAATTCCATATCATCGAGCAGAATTGGAATGGATGG
GCAAACAAGAAACCTCTGACACGTTGGGACAACTACTACGGGCTGACCCACTTTATCGAGGTCCCAT
ATGCGAAAGAGACAATCACAACGAAGAAAACTGCACCTAAAGCAGCTACCTCAAAACCGGCTAAGA
AGATCTCAGTTTCTCACAACAACATCAACCTTCAGAGCAACTTTGCAAAGAGAGGGTACAACCCAAA
GGGAGTGGTGATTCACAATGACGCCGGACGCTCTACTGCGCAACAGTATGAAAACAGCCTGAAGGG
CGCTAATCAGAACAGGCTGAATAACGGCATTGCCCATGCATATGCTGACAGCAGCCACATTTGGTAT
GCTATCGAACCCGAGAGAATCGCCTATCACACAGGCGATGGCGTTAAGAAGGGAACAGGCAATTAC
GACTACTTCGGTATTGAAGTATGCCAGAGCATGAGCGCAAGTGACAAAGAGTTCATGGCCAACGAA
CAGGTGGTACTGCAGGAGGTGGCTAGGTTGCTCAAGAAATGGAAGCTTCCCGCCAATCGGAATACC
GTGCGGCTCCATCTGGAATTCTCTGCCACATCTTGCCCTCATAGGAGCCTCACAATGCGGACCGGTTT
CAATCCGGTGACTCAGGGTAGACCCTCAGACGCCATCATCAAGAAGTTGAAGGATATCTGGATAAAA
GAGATTCGCGCATATATGGAGGGTAAAACCCCATCCATTAAGACTGTCGCCAATAAGCCCGGATCAG
CCAGTACCCCTGCAACCAAATCCAACTCCGATGGGTGGAAGGTCAATGCCTACAATACCTACTACAA
AACGGAACGGGCCTCCTTTACAGTGGGCTCTACTCGGATTGCTGTGCGTAGTACAGGACCCTTTACT
ACATGCCCGTTTACAGGGTGGCTGAACCCCGGAGTCACGATAACCTATGACGAGGTTATGGTGCAG
AACGGGTTTGTGTGGTTGGGCTATGATAGCTACAATGGGAGGAGGTATGTCCCCATTCGCACTGCCC
AAGGAACTCCTGGCAGCTCCAATTATGTGCTTGGCCCTCTGTGGGGCACGATTTCTGCGGCCGCCGG
AGGAGGCGGCTCCCACCACCACCACCATCATTGA
Construct 20: pcDNA3.1(+)-SS(hINS)-FLAG-hL0482-His
Aa sequence
MALWMRLLPLLALLALWGPDPAAAMKDYKDDDDKGGGGSGREAPKTQIYTVKKGDTLSAIALKYKTTV
SNIQNTNNIANPNLIFIGQKLKVPMTPLVEPKPKTVSSNNKSNSNSSTLNYLKTLENRGWDFDGSYGWQC
FDLVNVYWNHLYGHGLKGYGAKDIPYANNFNSEAKIYHNTPTFKAEPGDLVVFSGRFGGGYGHTAIVLN
GDYDGKLMKFQSLDQNWNNGGWRKAEVAHKVVHNYENDMIFIRPFKKAAAAGGGGSHHHHHH
Nt sequence
GAGACCCAAGCTGGCTAGCGTTTAAACTTAAGCTTGGTACCGAGCTCGGATCCGCCACCATGGCCCT
GTGGATGAGACTGCTGCCTCTGCTTGCTCTGCTGGCACTGTGGGGACCTGATCCTGCTGCTGCTATG
AAGGACTACAAAGACGATGACGACAAGGGAGGAGGCGGCTCCGGAAGAGAGGCTCCCAAAACACA
GATTTACACCGTCAAGAAGGGAGATACGCTTAGCGCCATTGCACTGAAATACAAGACCACCGTATCA
AACATCCAGAACACCAATAACATAGCCAATCCCAATCTCATCTTCATTGGCCAGAAGCTCAAGGTTCC
CATGACTCCACTGGTCGAACCGAAACCTAAGACAGTGTCCTCCAATAACAAGAGTAACAGCAACTCC
TCTACACTGAACTATCTCAAGACGTTGGAGAATCGCGGTTGGGACTTTGACGGCAGTTACGGCTGGC
AATGCTTCGACTTGGTGAATGTGTACTGGAATCACCTGTATGGGCATGGACTGAAAGGCTATGGGGC
CAAAGACATACCTTATGCTAACAATTTCAACTCTGAGGCCAAGATCTACCACAACACTCCTACCTTCAA
GGCAGAACCAGGAGATCTGGTTGTGTTTAGCGGACGGTTTGGCGGAGGCTATGGGCATACTGCCAT
TGTGCTTAATGGGGATTACGATGGCAAACTGATGAAATTCCAGTCACTGGACCAGAACTGGAACAAT
GGTGGTTGGCGAAAAGCGGAAGTAGCCCACAAGGTCGTCCACAATTACGAGAACGACATGATCTTC
ATCCGTCCCTTTAAGAAAGCGGCGGCCGCCGGAGGAGGCGGCTCCCACCACCACCACCATCATTGA
Construct 21: pcDNA3.1(+)-SS(hINS)-FLAG-hL0483-His
Aa sequence:
MALWMRLLPLLALLALWGPDPAAAMKDYKDDDDKGGGGSGAATHEHSAQWLNNYKKGYGYGPYPL
GINGGMHYGVDFFMNIGTPVKAISSGKIVEAGWSNYGGGNQIGLIENDGVHRQWYMHLSKYNVKVGD
YVKAGQIIGWSGSTGYSTAPHLHFQRMVNSFSNSTAQDPMPFLKSAGYGKAGGTVTPTPNTGWKTNKY
GTLYKSESASFTPNTDIITRTTGPFRSMPQSGVLKAGQTIHYDEVMKQDGHVWVGYTGNSGQRIYLPVRT
WNKSTNTLGVLWGTIKAAAGGGGSHHHHHH
Nt sequence
GAGACCCAAGCTGGCTAGCGTTTAAACTTAAGCTTGGTACCGAGCTCGGATCCGCCACCATGGCCCT
GTGGATGAGACTGCTGCCTCTGCTTGCTCTGCTGGCACTGTGGGGACCTGATCCTGCTGCTGCTATG
AAGGACTACAAAGACGATGACGACAAGGGAGGAGGCGGCTCCGGAGCAGCAACGCATGAGCATTC
CGCTCAATGGCTCAACAACTACAAGAAGGGTTACGGCTATGGCCCCTATCCTCTGGGGATAAACGGA
GGGATGCACTATGGAGTCGATTTCTTCATGAATATTGGCACTCCAGTGAAAGCGATTTCCAGCGGGA
AGATTGTGGAAGCTGGGTGGAGCAACTATGGTGGTGGCAATCAGATCGGACTGATTGAGAATGACG
GCGTGCACAGACAGTGGTATATGCATCTGAGTAAGTACAACGTCAAGGTGGGAGACTACGTCAAAG
CCGGGCAGATCATAGGATGGTCCGGCTCTACAGGTTACTCAACAGCGCCACATCTCCACTTTCAGCG
CATGGTGAATAGCTTCAGCAATTCAACCGCCCAAGATCCGATGCCCTTCCTGAAGTCTGCCGGCTATG
GGAAAGCTGGAGGAACAGTGACACCTACTCCCAATACCGGATGGAAAACGAACAAGTACGGCACTC
TGTACAAATCCGAATCTGCCAGTTTCACACCCAATACCGACATCATCACCCGAACCACTGGACCTTTTC
GGTCAATGCCGCAGTCTGGAGTACTGAAAGCAGGGCAAACTATCCACTATGACGAGGTGATGAAGC
AGGATGGCCACGTATGGGTGGGGTACACAGGCAATAGCGGCCAGAGGATTTACTTGCCAGTCCGTA
CCTGGAACAAAAGCACGAACACTCTTGGCGTTTTGTGGGGCACCATCAAGGCGGCCGCCGGAGGAG
GCGGCTCCCACCACCACCACCATCATTGA
Construct 22: pcDNA3.1(+)-SS(hINS)-FLAG-hL0484m-His
Aa sequence
MALWMRLLPLLALLALWGPDPAAAMKDYKDDDDKGGGGSGDQFLDEKEGNHTTAYRDGSGIWTICR
GATMVDGKPVIPGMRLSKEKCDQVNAIERDKALAWVERNIKVPLTEPQKAGIASFCPYNIGPGKCFPSTF
YKRLNAGDRKGACEAIRWWIKDGGRDCRIRSNNCYGQVIRRDQESALACWGIDQAAAGGGGSHHHHH
H
Nt sequence
GAGACCCAAGCTGGCTAGCGTTTAAACTTAAGCTTGGTACCGAGCTCGGATCCGCCACCATGGCCCT
GTGGATGAGACTGCTGCCTCTGCTTGCTCTGCTGGCACTGTGGGGACCTGATCCTGCTGCTGCTATG
AAGGACTACAAAGACGATGACGACAAGGGAGGAGGCGGCTCCGGAGATCAGTTTCTCGACGAGAA
GGAAGGCAATCACACCACTGCGTATAGGGATGGCTCTGGGATATGGACCATCTGTAGAGGCGCCAC
AATGGTCGATGGCAAACCCGTGATACCCGGAATGCGTCTGTCAAAGGAGAAGTGCGATCAGGTGAA
TGCCATCGAACGCGACAAAGCTCTGGCCTGGGTTGAGCGGAACATCAAGGTTCCACTGACGGAACC
ACAGAAAGCCGGAATTGCGTCCTTCTGTCCGTACAACATTGGCCCTGGAAAGTGCTTTCCCTCCACTT
TCTACAAGCGCCTGAATGCAGGGGACAGAAAAGGAGCCTGTGAGGCTATCAGGTGGTGGATCAAA
GATGGAGGTAGGGACTGTCGAATCAGGAGCAACAACTGCTATGGGCAAGTGATTCGGAGAGATCA
GGAGAGTGCTCTGGCTTGCTGGGGTATTGACCAGGCGGCCGCCGGAGGAGGCGGCTCCCACCACCA
CCACCATCATTGA
Construct 23: pcDNA3.1(+)-SS(hINS)-FLAG-hIL2-His
Aa sequence
MALWMRLLPLLALLALWGPDPAAAMKDYKDDDDKGGGGSGAPTSSSTKKTQLQLEHLLLDLQMILNGI
NNYKNPKLTRMLTFKFYMPKKATELKHLQCLEEELKPLEEVLNLAQSKNFHLRPRDLISNINVIVLELKGSET
TFMCEYADETATIVEFLNRWITFCQSIISTLTAAAGGGGSHHHHHH*
Nt sequence:
GAGACCCAAGCTGGCTAGCGTTTAAACTTAAGCTTGGTACCGAGCTCGGATCCGCCACCATGGCCCT
GTGGATGAGACTGCTGCCTCTGCTTGCTCTGCTGGCACTGTGGGGACCTGATCCTGCTGCTGCTATG
AAGGACTACAAAGACGATGACGACAAGGGAGGAGGCGGCTCCGGAGCTCCAACATCTTCTTCAACA
AAGAAAACACAGCTTCAGCTTGAACACCTTCTTCTTGATCTTCAGATGATTCTGAATGGAATCAACAA
TTACAAAAATCCAAAACTGACAAGAATGCTGACATTTAAATTTTACATGCCAAAGAAAGCAACAGAA
CTGAAACACCTTCAGTGCCTTGAAGAAGAACTGAAACCTCTGGAAGAAGTGCTGAATCTGGCTCAGA
GCAAAAATTTTCACCTGAGACCAAGAGATCTGATCAGCAACATCAATGTGATTGTGCTGGAACTGAA
AGGATCTGAAACAACATTCATGTGTGAATATGCTGATGAAACAGCAACAATTGTGGAATTTCTGAAC
AGATGGATCACATTTTGCCAGTCAATCATTTCAACACTGACAGCGGCCGCCGGAGGAGGCGGCTCCC
ACCACCACCACCATCATTGA
Construct 24: pcDNA3.1(+)-SS(hIL2)-FLAG-hIL2-His
Aa sequence
MYRMQLLSCIALSLALVTNSMKDYKDDDDKGGGGSGAPTSSSTKKTQLQLEHLLLDLQMILNGINNYKN
PKLTRMLTFKFYMPKKATELKHLQCLEEELKPLEEVLNLAQSKNFHLRPRDLISNINVIVLELKGSETTFMCE
YADETATIVEFLNRWITFCQSIISTLTAAAGGGGSHHHHHH
Nt sequence
GAGACCCAAGCTGGCTAGCGTTTAAACTTAAGCTTGGTACCGAGCTCGGATCCGCCACCATGTACCG
GATGCAGCTGCTGAGCTGTATCGCCCTGTCTCTGGCCCTGGTCACCAATTCTATGAAGGACTACAAA
GACGATGACGACAAGGGAGGAGGCGGCTCCGGAGCTCCAACATCTTCTTCAACAAAGAAAACACAG
CTTCAGCTTGAACACCTTCTTCTTGATCTTCAGATGATTCTGAATGGAATCAACAATTACAAAAATCCA
AAACTGACAAGAATGCTGACATTTAAATTTTACATGCCAAAGAAAGCAACAGAACTGAAACACCTTC
AGTGCCTTGAAGAAGAACTGAAACCTCTGGAAGAAGTGCTGAATCTGGCTCAGAGCAAAAATTTTCA
CCTGAGACCAAGAGATCTGATCAGCAACATCAATGTGATTGTGCTGGAACTGAAAGGATCTGAAACA
ACATTCATGTGTGAATATGCTGATGAAACAGCAACAATTGTGGAATTTCTGAACAGATGGATCACAT
TTTGCCAGTCAATCATTTCAACACTGACAGCGGCCGCCGGAGGAGGCGGCTCCCACCACCACCACCA
TCATTGA
Construct 25: PL084_pST4-T7-AGA-dEarI-hAg-Kozak-SS(IGkappa)-His-3C-hL0483(PM)-
FLAG-FI-A30LA70
Aa sequence:
MDMRAPAGIFGFLLVLFPGYRSHHHHHHLEVLFQGPAATHEHSAQWLNNYKKGYGYGPYPLGINGGM
HYGVDFFMNIGTPVKAISSGKIVEAGWSNYGGGNQIGLIENDGVHRQWYMHLSKYNVKVGDYVKAGQI
IGWSGSTGYSTAPHLHFORMVNSFSQSTAQDPMPFLKSAGYGKAGGTVTPTPNTGWKTNKYGTLYKSE
SASFTPNTDIITRTTGPFRSMPQSGVLKAGQTIHYDEVMKQDGHVWVGYTGNSGQRIYLPVRTWQKST
NTLGVLWGTIKSGGSDYKDDDDK*
Nt sequence:
AGAATAAACTAGTATTCTTCTGGTCCCCACAGACTCAGAGAGAACCCGCCACCATGGACATGCGGGC
CCCTGCCGGAATCTTCGGCTTTCTGCTGGTGCTGTTCCCCGGCTACAGAAGCCATCATCATCACCACC
ACCTCGAAGTATTGTTTCAGGGACCCGCCGCCACCCACGAGCACAGCGCTCAATGGCTGAACAACTA
CAAAAAGGGCTACGGCTATGGTCCTTATCCTCTGGGCATCAACGGCGGGATGCACTACGGCGTGGA
CTTCTTCATGAACATCGGCACCCCCGTGAAGGCCATCAGCAGCGGAAAGATCGTGGAAGCCGGCTG
GTCCAACTACGGCGGCGGCAACCAGATCGGACTGATCGAGAACGACGGCGTGCACAGACAGTGGTA
CATGCATCTGTCTAAGTACAATGTGAAGGTGGGCGACTACGTGAAAGCCGGCCAAATCATTGGATG
GAGCGGCTCTACCGGCTACAGCACAGCCCCACACCTGCACTTCCAGAGAATGGTGAACAGCTTCAGC
CAGTCCACAGCCCAGGACCCCATGCCTTTCCTGAAGAGCGCCGGCTACGGCAAGGCCGGAGGCACC
GTGACCCCAACACCTAATACCGGCTGGAAGACCAACAAGTACGGCACCCTGTACAAGTCTGAAAGCG
CTTCTTTTACCCCTAACACTGATATCATCACAAGAACCACCGGCCCTTTTAGAAGCATGCCCCAGAGC
GGCGTCCTCAAAGCTGGACAGACCATCCACTACGACGAGGTGATGAAACAGGATGGCCACGTGTGG
GTCGGCTACACCGGAAATAGCGGCCAGCGGATCTACCTGCCTGTGCGGACATGGCAGAAGTCCACC
AACACACTGGGAGTTCTGTGGGGCACAATCAAGTCAGGAGGTTCAGATTACAAGGATGACGACGAT
AAGTGACTCGAGCTGGTACTGCATGCACGCAATGCTAGCTGCCCCTTTCCCGTCCTGGGTACCCCGA
GTCTCCCCCGACCTCGGGTCCCAGGTATGCTCCCACCTCCACCTGCCCCACTCACCACCTCTGCTAGTT
CCAGACACCTCCCAAGCACGCAGCAATGCAGCTCAAAACGCTTAGCCTAGCCACACCCCCACGGGAA
ACAGCAGTGATTAACCTTTAGCAATAAACGAAAGTTTAACTAAGCTATACTAACCCCAGGGTTGGTCA
ATTTCGTGCCAGCCACACCCTGGAGCTAGCAAAAAAAAAAAAAAAAAAAAAAAAAAAAAAGCATAT
GACTAAAAAAAAAAAAAAAAAAAAAAAAAAAAAAAAAAAAAAAAAAAAAAAAAAAAAAAAAAAAA
AAAAAAAAA

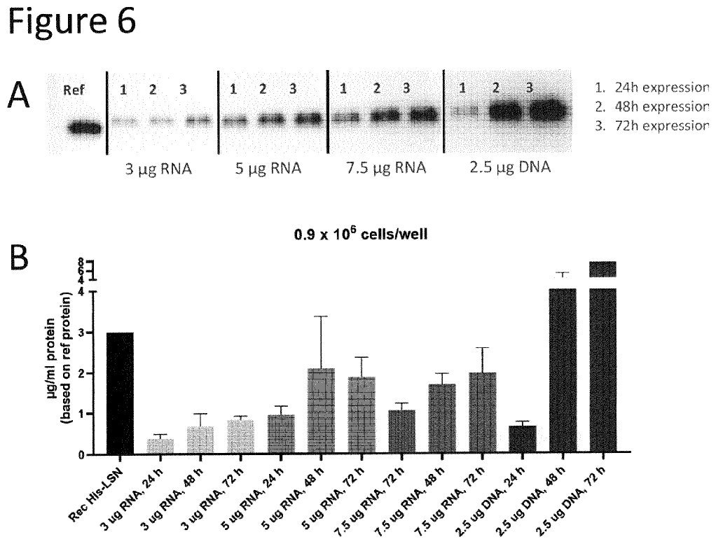

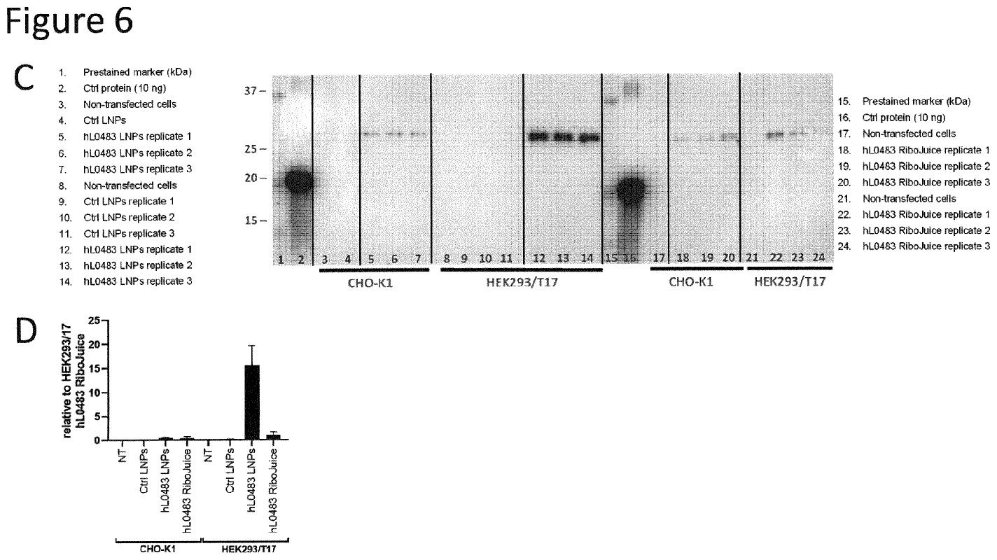

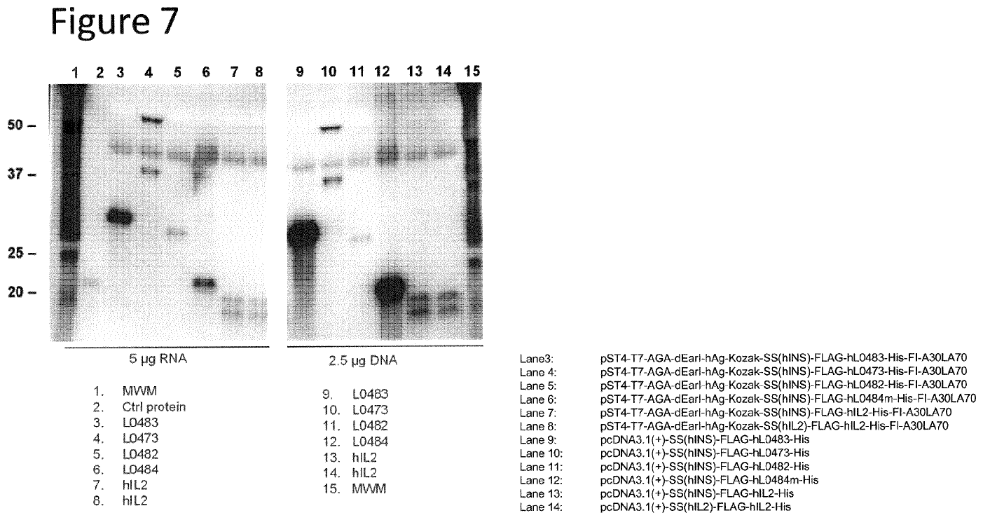

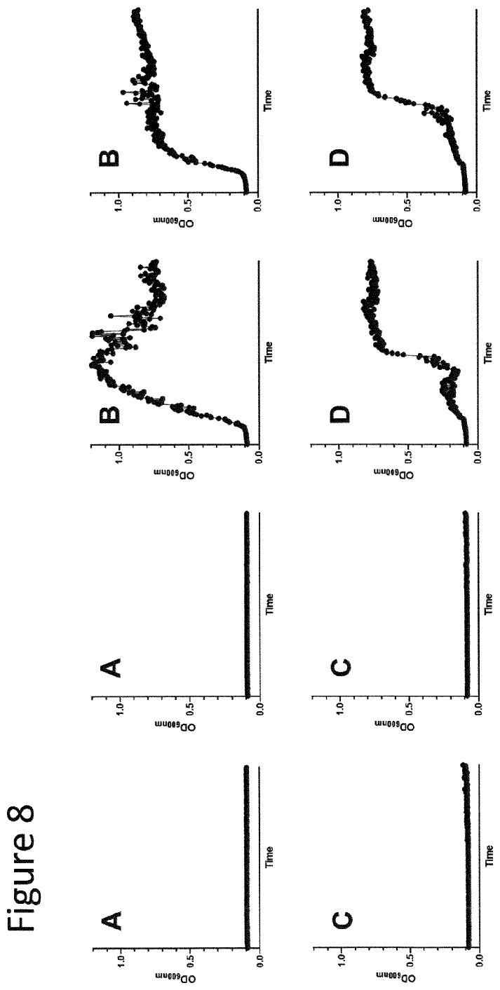

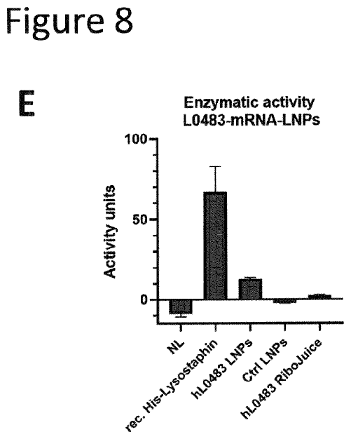

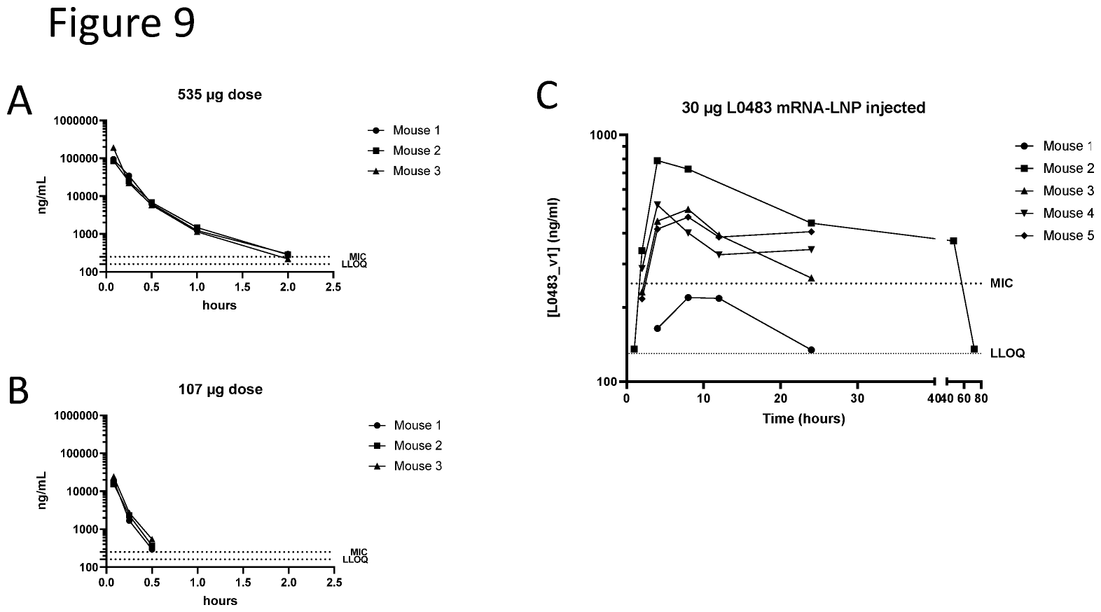

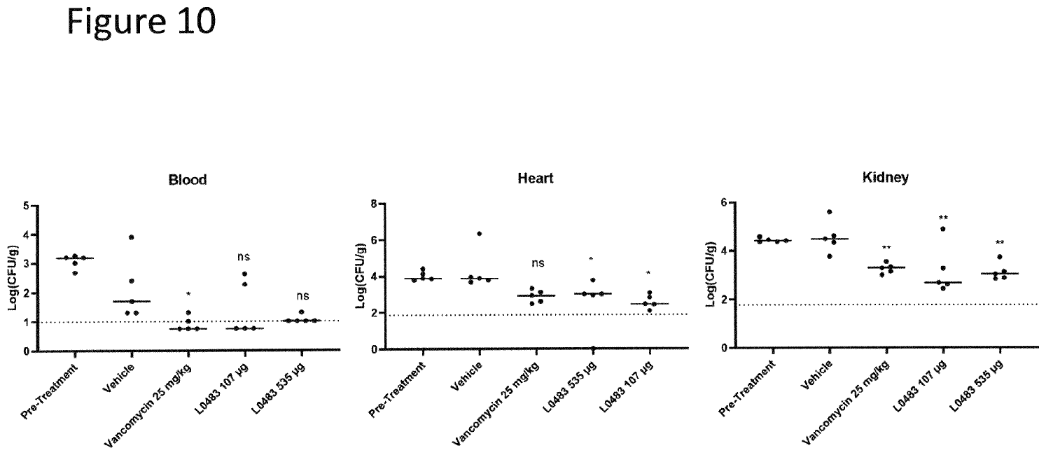

Endolysin expression was seen for DNA as well as for mRNA transfected HEK293T/17 cells. A dosing and time course experiment of a prototype endolysin expressed from an mRNA construct revealed that expression was dose, as well as time dependent (FIGS. 6A and 6B). The highest expression levels from mRNA were in the range of 2 μg/ml.

For the determination of pharmacokinetic parameters in mice, an LNP-formulated mRNA was generated and tested for expression in mammalian cells. Transfection of 1.5 μg of LNP-mRNA led to detectable lysin levels in the cell supernatants. The levels were determined to be in the ng/ml range (FIG. 6C+D).

Expression of lysin variants from mRNA or DNA transfections is shown in FIG. 7. 5 μg of mRNA or 2.5 μg of DNA were transfected into HEK293T/17 cells and supernatants were analyzed by Western Blot, demonstrating expression from DNA as well as from mRNA for different lysin variants.

### Example 10: Bactericidal Activity of Lysins Encoded in and Secreted from HEK293T/17 Cells

Supernatants from DNA or RNA transfected cells are concentrated (5 fold) using VivaSpin 500 concentrators (Sartorius VS0102) and tested for bactericidal activity in a 96 well plate. For this purpose, 60 μl of supernatants (concentrated or not concentrated) are mixed with S. aureus ATCC® 43300™ in 40 μl cation-adjusted Müller Hinton broth (Merck 90922), supplemented with 10% horse serum (Thermo Scientific 26050070), such that the reaction contains 5×10⁵ cfu/ml. The optical density at 600 nm (OD600ₙₘ) of these solutions is monitored in a plate reader (TECAN Infinite 200) over the course of 24 hours to quantify bacterial growth. Bacterial growth, or the inhibition thereof, reveals the levels of active lysin in the supernatant tested. Supernatants of mock transfected HEK293T/17 cells serve as a control to rule out an effect of the culture medium on lytic activity.

When testing the non-concentrated supernatants, a clear delay in outgrowth of staphylococci can be observed, meaning that bacterial killing occurs but due to the low concentration of lysin complete eradication is not achieved. The complete inhibition of staphylococcal outgrowth indicates that the level of expressed and active lysins is sufficiently high in the concentrated supernatant to lyse all S. aureus bacteria in the culture (FIG. 8A-D).

The enzymatic activity of RNA expressed lysins secreted in HEK293 supernatants was also monitored using a dye release assay. Crude peptidoglycan extract, obtained by lysis of staphylococci through heat shock, was labelled with remazol brilliant blue (RBB, Sigma, R⁸⁰⁰¹-25G) (Farris et al, J Vis Exp, 2016). After 5 hours of incubation of this substrate with the lysin to be tested, the reaction is stopped by the addition of ethanol, centrifuged down and enzymatic activity is assessed by measuring the release of the RBB dye at OD595 nm. As a positive control, recombinant lysin was spiked into non lipofected supernatant at 3 μg/ml. Non-lipofected supernatant and supernatant from a luciferase mRNA LNP transfection served as negative controls. In FIG. 8E, enzymatic activity is seen for L⁰⁴⁸³ mRNA-LNP supernatants. The lower levels of activity compared to the spiked recombinant protein, are due to the lower concentration in the supernatant.

These experiments reveal DNA and mRNA-encoded lysins which can be expressed and secreted in an active form in human cells resulting in successful lysis of S. aureus (FIG. 8).

### Example 11: Pharmacokinetics and Efficacy of mRNA Expressed Lysin and Recombinant Lysin In Vivo

To compare the in vivo pharmacokinetics of an mRNA-delivered lysin and a recombinantly expressed and administered lysin, mice were injected with lipid-nanoparticle (LNP) formulated mRNA and 2 different doses of recombinant lysin. Expression levels were determined in mouse plasma by quantitation in a sandwich ELISA.

Specific-pathogen-free male CD1 mice (Charles River) of 11-15 g at arrival, were left to acclimatize for at least 7 days prior to a single bolus application of different doses of LNP-mRNA or recombinant protein. 5 mice were administered with a dose of 30 μg LNP-mRNA and two groups of 3 mice each with recombinant lysin (535 μg and 107 μg per mouse, respectively). A blood microsample (^{˜}20 μl) was collected from each mouse by tail vein puncture into a capillary and separated for plasma collection at timepoints 1, 2, 4, 8, 12, 24, 48 and 72 hours post application for the LNP-mRNA groups and 0.08, 0.25, 0.5, 1, 2, 4, 8, 24 hours post application for the recombinant lysin. The plasma samples were then analyzed for their lysin content using a sandwich ELISA. Briefly, a rabbit antibody against the HRV3C cleavage site (Abcam PA1-118) was used as capturing antibody and a HRP coupled anti-flag antibody (Thermo Fisher A8592-0.2 MG) was used as detection antibody.

The analyses of the plasma samples after administration of recombinant protein revealed a poor PK profile for both doses tested (FIG. 9A). Lysin in plasma could be detected for only 2 hours post treatment in the high dose; for the low dose, no lysin could be detected 30 min after administration. An important parameter for efficacy of treatment is the time the plasma concentration of lysin above MIC (minimum inhibitory concentration determined to be 250 ng/ml for this lysin). In the high dose group, time above MIC was approximately 100 min, in the low dose group time above MIC was only 30 min.

The profile was very different for the LNP-mRNA administered lysin (FIG. 9B). Cmax, ranged between 787 and 219 ng/ml depending on the mouse, hence lower than determined for the recombinant protein. Tmax ranged between 4 and 12 hours depending on the mouse (Table 7). The AUC values were comparable in the mRNA groups and in the recombinant protein groups. For maximal efficacy, an endolysin plasma level above MIC is desired, which was seen for 4 of the 5 mice and time over MIC was over 24 hours. Although Cmax was lower when mRNA was applied, time over MIC was much longer, which we postulate will lead to better efficacy in vivo.

TABLE 7
PK parameters determined for recombinant lysin
application and mRNA-LNP application

| | 30 μg | 535 μg | 107 μg |
|---|---|---|---|
| | mRNA dose | protein dose | protein dose |
| Cmax (ng/ml) | 219-787 | 123294 | 19672 |
| Tmax (h) | 4-12 | 0.08 | 0.08 |
| AUC (ng*h*ml⁻¹) | 1247-13166 | 19141 | 2085 |
| Time over MIC (h) | 24-48 | 1.5 | 0.5 |

The PK data generated with LNP-mRNA clearly shows good bioavailability of the lysin when administered as mRNA. In previous experiments, these PK parameters were determined for a lysin administered i.v. as recombinant protein. In parallel to assessing the bioavailability of the recombinant protein, an in vivo efficacy study was performed, with bacterial burden in different tissues serving as readout.

Specific-pathogen-free male CD1 mice (Charles River) of 11-15g at arrival, were left to acclimatize for at least 7 days prior to IV administration of 5×10⁷ cfu of bacteria (S. aureus NCTC8189). One hour post-infection, non-treated control mice were euthanized and the dose groups (5 mice per group) were as follows: 535 μg per mouse, 107 μg per mouse, Vancomycin at 25 mg/kg and a mock-treatment control (vehicle only), all administered i.v. q12h. Mice were euthanized 24 hours post treatment and bacterial burden in blood, heart and kidney was determined by quantitative culture on plates.

The PK data did not look promising, especially the rapid decline in plasma concentration and the time over MIC being very short, nevertheless efficacy was tested using a doses of 535 μg or 107 μg per mouse. Interestingly, despite the suboptimal bioavailability and rapid elimination of the lysin from plasma, efficacy could be seen. Bacterial burden was decreased compared to vehicle only in blood as well as in heart and kidneys and in the range of vancomycin efficacy (FIG. 10). No dose dependent effect of the lysin was observed. These results clearly show that the lysin is efficacious in this model, even at very low plasma concentrations.

The PK data generated for the LNP-mRNA administered lysin demonstrates longer time over MIC, indicating longer bioavailability than the recombinant version of the lysin. Rotolo et al (poster ASM microbe 2016) observed that a ratio of AUC/MIC above 1 was required for maximal efficacy (>3-log reduction in bacterial burden in a mouse thigh infection model). This ratio was also taken as basis for dose selection in a phase 2 clinical study of an anti-staphylococcal lysin (Fowler et al, J. Clin Invest, 2020), where positive responses were seen. Taking these results together, we postulate that the LNP-mRNA administration of the lysin should lead to an even higher efficacy than seen for the recombinant protein.

1-119. (canceled)

120. A composition or medical preparation comprising RNA encoding an amino acid sequence comprising a peptidoglycan hydrolase.

121. A method of treating bacterial infection in a subject comprising administering RNA encoding an amino acid sequence comprising a peptidoglycan hydrolase to the subject.

122. The composition or medical preparation of claim 120 or the method of claim 121, wherein the peptidoglycan hydrolase:
(a) breaks down peptidoglycan in bacterial cell wall;
(b) is derived from a bacteriophage;
(c) is or is derived from an endolysin; and/or
(d) is secreted peptidoglycan hydrolase.

123. The composition, medical preparation or method of any one of claims 120 to 122, wherein:
(a) the peptidoglycan hydrolase is modified so as to reduce glycosylation, wherein optionally the peptidoglycan hydrolase is modified by removing glycosylation sites; and/or
(b) the peptidoglycan hydrolase is modified so as to reduce immunogenicity, wherein optionally the peptidoglycan hydrolase is modified by removing T cell epitopes.

124. The composition, medical preparation or method of any one of claims 120 to 123, wherein the amino acid sequence comprising a peptidoglycan hydrolase comprises or is fused to a signal peptide, wherein the signal peptide is optionally cleaved off during secretion or export, and/or wherein the signal peptide optionally comprises the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10.

125. The composition, medical preparation or method of any one of claims 120 to 124, wherein the amino acid sequence comprising a peptidoglycan hydrolase comprises an extended pharmacokinetic (PK) polypeptide and/or is fused to a pharmacokinetic modifying group, wherein the pharmacokinetic modifying group optionally comprises:
(a) a moiety which is heterologous to the peptidoglycan hydrolase;
(b) a moiety selected from the group consisting of albumin, an immunoglobulin fragment, transferrin, Fn3, a functional variant thereof, or a functional fragment of the albumin, immunoglobulin fragment, transferrin, Fn3 or the functional variant thereof; and/or
(c) human albumin, a functional variant thereof, or a functional fragment of the human albumin or the functional variant thereof.

126. The composition, medical preparation or method of any one of claims 120 to 125, wherein the RNA comprises a modified nucleoside in place of uridine or in place of each uridine, wherein the modified nucleoside is optionally selected from pseudouridine (ψ), N1-methyl-pseudouridine (m1ψ), and 5-methyl-uridine (m5U), wherein the modified nucleoside preferably is N1-methyl-pseudouridine (m1ψ).

127. The composition, medical preparation or method of any one of claims 120 to 126, wherein the RNA, optionally mRNA or self-amplifying RNA, comprises:
(a) a cap0 5′ cap, optionally m₂^{7,2′O}G(5′)ppSp(5′) G (in particular its D1 diastereomer), or a cap1 5′ cap, optionally m₂^{7,3-O}Gppp(m₁^{2′-O})ApG;
(b) a 5′ UTR, optionally comprising the nucleotide sequence of SEQ ID NO: 11, or a nucleotide sequence having at least 99%, 98%, 97%, 96%, 95%, 90%, 85%, or 80% identity to the nucleotide sequence of SEQ ID NO: 11;
(c) a 3′ UTR, optionally comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12; and/or
(d) a poly-A sequence, optionally comprising at least 100 nucleotides or optionally comprising or consisting of the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

128. The composition, medical preparation or method of any one of claims 120 to 127, wherein:
(a) the amino acid sequence comprising a peptidoglycan hydrolase comprises the amino acid sequence of SEQ ID NO: 1 or 4, an amino acid sequence having at least 99%, 98%, 97%, 96%, 95%, 90%, 85%, or 80% identity to the amino acid sequence of SEQ ID NO: 1 or 4, or a fragment of the amino acid sequence of SEQ ID NO: 1 or 4, or the amino acid sequence having at least 99%, 98%, 97%, 96%, 95%, 90%, 85%, or 80% identity to the amino acid sequence of SEQ ID NO: 1 or 4;
(b) the RNA encoding an amino acid sequence comprising a peptidoglycan hydrolase comprises the nucleotide sequence of SEQ ID NO: 2, 3, 5, 6, 7, 8 or 9, a nucleotide sequence having at least 99%, 98%, 97%, 96%, 95%, 90%, 85%, or 80% identity to the nucleotide sequence of SEQ ID NO: 2, 3, 5, 6, 7, 8 or 9, or a fragment of the nucleotide sequence of SEQ ID NO: 2, 3, 5, 6, 7, 8 or 9 or the nucleotide sequence having at least 99%, 98%, 97%, 96%, 95%, 90%, 85%, or 80% identity to the nucleotide sequence of SEQ ID NO: 2, 3, 5, 6, 7, 8 or 9; and/or
(c) the amino acid sequence comprising a peptidoglycan hydrolase is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

129. The composition or medical preparation of any one of claims 120 to 128, wherein the RNA:
(a) is formulated as a liquid, formulated as a solid, or a combination thereof;
(b) is formulated for injection;
(c) is formulated for intravenous administration; and/or
(d) is formulated or is to be formulated as lipid particles, wherein the RNA lipid particles optionally are lipid nanoparticles (LNP).

130. The composition or medical preparation of any one of claims 120 to 129, which is a pharmaceutical composition, optionally further comprising one or more pharmaceutically acceptable carriers, diluents and/or excipients.

131. The composition or medical preparation of any one of claims 120 to 129, wherein the medical preparation is a kit, optionally further comprising instructions for use of the RNA for treating or preventing bacterial infection.

132. The composition or medical preparation of any one of claims 120 to 131, which is for administration to a human.

133. The composition or medical preparation of any one of claims 120 to 132 for pharmaceutical use, wherein the pharmaceutical use optionally comprises a therapeutic or prophylactic treatment of a disease or disorder, preferably comprising treating or preventing bacterial infection.

134. The method of any one of claims 121 to 128, wherein the RNA:
(a) is formulated as a liquid, formulated as a solid, or a combination thereof;
(b) is administered by injection;
(c) is administered by intravenous administration; and/or
(d) is formulated as lipid particles, wherein the RNA lipid particles optionally are lipid nanoparticles (LNP).

135. The method of any one of claims 121 to 128 and 134, wherein the RNA is formulated as a pharmaceutical composition, optionally further comprising one or more pharmaceutically acceptable carriers, diluents and/or excipients.

136. The method of any one of claims 121 to 128, 134 and 135, wherein the subject is a human.

137. The method of any one of claims 121 to 128 and 134 to 135, wherein the infection is a chronic infection.

138. The method of any one of claims 121 to 128 and 134 to 137, wherein the bacteria causing infection are:
(a) multi-resistant bacteria; and/or
(b) Gram-negative bacteria, Gram-positive bacteria, or both.

139. The method of any one of claims 121 to 128 and 134 to 138, wherein the bacteria are selected from Acinetobacter baumannii, Pseudomonas aeruginosa, Enterobacteriaceae, Porphyromonas gingivalis, Helicobacter pylori, Chlamydia trachomatis, Enterobacteriaceae, E. coli, Klebsiella pneumoniae, Salmonella, Shigella, Borellia, Campylobacter jejuni, Neisseria gonorrhoeae, Chlamydia trachomatis, Vibrio cholerae, and Fusobacterium nucleatum.

140. The method of any one of claims 121 to 128 and 134 to 139, wherein the bacteria are resistant to carbapenem.

141. The method of any one of claims 121 to 128 and 134 to 138, wherein the bacteria are selected from Staphylococci, Streptococci, Staphylococcus aureus, Streptococcus pneumoniae, Clostridioides difficile, Cutibacterium acnes, Mycobacterium tuberculosis, Gardnerella, Enterococcus faecalis, Enterococcus faecium, Lactobacillus iners, Bacillus subtilis and Bacillus anthracis.

142. The method of any one of claims 121 to 128 and 134 to 141, wherein the bacteria are resistant to methicillin and vancomycin or non-susceptible to penicillin.

143. The method of any one of claims 121 to 128, 134 to 138, 141, and 142, wherein the bacteria are methicillin-resistant Staphylococcus aureus (MRSA).


SEQUENCE LISTING (141 sequences)

SEQ ID NO: 1 - protein, 145 aa, synthetic construct
  REGION 1..145: note = Peptidoglycan Hydrolase
MRTNNIDAIKEHEGLRLVAYLDSVGVWTIGYGDTGPDVVKGLTITKEEAEKRLRKRLVEFEGYVNTYVKVPLKQHQFDALVSLVYNIGPTNFKTSTLLKKLNAGDYIGAADQFLVWNKGRVDGKLVVIKGLANRRAKERKQFLGE

SEQ ID NO: 2 - RNA, 438 nt, synthetic construct
  misc_feature 1..438: note = Nucleotide sequence encoding Peptidoglycan Hydrolase
  source 1..438: mol_type = other RNA
atgcgaacgaataatatcgacgccattaaagagcacgaggggctacgtcttgtagcctacctcgactctgtaggtgtatggaccattggttatggtgacactggccctgatgtagttaagggccttactattaccaaagaagaggccgagaagcgccttaggaagcgtctggtggagtttgaagggtatgtcaatacctacgtcaaggttccgctcaaacagcatcaattcgacgctctggtgagtttggtgtacaacattggtcctactaacttcaagacctcgacgttgcttaagaagcttaacgctggggattatattggagcggctgatcagttcctagtgtggaacaaaggccgagtagatgggaagcttgttgtgattaagggtcttgctaataggcgggccaaagaacgtaaacaattccttggagaatga

SEQ ID NO: 3 - RNA, 438 nt, synthetic construct
  misc_feature 1..438: note = Nucleotide sequence encoding Peptidoglycan Hydrolase
  source 1..438: mol_type = other RNA
atgagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatga

SEQ ID NO: 4 - protein, 170 aa, synthetic construct
  REGION 1..170: note = Peptidoglycan Hydrolase
MRVMAPRTLILLLSGALALTETWAGSRTNNIDAIKEHEGLRLVAYLDSVGVWTIGYGDTGPDVVKGLTITKEEAEKRLRKRLVEFEGYVNTYVKVPLKQHQFDALVSLVYNIGPTNFKTSTLLKKLNAGDYIGAADQFLVWNKGRVDGKLVVIKGLANRRAKERKQFLGE

SEQ ID NO: 5 - RNA, 513 nt, synthetic construct
  misc_feature 1..513: note = Nucleotide sequence encoding Peptidoglycan Hydrolase
  source 1..513: mol_type = other RNA
atgagagtgatggcccccagaaccctgatcctgctgctgtctggcgccctggccctgacagagacatgggccggaagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatga

SEQ ID NO: 6 - RNA, 899 nt, synthetic construct
  misc_feature 1..899: note = Nucleotide sequence encoding Peptidoglycan Hydrolase
  source 1..899: mol_type = other RNA
agaataaactagtattcttctggtccccacagactcagagagaacccgccaccatgagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 7 - RNA, 974 nt, synthetic construct
  misc_feature 1..974: note = Nucleotide sequence encoding Peptidoglycan Hydrolase
  source 1..974: mol_type = other RNA
agaataaactagtattcttctggtccccacagactcagagagaacccgccaccatgagagtgatggcccccagaaccctgatcctgctgctgtctggcgccctggccctgacagagacatgggccggaagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 8 - RNA, 8057 nt, synthetic construct
  misc_feature 1..8057: note = Nucleotide sequence encoding Peptidoglycan Hydrolase
  source 1..8057: mol_type = other RNA
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaggctttgagcgagagcgccactatggtgtacaacgaaagggagttcgtcaacaggaaactataccatattgccgttcacggaccctcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggtagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctggagaacccgattcctccaccgcgcccaaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggctgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccactagtatgagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgaggcggccgcaggagcttaattcgacgaataattggatttttattttattttgcaattggtttttaatatttccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 9 - RNA, 8132 nt, synthetic construct
  misc_feature 1..8132: note = Nucleotide sequence encoding Peptidoglycan Hydrolase
  source 1..8132: mol_type = other RNA
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaggctttgagcgagagcgccactatggtgtacaacgaaagggagttcgtcaacaggaaactataccatattgccgttcacggaccctcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggtagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctggagaacccgattcctccaccgcgcccaaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggctgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccactagtatgagagtgatggcccccagaaccctgatcctgctgctgtctggcgccctggccctgacagagacatgggccggaagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgaggcggccgcaggagcttaattcgacgaataattggatttttattttattttgcaattggtttttaatatttccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 10 - protein, 26 aa, synthetic construct
  REGION 1..26: note = Secretion signal
MRVMAPRTLILLLSGALALTETWAGS

SEQ ID NO: 11 - RNA, 40 nt, synthetic construct
  misc_feature 1..40: note = 5'-UTR
  source 1..40: mol_type = other RNA
attcttctggtccccacagactcagagagaacccgccacc

SEQ ID NO: 12 - RNA, 278 nt, synthetic construct
  misc_feature 1..278: note = 3'-UTR
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 13 - RNA, 110 nt, synthetic construct
  misc_feature 1..110: note = A30L70
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 14 - protein, 492 aa, synthetic construct
  REGION 1..492: note = Peptidoglycan Hydrolase
MAKTKKQIEQRLQAYAKGTVDSPYRITKPTSYDPSFGVMEVGAIDADRYYHAQCQDLITDYVLWLTDNKVRPWGNAKDQINQSYGTGFKIHKNLPSTVAKTGWIAVFTSGTYSQYGHIGIVYSPGDTNKFHIIEQNWNGWANKKPLTRWDNYYGLTHFIEVPYAKETITTKKTAPKAATSKPAKKISVSHNNINLQSNFAKRGYNPKGVVIHNDAGRSTAQQYENSLKGANQNRLNNGIAHAYADSSHIWYAIEPERIAYHTGDGVKKGTGNYDYFGIEVCQSMSASDKEFMANEQVVLQEVARLLKKWKLPANRNTVRLHLEFSATSCPHRSLTMRTGFNPVTQGRPSDAIIKKLKDIWIKEIRAYMEGKTPSIKTVANKPGSASTPATKSNSDGWKVNAYNTYYKTERASFTVGSTRIAVRSTGPFTTCPFTGWLNPGVTITYDEVMVQNGFVWLGYDSYNGRRYVPIRTAQGTPGSSNYVLGPLWGTIS

SEQ ID NO: 15 - RNA, 1479 nt, synthetic construct
  misc_feature 1..1479: note = Nucleotide sequence encoding Peptidoglycan Hydrolase
  source 1..1479: mol_type = other RNA
atggctaagactaagaaacaaatcgagcaaagattgcaagcttatgctaaaggtactgttgattctccttacagaattactaagccaacttcttatgatccttctttcggtgttatggaagttggtgctattgatgctgatagatactatcatgctcaatgtcaagatttgatcactgattacgttttgtggttgactgataacaaagttagaccatggggtaacgctaaggatcaaattaatcaatcttacggtactggttttaagatccacaagaatttgccttctactgttgctaaaactggttggattgctgttttcacttctggtacttactctcaatatggtcatattggtattgtttactctccaggagatactaacaagttccacatcatcgaacaaaactggaacggttgggctaacaagaaaccattgactagatgggataactactacggtttgactcatttcatcgaagttccttacgctaaggagactatcactactaagaaaactgctccaaaggctgctacttctaaacctgctaagaaaatttctgtttctcataacaacattaacttgcaatctaattttgctaaaagaggttacaacccaaagggtgttgttattcacaatgatgctggtagatctactgctcaacaatacgaaaattctttgaagggtgctaaccaaaatagattgaacaatggtattgctcatgcttacgctgattcttctcacatttggtatgctattgaacctgagagaattgcttaccatactggagatggtgttaagaaaggtactggtaactacgattacttcggtatcgaagtttgtcaatctatgtctgcttctgataaagagtttatggctaatgagcaagttgttttgcaagaggttgctagattgttgaagaaatggaagttgccagctaacagaaacactgttagattgcatttggaattttctgctacttcttgtcctcacagatctttgactatgagaactggtttcaatccagttactcaaggtagaccttctgatgctattattaagaaattgaaggatatctggatcaaggaaatcagagcttacatggagggtaaaactccatctattaaaactgttgctaacaagccaggttctgcttctactcctgctactaaatctaattctgatggttggaaggttaacgcttacaacacttactacaagactgagagagcttcttttactgttggttctactagaattgctgttagatctactggtccattcactacttgtcctttcactggttggttgaacccaggtgttactatcacttacgatgaagttatggttcaaaacggtttcgtttggttgggttacgattcttataatggtagaagatacgttccaattagaactgctcaaggtactcctggttcttctaattatgttttgggtcctttgtggggtactatttcttaa

SEQ ID NO: 16 - RNA, 1479 nt, synthetic construct
  misc_feature 1..1479: note = Nucleotide sequence encoding Peptidoglycan Hydrolase
  source 1..1479: mol_type = other RNA
atggccaaaaccaagaaacagatagaacagagactgcaagcttacgcaaaggggaccgtggattccccttaccgaatcaccaagccaaccagctacgacccctccttcggtgtcatggaggttggcgctatagatgcggatcgctactatcatgcccagtgtcaagatctgataactgactatgtactttggctcactgacaacaaagtaagaccttggggtaatgccaaggaccagattaaccagagctatgggactggtttcaaaatccacaagaatctgcccagtaccgtggctaaaacaggatggattgccgtgtttacatccgggacctactcacagtacggccacatcggcatcgtgtacagtccaggagataccaacaaattccatatcatcgagcagaattggaatggatgggcaaacaagaaacctctgacacgttgggacaactactacgggctgacccactttatcgaggtcccatatgcgaaagagacaatcacaacgaagaaaactgcacctaaagcagctacctcaaaaccggctaagaagatctcagtttctcacaacaacatcaaccttcagagcaactttgcaaagagagggtacaacccaaagggagtggtgattcacaatgacgccggacgctctactgcgcaacagtatgaaaacagcctgaagggcgctaatcagaacaggctgaataacggcattgcccatgcatatgctgacagcagccacatttggtatgctatcgaacccgagagaatcgcctatcacacaggcgatggcgttaagaagggaacaggcaattacgactacttcggtattgaagtatgccagagcatgagcgcaagtgacaaagagttcatggccaacgaacaggtggtactgcaggaggtggctaggttgctcaagaaatggaagcttcccgccaatcggaataccgtgcggctccatctggaattctctgccacatcttgccctcataggagcctcacaatgcggaccggtttcaatccggtgactcagggtagaccctcagacgccatcatcaagaagttgaaggatatctggataaaagagattcgcgcatatatggagggtaaaaccccatccattaagactgtcgccaataagcccggatcagccagtacccctgcaaccaaatccaactccgatgggtggaaggtcaatgcctacaatacctactacaaaacggaacgggcctcctttacagtgggctctactcggattgctgtgcgtagtacaggaccctttactacatgcccgtttacagggtggctgaaccccggagtcacgataacctatgacgaggttatggtgcagaacgggtttgtgtggttgggctatgatagctacaatgggaggaggtatgtccccattcgcactgcccaaggaactcctggcagctccaattatgtgcttggccctctgtggggcacgatttcttga

SEQ ID NO: 17 - protein, 481 aa, synthetic construct
  REGION 1..481: note = Peptidoglycan Hydrolase
MQAKLTKKEFIEWLKTSEGKQFNADLWYGFQCFDYANAAWKVLFGLLLKGLGAKDIPFANNFDGLATVYQNTPDFLAQPGDMVVFGSNYGAGYGHVAWVIEATLDYIIVYEQNWLGGGWTDGIEQPGWGWEKVTRRQHAYDFPMWFIRPNFKSEIAPRSVQSPTQAPKKETAKPQPKAVELKIIKDVVKGYDLPKRGSNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNDSSASSNTVKPVASAWKRNKYGTYYMEESARFTNGNQPITVRKVGPFLYCPVGYQFQPGGYCDYTEVMLQDGHVWVGYTWEGQRYYLPIRTWNGSAPPNQILGDLWGEIS

SEQ ID NO: 18 - RNA, 1446 nt, synthetic construct
  misc_feature 1..1446: note = Nucleotide sequence encoding Peptidoglycan Hydrolase
  source 1..1446: mol_type = other RNA
atgcaggctaagttgactaagaaagagtttattgagtggttgaagacttctgaaggtaaacaattcaacgctgatttgtggtatggttttcaatgtttcgattacgctaatgctgcttggaaagttttgtttggtttgttgttgaagggtttgggtgctaaggatatccctttcgctaacaacttcgatggtttggctactgtttaccaaaacactccagatttcttggctcaacctggagatatggttgttttcggttctaattatggtgctggttacggtcatgttgcttgggttattgaagctactttggattacatcatcgtttacgagcaaaactggttgggtggtggttggactgatggtattgaacaaccaggttggggttgggagaaggttactagaagacaacacgcttatgattttccaatgtggttcatcagacctaacttcaagtctgaaatcgctcctagatctgttcaatctccaactcaagctcctaagaaagaaactgctaagccacaacctaaagctgttgagttgaagattattaaggatgttgttaagggttacgatttgccaaagagaggttctaaccctaagggtatcgttatccataacgatgctggttctaagggtgctactgctgaagcttatagaaacggtttggttaatgctccattgtccagattggaagctggtattgctcattcttacgtttctggtaacactgtttggcaagctttggatgaatctcaagttggttggcacactgctaaccaattgggtaataagtactattacggtattgaggtttgtcaatctatgggtgctgataacgctactttcttgaagaacgaacaagctactttccaagagtgtgctagattgttgaagaaatggggtttgccagctaacagaaacactatcagattgcacaacgagtttacttctacttcttgtcctcatagatcttctgttttgcacactggtttcgatccagttactagaggtttgttgcctgaggataagagattgcaattgaaggattacttcatcaagcaaatcagagcttacatggatggtaaaattccagttgctactgtttctaacgattcttctgcttcttctaatactgttaaacctgttgcttctgcttggaagagaaacaagtatggtacttattacatggaagagtctgctagattcactaacggtaatcaaccaattactgttagaaaggttggtccatttttgtattgtcctgttggttaccaattccaacctggtggttattgtgattacactgaagttatgttgcaagatggtcatgtttgggttggttacacttgggagggtcaaagatattacttgccaattagaacttggaacggttctgctccacctaatcaaattttgggagatttgtggggtgaaatttcttaa

SEQ ID NO: 19 - RNA, 1446 nt, synthetic construct
  misc_feature 1..1446: note = Nucleotide sequence encoding Peptidoglycan Hydrolase
  source 1..1446: mol_type = other RNA
atgcaagcgaaactgaccaagaaggagtttatagagtggctgaaaacctctgagggtaaacagttcaacgccgatctgtggtatggtttccagtgctttgattacgcaaacgctgcttggaaagtgctgtttggcctgctcctgaagggactcggtgccaaagacatcccctttgccaacaatttcgacgggctggcaaccgtctaccagaatactcctgactttctcgctcagccaggggacatggtcgtgttcggctccaattatggggcaggctatggacacgtagcatgggtgatagaagccactctggactacataatcgtctacgagcaaaattggcttggcggtgggtggaccgatggcattgagcagcctggctggggatgggagaaggttactcgtagacagcatgcttacgactttcccatgtggttcatccgtcccaacttcaagtccgagattgcgcccagaagcgtccagtctccaacccaagctccgaagaaggaaacagccaagccacagcctaaggccgttgaactgaagattattaaggatgtggtgaagggatatgacctcccgaaaagagggagcaatcccaaagggattgtgatccacaacgatgccgggtctaaaggggcaacagcagaagcgtataggaacgggttggtgaatgcccctctcagtcgcttggaggccggaattgcacactcatatgtgtccggaaatacggtttggcaagctctcgacgaaagccaagtaggctggcatacagccaaccagctgggcaacaaatactactatggcatcgaggtgtgccagagtatgggcgcagataatgccacgttcctgaagaacgagcaggctacctttcaagagtgtgcgcggttgctgaagaagtggggactgccagctaatcgcaacacaatcaggttgcacaacgagtttacgagtactagctgtccacacagaagtagcgtgctgcacacaggattcgatcctgtgacaaggggactgctgcccgaagataagcggctgcagctgaaagactacttcatcaaacagattcgggcctatatggacggaaagattcccgtcgctactgttagcaatgacagctctgcctcatcaaataccgtgaaaccggtagcaagcgcctggaaaaggaacaagtatggcacttattacatggaggaatccgcccgatttaccaacggtaatcagcccatcacagtccgcaaggtcgggccattcctgtactgtcccgtaggctaccagtttcagcctggtgggtactgcgactataccgaggtgatgctgcaagatggccatgtctgggttggctatacctgggaagggcagcgctactacttgccaatacggacttggaatggttctgccccacccaaccagatcctcggagatctgtggggcgaaatctcctga

SEQ ID NO: 20 - protein, 481 aa, synthetic construct
  REGION 1..481: note = Peptidoglycan Hydrolase
MQAKLTKKEFIEWLKTSEGKQFNVDLWYGFQCFDYANAGWKVLFGLLLKGLGAKDIPFANNFDGLATVYQNTPDFLAKPGDMVVFGSNYGAGYGHVAWVIEATLDYIIVYEQNWLGGGWTDRIEQPGWGWEKVTRRQHALRFPYVVYPSYPKSETAPRSIQSPTQASKKETAKPQPKAVELKIIKDVVKGYDLPKRGGNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNESSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 21 - RNA, 1446 nt, synthetic construct
  misc_feature 1..1446: note = Nucleotide sequence encoding Peptidoglycan Hydrolase
  source 1..1446: mol_type = other RNA
atgcaggctaagttgactaagaaagagtttattgagtggttgaagacttctgaaggtaaacaattcaacgttgatttgtggtacggttttcaatgtttcgattatgctaatgccggttggaaagttttgtttggtttgttgttgaagggtttgggtgctaaggatatccctttcgctaacaacttcgatggtttggctactgtttaccaaaacactccagatttcttggctaagcctggagatatggttgttttcggttctaattacggtgctggttatggtcatgttgcttgggttattgaagctactttggattacatcatcgtttacgagcaaaattggttgggtggtggttggactgatagaattgaacaaccaggttggggttgggagaaggttactagaagacaacacgctttgagattcccttatgttgtttacccatcttatcctaaatctgaaactgctccaagatctattcaatctcctactcaagcttctaagaaagaaactgctaagccacaacctaaagctgttgagttgaagattattaaggatgttgttaagggttacgatttgccaaagagaggtggtaaccctaagggtatcgttatccataacgatgctggttctaaaggtgctactgctgaagcttacagaaacggtttggttaatgctccattgtccagattggaagctggtattgctcattcttatgtttctggtaacactgtttggcaagctttggatgaatctcaagttggttggcacactgctaaccaattgggtaataagtactattacggtattgaggtttgtcaatctatgggtgctgataacgctactttcttgaagaacgaacaagctactttccaagagtgtgctagattgttgaagaaatggggtttgccagctaacagaaacactatcagattgcacaacgagtttacttctacttcttgtcctcatagatcttctgttttgcacactggtttcgatccagttactagaggtttgttgcctgaggataagagattgcaattgaaggattacttcatcaagcaaatcagagcttacatggatggtaaaatcccagttgctactgtttctaacgaatcttctgcttcttctaatactgttaaacctgttgcttctgcttggaagagaaacaaatacggtacttactacatggaagagtctgctagattcactaacggtaatcaaccaattactgttagaaaggttggtccatttttgtcttgtcctgttggttaccaattccaacctggtggttactgtgattatactgaagttatgttgcaagatggtcatgtttgggttggttatacttgggagggtcaaagatattacttgccaattagaacttggaacggttctgctccacctaatcaaattttgggagatttgtggggtgaaatttcttaa

SEQ ID NO: 22 - RNA, 1446 nt, synthetic construct
  misc_feature 1..1446: note = Nucleotide sequence encoding Peptidoglycan Hydrolase
  source 1..1446: mol_type = other RNA
atgcaggccaaactgaccaagaaggagttcatcgaatggctcaagacttccgagggcaagcagttcaatgtggacctctggtatggctttcaatgctttgattatgccaatgctgggtggaaggtcctgtttggccttctcctgaaaggactgggtgcaaaagacatccccttcgccaacaacttcgatggactggctaccgtttaccagaataccccggactttctggccaagcctggagatatggtggtgttcggaagcaactatggggcaggatatggccatgtagcctgggtgatagaagctacgttggactacatcatcgtgtacgagcaaaattggctgggcggcggctggactgaccgaattgagcaacctgggtggggatgggagaaagtgaccagacgtcagcacgctctgcgcttcccctatgtagtgtacccctcttacccgaagtctgagacagctccccggtccattcagagcccaacacaggcgagcaagaaagagactgccaaaccacaacccaaagcggttgaactgaagatcatcaaagacgtcgtgaaagggtatgatctgcccaagagaggcggaaatccaaaggggattgtgatccacaatgacgccgggagcaaaggtgcaactgctgaagcttaccgcaatgggctggtcaacgcacctttgtcccgactggaagccggcattgctcatagctatgtgtctggcaacactgtttggcaagcactggacgaatcacaggtcggctggcatactgccaatcagctcggaaacaagtactactacgggatagaggtctgtcagtccatgggcgccgataacgcgactttcctgaagaatgagcaggccacattccaggaatgcgctcggttgttgaagaaatggggccttcctgcaaacaggaacacaatcaggctgcacaatgagttcacatccacgtcttgtccacacaggtctagcgtgctgcacacaggttttgatcccgtgacgagagggcttctccccgaagataaacgtctgcagctcaaggattacttcatcaagcagataagggcctatatggacgggaaaattccggtagctaccgtcagcaacgagagtagcgcatcatccaacaccgttaagccagtggcctcagcctggaagcgcaacaaatacggtacctattacatggaggaatctgcaagattcacaaatggtaatcagcctattaccgtgcggaaagtcggcccttttctcagctgtcctgttggatatcagtttcagccaggcggttactgcgactataccgaagtaatgctgcaagatgggcatgtgtgggttgggtacacatgggaaggccaacggtactatctgcccattcgcacctggaacggcagtgccccacccaaccagatccttggagatctgtggggtgagatttcatga

SEQ ID NO: 23 - protein, 495 aa, synthetic construct
  REGION 1..495: note = Peptidoglycan Hydrolase
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 24 - RNA, 1488 nt, synthetic construct
  misc_feature 1..1488: note = Nucleotide sequence encoding Peptidoglycan Hydrolase
  source 1..1488: mol_type = other RNA
atggctaagactcaagctgagattaataagagattggatgcttacgctaagggtactgttgattctccatacagagttaagaaagctacttcttatgatccttctttcggtgttatggaagctggtgctattgatgctgatggttactatcatgctcaatgtcaagatttgatcactgattacgttttgtggttgactgataacaaggttagaacttggggtaacgctaaggatcaaattaagcaatcttacggtactggttttaaaatccatgaaaacaagccatctactgttcctaagaaaggttggattgctgttttcacttctggttcttacgagcaatggggtcacattggtattgtttacgatggtggtaacacttctacttttactattttggaacaaaactggaatggttacgctaacaagaaaccaactaagagagttgataactactacggtttgactcacttcattgaaattcctgttaaggctggtactactgttaagaaagagactgctaagaaatctgcttctaaaactccagctcctaagaaaaaggctactttgaaagtttctaagaaccatatcaactacactatggataagagaggtaaaaagccagagggtatggttattcacaacgatgctggtagatcttctggtcaacaatacgagaactctttggctaatgctggttacgctagatatgctaacggtattgctcattactatggttctgaaggttatgtttgggaggctattgatgctaaaaatcaaattgcttggcacactggagatggtactggtgctaactctggtaattttagattcgctggtattgaagtttgtcaatctatgtctgcttctgatgctcaatttttgaagaacgagcaagctgttttccaattcactgctgaaaaattcaaggagtggggtttgactcctaacagaaagactgttagattgcatatggaatttgttccaactgcttgtcctcatagatctatggttttgcacactggtttcaatccagttactcaaggtagaccttctcaagctatcatgaacaaattgaaggattacttcatcaagcaaattaagaattacatggataagggtacttcttcttctactgttgttaaagatggtaaaacttcttctgcttctactccagctactagacctgttactggttcttggaaaaagaaccaatacggtacttggtataaaccagagaatgctacttttgttaacggtaatcaacctattgttactagaattggttctccattcttgaacgctcctgttggtggtaatttgccagctggtgctactatcgtttacgatgaagtttgtatccaagctggtcatatttggattggttacaacgcttataacggtaatagagtttattgtccagttagaacttgtcaaggtgttccacctaatcaaattcctggtgttgcttggggtgttttcaagtaa

SEQ ID NO: 25 - RNA, 1488 nt, synthetic construct
  misc_feature 1..1488: note = Nucleotide sequence encoding Peptidoglycan Hydrolase
  source 1..1488: mol_type = other RNA
atggccaagacacaggccgagatcaacaagcggctggacgcctatgccaagggcacagtggatagcccctacagagtgaagaaggccaccagctacgaccccagcttcggcgttatggaagccggcgctattgacgccgacggctattatcatgcccagtgccaggacctgatcaccgactatgtgctgtggctgaccgacaacaaagtgcggacatggggcaacgccaaggaccagatcaagcagagctacggcaccggcttcaagatccacgagaacaagcctagcaccgtgcctaagaaaggctggatcgccgtgtttaccagcggcagctatgagcagtggggccacatcggcatcgtgtacgatggcggcaataccagcaccttcaccatcctggaacagaactggaacggctacgccaacaagaaacccaccaagagagtggacaactactacggcctgacacacttcatcgagatccccgtgaaggccggcaccaccgtgaagaaagagacagccaagaagtccgccagcaagacccctgctcctaagaagaaagccacactgaaggtgtccaagaaccacatcaactacaccatggacaagcggggcaaaaagcccgaaggcatggtcatccacaacgacgccggcagatctagcggccagcagtacgaaaacagcctggccaatgccggctatgccagatacgccaatggaatcgcccactactatggcagcgagggctatgtgtgggaagccatcgacgccaagaatcagatcgcctggcatacaggcgacggaaccggcgccaattccggcaatttcagattcgccggcatcgaagtgtgccagagcatgtctgccagcgacgcccagttcctgaagaatgagcaggccgtgttccagtttaccgccgagaagttcaaagagtggggactgacccctaacagaaagaccgtgcggctgcacatggaattcgtgcctaccgcatgtccccaccgctctatggtgctgcacaccggctttaaccccgtgacacagggcagacctagccaggccatcatgaacaagctgaaggactacttcattaagcagatcaagaactacatggataagggcaccagcagcagcaccgtggtcaaggatggcaagaccagcagcgcctctacaccagccacaagacctgtgacaggcagctggaagaagaaccagtacggcacctggtacaagcccgagaacgccaccttcgtgaacggcaaccagcctatcgtgaccagaatcggcagccccttcctgaatgcccctgtcggaggaaatcttcctgctggcgccaccatcgtgtatgacgaagtctgtattcaggccggccacatctggatcggctacaacgcctacaacggcaacagagtgtactgccccgtgcggacttgtcagggcgttccacctaatcagattccaggcgtggcctggggcgtgttcaaatga

SEQ ID NO: 26 - protein, 215 aa, synthetic construct
  REGION 1..215: note = Peptidoglycan Hydrolase
REAPKTQIYTVKKGDTLSAIALKYKTTVSNIQNTNNIANPNLIFIGQKLKVPMTPLVEPKPKTVSSNNKSNSNSSTLNYLKTLENRGWDFDGSYGWQCFDLVNVYWNHLYGHGLKGYGAKDIPYANNFNSEAKIYHNTPTFKAEPGDLVVFSGRFGGGYGHTAIVLNGDYDGKLMKFQSLDQNWNNGGWRKAEVAHKVVHNYENDMIFIRPFKKA

SEQ ID NO: 27 - RNA, 648 nt, synthetic construct
  misc_feature 1..648: note = Nucleotide sequence encoding Peptidoglycan Hydrolase
  source 1..648: mol_type = other RNA
agagaggctcctaagactcaaatctacactgttaagaaaggagatactttgtctgctatcgctttgaagtacaagactactgtttctaacatccaaaacactaacaacatcgctaacccaaatttgattttcattggtcaaaagttgaaagttccaatgactcctttggttgaaccaaagcctaagactgtttcttctaacaataagtctaactctaattcttctactttgaactacttgaagactttggagaatagaggttgggattttgatggttcttatggttggcaatgtttcgatttggttaacgtttactggaatcatttgtatggtcacggtttgaagggttacggtgctaaggatatcccttacgctaacaacttcaactctgaagctaagatctaccataatactccaactttcaaagctgagcctggagatttggttgttttctctggtagattcggtggtggttacggtcacactgctattgttttgaacggagattacgatggtaaattgatgaagttccaatctttggatcaaaattggaacaatggtggttggagaaaggctgaagttgctcataaggttgttcacaactacgagaacgatatgattttcattagaccattcaagaaagcttaa

SEQ ID NO: 28 - RNA, 648 nt, synthetic construct
  misc_feature 1..648: note = Nucleotide sequence encoding Peptidoglycan Hydrolase
  source 1..648: mol_type = other RNA
agagaggctcccaaaacacagatttacaccgtcaagaagggagatacgcttagcgccattgcactgaaatacaagaccaccgtatcaaacatccagaacaccaataacatagccaatcccaatctcatcttcattggccagaagctcaaggttcccatgactccactggtcgaaccgaaacctaagacagtgtcctccaataacaagagtaacagcaactcctctacactgaactatctcaagacgttggagaatcgcggttgggactttgacggcagttacggctggcaatgcttcgacttggtgaatgtgtactggaatcacctgtatgggcatggactgaaaggctatggggccaaagacataccttatgctaacaatttcaactctgaggccaagatctaccacaacactcctaccttcaaggcagaaccaggagatctggttgtgtttagcggacggtttggcggaggctatgggcatactgccattgtgcttaatggggattacgatggcaaactgatgaaattccagtcactggaccagaactggaacaatggtggttggcgaaaagcggaagtagcccacaaggtcgtccacaattacgagaacgacatgatcttcatccgtccctttaagaaagcgtga

SEQ ID NO: 29 - protein, 246 aa, synthetic construct
  REGION 1..246: note = Peptidoglycan Hydrolase
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 30 - RNA, 741 nt, synthetic construct
  misc_feature 1..741: note = Nucleotide sequence encoding Peptidoglycan Hydrolase
  source 1..741: mol_type = other RNA
gctgctactcatgagcactctgctcaatggttgaacaattacaagaaaggttacggttatggtccatatcctttgggtattaacggtggtatgcattacggtgttgatttctttatgaacatcggtactcctgttaaggctatttcttctggtaaaattgttgaagctggttggtctaactatggtggtggtaatcaaattggtttgattgagaacgatggtgttcatagacaatggtacatgcacttgtctaagtacaacgttaaggttggagattacgttaaggctggtcaaattattggttggtctggttctactggttattctactgctccacatttgcacttccaaagaatggttaactctttctctaattctactgctcaagatccaatgcctttcttgaagtctgctggttacggtaaagctggtggtactgttactccaactcctaacactggttggaagactaacaagtacggtactttgtacaagtctgaatctgcttcttttactccaaacactgatatcatcactagaactactggtccattcagatctatgcctcaatctggtgttttgaaggctggtcaaactatccattacgatgaggttatgaagcaagatggtcacgtttgggttggttacactggtaattctggtcaaagaatctacttgcctgttagaacttggaacaagtctactaatactttgggtgttttgtggggtactattaaataa

SEQ ID NO: 31 - RNA, 741 nt, synthetic construct
  misc_feature 1..741: note = Nucleotide sequence encoding Peptidoglycan Hydrolase
  source 1..741: mol_type = other RNA
gcagcaacgcatgagcattccgctcaatggctcaacaactacaagaagggttacggctatggcccctatcctctggggataaacggagggatgcactatggagtcgatttcttcatgaatattggcactccagtgaaagcgatttccagcgggaagattgtggaagctgggtggagcaactatggtggtggcaatcagatcggactgattgagaatgacggcgtgcacagacagtggtatatgcatctgagtaagtacaacgtcaaggtgggagactacgtcaaagccgggcagatcataggatggtccggctctacaggttactcaacagcgccacatctccactttcagcgcatggtgaatagcttcagcaattcaaccgcccaagatccgatgcccttcctgaagtctgccggctatgggaaagctggaggaacagtgacacctactcccaataccggatggaaaacgaacaagtacggcactctgtacaaatccgaatctgccagtttcacacccaataccgacatcatcacccgaaccactggaccttttcggtcaatgccgcagtctggagtactgaaagcagggcaaactatccactatgacgaggtgatgaagcaggatggccacgtatgggtggggtacacaggcaatagcggccagaggatttacttgccagtccgtacctggaacaaaagcacgaacactcttggcgttttgtggggcaccatcaagtga

SEQ ID NO: 32 - protein, 150 aa, synthetic construct
  REGION 1..150: note = Peptidoglycan Hydrolase
DQFLDEKEGNHTTAYRDGSGIWTICRGATMVDGKPVIPGMRLSKEKCDQVNAIERDKALAWVERNIKVPLTEPQKAGIASFCPYNIGPGKCFPSTFYKRLNAGDRKGACEAIRWWIKDGGRDCRIRSNNCYGQVIRRDQESALACWGIDQ

SEQ ID NO: 33 - RNA, 453 nt, synthetic construct
  misc_feature 1..453: note = Nucleotide sequence encoding Peptidoglycan Hydrolase
  source 1..453: mol_type = other RNA
gatcaattcttggatgaaaaagagggtaatcatactactgcttatagagatggttctggtatttggactatttgtagaggtgctactatggttgatggtaaaccagttattcctggtatgagattgtctaaggaaaagtgtgatcaagttaacgctattgagagagataaggctttggcttgggttgaaagaaatattaaggttccattgactgagcctcaaaaagctggtattgcttctttttgtccatacaacattggtcctggtaaatgtttcccatctactttctacaagagattgaatgctggagatagaaagggtgcttgtgaagctattagatggtggattaaggatggtggtagagattgtagaattagatctaacaactgttacggtcaagttattagaagagatcaagaatctgctttggcttgttggggtattgatcaataa

SEQ ID NO: 34 - RNA, 453 nt, synthetic construct
  misc_feature 1..453: note = Nucleotide sequence encoding Peptidoglycan Hydrolase
  source 1..453: mol_type = other RNA
gatcagtttctcgacgagaaggaaggcaatcacaccactgcgtatagggatggctctgggatatggaccatctgtagaggcgccacaatggtcgatggcaaacccgtgatacccggaatgcgtctgtcaaaggagaagtgcgatcaggtgaatgccatcgaacgcgacaaagctctggcctgggttgagcggaacatcaaggttccactgacggaaccacagaaagccggaattgcgtccttctgtccgtacaacattggccctggaaagtgctttccctccactttctacaagcgcctgaatgcaggggacagaaaaggagcctgtgaggctatcaggtggtggatcaaagatggaggtagggactgtcgaatccggagcaacaactgctatgggcaagtgattcggagagatcaggagagtgctctggcttgctggggtattgaccagtga

SEQ ID NO: 35 - protein, 287 aa, synthetic construct
  REGION 1..287: note = Peptidoglycan Hydrolase
MRYIPAAHHSAGSNKPVNRVVIHATCPDVGFPSASRKGRAVSTANYFASPSSGGSAHYVCDIGETVQCLSESTIGWHAPPNPHSLGIEICADGGSHASFRVPGHAYTREQWLDPRVWPAVERAAVLCRQLCDKHGVPKRKLSVADLKAGRRGICGHTDVTDAWHQSDHDDPGPWFPWDKFMAVVNGHGGGSSSEELTVADVKALHNQIKQLSAQLSGSVNKLHHDVGVVQVQNGDLSKRVDALSWVKNPVTGKLWRTKDALWSVWYYVLECRSRIDRLESAVNGLKK

SEQ ID NO: 36 - RNA, 864 nt, synthetic construct
  misc_feature 1..864: note = Nucleotide sequence encoding Peptidoglycan Hydrolase
  source 1..864: mol_type = other RNA
atgagatatattcctgctgctcatcactctgctggttctaacaagccagttaatagagttgttattcatgctacttgtccagatgttggttttccttctgcttccagaaaaggtagagctgtttctactgctaactacttcgcttctccttcttctggtggttctgctcattatgtttgtgatattggtgaaactgttcaatgtttgtctgagtctactattggttggcatgctccacctaatccacactctttgggtattgaaatttgtgctgatggtggttctcatgcttctttcagagttcctggtcacgcttacactagagaacaatggttggatccaagagtttggcctgctgttgagagagctgctgttttgtgtagacaattgtgtgataagcatggtgttccaaagagaaaattgtctgttgctgatttgaaagctggtagaagaggtatttgtggtcacactgatgttactgatgcttggcatcaatctgatcacgatgatccaggtccttggtttccttgggataagttcatggctgttgttaacggtcatggtggtggttcttcttctgaagagttgactgttgctgatgttaaggctttgcacaaccaaattaaacaattgtctgctcaattgtctggttccgttaacaaattgcatcacgatgttggtgttgttcaagttcaaaatggagatttgtctaagagagttgatgctttgtcttgggttaaaaacccagttactggtaaattgtggagaactaaagatgctttgtggtctgtttggtactatgttttggaatgtagatccagaattgatagattggagtctgctgttaatggtttgaagaaataa

SEQ ID NO: 37 - RNA, 864 nt, synthetic construct
  misc_feature 1..864: note = Nucleotide sequence encoding Peptidoglycan Hydrolase
  source 1..864: mol_type = other RNA
atgcgctatattcctgctgcacaccattctgcagggagcaacaagcccgtgaatcgcgttgtcatccatgccacttgtcccgatgtgggctttccctccgcctcacggaaaggaagggcagtgagcactgccaactacttcgccagtccgtcctcaggcgggtcagcccactatgtgtgcgacataggggagacagtacagtgtctcagcgaaagcacgattgggtggcatgccccaccgaatccacactctttggggatcgagatttgcgcagatggaggttctcatgcctcttttcgcgtgccaggccatgcatacaccagagagcagtggctcgatccacgagtttggcccgcagttgaacgagctgctgtgctgtgccggcaattgtgcgacaagcatggcgtacccaaacggaaactgagcgttgcggacctgaaagccgggaggaggggtatatgcggacataccgacgtgactgacgcgtggcaccagagtgaccacgatgaccctgggccttggtttccttgggacaagttcatggcggtggtaaacggacatggtggcggcagtagtagcgaggaactgaccgttgccgatgtcaaggccctgcacaaccagatcaagcagctgtctgctcaactctccggaagcgtgaacaagctgcaccacgatgtgggagtcgtccaagtgcagaatggcgacctctccaagagagtggatgccttgagctgggtcaagaatcccgttacaggcaaactttggcgtaccaaagatgccctgtggagcgtctggtactatgtactggagtgtcggtccaggatcgatagactggagtcagctgtcaatggtctgaagaagtga

SEQ ID NO: 38 - protein, 484 aa, synthetic construct
  REGION 1..484: note = Peptidoglycan Hydrolase
MLMTKNQAEKWFDNSLGKQFNPDGWYGFQCYDYANMFFMLATGERLQGLYAYNIPFDNKAKIEKYGQIIKNYDSFLPQKLDIVVFPSKYGGGAGHVEIVESANLNTFTSFGQNWNGKGWTNGVAQPGWGPETVTRHVHYYDNPMYFIRLNFPNNLSVGNKAKGIIKQATTKKEAVIKPKKIMLVAGHGYNDPGAVGNGTNERDFIRKYITPNIAKYLRHAGHEVALYGGSSQSQDMYQDTAYGVNVGNKKDYGLYWVKSQGYDIVLEIHLDAAGESASGGHVIISSQFNADTIDKSIQDVIKNNLGQIRGVTPRNDLLNVNVSAEININYRLSELGFITNKNDMDWIKKNYDLYSKLIAGAIHGKPIGGLVAGNVKTSAKNKKNPPVPAGYTLDKNNVPYKKEQGNYTVANVKGNNVRDGYSTNSRITGVLPNNTTITYDGAYCINGYRWITYIANSGQRRYIATGEVDKAGNRISSFGKFSTI

SEQ ID NO: 39 - RNA, 1455 nt, synthetic construct
  misc_feature 1..1455: note = Nucleotide sequence encoding Peptidoglycan Hydrolase
  source 1..1455: mol_type = other RNA
atgttgatgactaagaaccaagctgaaaaatggttcgataactctttgggtaaacaattcaatcctgatggttggtacggtttccaatgttacgattacgctaacatgtttttcatgttggctactggtgaaagattgcaaggtttgtacgcttacaacatcccattcgataataaggctaagatcgagaagtacggtcaaattattaagaactacgattctttcttgccacaaaagttggatatcgttgttttcccttctaaatacggtggtggtgctggtcatgttgaaattgttgagtctgctaacttgaatacttttacttctttcggtcaaaactggaatggtaaaggttggactaacggtgttgctcaaccaggttggggtcctgaaactgttactagacatgttcactactacgataacccaatgtacttcatcagattgaatttccctaacaatttgtctgttggtaataaggctaagggtattattaagcaagctactactaagaaagaggctgttattaaaccaaagaaaattatgttggttgctggtcacggttataacgatcctggtgctgttggtaacggtactaacgaaagagatttcattagaaagtacatcactcctaacatcgctaagtatttgagacatgctggtcacgaggttgctttgtacggtggttcttctcaatctcaagatatgtaccaagatactgcttatggtgttaacgttggtaataagaaagattacggtttgtactgggttaagtctcaaggttacgatatcgttttggaaatccatttggatgctgctggtgaatctgcttctggtggtcacgttatcatctcttctcaattcaacgctgatactatcgataagtctatccaagatgttattaaaaacaatttgggtcaaattagaggtgttactccaagaaacgatttgttgaacgttaacgtttctgctgaaattaacatcaactacagattgtctgagttgggttttattactaacaaaaacgatatggattggattaagaaaaactacgatttgtattctaagttgattgctggtgctattcatggtaaacctattggtggtttggttgctggtaacgttaagacttctgctaaaaacaagaaaaatccacctgttccagctggttacactttggataagaacaatgttccttacaagaaagaacagggtaactacactgttgctaacgttaagggtaacaacgttagagatggttactctactaactccagaattactggtgttttgccaaacaacactactatcacttacgatggtgcttactgtatcaacggttacagatggatcacttacatcgctaattctggtcaaagaagatatattgctactggtgaagttgataaggctggtaatagaatttcttcttttggtaaattctctactatctaa

SEQ ID NO: 40 - RNA, 1455 nt, synthetic construct
  misc_feature 1..1455: note = Nucleotide sequence encoding Peptidoglycan Hydrolase
  source 1..1455: mol_type = other RNA
atgctgatgaccaagaaccaggccgagaagtggttcgacaacagcctgggcaagcagttcaaccccgatggatggtacggcttccagtgctacgactacgccaacatgttcttcatgctggccaccggcgagagactgcagggcctgtacgcctacaacatccccttcgataacaaggccaagatcgagaagtacggccagatcatcaagaactacgacagcttcctgcctcagaaactggacatcgtggtgttccccagcaaatatggcggcggagccggacacgtggaaatcgtggaaagcgccaacctgaacaccttcaccagcttcggccagaactggaacggcaaaggctggaccaatggcgtggcacaacctggctggggacctgagacagtgacaagacacgtgcactactacgacaaccccatgtacttcatccggctgaacttccccaacaacctgagcgtgggcaacaaagccaagggcatcatcaagcaggccaccaccaagaaagaagccgtgatcaagcccaagaaaatcatgctggtggctggccacggctacaacgatcctggcgctgttggcaacggcaccaacgagagagacttcatcagaaagtacatcacgcccaatatcgccaagtacctgaggcacgccggacatgaggttgccctgtatggcggatctagccagagccaggacatgtaccaggataccgcctacggcgtgaacgtgggaaacaagaaggactacggcctgtactgggtcaagagccagggctacgacatcgtgctggaaatccacctggatgccgctggcgaatctgcctctggcggacatgtgatcatcagcagccagttcaatgccgacaccatcgacaagagcatccaggatgtgatcaagaacaacctgggccagatccggggcgtgacccctagaaacgatctgctgaatgtgaacgtgtccgccgagatcaacatcaactaccggctgagcgagctgggcttcatcaccaacaagaacgacatggactggatcaagaagaattacgacctctacagcaagctgatcgctggcgccatccacggcaagcctattggaggactggtggccggcaacgtgaaaaccagcgccaagaacaagaaaaaccctcctgtgcctgccggctacaccctggacaagaacaatgtgccctacaagaaagagcagggcaactacaccgtggccaacgtgaagggaaacaacgtgcgcgacggctacagcaccaacagcagaattacaggcgtgctgcccaacaacaccacaatcacctacgacggcgcctactgcatcaacggctatcggtggatcacctacattgccaactccggccagcggcggtatattgccacaggcgaagtggacaaggccggcaatagaatcagctccttcggcaagttcagcaccatctaa

SEQ ID NO: 41 - protein, 206 aa, synthetic construct
  REGION 1..206: note = Peptidoglycan Hydrolase
TQNYVVKKGDYLGAIAKKFNTTVSKIQKLNNIKNPNFILIGQVLKVPFISSNNSSTNTNRRNNSSSTLNYLNSLAYKGWDFDGAYGNQCFDLVNVYWNHLYGHGLRGYGAKDIPFANNFRGEATVYRNTPTFKAELGDVAVFSGRYGGGFGHTAIVLNGNYDGKLMKFQSLDQNWYGGDSRRADIAHKVVHNYDYDMYFIRPYKKA

SEQ ID NO: 42 - RNA, 621 nt, synthetic construct
  misc_feature 1..621: note = Nucleotide sequence encoding Peptidoglycan Hydrolase
  source 1..621: mol_type = other RNA
actcaaaactacgttgttaagaaaggagattatttgggtgctatcgctaagaaattcaatactactgtttctaagatccaaaagttgaacaacattaagaacccaaacttcatcttgatcggtcaagttttgaaagttcctttcatttcttctaacaactcttctactaacactaacagaagaaacaactcttcttctactttgaactacttgaactctttggcttataagggttgggattttgatggtgcttacggtaaccaatgtttcgatttggttaacgtttactggaatcatttgtatggtcacggtttgagaggttatggtgctaaggatattccatttgctaacaatttcagaggtgaagctactgtttacagaaacactcctacttttaaagctgagttgggagatgttgctgttttctctggtagatacggtggtggtttcggtcatactgctatcgttttgaacggtaactacgatggtaaattgatgaagttccaatctttggatcaaaactggtacggtggagattctcgtagagctgatatcgctcataaggttgttcacaactacgattacgatatgtacttcatcagaccttacaagaaagcttaa

SEQ ID NO: 43 - RNA, 621 nt, synthetic construct
  misc_feature 1..621: note = Nucleotide sequence encoding Peptidoglycan Hydrolase
  source 1..621: mol_type = other RNA
acacagaattacgtggtgaagaagggcgattacctgggtgcgatcgccaagaagttcaacaccacagtgagcaagatccagaaactcaacaacatcaagaatcccaacttcatcctgattggccaagtgctgaaagtaccgttcataagctctaacaactcaagcaccaatacgaaccgccggaacaactccagtagcacactgaactacctgaattccctcgcatacaaagggtgggatttcgatggagcctatggcaatcagtgttttgacctcgtcaacgtgtactggaatcacctctatggtcatgggctgagagggtacggtgctaaggacataccttttgccaataattttcgcggcgaagctactgtgtatcggaataccccaaccttcaaagcagagcttggagatgttgctgttttctcagggaggtatggaggaggctttggccatactgccattgtcctgaatggcaactatgacgggaaactgatgaaattccagtctttggaccagaattggtatggcggggatagtagacgtgccgatattgcgcacaaggttgtccacaactatgactacgacatgtactttatccgaccttacaagaaggcctga

SEQ ID NO: 44 - protein, 291 aa, synthetic construct
  REGION 1..291: note = Peptidoglycan Hydrolase
MNKVDAWLSKHDLKNLTTLIVVGAFILFLILLFMLMNHSDQGSNRITVSENAELRTDPNAAYPVIYQVEKGDTFTRLSKSGKWIEVESRDGSEKGWIAGWHTSLNIEADSNKKVNPLKNKVIVLDPGHGGNDQGASSNTSKKSLEKNYTLKTAKELRRALEKAGATVKLTRSDDTYVSLDDRKLNGDAYISIHNDSLDSPNANGATVYWYKNNQEALAETLNTNIQKKAMLSNRGTRQENFQVLRQTNKPAVLLELGYISNPTDEVMIRNQIHRQVVEEAVVDGLKQYFAS

SEQ ID NO: 45 - RNA, 876 nt, synthetic construct
  misc_feature 1..876: note = Nucleotide sequence encoding Peptidoglycan Hydrolase
  source 1..876: mol_type = other RNA
atgaacaaggttgatgcttggttgtctaagcatgatttgaaaaatttgactactttgatcgttgttggtgcttttattttgttcttgatcttgttgtttatgttgatgaaccactctgatcaaggttctaacagaattactgtttctgaaaatgctgagttgagaactgatccaaatgctgcttacccagttatctaccaagttgaaaagggagatactttcactagattgtctaagtctggtaaatggattgaagttgagtccagagatggttctgaaaaaggttggattgctggttggcatacttctttgaacattgaggctgattctaacaagaaagttaacccattgaagaacaaagttattgttttggatcctggtcacggtggtaacgatcaaggtgcttcttctaatacttctaagaagtctttggaaaagaactacactttgaagactgctaaagaattgcgtagagctttggaaaaggctggtgctactgttaagttgactagatctgatgatacttacgtttctttggatgatagaaagttgaacggagatgcttacatctctatccataacgattctttggattctcctaacgctaatggtgctactgtttactggtacaagaacaaccaagaagctttggctgagactttgaacactaacatccaaaagaaagctatgttgtctaacagaggtactagacaagaaaacttccaagttttgagacaaactaacaagccagctgttttgttggaattgggttacatttctaaccctactgatgaggttatgattagaaatcaaatccacagacaagttgttgaagaggctgttgttgatggtttgaaacaatatttcgcttcttaa

SEQ ID NO: 46 - RNA, 876 nt, synthetic construct
  misc_feature 1..876: note = Nucleotide sequence encoding Peptidoglycan Hydrolase
  source 1..876: mol_type = other RNA
atgaataaggtggatgcttggctcagcaaacacgaccttaagaacctcactaccttgatcgttgtaggagccttcattctgttcctgatactgctgttcatgctgatgaatcactctgatcaggggtcaaaccgcataactgtttccgagaatgccgaacttcggacagatccaaacgccgcctatccggtcatctatcaggtcgagaaaggggacactttcaccagactgtctaaaagcggcaaatggattgaggtcgagagtagagatgggagtgagaaggggtggatagcaggatggcacacatccctgaacattgaagccgatagcaacaagaaagtgaaccctctcaagaacaaagtgatcgtgctggatcctggtcatggtggcaacgatcagggcgcttcaagcaataccagcaagaagtcactggagaagaattacacgctgaaaacagccaaggaactgcgacgggctcttgagaaagcaggagctacggtgaaactgacacggtctgacgacacctatgtgagtctggacgacaggaagctgaatggcgacgcgtatatctccattcacaatgacagcctggattctcccaatgcaaatggggctaccgtctactggtacaagaacaaccaagaggccctggcagaaaccctcaataccaacatccagaagaaggcgatgttgtccaataggggtactaggcaggaaaactttcaggtgcttagacagacaaacaaacccgcagttctgttggaactcggctacatctccaacccaacagacgaggtgatgatccgcaatcagattcaccgtcaagtagtggaagaagcagtggtcgatggcctgaaacagtactttgccagttga

SEQ ID NO: 47 - protein, 27 aa, synthetic construct
  REGION 1..27: note = Secretion signal
MGGAAARLGAVILFVVIVGLHGVRSKY

SEQ ID NO: 48 - protein, 25 aa, synthetic construct
  REGION 1..25: note = Secretion signal
MGRLTSGVGTAALLVVAVGLRVVCA

SEQ ID NO: 49 - protein, 28 aa, synthetic construct
  REGION 1..28: note = Secretion signal
MGRLTSGVGTAALLVVAVGLRVVCAKYA

SEQ ID NO: 50 - protein, 19 aa, synthetic construct
  REGION 1..19: note = Secretion signal
MFVFLVLLPLVSSQCVNLT

SEQ ID NO: 51 - protein, 21 aa, synthetic construct
  REGION 1..21: note = Secretion signal
MDWIWRILFLVGAATGAHSQM

SEQ ID NO: 52 - protein, 19 aa, synthetic construct
  REGION 1..19: note = Secretion signal
MDWTWRVFCLLAVAPGAHS

SEQ ID NO: 53 - protein, 20 aa, synthetic construct
  REGION 1..20: note = Secretion signal
METPAQLLFLLLLWLPDTTG

SEQ ID NO: 54 - protein, 18 aa, synthetic construct
  REGION 1..18: note = Secretion signal
MDWTWILFLVAAATRVHS

SEQ ID NO: 55 - protein, 24 aa, synthetic construct
  REGION 1..24: note = Secretion signal
MLGSNSGQRVVFTILLLLVAPAYS

SEQ ID NO: 56 - protein, 17 aa, synthetic construct
  REGION 1..17: note = Secretion signal
MKCLLYLAFLFIGVNCA

SEQ ID NO: 57 - protein, 18 aa, synthetic construct
  REGION 1..18: note = Secretion signal
MDWTWILFLVAAATRVHS

SEQ ID NO: 58 - protein, 19 aa, synthetic construct
  REGION 1..19: note = Secretion signal
ETPAQLLFLLLLWLPDTTG

SEQ ID NO: 59 - protein, 24 aa, synthetic construct
  REGION 1..24: note = Secretion signal
MLGSNSGQRVVFTILLLLVAPAYS

SEQ ID NO: 60 - protein, 17 aa, synthetic construct
  REGION 1..17: note = Secretion signal
MKCLLYLAFLFIGVNCA

SEQ ID NO: 61 - protein, 15 aa, synthetic construct
  REGION 1..15: note = Secretion signal
MWLVSLAIVTACAGA

SEQ ID NO: 62 - protein, 15 aa, synthetic construct
  REGION 1..15: note = Secretion signal
MFVFLVLLPLVSSQC

SEQ ID NO: 63 - protein, 595 aa, synthetic construct
  REGION 1..595: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMAKTKKQIEQRLQAYAKGTVDSPYRITKPTSYDPSFGVMEVGAIDADRYYHAQCQDLITDYVLWLTDNKVRPWGNAKDQINQSYGTGFKIHKNLPSTVAKTGWIAVFTSGTYSQYGHIGIVYSPGDTNKFHIIEQNWNGWANKKPLTRWDNYYGLTHFIEVPYAKETITTKKTAPKAATSKPAKKISVSHNNINLQSNFAKRGYNPKGVVIHNDAGRSTAQQYENSLKGANQNRLNNGIAHAYADSSHIWYAIEPERIAYHTGDGVKKGTGNYDYFGIEVCQSMSASDKEFMANEQVVLQEVARLLKKWKLPANRNTVRLHLEFSATSCPHRSLTMRTGFNPVTQGRPSDAIIKKLKDIWIKEIRAYMEGKTPSIKTVANKPGSASTPATKSNSDGWKVNAYNTYYKTERASFTVGSTRIAVRSTGPFTTCPFTGWLNPGVTITYDEVMVQNGFVWLGYDSYNGRRYVPIRTAQGTPGSSNYVLGPLWGTIS

SEQ ID NO: 64 - DNA, 1788 bp, synthetic construct
  misc_feature 1..1788: note = Nucleotide sequence encoding Peptidoglycan Hydrolase
  source 1..1788: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgcctttctctaactctactaacaacggtttgttgtttattaacactactatcgcttctattgctgctaaagaagagggtgtttctttggaaaagagagaagctgaggctcatcaccatcaccatcacttggaggttttgttccaaggtccaatggctaagactaagaaacaaatcgagcaaagattgcaagcttatgctaaaggtactgttgattctccttacagaattactaagccaacttcttatgatccttctttcggtgttatggaagttggtgctattgatgctgatagatactatcatgctcaatgtcaagatttgatcactgattacgttttgtggttgactgataacaaagttagaccatggggtaacgctaaggatcaaattaatcaatcttacggtactggttttaagatccacaagaatttgccttctactgttgctaaaactggttggattgctgttttcacttctggtacttactctcaatatggtcatattggtattgtttactctccaggagatactaacaagttccacatcatcgaacaaaactggaacggttgggctaacaagaaaccattgactagatgggataactactacggtttgactcatttcatcgaagttccttacgctaaggagactatcactactaagaaaactgctccaaaggctgctacttctaaacctgctaagaaaatttctgtttctcataacaacattaacttgcaatctaattttgctaaaagaggttacaacccaaagggtgttgttattcacaatgatgctggtagatctactgctcaacaatacgaaaattctttgaagggtgctaaccaaaatagattgaacaatggtattgctcatgcttacgctgattcttctcacatttggtatgctattgaacctgagagaattgcttaccatactggagatggtgttaagaaaggtactggtaactacgattacttcggtatcgaagtttgtcaatctatgtctgcttctgataaagagtttatggctaatgagcaagttgttttgcaagaggttgctagattgttgaagaaatggaagttgccagctaacagaaacactgttagattgcatttggaattttctgctacttcttgtcctcacagatctttgactatgagaactggtttcaatccagttactcaaggtagaccttctgatgctattattaagaaattgaaggatatctggatcaaggaaatcagagcttacatggagggtaaaactccatctattaaaactgttgctaacaagccaggttctgcttctactcctgctactaaatctaattctgatggttggaaggttaacgcttacaacacttactacaagactgagagagcttcttttactgttggttctactagaattgctgttagatctactggtccattcactacttgtcctttcactggttggttgaacccaggtgttactatcacttacgatgaagttatggttcaaaacggtttcgtttggttgggttacgattcttataatggtagaagatacgttccaattagaactgctcaaggtactcctggttcttctaattatgttttgggtcctttgtggggtactatttcttaa

SEQ ID NO: 65 - protein, 584 aa, synthetic construct
  REGION 1..584: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMQAKLTKKEFIEWLKTSEGKQFNADLWYGFQCFDYANAAWKVLFGLLLKGLGAKDIPFANNFDGLATVYQNTPDFLAQPGDMVVFGSNYGAGYGHVAWVIEATLDYIIVYEQNWLGGGWTDGIEQPGWGWEKVTRRQHAYDFPMWFIRPNFKSEIAPRSVQSPTQAPKKETAKPQPKAVELKIIKDVVKGYDLPKRGSNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNDSSASSNTVKPVASAWKRNKYGTYYMEESARFTNGNQPITVRKVGPFLYCPVGYQFQPGGYCDYTEVMLQDGHVWVGYTWEGQRYYLPIRTWNGSAPPNQILGDLWGEIS

SEQ ID NO: 66 - DNA, 1755 bp, synthetic construct
  misc_feature 1..1755: note = Nucleotide sequence encoding Peptidoglycan Hydrolase
  source 1..1755: mol_type = other DNA
atgagatttccatctattttcactgctgttttgtttgctgcttcttctgctttggctgctccagttaacactactactgaagatgagactgctcaaattcctgctgaagctgttattggttattctgatttggagggagattttgatgttgctgttttgccattctctaactctactaacaacggtttgttgtttattaacactactatcgcttctattgctgctaaggaagagggtgtttctttggaaaagagagaagctgaggctcatcaccatcaccatcacttggaggttttgttccaaggtcctatgcaggctaagttgactaagaaagagtttattgagtggttgaagacttctgaaggtaaacaattcaacgctgatttgtggtatggttttcaatgtttcgattacgctaatgctgcttggaaagttttgtttggtttgttgttgaagggtttgggtgctaaggatatccctttcgctaacaacttcgatggtttggctactgtttaccaaaacactccagatttcttggctcaacctggagatatggttgttttcggttctaattatggtgctggttacggtcatgttgcttgggttattgaagctactttggattacatcatcgtttacgagcaaaactggttgggtggtggttggactgatggtattgaacaaccaggttggggttgggagaaggttactagaagacaacacgcttatgattttccaatgtggttcatcagacctaacttcaagtctgaaatcgctcctagatctgttcaatctccaactcaagctcctaagaaagaaactgctaagccacaacctaaagctgttgagttgaagattattaaggatgttgttaagggttacgatttgccaaagagaggttctaaccctaagggtatcgttatccataacgatgctggttctaagggtgctactgctgaagcttatagaaacggtttggttaatgctccattgtccagattggaagctggtattgctcattcttacgtttctggtaacactgtttggcaagctttggatgaatctcaagttggttggcacactgctaaccaattgggtaataagtactattacggtattgaggtttgtcaatctatgggtgctgataacgctactttcttgaagaacgaacaagctactttccaagagtgtgctagattgttgaagaaatggggtttgccagctaacagaaacactatcagattgcacaacgagtttacttctacttcttgtcctcatagatcttctgttttgcacactggtttcgatccagttactagaggtttgttgcctgaggataagagattgcaattgaaggattacttcatcaagcaaatcagagcttacatggatggtaaaattccagttgctactgtttctaacgattcttctgcttcttctaatactgttaaacctgttgcttctgcttggaagagaaacaagtatggtacttattacatggaagagtctgctagattcactaacggtaatcaaccaattactgttagaaaggttggtccatttttgtattgtcctgttggttaccaattccaacctggtggttattgtgattacactgaagttatgttgcaagatggtcatgtttgggttggttacacttgggagggtcaaagatattacttgccaattagaacttggaacggttctgctccacctaatcaaattttgggagatttgtggggtgaaatttcttaa

SEQ ID NO: 67 - protein, 584 aa, synthetic construct
  REGION 1..584: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMQAKLTKKEFIEWLKTSEGKQFNVDLWYGFQCFDYANAGWKVLFGLLLKGLGAKDIPFANNFDGLATVYQNTPDFLAKPGDMVVFGSNYGAGYGHVAWVIEATLDYIIVYEQNWLGGGWTDRIEQPGWGWEKVTRRQHALRFPYVVYPSYPKSETAPRSIQSPTQASKKETAKPQPKAVELKIIKDVVKGYDLPKRGGNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNESSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 68 - DNA, 1755 bp, synthetic construct
  misc_feature 1..1755: note = Nucleotide sequence encoding Peptidoglycan Hydrolase
  source 1..1755: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgccattctctaactctactaacaacggtttgttgtttattaacactactatcgcttctattgctgctaaggaagagggtgtttctttggaaaagagagaagctgaggctcatcaccatcaccatcacttggaggttttgttccaaggtcctatgcaggctaagttgactaagaaagagtttattgagtggttgaagacttctgaaggtaaacaattcaacgttgatttgtggtacggttttcaatgtttcgattatgctaatgccggttggaaagttttgtttggtttgttgttgaagggtttgggtgctaaggatatccctttcgctaacaacttcgatggtttggctactgtttaccaaaacactccagatttcttggctaagcctggagatatggttgttttcggttctaattacggtgctggttatggtcatgttgcttgggttattgaagctactttggattacatcatcgtttacgagcaaaattggttgggtggtggttggactgatagaattgaacaaccaggttggggttgggagaaggttactagaagacaacacgctttgagattcccttatgttgtttacccatcttatcctaaatctgaaactgctccaagatctattcaatctcctactcaagcttctaagaaagaaactgctaagccacaacctaaagctgttgagttgaagattattaaggatgttgttaagggttacgatttgccaaagagaggtggtaaccctaagggtatcgttatccataacgatgctggttctaaaggtgctactgctgaagcttacagaaacggtttggttaatgctccattgtccagattggaagctggtattgctcattcttatgtttctggtaacactgtttggcaagctttggatgaatctcaagttggttggcacactgctaaccaattgggtaataagtactattacggtattgaggtttgtcaatctatgggtgctgataacgctactttcttgaagaacgaacaagctactttccaagagtgtgctagattgttgaagaaatggggtttgccagctaacagaaacactatcagattgcacaacgagtttacttctacttcttgtcctcatagatcttctgttttgcacactggtttcgatccagttactagaggtttgttgcctgaggataagagattgcaattgaaggattacttcatcaagcaaatcagagcttacatggatggtaaaatcccagttgctactgtttctaacgaatcttctgcttcttctaatactgttaaacctgttgcttctgcttggaagagaaacaaatacggtacttactacatggaagagtctgctagattcactaacggtaatcaaccaattactgttagaaaggttggtccatttttgtcttgtcctgttggttaccaattccaacctggtggttactgtgattatactgaagttatgttgcaagatggtcatgtttgggttggttatacttgggagggtcaaagatattacttgccaattagaacttggaacggttctgctccacctaatcaaattttgggagatttgtggggtgaaatttcttaa

SEQ ID NO: 69 - protein, 598 aa, synthetic construct
  REGION 1..598: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 70 - DNA, 1797 bp, synthetic construct
  misc_feature 1..1797: note = Nucleotide sequence encoding Peptidoglycan Hydrolase
  source 1..1797: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgccattctctaactctactaacaacggtttgttgtttattaacactactatcgcttctattgctgctaaggaagagggtgtttctttggagaaaagagaagctgaggctcatcaccatcaccatcacttggaagttttgttccaaggtcctatggctaagactcaagctgagattaataagagattggatgcttacgctaagggtactgttgattctccatacagagttaagaaagctacttcttatgatccttctttcggtgttatggaagctggtgctattgatgctgatggttactatcatgctcaatgtcaagatttgatcactgattacgttttgtggttgactgataacaaggttagaacttggggtaacgctaaggatcaaattaagcaatcttacggtactggttttaaaatccatgaaaacaagccatctactgttcctaagaaaggttggattgctgttttcacttctggttcttacgagcaatggggtcacattggtattgtttacgatggtggtaacacttctacttttactattttggaacaaaactggaatggttacgctaacaagaaaccaactaagagagttgataactactacggtttgactcacttcattgaaattcctgttaaggctggtactactgttaagaaagagactgctaagaaatctgcttctaaaactccagctcctaagaaaaaggctactttgaaagtttctaagaaccatatcaactacactatggataagagaggtaaaaagccagagggtatggttattcacaacgatgctggtagatcttctggtcaacaatacgagaactctttggctaatgctggttacgctagatatgctaacggtattgctcattactatggttctgaaggttatgtttgggaggctattgatgctaaaaatcaaattgcttggcacactggagatggtactggtgctaactctggtaattttagattcgctggtattgaagtttgtcaatctatgtctgcttctgatgctcaatttttgaagaacgagcaagctgttttccaattcactgctgaaaaattcaaggagtggggtttgactcctaacagaaagactgttagattgcatatggaatttgttccaactgcttgtcctcatagatctatggttttgcacactggtttcaatccagttactcaaggtagaccttctcaagctatcatgaacaaattgaaggattacttcatcaagcaaattaagaattacatggataagggtacttcttcttctactgttgttaaagatggtaaaacttcttctgcttctactccagctactagacctgttactggttcttggaaaaagaaccaatacggtacttggtataaaccagagaatgctacttttgttaacggtaatcaacctattgttactagaattggttctccattcttgaacgctcctgttggtggtaatttgccagctggtgctactatcgtttacgatgaagtttgtatccaagctggtcatatttggattggttacaacgcttataacggtaatagagtttattgtccagttagaacttgtcaaggtgttccacctaatcaaattcctggtgttgcttggggtgttttcaagtaa

SEQ ID NO: 71 - protein, 318 aa, synthetic construct
  REGION 1..318: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPREAPKTQIYTVKKGDTLSAIALKYKTTVSNIQNTNNIANPNLIFIGQKLKVPMTPLVEPKPKTVSSNNKSNSNSSTLNYLKTLENRGWDFDGSYGWQCFDLVNVYWNHLYGHGLKGYGAKDIPYANNFNSEAKIYHNTPTFKAEPGDLVVFSGRFGGGYGHTAIVLNGDYDGKLMKFQSLDQNWNNGGWRKAEVAHKVVHNYENDMIFIRPFKKA

SEQ ID NO: 72 - DNA, 957 bp, synthetic construct
  misc_feature 1..957: note = Nucleotide sequence encoding Peptidoglycan Hydrolase
  source 1..957: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgcctttctctaactctactaacaacggtttgttgtttattaacactactatcgcttctattgctgctaaggaagagggtgtttctttggaaaagagagaagctgaggctcatcaccatcaccatcacttggaagttttgttccaaggtccaagagaggctcctaagactcaaatctacactgttaagaaaggagatactttgtctgctatcgctttgaagtacaagactactgtttctaacatccaaaacactaacaacatcgctaacccaaatttgattttcattggtcaaaagttgaaagttccaatgactcctttggttgaaccaaagcctaagactgtttcttctaacaataagtctaactctaattcttctactttgaactacttgaagactttggagaatagaggttgggattttgatggttcttatggttggcaatgtttcgatttggttaacgtttactggaatcatttgtatggtcacggtttgaagggttacggtgctaaggatatcccttacgctaacaacttcaactctgaagctaagatctaccataatactccaactttcaaagctgagcctggagatttggttgttttctctggtagattcggtggtggttacggtcacactgctattgttttgaacggagattacgatggtaaattgatgaagttccaatctttggatcaaaattggaacaatggtggttggagaaaggctgaagttgctcataaggttgttcacaactacgagaacgatatgattttcattagaccattcaagaaagcttaa

SEQ ID NO: 73 - protein, 349 aa, synthetic construct
  REGION 1..349: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 74 - DNA, 1050 bp, synthetic construct
  misc_feature 1..1050: note = Nucleotide sequence encoding Peptidoglycan Hydrolase
  source 1..1050: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgcctttctctaactctactaacaacggtttgttgtttattaacactactatcgcttctattgctgctaaggaagagggtgtttctttggaaaagagagaagctgaggctcatcaccatcaccatcacttggaagttttgttccaaggtccagctgctactcatgagcactctgctcaatggttgaacaattacaagaaaggttacggttatggtccatatcctttgggtattaacggtggtatgcattacggtgttgatttctttatgaacatcggtactcctgttaaggctatttcttctggtaaaattgttgaagctggttggtctaactatggtggtggtaatcaaattggtttgattgagaacgatggtgttcatagacaatggtacatgcacttgtctaagtacaacgttaaggttggagattacgttaaggctggtcaaattattggttggtctggttctactggttattctactgctccacatttgcacttccaaagaatggttaactctttctctaattctactgctcaagatccaatgcctttcttgaagtctgctggttacggtaaagctggtggtactgttactccaactcctaacactggttggaagactaacaagtacggtactttgtacaagtctgaatctgcttcttttactccaaacactgatatcatcactagaactactggtccattcagatctatgcctcaatctggtgttttgaaggctggtcaaactatccattacgatgaggttatgaagcaagatggtcacgtttgggttggttacactggtaattctggtcaaagaatctacttgcctgttagaacttggaacaagtctactaatactttgggtgttttgtggggtactattaaataa

SEQ ID NO: 75 - protein, 253 aa, synthetic construct
  REGION 1..253: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPDQFLDEKEGNHTTAYRDGSGIWTICRGATMVDGKPVIPGMRLSKEKCDQVNAIERDKALAWVERNIKVPLTEPQKAGIASFCPYNIGPGKCFPSTFYKRLNAGDRKGACEAIRWWIKDGGRDCRIRSNNCYGQVIRRDQESALACWGIDQ

SEQ ID NO: 76 - DNA, 762 bp, synthetic construct
  misc_feature 1..762: note = Nucleotide sequence encoding Peptidoglycan Hydrolase
  source 1..762: mol_type = other DNA
atgagatttccttctattttcactgctgttttgtttgctgcttcttctgctttggctgctcctgttaacactactactgaagatgagactgctcaaattccagctgaagctgttattggttactctgatttggagggagattttgatgttgctgttttgcctttctctaactctactaacaacggtttgttgttcattaacactactatcgcttctatcgctgctaaggaagagggtgtttctttggaaaagagagaagctgaggctcatcaccatcaccatcacttggaggttttgtttcaaggtccagatcaattcttggatgaaaaagagggtaatcatactactgcttatagagatggttctggtatttggactatttgtagaggtgctactatggttgatggtaaaccagttattcctggtatgagattgtctaaggaaaagtgtgatcaagttaacgctattgagagagataaggctttggcttgggttgaaagaaatattaaggttccattgactgagcctcaaaaagctggtattgcttctttttgtccatacaacattggtcctggtaaatgtttcccatctactttctacaagagattgaatgctggagatagaaagggtgcttgtgaagctattagatggtggattaaggatggtggtagagattgtagaattagatctaacaactgttacggtcaagttattagaagagatcaagaatctgctttggcttgttggggtattgatcaataa

SEQ ID NO: 77 - protein, 390 aa, synthetic construct
  REGION 1..390: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMRYIPAAHHSAGSNKPVNRVVIHATCPDVGFPSASRKGRAVSTANYFASPSSGGSAHYVCDIGETVQCLSESTIGWHAPPNPHSLGIEICADGGSHASFRVPGHAYTREQWLDPRVWPAVERAAVLCRQLCDKHGVPKRKLSVADLKAGRRGICGHTDVTDAWHQSDHDDPGPWFPWDKFMAVVNGHGGGSSSEELTVADVKALHNQIKQLSAQLSGSVNKLHHDVGVVQVQNGDLSKRVDALSWVKNPVTGKLWRTKDALWSVWYYVLECRSRIDRLESAVNGLKK

SEQ ID NO: 78 - DNA, 1173 bp, synthetic construct
  misc_feature 1..1173: note = Nucleotide sequence encoding Peptidoglycan Hydrolase
  source 1..1173: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgcctttctctaactctactaacaacggtttgttgtttattaacactactatcgcttctattgctgctaaggaagagggtgtttctttggaaaagagagaagctgaggctcatcaccatcaccatcacttggaggttttgttccaaggtccaatgagatatattcctgctgctcatcactctgctggttctaacaagccagttaatagagttgttattcatgctacttgtccagatgttggttttccttctgcttccagaaaaggtagagctgtttctactgctaactacttcgcttctccttcttctggtggttctgctcattatgtttgtgatattggtgaaactgttcaatgtttgtctgagtctactattggttggcatgctccacctaatccacactctttgggtattgaaatttgtgctgatggtggttctcatgcttctttcagagttcctggtcacgcttacactagagaacaatggttggatccaagagtttggcctgctgttgagagagctgctgttttgtgtagacaattgtgtgataagcatggtgttccaaagagaaaattgtctgttgctgatttgaaagctggtagaagaggtatttgtggtcacactgatgttactgatgcttggcatcaatctgatcacgatgatccaggtccttggtttccttgggataagttcatggctgttgttaacggtcatggtggtggttcttcttctgaagagttgactgttgctgatgttaaggctttgcacaaccaaattaaacaattgtctgctcaattgtctggttccgttaacaaattgcatcacgatgttggtgttgttcaagttcaaaatggagatttgtctaagagagttgatgctttgtcttgggttaaaaacccagttactggtaaattgtggagaactaaagatgctttgtggtctgtttggtactatgttttggaatgtagatccagaattgatagattggagtctgctgttaatggtttgaagaaataa

SEQ ID NO: 79 - protein, 587 aa, synthetic construct
  REGION 1..587: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMLMTKNQAEKWFDNSLGKQFNPDGWYGFQCYDYANMFFMLATGERLQGLYAYNIPFDNKAKIEKYGQIIKNYDSFLPQKLDIVVFPSKYGGGAGHVEIVESANLNTFTSFGQNWNGKGWTNGVAQPGWGPETVTRHVHYYDNPMYFIRLNFPNNLSVGNKAKGIIKQATTKKEAVIKPKKIMLVAGHGYNDPGAVGNGTNERDFIRKYITPNIAKYLRHAGHEVALYGGSSQSQDMYQDTAYGVNVGNKKDYGLYWVKSQGYDIVLEIHLDAAGESASGGHVIISSQFNADTIDKSIQDVIKNNLGQIRGVTPRNDLLNVNVSAEININYRLSELGFITNKNDMDWIKKNYDLYSKLIAGAIHGKPIGGLVAGNVKTSAKNKKNPPVPAGYTLDKNNVPYKKEQGNYTVANVKGNNVRDGYSTNSRITGVLPNNTTITYDGAYCINGYRWITYIANSGQRRYIATGEVDKAGNRISSFGKFSTI

SEQ ID NO: 80 - DNA, 1764 bp, synthetic construct
  misc_feature 1..1764: note = Nucleotide sequence encoding Peptidoglycan Hydrolase
  source 1..1764: mol_type = other DNA
atgagatttccatctattttcactgctgttttgttcgctgcttcttctgctttggctgctccagttaacactactactgaagatgagactgctcaaattcctgctgaagctgttattggttactctgatttggagggagattttgatgttgctgttttgcctttctctaactctactaacaacggtttgttgttcattaacactactatcgcttctatcgctgctaaggaagagggtgtttctttggaaaagagagaagctgaggctcatcaccatcaccatcacttggaggttttgtttcaaggtccaatgttgatgactaagaaccaagctgaaaaatggttcgataactctttgggtaaacaattcaatcctgatggttggtacggtttccaatgttacgattacgctaacatgtttttcatgttggctactggtgaaagattgcaaggtttgtacgcttacaacatcccattcgataataaggctaagatcgagaagtacggtcaaattattaagaactacgattctttcttgccacaaaagttggatatcgttgttttcccttctaaatacggtggtggtgctggtcatgttgaaattgttgagtctgctaacttgaatacttttacttctttcggtcaaaactggaatggtaaaggttggactaacggtgttgctcaaccaggttggggtcctgaaactgttactagacatgttcactactacgataacccaatgtacttcatcagattgaatttccctaacaatttgtctgttggtaataaggctaagggtattattaagcaagctactactaagaaagaggctgttattaaaccaaagaaaattatgttggttgctggtcacggttataacgatcctggtgctgttggtaacggtactaacgaaagagatttcattagaaagtacatcactcctaacatcgctaagtatttgagacatgctggtcacgaggttgctttgtacggtggttcttctcaatctcaagatatgtaccaagatactgcttatggtgttaacgttggtaataagaaagattacggtttgtactgggttaagtctcaaggttacgatatcgttttggaaatccatttggatgctgctggtgaatctgcttctggtggtcacgttatcatctcttctcaattcaacgctgatactatcgataagtctatccaagatgttattaaaaacaatttgggtcaaattagaggtgttactccaagaaacgatttgttgaacgttaacgtttctgctgaaattaacatcaactacagattgtctgagttgggttttattactaacaaaaacgatatggattggattaagaaaaactacgatttgtattctaagttgattgctggtgctattcatggtaaacctattggtggtttggttgctggtaacgttaagacttctgctaaaaacaagaaaaatccacctgttccagctggttacactttggataagaacaatgttccttacaagaaagaacagggtaactacactgttgctaacgttaagggtaacaacgttagagatggttactctactaactccagaattactggtgttttgccaaacaacactactatcacttacgatggtgcttactgtatcaacggttacagatggatcacttacatcgctaattctggtcaaagaagatatattgctactggtgaagttgataaggctggtaatagaatttcttcttttggtaaattctctactatctaa

SEQ ID NO: 81 - protein, 309 aa, synthetic construct
  REGION 1..309: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPTQNYVVKKGDYLGAIAKKFNTTVSKIQKLNNIKNPNFILIGQVLKVPFISSNNSSTNTNRRNNSSSTLNYLNSLAYKGWDFDGAYGNQCFDLVNVYWNHLYGHGLRGYGAKDIPFANNFRGEATVYRNTPTFKAELGDVAVFSGRYGGGFGHTAIVLNGNYDGKLMKFQSLDQNWYGGDSRRADIAHKVVHNYDYDMYFIRPYKKA

SEQ ID NO: 82 - DNA, 930 bp, synthetic construct
  misc_feature 1..930: note = Nucleotide sequence encoding Peptidoglycan Hydrolase
  source 1..930: mol_type = other DNA
atgagatttccatctattttcactgctgttttgtttgctgcttcttctgctttggctgctccagttaacactactactgaagatgagactgctcaaattcctgctgaagctgttattggttattctgatttggagggagattttgatgttgctgttttgccattctctaactctactaacaacggtttgttgttcattaacactactatcgcttctatcgctgctaaggaagagggtgtttctttggaaaagagagaagctgaggctcatcaccatcaccatcacttggaggttttgtttcaaggtcctactcaaaactacgttgttaagaaaggagattatttgggtgctatcgctaagaaattcaatactactgtttctaagatccaaaagttgaacaacattaagaacccaaacttcatcttgatcggtcaagttttgaaagttcctttcatttcttctaacaactcttctactaacactaacagaagaaacaactcttcttctactttgaactacttgaactctttggcttataagggttgggattttgatggtgcttacggtaaccaatgtttcgatttggttaacgtttactggaatcatttgtatggtcacggtttgagaggttatggtgctaaggatattccatttgctaacaatttcagaggtgaagctactgtttacagaaacactcctacttttaaagctgagttgggagatgttgctgttttctctggtagatacggtggtggtttcggtcatactgctatcgttttgaacggtaactacgatggtaaattgatgaagttccaatctttggatcaaaactggtacggtggagattctcgtagagctgatatcgctcataaggttgttcacaactacgattacgatatgtacttcatcagaccttacaagaaagcttaa

SEQ ID NO: 83 - protein, 394 aa, synthetic construct
  REGION 1..394: note = Peptidoglycan Hydrolase
MRFPSIFTAVLFAASSALAAPVNTTTEDETAQIPAEAVIGYSDLEGDFDVAVLPFSNSTNNGLLFINTTIASIAAKEEGVSLEKREAEAHHHHHHLEVLFQGPMNKVDAWLSKHDLKNLTTLIVVGAFILFLILLFMLMNHSDQGSNRITVSENAELRTDPNAAYPVIYQVEKGDTFTRLSKSGKWIEVESRDGSEKGWIAGWHTSLNIEADSNKKVNPLKNKVIVLDPGHGGNDQGASSNTSKKSLEKNYTLKTAKELRRALEKAGATVKLTRSDDTYVSLDDRKLNGDAYISIHNDSLDSPNANGATVYWYKNNQEALAETLNTNIQKKAMLSNRGTRQENFQVLRQTNKPAVLLELGYISNPTDEVMIRNQIHRQVVEEAVVDGLKQYFAS

SEQ ID NO: 84 - DNA, 1185 bp, synthetic construct
  misc_feature 1..1185: note = Nucleotide sequence encoding Peptidoglycan Hydrolase
  source 1..1185: mol_type = other DNA
atgagatttccatctattttcactgctgttttgtttgctgcttcttctgctttggctgctccagttaacactactactgaagatgagactgctcaaattcctgctgaagctgttattggttactctgatttggagggagattttgatgttgctgttttgccattctctaactctactaacaacggtttgttgtttattaacactactatcgcttctattgctgctaaggaagagggtgtttctttggaaaagagagaagctgaggctcatcaccatcaccatcacttggaggttttgttccaaggtcctatgaacaaggttgatgcttggttgtctaagcatgatttgaaaaatttgactactttgatcgttgttggtgcttttattttgttcttgatcttgttgtttatgttgatgaaccactctgatcaaggttctaacagaattactgtttctgaaaatgctgagttgagaactgatccaaatgctgcttacccagttatctaccaagttgaaaagggagatactttcactagattgtctaagtctggtaaatggattgaagttgagtccagagatggttctgaaaaaggttggattgctggttggcatacttctttgaacattgaggctgattctaacaagaaagttaacccattgaagaacaaagttattgttttggatcctggtcacggtggtaacgatcaaggtgcttcttctaatacttctaagaagtctttggaaaagaactacactttgaagactgctaaagaattgcgtagagctttggaaaaggctggtgctactgttaagttgactagatctgatgatacttacgtttctttggatgatagaaagttgaacggagatgcttacatctctatccataacgattctttggattctcctaacgctaatggtgctactgtttactggtacaagaacaaccaagaagctttggctgagactttgaacactaacatccaaaagaaagctatgttgtctaacagaggtactagacaagaaaacttccaagttttgagacaaactaacaagccagctgttttgttggaattgggttacatttctaaccctactgatgaggttatgattagaaatcaaatccacagacaagttgttgaagaggctgttgttgatggtttgaaacaatatttcgcttcttaa

SEQ ID NO: 85 - protein, 530 aa, synthetic construct
  REGION 1..530: note = Peptidoglycan Hydrolase
MDWTWRVFCLLAVAPGAHSMKHHHHHHSAGLEVLFQGPMAKTKKQIEQRLQAYAKGTVDSPYRITKPTSYDPSFGVMEVGAIDADRYYHAQCQDLITDYVLWLTDNKVRPWGNAKDQINQSYGTGFKIHKNLPSTVAKTGWIAVFTSGTYSQYGHIGIVYSPGDTNKFHIIEQNWNGWANKKPLTRWDNYYGLTHFIEVPYAKETITTKKTAPKAATSKPAKKISVSHNNINLQSNFAKRGYNPKGVVIHNDAGRSTAQQYENSLKGANQNRLNNGIAHAYADSSHIWYAIEPERIAYHTGDGVKKGTGNYDYFGIEVCQSMSASDKEFMANEQVVLQEVARLLKKWKLPANRNTVRLHLEFSATSCPHRSLTMRTGFNPVTQGRPSDAIIKKLKDIWIKEIRAYMEGKTPSIKTVANKPGSASTPATKSNSDGWKVNAYNTYYKTERASFTVGSTRIAVRSTGPFTTCPFTGWLNPGVTITYDEVMVQNGFVWLGYDSYNGRRYVPIRTAQGTPGSSNYVLGPLWGTIS

SEQ ID NO: 86 - DNA, 2044 bp, synthetic construct
  misc_feature 1..2044: note = Nucleotide sequence encoding Peptidoglycan Hydrolase
  source 1..2044: mol_type = other DNA
actagtattcttctggtccccacagactcagagagaacccgccaccatggattggacgtggcgagttttctgtctgctggctgttgccccaggcgcgcattctatgaagcatcaccaccatcaccactcagcagggctcgaagtcctgttccaaggaccaatggccaaaaccaagaaacagatagaacagagactgcaagcttacgcaaaggggaccgtggattccccttaccgaatcaccaagccaaccagctacgacccctccttcggtgtcatggaggttggcgctatagatgcggatcgctactatcatgcccagtgtcaagatctgataactgactatgtactttggctcactgacaacaaagtaagaccttggggtaatgccaaggaccagattaaccagagctatgggactggtttcaaaatccacaagaatctgcccagtaccgtggctaaaacaggatggattgccgtgtttacatccgggacctactcacagtacggccacatcggcatcgtgtacagtccaggagataccaacaaattccatatcatcgagcagaattggaatggatgggcaaacaagaaacctctgacacgttgggacaactactacgggctgacccactttatcgaggtcccatatgcgaaagagacaatcacaacgaagaaaactgcacctaaagcagctacctcaaaaccggctaagaagatctcagtttctcacaacaacatcaaccttcagagcaactttgcaaagagagggtacaacccaaagggagtggtgattcacaatgacgccggacgctctactgcgcaacagtatgaaaacagcctgaagggcgctaatcagaacaggctgaataacggcattgcccatgcatatgctgacagcagccacatttggtatgctatcgaacccgagagaatcgcctatcacacaggcgatggcgttaagaagggaacaggcaattacgactacttcggtattgaagtatgccagagcatgagcgcaagtgacaaagagttcatggccaacgaacaggtggtactgcaggaggtggctaggttgctcaagaaatggaagcttcccgccaatcggaataccgtgcggctccatctggaattctctgccacatcttgccctcataggagcctcacaatgcggaccggtttcaatccggtgactcagggtagaccctcagacgccatcatcaagaagttgaaggatatctggataaaagagattcgcgcatatatggagggtaaaaccccatccattaagactgtcgccaataagcccggatcagccagtacccctgcaaccaaatccaactccgatgggtggaaggtcaatgcctacaatacctactacaaaacggaacgggcctcctttacagtgggctctactcggattgctgtgcgtagtacaggaccctttactacatgcccgtttacagggtggctgaaccccggagtcacgataacctatgacgaggttatggtgcagaacgggtttgtgtggttgggctatgatagctacaatgggaggaggtatgtccccattcgcactgcccaaggaactcctggcagctccaattatgtgcttggccctctgtggggcacgatttcttgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 87 - protein, 519 aa, synthetic construct
  REGION 1..519: note = Peptidoglycan Hydrolase
MDWTWRVFCLLAVAPGAHSMKHHHHHHSAGLEVLFQGPMQAKLTKKEFIEWLKTSEGKQFNADLWYGFQCFDYANAAWKVLFGLLLKGLGAKDIPFANNFDGLATVYQNTPDFLAQPGDMVVFGSNYGAGYGHVAWVIEATLDYIIVYEQNWLGGGWTDGIEQPGWGWEKVTRRQHAYDFPMWFIRPNFKSEIAPRSVQSPTQAPKKETAKPQPKAVELKIIKDVVKGYDLPKRGSNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNDSSASSNTVKPVASAWKRNKYGTYYMEESARFTNGNQPITVRKVGPFLYCPVGYQFQPGGYCDYTEVMLQDGHVWVGYTWEGQRYYLPIRTWNGSAPPNQILGDLWGEIS

SEQ ID NO: 88 - DNA, 2011 bp, synthetic construct
  misc_feature 1..2011: note = Nucleotide sequence encoding Peptidoglycan Hydrolase
  source 1..2011: mol_type = other DNA
actagtattcttctggtccccacagactcagagagaacccgccaccatggattggacatggcgagtgttctgccttcttgctgttgcacctggtgcccattccatgaaacatcaccaccatcaccattcagctggccttgaagtgcttttccagggacctatgcaagcgaaactgaccaagaaggagtttatagagtggctgaaaacctctgagggtaaacagttcaacgccgatctgtggtatggtttccagtgctttgattacgcaaacgctgcttggaaagtgctgtttggcctgctcctgaagggactcggtgccaaagacatcccctttgccaacaatttcgacgggctggcaaccgtctaccagaatactcctgactttctcgctcagccaggggacatggtcgtgttcggctccaattatggggcaggctatggacacgtagcatgggtgatagaagccactctggactacataatcgtctacgagcaaaattggcttggcggtgggtggaccgatggcattgagcagcctggctggggatgggagaaggttactcgtagacagcatgcttacgactttcccatgtggttcatccgtcccaacttcaagtccgagattgcgcccagaagcgtccagtctccaacccaagctccgaagaaggaaacagccaagccacagcctaaggccgttgaactgaagattattaaggatgtggtgaagggatatgacctcccgaaaagagggagcaatcccaaagggattgtgatccacaacgatgccgggtctaaaggggcaacagcagaagcgtataggaacgggttggtgaatgcccctctcagtcgcttggaggccggaattgcacactcatatgtgtccggaaatacggtttggcaagctctcgacgaaagccaagtaggctggcatacagccaaccagctgggcaacaaatactactatggcatcgaggtgtgccagagtatgggcgcagataatgccacgttcctgaagaacgagcaggctacctttcaagagtgtgcgcggttgctgaagaagtggggactgccagctaatcgcaacacaatcaggttgcacaacgagtttacgagtactagctgtccacacagaagtagcgtgctgcacacaggattcgatcctgtgacaaggggactgctgcccgaagataagcggctgcagctgaaagactacttcatcaaacagattcgggcctatatggacggaaagattcccgtcgctactgttagcaatgacagctctgcctcatcaaataccgtgaaaccggtagcaagcgcctggaaaaggaacaagtatggcacttattacatggaggaatccgcccgatttaccaacggtaatcagcccatcacagtccgcaaggtcgggccattcctgtactgtcccgtaggctaccagtttcagcctggtgggtactgcgactataccgaggtgatgctgcaagatggccatgtctgggttggctatacctgggaagggcagcgctactacttgccaatacggacttggaatggttctgccccacccaaccagatcctcggagatctgtggggcgaaatctcctgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 89 - protein, 519 aa, synthetic construct
  REGION 1..519: note = Peptidoglycan Hydrolase
MDWTWRVFCLLAVAPGAHSMKHHHHHHSAGLEVLFQGPMQAKLTKKEFIEWLKTSEGKQFNVDLWYGFQCFDYANAGWKVLFGLLLKGLGAKDIPFANNFDGLATVYQNTPDFLAKPGDMVVFGSNYGAGYGHVAWVIEATLDYIIVYEQNWLGGGWTDRIEQPGWGWEKVTRRQHALRFPYVVYPSYPKSETAPRSIQSPTQASKKETAKPQPKAVELKIIKDVVKGYDLPKRGGNPKGIVIHNDAGSKGATAEAYRNGLVNAPLSRLEAGIAHSYVSGNTVWQALDESQVGWHTANQLGNKYYYGIEVCQSMGADNATFLKNEQATFQECARLLKKWGLPANRNTIRLHNEFTSTSCPHRSSVLHTGFDPVTRGLLPEDKRLQLKDYFIKQIRAYMDGKIPVATVSNESSASSNTVKPVASAWKRNKYGTYYMEESARFTNGNQPITVRKVGPFLSCPVGYQFQPGGYCDYTEVMLQDGHVWVGYTWEGQRYYLPIRTWNGSAPPNQILGDLWGEIS

SEQ ID NO: 90 - DNA, 2011 bp, synthetic construct
  misc_feature 1..2011: note = Nucleotide sequence encoding Peptidoglycan Hydrolase
  source 1..2011: mol_type = other DNA
actagtattcttctggtccccacagactcagagagaacccgccaccatggactggacctggagagtcttttgcctccttgcagttgctcctggtgcccacagtatgaagcatcaccatcatcaccacagtgcgggactggaggtgttgtttcagggaccaatgcaggccaaactgaccaagaaggagttcatcgaatggctcaagacttccgagggcaagcagttcaatgtggacctctggtatggctttcaatgctttgattatgccaatgctgggtggaaggtcctgtttggccttctcctgaaaggactgggtgcaaaagacatccccttcgccaacaacttcgatggactggctaccgtttaccagaataccccggactttctggccaagcctggagatatggtggtgttcggaagcaactatggggcaggatatggccatgtagcctgggtgatagaagctacgttggactacatcatcgtgtacgagcaaaattggctgggcggcggctggactgaccgaattgagcaacctgggtggggatgggagaaagtgaccagacgtcagcacgctctgcgcttcccctatgtagtgtacccctcttacccgaagtctgagacagctccccggtccattcagagcccaacacaggcgagcaagaaagagactgccaaaccacaacccaaagcggttgaactgaagatcatcaaagacgtcgtgaaagggtatgatctgcccaagagaggcggaaatccaaaggggattgtgatccacaatgacgccgggagcaaaggtgcaactgctgaagcttaccgcaatgggctggtcaacgcacctttgtcccgactggaagccggcattgctcatagctatgtgtctggcaacactgtttggcaagcactggacgaatcacaggtcggctggcatactgccaatcagctcggaaacaagtactactacgggatagaggtctgtcagtccatgggcgccgataacgcgactttcctgaagaatgagcaggccacattccaggaatgcgctcggttgttgaagaaatggggccttcctgcaaacaggaacacaatcaggctgcacaatgagttcacatccacgtcttgtccacacaggtctagcgtgctgcacacaggttttgatcccgtgacgagagggcttctccccgaagataaacgtctgcagctcaaggattacttcatcaagcagataagggcctatatggacgggaaaattccggtagctaccgtcagcaacgagagtagcgcatcatccaacaccgttaagccagtggcctcagcctggaagcgcaacaaatacggtacctattacatggaggaatctgcaagattcacaaatggtaatcagcctattaccgtgcggaaagtcggcccttttctcagctgtcctgttggatatcagtttcagccaggcggttactgcgactataccgaagtaatgctgcaagatgggcatgtgtgggttgggtacacatgggaaggccaacggtactatctgcccattcgcacctggaacggcagtgccccacccaaccagatccttggagatctgtggggtgagatttcatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 91 - protein, 533 aa, synthetic construct
  REGION 1..533: note = Peptidoglycan Hydrolase
MDWTWRVFCLLAVAPGAHSMKHHHHHHSAGLEVLFQGPMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 92 - DNA, 2053 bp, synthetic construct
  misc_feature 1..2053: note = Nucleotide sequence encoding Peptidoglycan Hydrolase
  source 1..2053: mol_type = other DNA
actagtattcttctggtccccacagactcagagagaacccgccaccatggactggacttggcgggtcttctgtttgcttgccgtcgcccccggagcacactctatgaagcaccaccaccatcaccactctgccggcctggaagtgctgtttcagggccctatggccaagacacaggccgagatcaacaagcggctggacgcctatgccaagggcacagtggatagcccctacagagtgaagaaggccaccagctacgaccccagcttcggcgttatggaagccggcgctattgacgccgacggctattatcatgcccagtgccaggacctgatcaccgactatgtgctgtggctgaccgacaacaaagtgcggacatggggcaacgccaaggaccagatcaagcagagctacggcaccggcttcaagatccacgagaacaagcctagcaccgtgcctaagaaaggctggatcgccgtgtttaccagcggcagctatgagcagtggggccacatcggcatcgtgtacgatggcggcaataccagcaccttcaccatcctggaacagaactggaacggctacgccaacaagaaacccaccaagagagtggacaactactacggcctgacacacttcatcgagatccccgtgaaggccggcaccaccgtgaagaaagagacagccaagaagtccgccagcaagacccctgctcctaagaagaaagccacactgaaggtgtccaagaaccacatcaactacaccatggacaagcggggcaaaaagcccgaaggcatggtcatccacaacgacgccggcagatctagcggccagcagtacgaaaacagcctggccaatgccggctatgccagatacgccaatggaatcgcccactactatggcagcgagggctatgtgtgggaagccatcgacgccaagaatcagatcgcctggcatacaggcgacggaaccggcgccaattccggcaatttcagattcgccggcatcgaagtgtgccagagcatgtctgccagcgacgcccagttcctgaagaatgagcaggccgtgttccagtttaccgccgagaagttcaaagagtggggactgacccctaacagaaagaccgtgcggctgcacatggaattcgtgcctaccgcatgtccccaccgctctatggtgctgcacaccggctttaaccccgtgacacagggcagacctagccaggccatcatgaacaagctgaaggactacttcattaagcagatcaagaactacatggataagggcaccagcagcagcaccgtggtcaaggatggcaagaccagcagcgcctctacaccagccacaagacctgtgacaggcagctggaagaagaaccagtacggcacctggtacaagcccgagaacgccaccttcgtgaacggcaaccagcctatcgtgaccagaatcggcagccccttcctgaatgcccctgtcggaggaaatcttcctgctggcgccaccatcgtgtatgacgaagtctgtattcaggccggccacatctggatcggctacaacgcctacaacggcaacagagtgtactgccccgtgcggacttgtcagggcgttccacctaatcagattccaggcgtggcctggggcgtgttcaaatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 93 - protein, 253 aa, synthetic construct
  REGION 1..253: note = Peptidoglycan Hydrolase
MDWTWRVFCLLAVAPGAHSMKHHHHHHSAGLEVLFQGPREAPKTQIYTVKKGDTLSAIALKYKTTVSNIQNTNNIANPNLIFIGQKLKVPMTPLVEPKPKTVSSNNKSNSNSSTLNYLKTLENRGWDFDGSYGWQCFDLVNVYWNHLYGHGLKGYGAKDIPYANNFNSEAKIYHNTPTFKAEPGDLVVFSGRFGGGYGHTAIVLNGDYDGKLMKFQSLDQNWNNGGWRKAEVAHKVVHNYENDMIFIRPFKKA

SEQ ID NO: 94 - DNA, 1213 bp, synthetic construct
  misc_feature 1..1213: note = Nucleotide sequence encoding Peptidoglycan Hydrolase
  source 1..1213: mol_type = other DNA
actagtattcttctggtccccacagactcagagagaacccgccaccatggattggacatggagggtgttttgtctgctcgcagttgctccaggtgcacatagcatgaaacaccaccatcaccaccattctgctgggcttgaggtgctgtttcaaggacctagagaggctcccaaaacacagatttacaccgtcaagaagggagatacgcttagcgccattgcactgaaatacaagaccaccgtatcaaacatccagaacaccaataacatagccaatcccaatctcatcttcattggccagaagctcaaggttcccatgactccactggtcgaaccgaaacctaagacagtgtcctccaataacaagagtaacagcaactcctctacactgaactatctcaagacgttggagaatcgcggttgggactttgacggcagttacggctggcaatgcttcgacttggtgaatgtgtactggaatcacctgtatgggcatggactgaaaggctatggggccaaagacataccttatgctaacaatttcaactctgaggccaagatctaccacaacactcctaccttcaaggcagaaccaggagatctggttgtgtttagcggacggtttggcggaggctatgggcatactgccattgtgcttaatggggattacgatggcaaactgatgaaattccagtcactggaccagaactggaacaatggtggttggcgaaaagcggaagtagcccacaaggtcgtccacaattacgagaacgacatgatcttcatccgtccctttaagaaagcgtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 95 - protein, 284 aa, synthetic construct
  REGION 1..284: note = Peptidoglycan Hydrolase
MDWTWRVFCLLAVAPGAHSMKHHHHHHSAGLEVLFQGPAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 96 - DNA, 1306 bp, synthetic construct
  misc_feature 1..1306: note = Nucleotide sequence encoding Peptidoglycan Hydrolase
  source 1..1306: mol_type = other DNA
actagtattcttctggtccccacagactcagagagaacccgccaccatggattggacctggagagtgttttgccttctcgctgttgcccctggtgcccattccatgaagcaccaccaccaccatcatagtgcagggctggaagttctgtttcagggtccagcagcaacgcatgagcattccgctcaatggctcaacaactacaagaagggttacggctatggcccctatcctctggggataaacggagggatgcactatggagtcgatttcttcatgaatattggcactccagtgaaagcgatttccagcgggaagattgtggaagctgggtggagcaactatggtggtggcaatcagatcggactgattgagaatgacggcgtgcacagacagtggtatatgcatctgagtaagtacaacgtcaaggtgggagactacgtcaaagccgggcagatcataggatggtccggctctacaggttactcaacagcgccacatctccactttcagcgcatggtgaatagcttcagcaattcaaccgcccaagatccgatgcccttcctgaagtctgccggctatgggaaagctggaggaacagtgacacctactcccaataccggatggaaaacgaacaagtacggcactctgtacaaatccgaatctgccagtttcacacccaataccgacatcatcacccgaaccactggaccttttcggtcaatgccgcagtctggagtactgaaagcagggcaaactatccactatgacgaggtgatgaagcaggatggccacgtatgggtggggtacacaggcaatagcggccagaggatttacttgccagtccgtacctggaacaaaagcacgaacactcttggcgttttgtggggcaccatcaagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 97 - protein, 188 aa, synthetic construct
  REGION 1..188: note = Peptidoglycan Hydrolase
MDWTWRVFCLLAVAPGAHSMKHHHHHHSAGLEVLFQGPDQFLDEKEGNHTTAYRDGSGIWTICRGATMVDGKPVIPGMRLSKEKCDQVNAIERDKALAWVERNIKVPLTEPQKAGIASFCPYNIGPGKCFPSTFYKRLNAGDRKGACEAIRWWIKDGGRDCRIRSNNCYGQVIRRDQESALACWGIDQ

SEQ ID NO: 98 - DNA, 1018 bp, synthetic construct
  misc_feature 1..1018: note = Nucleotide sequence encoding Peptidoglycan Hydrolase
  source 1..1018: mol_type = other DNA
actagtattcttctggtccccacagactcagagagaacccgccaccatggactggacatggcgcgtgttttgccttctcgccgtagcacctggtgcacacagcatgaagcatcaccatcaccatcactctgcaggcttggaagtcctgttccaagggcctgatcagtttctcgacgagaaggaaggcaatcacaccactgcgtatagggatggctctgggatatggaccatctgtagaggcgccacaatggtcgatggcaaacccgtgatacccggaatgcgtctgtcaaaggagaagtgcgatcaggtgaatgccatcgaacgcgacaaagctctggcctgggttgagcggaacatcaaggttccactgacggaaccacagaaagccggaattgcgtccttctgtccgtacaacattggccctggaaagtgctttccctccactttctacaagcgcctgaatgcaggggacagaaaaggagcctgtgaggctatcaggtggtggatcaaagatggaggtagggactgtcgaatccggagcaacaactgctatgggcaagtgattcggagagatcaggagagtgctctggcttgctggggtattgaccagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 99 - protein, 325 aa, synthetic construct
  REGION 1..325: note = Peptidoglycan Hydrolase
MDWTWRVFCLLAVAPGAHSMKHHHHHHSAGLEVLFQGPMRYIPAAHHSAGSNKPVNRVVIHATCPDVGFPSASRKGRAVSTANYFASPSSGGSAHYVCDIGETVQCLSESTIGWHAPPNPHSLGIEICADGGSHASFRVPGHAYTREQWLDPRVWPAVERAAVLCRQLCDKHGVPKRKLSVADLKAGRRGICGHTDVTDAWHQSDHDDPGPWFPWDKFMAVVNGHGGGSSSEELTVADVKALHNQIKQLSAQLSGSVNKLHHDVGVVQVQNGDLSKRVDALSWVKNPVTGKLWRTKDALWSVWYYVLECRSRIDRLESAVNGLKK

SEQ ID NO: 100 - DNA, 1429 bp, synthetic construct
  misc_feature 1..1429: note = Nucleotide sequence encoding Peptidoglycan Hydrolase
  source 1..1429: mol_type = other DNA
actagtattcttctggtccccacagactcagagagaacccgccaccatggactggacatggagagtgttctgtctgcttgctgtggcaccaggcgctcactccatgaaacaccaccaccatcaccattctgctggtctggaagtgcttttccagggacctatgcgctatattcctgctgcacaccattctgcagggagcaacaagcccgtgaatcgcgttgtcatccatgccacttgtcccgatgtgggctttccctccgcctcacggaaaggaagggcagtgagcactgccaactacttcgccagtccgtcctcaggcgggtcagcccactatgtgtgcgacataggggagacagtacagtgtctcagcgaaagcacgattgggtggcatgccccaccgaatccacactctttggggatcgagatttgcgcagatggaggttctcatgcctcttttcgcgtgccaggccatgcatacaccagagagcagtggctcgatccacgagtttggcccgcagttgaacgagctgctgtgctgtgccggcaattgtgcgacaagcatggcgtacccaaacggaaactgagcgttgcggacctgaaagccgggaggaggggtatatgcggacataccgacgtgactgacgcgtggcaccagagtgaccacgatgaccctgggccttggtttccttgggacaagttcatggcggtggtaaacggacatggtggcggcagtagtagcgaggaactgaccgttgccgatgtcaaggccctgcacaaccagatcaagcagctgtctgctcaactctccggaagcgtgaacaagctgcaccacgatgtgggagtcgtccaagtgcagaatggcgacctctccaagagagtggatgccttgagctgggtcaagaatcccgttacaggcaaactttggcgtaccaaagatgccctgtggagcgtctggtactatgtactggagtgtcggtccaggatcgatagactggagtcagctgtcaatggtctgaagaagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 101 - protein, 522 aa, synthetic construct
  REGION 1..522: note = Peptidoglycan Hydrolase
MDWTWRVFCLLAVAPGAHSMKHHHHHHSAGLEVLFQGPMLMTKNQAEKWFDNSLGKQFNPDGWYGFQCYDYANMFFMLATGERLQGLYAYNIPFDNKAKIEKYGQIIKNYDSFLPQKLDIVVFPSKYGGGAGHVEIVESANLNTFTSFGQNWNGKGWTNGVAQPGWGPETVTRHVHYYDNPMYFIRLNFPNNLSVGNKAKGIIKQATTKKEAVIKPKKIMLVAGHGYNDPGAVGNGTNERDFIRKYITPNIAKYLRHAGHEVALYGGSSQSQDMYQDTAYGVNVGNKKDYGLYWVKSQGYDIVLEIHLDAAGESASGGHVIISSQFNADTIDKSIQDVIKNNLGQIRGVTPRNDLLNVNVSAEININYRLSELGFITNKNDMDWIKKNYDLYSKLIAGAIHGKPIGGLVAGNVKTSAKNKKNPPVPAGYTLDKNNVPYKKEQGNYTVANVKGNNVRDGYSTNSRITGVLPNNTTITYDGAYCINGYRWITYIANSGQRRYIATGEVDKAGNRISSFGKFSTI

SEQ ID NO: 102 - DNA, 2020 bp, synthetic construct
  misc_feature 1..2020: note = Nucleotide sequence encoding Peptidoglycan Hydrolase
  source 1..2020: mol_type = other DNA
actagtattcttctggtccccacagactcagagagaacccgccaccatggactggacttggcgggtcttctgtttgcttgccgtcgcccccggagcacactctatgaagcaccaccaccatcaccactctgccggcctggaagtgctgtttcagggccctatgctgatgaccaagaaccaggccgagaagtggttcgacaacagcctgggcaagcagttcaaccccgatggatggtacggcttccagtgctacgactacgccaacatgttcttcatgctggccaccggcgagagactgcagggcctgtacgcctacaacatccccttcgataacaaggccaagatcgagaagtacggccagatcatcaagaactacgacagcttcctgcctcagaaactggacatcgtggtgttccccagcaaatatggcggcggagccggacacgtggaaatcgtggaaagcgccaacctgaacaccttcaccagcttcggccagaactggaacggcaaaggctggaccaatggcgtggcacaacctggctggggacctgagacagtgacaagacacgtgcactactacgacaaccccatgtacttcatccggctgaacttccccaacaacctgagcgtgggcaacaaagccaagggcatcatcaagcaggccaccaccaagaaagaagccgtgatcaagcccaagaaaatcatgctggtggctggccacggctacaacgatcctggcgctgttggcaacggcaccaacgagagagacttcatcagaaagtacatcacgcccaatatcgccaagtacctgaggcacgccggacatgaggttgccctgtatggcggatctagccagagccaggacatgtaccaggataccgcctacggcgtgaacgtgggaaacaagaaggactacggcctgtactgggtcaagagccagggctacgacatcgtgctggaaatccacctggatgccgctggcgaatctgcctctggcggacatgtgatcatcagcagccagttcaatgccgacaccatcgacaagagcatccaggatgtgatcaagaacaacctgggccagatccggggcgtgacccctagaaacgatctgctgaatgtgaacgtgtccgccgagatcaacatcaactaccggctgagcgagctgggcttcatcaccaacaagaacgacatggactggatcaagaagaattacgacctctacagcaagctgatcgctggcgccatccacggcaagcctattggaggactggtggccggcaacgtgaaaaccagcgccaagaacaagaaaaaccctcctgtgcctgccggctacaccctggacaagaacaatgtgccctacaagaaagagcagggcaactacaccgtggccaacgtgaagggaaacaacgtgcgcgacggctacagcaccaacagcagaattacaggcgtgctgcccaacaacaccacaatcacctacgacggcgcctactgcatcaacggctatcggtggatcacctacattgccaactccggccagcggcggtatattgccacaggcgaagtggacaaggccggcaatagaatcagctccttcggcaagttcagcaccatctaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 103 - protein, 244 aa, synthetic construct
  REGION 1..244: note = Peptidoglycan Hydrolase
MDWTWRVFCLLAVAPGAHSMKHHHHHHSAGLEVLFQGPTQNYVVKKGDYLGAIAKKFNTTVSKIQKLNNIKNPNFILIGQVLKVPFISSNNSSTNTNRRNNSSSTLNYLNSLAYKGWDFDGAYGNQCFDLVNVYWNHLYGHGLRGYGAKDIPFANNFRGEATVYRNTPTFKAELGDVAVFSGRYGGGFGHTAIVLNGNYDGKLMKFQSLDQNWYGGDSRRADIAHKVVHNYDYDMYFIRPYKKA

SEQ ID NO: 104 - DNA, 1186 bp, synthetic construct
  misc_feature 1..1186: note = Nucleotide sequence encoding Peptidoglycan Hydrolase
  source 1..1186: mol_type = other DNA
actagtattcttctggtccccacagactcagagagaacccgccaccatggactggacttggagggtgttctgcctgttggctgtagcaccaggagcacactccatgaaacatcaccatcatcaccactctgccggacttgaggtcctgtttcaaggtcccacacagaattacgtggtgaagaagggcgattacctgggtgcgatcgccaagaagttcaacaccacagtgagcaagatccagaaactcaacaacatcaagaatcccaacttcatcctgattggccaagtgctgaaagtaccgttcataagctctaacaactcaagcaccaatacgaaccgccggaacaactccagtagcacactgaactacctgaattccctcgcatacaaagggtgggatttcgatggagcctatggcaatcagtgttttgacctcgtcaacgtgtactggaatcacctctatggtcatgggctgagagggtacggtgctaaggacataccttttgccaataattttcgcggcgaagctactgtgtatcggaataccccaaccttcaaagcagagcttggagatgttgctgttttctcagggaggtatggaggaggctttggccatactgccattgtcctgaatggcaactatgacgggaaactgatgaaattccagtctttggaccagaattggtatggcggggatagtagacgtgccgatattgcgcacaaggttgtccacaactatgactacgacatgtactttatccgaccttacaagaaggcctgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 105 - protein, 329 aa, synthetic construct
  REGION 1..329: note = Peptidoglycan Hydrolase
MDWTWRVFCLLAVAPGAHSMKHHHHHHSAGLEVLFQGPMNKVDAWLSKHDLKNLTTLIVVGAFILFLILLFMLMNHSDQGSNRITVSENAELRTDPNAAYPVIYQVEKGDTFTRLSKSGKWIEVESRDGSEKGWIAGWHTSLNIEADSNKKVNPLKNKVIVLDPGHGGNDQGASSNTSKKSLEKNYTLKTAKELRRALEKAGATVKLTRSDDTYVSLDDRKLNGDAYISIHNDSLDSPNANGATVYWYKNNQEALAETLNTNIQKKAMLSNRGTRQENFQVLRQTNKPAVLLELGYISNPTDEVMIRNQIHRQVVEEAVVDGLKQYFAS

SEQ ID NO: 106 - DNA, 1441 bp, synthetic construct
  misc_feature 1..1441: note = Nucleotide sequence encoding Peptidoglycan Hydrolase
  source 1..1441: mol_type = other DNA
actagtattcttctggtccccacagactcagagagaacccgccaccatggactggacttggcgcgtgttttgcctgctcgccgtagctcctggagcccatagcatgaagcaccatcatcaccaccattcagcgggattggaggttctctttcaaggccccatgaataaggtggatgcttggctcagcaaacacgaccttaagaacctcactaccttgatcgttgtaggagccttcattctgttcctgatactgctgttcatgctgatgaatcactctgatcaggggtcaaaccgcataactgtttccgagaatgccgaacttcggacagatccaaacgccgcctatccggtcatctatcaggtcgagaaaggggacactttcaccagactgtctaaaagcggcaaatggattgaggtcgagagtagagatgggagtgagaaggggtggatagcaggatggcacacatccctgaacattgaagccgatagcaacaagaaagtgaaccctctcaagaacaaagtgatcgtgctggatcctggtcatggtggcaacgatcagggcgcttcaagcaataccagcaagaagtcactggagaagaattacacgctgaaaacagccaaggaactgcgacgggctcttgagaaagcaggagctacggtgaaactgacacggtctgacgacacctatgtgagtctggacgacaggaagctgaatggcgacgcgtatatctccattcacaatgacagcctggattctcccaatgcaaatggggctaccgtctactggtacaagaacaaccaagaggccctggcagaaaccctcaataccaacatccagaagaaggcgatgttgtccaataggggtactaggcaggaaaactttcaggtgcttagacagacaaacaaacccgcagttctgttggaactcggctacatctccaacccaacagacgaggtgatgatccgcaatcagattcaccgtcaagtagtggaagaagcagtggtcgatggcctgaaacagtactttgccagttgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 107 - protein, 153 aa, synthetic construct
  REGION 1..153: note = Peptidoglycan Hydrolase
MDYKDDDDKRTNNIDAIKEHEGLRLVAYLDSVGVWTIGYGDTGPDVVKGLTITKEEAEKRLRKRLVEFEGYVNTYVKVPLKQHQFDALVSLVYNIGPTNFKTSTLLKKLNAGDYIGAADQFLVWNKGRVDGKLVVIKGLANRRAKERKQFLGE

SEQ ID NO: 108 - DNA, 923 bp, synthetic construct
  misc_feature 1..923: note = Nucleotide sequence encoding Peptidoglycan Hydrolase
  source 1..923: mol_type = other DNA
agaataaactagtattcttctggtccccacagactcagagagaacccgccaccatggactacaaagacgatgacgacaagagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 109 - protein, 178 aa, synthetic construct
  REGION 1..178: note = Peptidoglycan Hydrolase
MRVMAPRTLILLLSGALALTETWAGSDYKDDDDKRTNNIDAIKEHEGLRLVAYLDSVGVWTIGYGDTGPDVVKGLTITKEEAEKRLRKRLVEFEGYVNTYVKVPLKQHQFDALVSLVYNIGPTNFKTSTLLKKLNAGDYIGAADQFLVWNKGRVDGKLVVIKGLANRRAKERKQFLGE

SEQ ID NO: 110 - DNA, 998 bp, synthetic construct
  misc_feature 1..998: note = Nucleotide sequence encoding Peptidoglycan Hydrolase
  source 1..998: mol_type = other DNA
agaataaactagtattcttctggtccccacagactcagagagaacccgccaccatgagagtgatggcccccagaaccctgatcctgctgctgtctggcgccctggccctgacagagacatgggccggaagcgactacaaagacgatgacgacaagagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 111 - RNA, 932 nt, synthetic construct
  source 1..932: mol_type = other RNA
agaataaactagtattcttctggtccccacagactcagagagaacccgccaccatggtgagcggctggcggctgttcaagaagattagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 112 - protein, 156 aa, synthetic construct
MVSGWRLFKKISRTNNIDAIKEHEGLRLVAYLDSVGVWTIGYGDTGPDVVKGLTITKEEAEKRLRKRLVEFEGYVNTYVKVPLKQHQFDALVSLVYNIGPTNFKTSTLLKKLNAGDYIGAADQFLVWNKGRVDGKLVVIKGLANRRAKERKQFLGE

SEQ ID NO: 113 - RNA, 1007 nt, synthetic construct
  source 1..1007: mol_type = other RNA
agaataaactagtattcttctggtccccacagactcagagagaacccgccaccatgagagtgatggcccccagaaccctgatcctgctgctgtctggcgccctggccctgacagagacatgggccggaagcgtgagcggctggcggctgttcaagaagattagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 114 - protein, 181 aa, synthetic construct
MRVMAPRTLILLLSGALALTETWAGSVSGWRLFKKISRTNNIDAIKEHEGLRLVAYLDSVGVWTIGYGDTGPDVVKGLTITKEEAEKRLRKRLVEFEGYVNTYVKVPLKQHQFDALVSLVYNIGPTNFKTSTLLKKLNAGDYIGAADQFLVWNKGRVDGKLVVIKGLANRRAKERKQFLGE

SEQ ID NO: 115 - RNA, 8090 nt, synthetic construct
  source 1..8090: mol_type = other RNA
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaggctttgagcgagagcgccactatggtgtacaacgaaagggagttcgtcaacaggaaactataccatattgccgttcacggaccctcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggtagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctggagaacccgattcctccaccgcgcccaaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggctgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccactagtatggtgagcggctggcggctgttcaagaagattagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgaggcggccgcaggagcttaattcgacgaataattggatttttattttattttgcaattggtttttaatatttccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 116 - RNA, 8165 nt, synthetic construct
  source 1..8165: mol_type = other RNA
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaggctttgagcgagagcgccactatggtgtacaacgaaagggagttcgtcaacaggaaactataccatattgccgttcacggaccctcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggtagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctggagaacccgattcctccaccgcgcccaaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtcgaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggctgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattatagcgcactattatagcaccactagtatgagagtgatggcccccagaaccctgatcctgctgctgtctggcgccctggccctgacagagacatgggccggaagcgtgagcggctggcggctgttcaagaagattagcagaaccaacaacatcgatgccatcaaagaacatgaaggactgagactggtggcttacctggattctgtgggagtgtggacaattggatatggagacacaggacctgacgtggtgaagggactgacaatcacaaaggaagaagccgaaaagagactgagaaagagactggtggaatttgaaggatatgtgaacacctatgtgaaagtgcctctgaaacagcaccagtttgatgctctggtgagcctggtgtacaacattggacccacaaacttcaagacaagcacactgctgaagaagctgaatgctggagattacattggagctgctgatcagttcctggtgtggaacaaaggcagagtggatggcaaactggtggtgatcaaaggcctggccaacaggagagccaaagaaagaaaacagttcctgggagaatgatgactcgaggcggccgcaggagcttaattcgacgaataattggatttttattttattttgcaattggtttttaatatttccaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 117 - RNA, 1313 nt, synthetic construct
  source 1..1313: mol_type = other RNA
agaataaactagtattcttctggtccccacagactcagagagaacccgccaccatggattggacctggagagtgttttgccttctcgctgttgcccctggtgcccattccatgaagcaccaccaccaccatcatagtgcagggctggaagttctgtttcagggtccagcagcaacgcatgagcattccgctcaatggctcaacaactacaagaagggttacggctatggcccctatcctctggggataaacggagggatgcactatggagtcgatttcttcatgaatattggcactccagtgaaagcgatttccagcgggaagattgtggaagctgggtggagcaactatggtggtggcaatcagatcggactgattgagaatgacggcgtgcacagacagtggtatatgcatctgagtaagtacaacgtcaaggtgggagactacgtcaaagccgggcagatcataggatggtccggctctacaggttactcaacagcgccacatctccactttcagcgcatggtgaatagcttcagcaattcaaccgcccaagatccgatgcccttcctgaagtctgccggctatgggaaagctggaggaacagtgacacctactcccaataccggatggaaaacgaacaagtacggcactctgtacaaatccgaatctgccagtttcacacccaataccgacatcatcacccgaaccactggaccttttcggtcaatgccgcagtctggagtactgaaagcagggcaaactatccactatgacgaggtgatgaagcaggatggccacgtatgggtggggtacacaggcaatagcggccagaggatttacttgccagtccgtacctggaacaaaagcacgaacactcttggcgttttgtggggcaccatcaagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 118 - DNA, 914 bp, synthetic construct
  source 1..914: mol_type = other DNA
gagacccaagctggctagcgtttaaacttaagcttggtaccgagctcggatccgccaccatggattggacctggagagtgttttgccttctcgctgttgcccctggtgcccattccatgaagcaccaccaccaccatcatagtgcagggctggaagttctgtttcagggtccagcagcaacgcatgagcattccgctcaatggctcaacaactacaagaagggttacggctatggcccctatcctctggggataaacggagggatgcactatggagtcgatttcttcatgaatattggcactccagtgaaagcgatttccagcgggaagattgtggaagctgggtggagcaactatggtggtggcaatcagatcggactgattgagaatgacggcgtgcacagacagtggtatatgcatctgagtaagtacaacgtcaaggtgggagactacgtcaaagccgggcagatcataggatggtccggctctacaggttactcaacagcgccacatctccactttcagcgcatggtgaatagcttcagcaattcaaccgcccaagatccgatgcccttcctgaagtctgccggctatgggaaagctggaggaacagtgacacctactcccaataccggatggaaaacgaacaagtacggcactctgtacaaatccgaatctgccagtttcacacccaataccgacatcatcacccgaaccactggaccttttcggtcaatgccgcagtctggagtactgaaagcagggcaaactatccactatgacgaggtgatgaagcaggatggccacgtatgggtggggtacacaggcaatagcggccagaggatttacttgccagtccgtacctggaacaaaagcacgaacactcttggcgttttgtggggcaccatcaagtga

SEQ ID NO: 119 - protein, 546 aa, synthetic construct
MALWMRLLPLLALLALWGPDPAAAMKDYKDDDDKGGGGSGMAKTKKQIEQRLQAYAKGTVDSPYRITKPTSYDPSFGVMEVGAIDADRYYHAQCQDLITDYVLWLTDNKVRPWGNAKDQINQSYGTGFKIHKNLPSTVAKTGWIAVFTSGTYSQYGHIGIVYSPGDTNKFHIIEQNWNGWANKKPLTRWDNYYGLTHFIEVPYAKETITTKKTAPKAATSKPAKKISVSHNNINLQSNFAKRGYNPKGVVIHNDAGRSTAQQYENSLKGANQNRLNNGIAHAYADSSHIWYAIEPERIAYHTGDGVKKGTGNYDYFGIEVCQSMSASDKEFMANEQVVLQEVARLLKKWKLPANRNTVRLHLEFSATSCPHRSLTMRTGFNPVTQGRPSDAIIKKLKDIWIKEIRAYMEGKTPSIKTVANKPGSASTPATKSNSDGWKVNAYNTYYKTERASFTVGSTRIAVRSTGPFTTCPFTGWLNPGVTITYDEVMVQNGFVWLGYDSYNGRRYVPIRTAQGTPGSSNYVLGPLWGTISAAAGGGGSHHHHHH

SEQ ID NO: 120 - RNA, 2099 nt, synthetic construct
  source 1..2099: mol_type = other RNA
agaataaactagtattcttctggtccccacagactcagagagaacccgccaccatggccctgtggatgagactgctgcctctgcttgctctgctggcactgtggggacctgatcctgctgctgctatgaaggactacaaagacgatgacgacaagggaggaggcggctccggaatggccaaaaccaagaaacagatagaacagagactgcaagcttacgcaaaggggaccgtggattccccttaccgaatcaccaagccaaccagctacgacccctccttcggtgtcatggaggttggcgctatagatgcggatcgctactatcatgcccagtgtcaagatctgataactgactatgtactttggctcactgacaacaaagtaagaccttggggtaatgccaaggaccagattaaccagagctatgggactggtttcaaaatccacaagaatctgcccagtaccgtggctaaaacaggatggattgccgtgtttacatccgggacctactcacagtacggccacatcggcatcgtgtacagtccaggagataccaacaaattccatatcatcgagcagaattggaatggatgggcaaacaagaaacctctgacacgttgggacaactactacgggctgacccactttatcgaggtcccatatgcgaaagagacaatcacaacgaagaaaactgcacctaaagcagctacctcaaaaccggctaagaagatctcagtttctcacaacaacatcaaccttcagagcaactttgcaaagagagggtacaacccaaagggagtggtgattcacaatgacgccggacgctctactgcgcaacagtatgaaaacagcctgaagggcgctaatcagaacaggctgaataacggcattgcccatgcatatgctgacagcagccacatttggtatgctatcgaacccgagagaatcgcctatcacacaggcgatggcgttaagaagggaacaggcaattacgactacttcggtattgaagtatgccagagcatgagcgcaagtgacaaagagttcatggccaacgaacaggtggtactgcaggaggtggctaggttgctcaagaaatggaagcttcccgccaatcggaataccgtgcggctccatctggaattctctgccacatcttgccctcataggagcctcacaatgcggaccggtttcaatccggtgactcagggtagaccctcagacgccatcatcaagaagttgaaggatatctggataaaagagattcgcgcatatatggagggtaaaaccccatccattaagactgtcgccaataagcccggatcagccagtacccctgcaaccaaatccaactccgatgggtggaaggtcaatgcctacaatacctactacaaaacggaacgggcctcctttacagtgggctctactcggattgctgtgcgtagtacaggaccctttactacatgcccgtttacagggtggctgaaccccggagtcacgataacctatgacgaggttatggtgcagaacgggtttgtgtggttgggctatgatagctacaatgggaggaggtatgtccccattcgcactgcccaaggaactcctggcagctccaattatgtgcttggccctctgtggggcacgatttctgcggccgccggaggaggcggctcccaccaccaccaccatcattgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 121 - protein, 269 aa, synthetic construct
MALWMRLLPLLALLALWGPDPAAAMKDYKDDDDKGGGGSGREAPKTQIYTVKKGDTLSAIALKYKTTVSNIQNTNNIANPNLIFIGQKLKVPMTPLVEPKPKTVSSNNKSNSNSSTLNYLKTLENRGWDFDGSYGWQCFDLVNVYWNHLYGHGLKGYGAKDIPYANNFNSEAKIYHNTPTFKAEPGDLVVFSGRFGGGYGHTAIVLNGDYDGKLMKFQSLDQNWNNGGWRKAEVAHKVVHNYENDMIFIRPFKKAAAAGGGGSHHHHHH

SEQ ID NO: 122 - RNA, 1268 nt, synthetic construct
  source 1..1268: mol_type = other RNA
agaataaactagtattcttctggtccccacagactcagagagaacccgccaccatggccctgtggatgagactgctgcctctgcttgctctgctggcactgtggggacctgatcctgctgctgctatgaaggactacaaagacgatgacgacaagggaggaggcggctccggaagagaggctcccaaaacacagatttacaccgtcaagaagggagatacgcttagcgccattgcactgaaatacaagaccaccgtatcaaacatccagaacaccaataacatagccaatcccaatctcatcttcattggccagaagctcaaggttcccatgactccactggtcgaaccgaaacctaagacagtgtcctccaataacaagagtaacagcaactcctctacactgaactatctcaagacgttggagaatcgcggttgggactttgacggcagttacggctggcaatgcttcgacttggtgaatgtgtactggaatcacctgtatgggcatggactgaaaggctatggggccaaagacataccttatgctaacaatttcaactctgaggccaagatctaccacaacactcctaccttcaaggcagaaccaggagatctggttgtgtttagcggacggtttggcggaggctatgggcatactgccattgtgcttaatggggattacgatggcaaactgatgaaattccagtcactggaccagaactggaacaatggtggttggcgaaaagcggaagtagcccacaaggtcgtccacaattacgagaacgacatgatcttcatccgtccctttaagaaagcggcggccgccggaggaggcggctcccaccaccaccaccatcattgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 123 - protein, 300 aa, synthetic construct
MALWMRLLPLLALLALWGPDPAAAMKDYKDDDDKGGGGSGAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKAAAGGGGSHHHHHH

SEQ ID NO: 124 - RNA, 1361 nt, synthetic construct
  source 1..1361: mol_type = other RNA
agaataaactagtattcttctggtccccacagactcagagagaacccgccaccatggccctgtggatgagactgctgcctctgcttgctctgctggcactgtggggacctgatcctgctgctgctatgaaggactacaaagacgatgacgacaagggaggaggcggctccggagcagcaacgcatgagcattccgctcaatggctcaacaactacaagaagggttacggctatggcccctatcctctggggataaacggagggatgcactatggagtcgatttcttcatgaatattggcactccagtgaaagcgatttccagcgggaagattgtggaagctgggtggagcaactatggtggtggcaatcagatcggactgattgagaatgacggcgtgcacagacagtggtatatgcatctgagtaagtacaacgtcaaggtgggagactacgtcaaagccgggcagatcataggatggtccggctctacaggttactcaacagcgccacatctccactttcagcgcatggtgaatagcttcagcaattcaaccgcccaagatccgatgcccttcctgaagtctgccggctatgggaaagctggaggaacagtgacacctactcccaataccggatggaaaacgaacaagtacggcactctgtacaaatccgaatctgccagtttcacacccaataccgacatcatcacccgaaccactggaccttttcggtcaatgccgcagtctggagtactgaaagcagggcaaactatccactatgacgaggtgatgaagcaggatggccacgtatgggtggggtacacaggcaatagcggccagaggatttacttgccagtccgtacctggaacaaaagcacgaacactcttggcgttttgtggggcaccatcaaggcggccgccggaggaggcggctcccaccaccaccaccatcattgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 125 - protein, 204 aa, synthetic construct
MALWMRLLPLLALLALWGPDPAAAMKDYKDDDDKGGGGSGDQFLDEKEGNHTTAYRDGSGIWTICRGATMVDGKPVIPGMRLSKEKCDQVNAIERDKALAWVERNIKVPLTEPQKAGIASFCPYNIGPGKCFPSTFYKRLNAGDRKGACEAIRWWIKDGGRDCRIRSNNCYGQVIRRDQESALACWGIDQAAAGGGGSHHHHHH

SEQ ID NO: 126 - RNA, 1073 nt, synthetic construct
  source 1..1073: mol_type = other RNA
agaataaactagtattcttctggtccccacagactcagagagaacccgccaccatggccctgtggatgagactgctgcctctgcttgctctgctggcactgtggggacctgatcctgctgctgctatgaaggactacaaagacgatgacgacaagggaggaggcggctccggagatcagtttctcgacgagaaggaaggcaatcacaccactgcgtatagggatggctctgggatatggaccatctgtagaggcgccacaatggtcgatggcaaacccgtgatacccggaatgcgtctgtcaaaggagaagtgcgatcaggtgaatgccatcgaacgcgacaaagctctggcctgggttgagcggaacatcaaggttccactgacggaaccacagaaagccggaattgcgtccttctgtccgtacaacattggccctggaaagtgctttccctccactttctacaagcgcctgaatgcaggggacagaaaaggagcctgtgaggctatcaggtggtggatcaaagatggaggtagggactgtcgaatcaggagcaacaactgctatgggcaagtgattcggagagatcaggagagtgctctggcttgctggggtattgaccaggcggccgccggaggaggcggctcccaccaccaccaccatcattgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 127 - protein, 187 aa, synthetic construct
MALWMRLLPLLALLALWGPDPAAAMKDYKDDDDKGGGGSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTAAAGGGGSHHHHHH

SEQ ID NO: 128 - RNA, 1022 nt, synthetic construct
  source 1..1022: mol_type = other RNA
agaataaactagtattcttctggtccccacagactcagagagaacccgccaccatggccctgtggatgagactgctgcctctgcttgctctgctggcactgtggggacctgatcctgctgctgctatgaaggactacaaagacgatgacgacaagggaggaggcggctccggagctccaacatcttcttcaacaaagaaaacacagcttcagcttgaacaccttcttcttgatcttcagatgattctgaatggaatcaacaattacaaaaatccaaaactgacaagaatgctgacatttaaattttacatgccaaagaaagcaacagaactgaaacaccttcagtgccttgaagaagaactgaaacctctggaagaagtgctgaatctggctcagagcaaaaattttcacctgagaccaagagatctgatcagcaacatcaatgtgattgtgctggaactgaaaggatctgaaacaacattcatgtgtgaatatgctgatgaaacagcaacaattgtggaatttctgaacagatggatcacattttgccagtcaatcatttcaacactgacagcggccgccggaggaggcggctcccaccaccaccaccatcattgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 129 - protein, 183 aa, synthetic construct
MYRMQLLSCIALSLALVTNSMKDYKDDDDKGGGGSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTAAAGGGGSHHHHHH

SEQ ID NO: 130 - RNA, 1010 nt, synthetic construct
  source 1..1010: mol_type = other RNA
agaataaactagtattcttctggtccccacagactcagagagaacccgccaccatgtaccggatgcagctgctgagctgtatcgccctgtctctggccctggtcaccaattctatgaaggactacaaagacgatgacgacaagggaggaggcggctccggagctccaacatcttcttcaacaaagaaaacacagcttcagcttgaacaccttcttcttgatcttcagatgattctgaatggaatcaacaattacaaaaatccaaaactgacaagaatgctgacatttaaattttacatgccaaagaaagcaacagaactgaaacaccttcagtgccttgaagaagaactgaaacctctggaagaagtgctgaatctggctcagagcaaaaattttcacctgagaccaagagatctgatcagcaacatcaatgtgattgtgctggaactgaaaggatctgaaacaacattcatgtgtgaatatgctgatgaaacagcaacaattgtggaatttctgaacagatggatcacattttgccagtcaatcatttcaacactgacagcggccgccggaggaggcggctcccaccaccaccaccatcattgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 131 - DNA, 1700 bp, synthetic construct
  source 1..1700: mol_type = other DNA
gagacccaagctggctagcgtttaaacttaagcttggtaccgagctcggatccgccaccatggccctgtggatgagactgctgcctctgcttgctctgctggcactgtggggacctgatcctgctgctgctatgaaggactacaaagacgatgacgacaagggaggaggcggctccggaatggccaaaaccaagaaacagatagaacagagactgcaagcttacgcaaaggggaccgtggattccccttaccgaatcaccaagccaaccagctacgacccctccttcggtgtcatggaggttggcgctatagatgcggatcgctactatcatgcccagtgtcaagatctgataactgactatgtactttggctcactgacaacaaagtaagaccttggggtaatgccaaggaccagattaaccagagctatgggactggtttcaaaatccacaagaatctgcccagtaccgtggctaaaacaggatggattgccgtgtttacatccgggacctactcacagtacggccacatcggcatcgtgtacagtccaggagataccaacaaattccatatcatcgagcagaattggaatggatgggcaaacaagaaacctctgacacgttgggacaactactacgggctgacccactttatcgaggtcccatatgcgaaagagacaatcacaacgaagaaaactgcacctaaagcagctacctcaaaaccggctaagaagatctcagtttctcacaacaacatcaaccttcagagcaactttgcaaagagagggtacaacccaaagggagtggtgattcacaatgacgccggacgctctactgcgcaacagtatgaaaacagcctgaagggcgctaatcagaacaggctgaataacggcattgcccatgcatatgctgacagcagccacatttggtatgctatcgaacccgagagaatcgcctatcacacaggcgatggcgttaagaagggaacaggcaattacgactacttcggtattgaagtatgccagagcatgagcgcaagtgacaaagagttcatggccaacgaacaggtggtactgcaggaggtggctaggttgctcaagaaatggaagcttcccgccaatcggaataccgtgcggctccatctggaattctctgccacatcttgccctcataggagcctcacaatgcggaccggtttcaatccggtgactcagggtagaccctcagacgccatcatcaagaagttgaaggatatctggataaaagagattcgcgcatatatggagggtaaaaccccatccattaagactgtcgccaataagcccggatcagccagtacccctgcaaccaaatccaactccgatgggtggaaggtcaatgcctacaatacctactacaaaacggaacgggcctcctttacagtgggctctactcggattgctgtgcgtagtacaggaccctttactacatgcccgtttacagggtggctgaaccccggagtcacgataacctatgacgaggttatggtgcagaacgggtttgtgtggttgggctatgatagctacaatgggaggaggtatgtccccattcgcactgcccaaggaactcctggcagctccaattatgtgcttggccctctgtggggcacgatttctgcggccgccggaggaggcggctcccaccaccaccaccatcattga

SEQ ID NO: 132 - DNA, 869 bp, synthetic construct
  source 1..869: mol_type = other DNA
gagacccaagctggctagcgtttaaacttaagcttggtaccgagctcggatccgccaccatggccctgtggatgagactgctgcctctgcttgctctgctggcactgtggggacctgatcctgctgctgctatgaaggactacaaagacgatgacgacaagggaggaggcggctccggaagagaggctcccaaaacacagatttacaccgtcaagaagggagatacgcttagcgccattgcactgaaatacaagaccaccgtatcaaacatccagaacaccaataacatagccaatcccaatctcatcttcattggccagaagctcaaggttcccatgactccactggtcgaaccgaaacctaagacagtgtcctccaataacaagagtaacagcaactcctctacactgaactatctcaagacgttggagaatcgcggttgggactttgacggcagttacggctggcaatgcttcgacttggtgaatgtgtactggaatcacctgtatgggcatggactgaaaggctatggggccaaagacataccttatgctaacaatttcaactctgaggccaagatctaccacaacactcctaccttcaaggcagaaccaggagatctggttgtgtttagcggacggtttggcggaggctatgggcatactgccattgtgcttaatggggattacgatggcaaactgatgaaattccagtcactggaccagaactggaacaatggtggttggcgaaaagcggaagtagcccacaaggtcgtccacaattacgagaacgacatgatcttcatccgtccctttaagaaagcggcggccgccggaggaggcggctcccaccaccaccaccatcattga

SEQ ID NO: 133 - DNA, 962 bp, synthetic construct
  source 1..962: mol_type = other DNA
gagacccaagctggctagcgtttaaacttaagcttggtaccgagctcggatccgccaccatggccctgtggatgagactgctgcctctgcttgctctgctggcactgtggggacctgatcctgctgctgctatgaaggactacaaagacgatgacgacaagggaggaggcggctccggagcagcaacgcatgagcattccgctcaatggctcaacaactacaagaagggttacggctatggcccctatcctctggggataaacggagggatgcactatggagtcgatttcttcatgaatattggcactccagtgaaagcgatttccagcgggaagattgtggaagctgggtggagcaactatggtggtggcaatcagatcggactgattgagaatgacggcgtgcacagacagtggtatatgcatctgagtaagtacaacgtcaaggtgggagactacgtcaaagccgggcagatcataggatggtccggctctacaggttactcaacagcgccacatctccactttcagcgcatggtgaatagcttcagcaattcaaccgcccaagatccgatgcccttcctgaagtctgccggctatgggaaagctggaggaacagtgacacctactcccaataccggatggaaaacgaacaagtacggcactctgtacaaatccgaatctgccagtttcacacccaataccgacatcatcacccgaaccactggaccttttcggtcaatgccgcagtctggagtactgaaagcagggcaaactatccactatgacgaggtgatgaagcaggatggccacgtatgggtggggtacacaggcaatagcggccagaggatttacttgccagtccgtacctggaacaaaagcacgaacactcttggcgttttgtggggcaccatcaaggcggccgccggaggaggcggctcccaccaccaccaccatcattga

SEQ ID NO: 134 - DNA, 674 bp, synthetic construct
  source 1..674: mol_type = other DNA
gagacccaagctggctagcgtttaaacttaagcttggtaccgagctcggatccgccaccatggccctgtggatgagactgctgcctctgcttgctctgctggcactgtggggacctgatcctgctgctgctatgaaggactacaaagacgatgacgacaagggaggaggcggctccggagatcagtttctcgacgagaaggaaggcaatcacaccactgcgtatagggatggctctgggatatggaccatctgtagaggcgccacaatggtcgatggcaaacccgtgatacccggaatgcgtctgtcaaaggagaagtgcgatcaggtgaatgccatcgaacgcgacaaagctctggcctgggttgagcggaacatcaaggttccactgacggaaccacagaaagccggaattgcgtccttctgtccgtacaacattggccctggaaagtgctttccctccactttctacaagcgcctgaatgcaggggacagaaaaggagcctgtgaggctatcaggtggtggatcaaagatggaggtagggactgtcgaatcaggagcaacaactgctatgggcaagtgattcggagagatcaggagagtgctctggcttgctggggtattgaccaggcggccgccggaggaggcggctcccaccaccaccaccatcattga

SEQ ID NO: 135 - DNA, 623 bp, synthetic construct
  source 1..623: mol_type = other DNA
gagacccaagctggctagcgtttaaacttaagcttggtaccgagctcggatccgccaccatggccctgtggatgagactgctgcctctgcttgctctgctggcactgtggggacctgatcctgctgctgctatgaaggactacaaagacgatgacgacaagggaggaggcggctccggagctccaacatcttcttcaacaaagaaaacacagcttcagcttgaacaccttcttcttgatcttcagatgattctgaatggaatcaacaattacaaaaatccaaaactgacaagaatgctgacatttaaattttacatgccaaagaaagcaacagaactgaaacaccttcagtgccttgaagaagaactgaaacctctggaagaagtgctgaatctggctcagagcaaaaattttcacctgagaccaagagatctgatcagcaacatcaatgtgattgtgctggaactgaaaggatctgaaacaacattcatgtgtgaatatgctgatgaaacagcaacaattgtggaatttctgaacagatggatcacattttgccagtcaatcatttcaacactgacagcggccgccggaggaggcggctcccaccaccaccaccatcattga

SEQ ID NO: 136 - DNA, 611 bp, synthetic construct
  source 1..611: mol_type = other DNA
gagacccaagctggctagcgtttaaacttaagcttggtaccgagctcggatccgccaccatgtaccggatgcagctgctgagctgtatcgccctgtctctggccctggtcaccaattctatgaaggactacaaagacgatgacgacaagggaggaggcggctccggagctccaacatcttcttcaacaaagaaaacacagcttcagcttgaacaccttcttcttgatcttcagatgattctgaatggaatcaacaattacaaaaatccaaaactgacaagaatgctgacatttaaattttacatgccaaagaaagcaacagaactgaaacaccttcagtgccttgaagaagaactgaaacctctggaagaagtgctgaatctggctcagagcaaaaattttcacctgagaccaagagatctgatcagcaacatcaatgtgattgtgctggaactgaaaggatctgaaacaacattcatgtgtgaatatgctgatgaaacagcaacaattgtggaatttctgaacagatggatcacattttgccagtcaatcatttcaacactgacagcggccgccggaggaggcggctcccaccaccaccaccatcattga

SEQ ID NO: 137 - protein, 294 aa, synthetic construct
MDMRAPAGIFGFLLVLFPGYRSHHHHHHLEVLFQGPAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSQSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWQKSTNTLGVLWGTIKSGGSDYKDDDDK

SEQ ID NO: 138 - RNA, 1343 nt, synthetic construct
  source 1..1343: mol_type = other RNA
agaataaactagtattcttctggtccccacagactcagagagaacccgccaccatggacatgcgggcccctgccggaatcttcggctttctgctggtgctgttccccggctacagaagccatcatcatcaccaccacctcgaagtattgtttcagggacccgccgccacccacgagcacagcgctcaatggctgaacaactacaaaaagggctacggctatggtccttatcctctgggcatcaacggcgggatgcactacggcgtggacttcttcatgaacatcggcacccccgtgaaggccatcagcagcggaaagatcgtggaagccggctggtccaactacggcggcggcaaccagatcggactgatcgagaacgacggcgtgcacagacagtggtacatgcatctgtctaagtacaatgtgaaggtgggcgactacgtgaaagccggccaaatcattggatggagcggctctaccggctacagcacagccccacacctgcacttccagagaatggtgaacagcttcagccagtccacagcccaggaccccatgcctttcctgaagagcgccggctacggcaaggccggaggcaccgtgaccccaacacctaataccggctggaagaccaacaagtacggcaccctgtacaagtctgaaagcgcttcttttacccctaacactgatatcatcacaagaaccaccggcccttttagaagcatgccccagagcggcgtcctcaaagctggacagaccatccactacgacgaggtgatgaaacaggatggccacgtgtgggtcggctacaccggaaatagcggccagcggatctacctgcctgtgcggacatggcagaagtccaccaacacactgggagttctgtggggcacaatcaagtcaggaggttcagattacaaggatgacgacgataagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 139 - protein, 24 aa, synthetic construct
MALWMRLLPLLALLALWGPDPAAA

SEQ ID NO: 140 - protein, 20 aa, synthetic construct
MYRMQLLSCIALSLALVTNS

SEQ ID NO: 141 - protein, 22 aa, synthetic construct
MDMRAPAGIFGFLLVLFPGYRS